SEQUENCE LISTING (3 sequences)

SEQ ID NO: 1 - RNA, 36 nt, Artificial Sequence
  source: /note=“Description of Artificial Sequence Synthetic oligonucleotide”
gguggaugaaacucaguuuagcagccgaaaggcugc

SEQ ID NO: 2 - RNA, 22 nt, Artificial Sequence
  source: /note=“Description of Artificial Sequence Synthetic oligonucleotide”
uaaacugaguuucauccaccgg

SEQ ID NO: 3 - protein, 5 aa, Artificial Sequence
  source: /note=“Description of Artificial Sequence Synthetic peptide”
CREKA

(19) United States
(12) Patent Application Publication
(10) Pub. No.: US 2023/0277675 A1
(43) Pub. Date: Sep. 7, 2023

(54) SYSTEMIC DELIVERY OF OLIGONUCLEOTIDES

(71) Applicant: Dicerna Pharmaceuticals, Inc., Lexington, MA (US)

(72) Inventors: Weimin WANG, Waltham, MA (US); Bob Dale BROWN, Littleton, MA (US); Hongchuan YU, Chestnut Hill, MA (US); Xiaochuan CAI, Lexington, MA (US); Marc ABRAMS, Natick, MA (US)

(21) Appl. No.: 18/019,404

(22) PCT Filed: Jul. 21, 2021
(86) PCT No.: PCT/US2021/042469

§ 371 Date: Feb. 2, 2023

Related U.S. Application Data
(60) Provisional application No. 63/060,715, filed on Aug. 4, 2020.
(60) Provisional application No. 63/144,603, filed on Feb. 2, 2021.

(51) Int. Cl. A61K 47/54

(52) U.S. Cl. CPC A61K 47/545; A61K 47/548; A61K 47/542

(57) ABSTRACT

The disclosure provides oligonucleotide-ligand conjugates to facilitate the systemic delivery of oligonucleotides designed to prevent, limit or modulate the expression of mRNA molecules. The conjugates comprise nucleotides which are linked to lipid conjugate moieties or adamantyl groups.

### CROSS REFERENCE TO RELATED APPLICATIONS

This application claims priority to U.S. Provisional Patent Application No. 63/060,715, filed on 4 Aug. 2020, and U.S. Provisional Patent Application No. 63/144,603, filed on 2 Feb. 2021, the entire contents of which are incorporated herein by reference in their entireties.

### TECHNICAL FIELD OF THE DISCLOSURE

The present disclosure relates to nucleic acid-hydrophobic ligand conjugates and oligonucleotide-hydrophobic ligand conjugates. Specifically, the present disclosure relates to nucleic acid-lipid conjugates and oligonucleotide-lipid conjugates, methods to prepare them, their chemical configuration and methods useful to modulate the expression of a target gene in a cell using the conjugated nucleic acids and oligonucleotides according to the description provided herein. The disclosure also provides pharmaceutically acceptable compositions comprising the conjugates of the present description and methods of using said compositions in the treatment of various disorders.

### BACKGROUND OF THE DISCLOSURE

Regulation of gene expression by modified nucleic acids shows great potential as both a research tool in the laboratory and a therapeutic approach in the clinic. Several classes of oligonucleotide or nucleic acid-based therapeutics have been under the clinical investigation, including antisense oligo (ASO), short interfering RNA (siRNA), double-stranded nucleic acid(dsNA), aptamer, ribozyme, exon skipping or splice altering oligos, mRNA, and CRISPR. Chemical modifications play a key role in overcoming the hurdles facing oligonucleotide therapeutics, including improving nuclease stability, RNA-binding affinity, and pharmacokinetic properties of oligonucleotides. Various chemical modification strategies for oligonucleotides have been developed in the past three decades including modification of the sugars, nucleobases, and phosphodiester backbone (Deleavey and Darma, CHEM. BIOL. 2012, 19(8):937-54; Wan and Seth, J. MED. CHEM. 2016, 59(21):9645-67; and Egli and Manoharan, Acc. CHEM. RES. 2019, 54(4):1036-47).

siRNA or double-stranded nucleic acid(dsNA) based therapeutics been successfully used as an effective means of reducing the expression of specific target genes in the liver. Thus, these RNAi agents are uniquely useful for several therapeutic, diagnostic, and research applications for the modulation of target gene expression.

One of the obstacles preventing the widespread clinical use is the ability to deliver intact siRNA efficiently beyond the liver. Thus, an ongoing need exists in the art for the successful delivery of new and effective RNAi agents outside the liver to modulate the expression of a target gene in various tissues.

The present disclosure is directed to overcome this obstacle by designing novel oligonucleotide conjugates comprising hydrophobic ligands for systemic delivery.

### SUMMARY

The present application relates to novel nucleic acids, oligonucleotides or analogues thereof comprising hydrophobic ligands, including but not limited to adamantyl and lipid conjugates. The present disclosure relates to nucleic acid-lipid conjugates and oligonucleotide-lipid conjugates, which function to modulate the expression of a target gene in a cell, and methods of preparation and uses thereof. Lipophilic/hydrophobic moieties, such as fatty acids and adamantyl group when attached to these highly hydrophilic nucleic acids/oligonucleotides can substantially enhance plasma protein binding and consequently circulation half-life. The conjugated nucleic acids, oligonucleotides, and analogues thereof provided herein are stable and bind to RNA targets to elicit broad extrahepatic RNase H activity and are also useful in splice switching and RNAi. Incorporation of the hydrophobic moiety such as lipid facilitates systemic delivery of the novel nucleic acids, oligonucleotides, or analogues thereof into several tissues, including but not limited to, the CNS, muscle, adipose, and adrenal gland.

Suitable nucleic acid-hydrophobic ligand conjugates and oligonucleotide-hydrophobic ligand conjugates include nucleic acid inhibitor molecules, such as dsRNA inhibitor molecules, dsRNAi inhibitor molecules, antisense oligonucleotides, miRNA, ribozymes, antagomirs, aptamers, and single-stranded RNAi inhibitor molecules. In particular, the present disclosure provides nucleic acid-lipid conjugates, oligonucleotide-lipid conjugates, and analogues thereof, which find utility as modulators of intracellular RNA levels. Nucleic acid inhibitor molecules can modulate RNA expression through a diverse set of mechanisms, for example by RNA interference (RNAi). An advantage of the nucleic acid-hydrophobic ligand conjugates, oligonucleotide-hydrophobic ligand conjugates and analogues thereof provided herein is that a broad range of pharmacological activities is possible, consistent with the modulation of intracellular RNA levels. In addition, the description provides methods of using an effective amount of the conjugates described herein for the treatment or amelioration of a disease condition by modulating the intracellular RNA levels.

It has now been found that the nucleic acid-hydrophobic ligand conjugates of the present disclosure, and pharmaceutically acceptable compositions thereof, are effective as modulators of intracellular RNA levels. Such nucleic acid-lipid conjugates thereof comprising one or more lipid conjugates are represented by formula I or Ia:

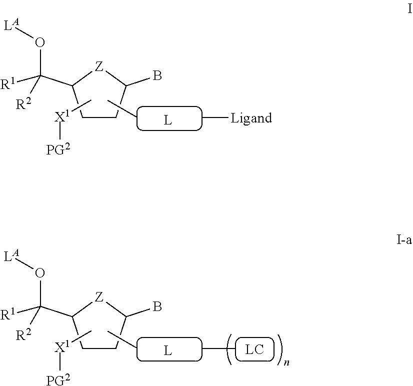

or a pharmaceutically acceptable salt thereof, wherein each variable is as defined and described herein.

In some embodiments, the nucleic acid-lipid conjugates are represented by formula I-b, I-c, I-Ib, I-Ic, I-d or I-e, I-Id or I-Ie:

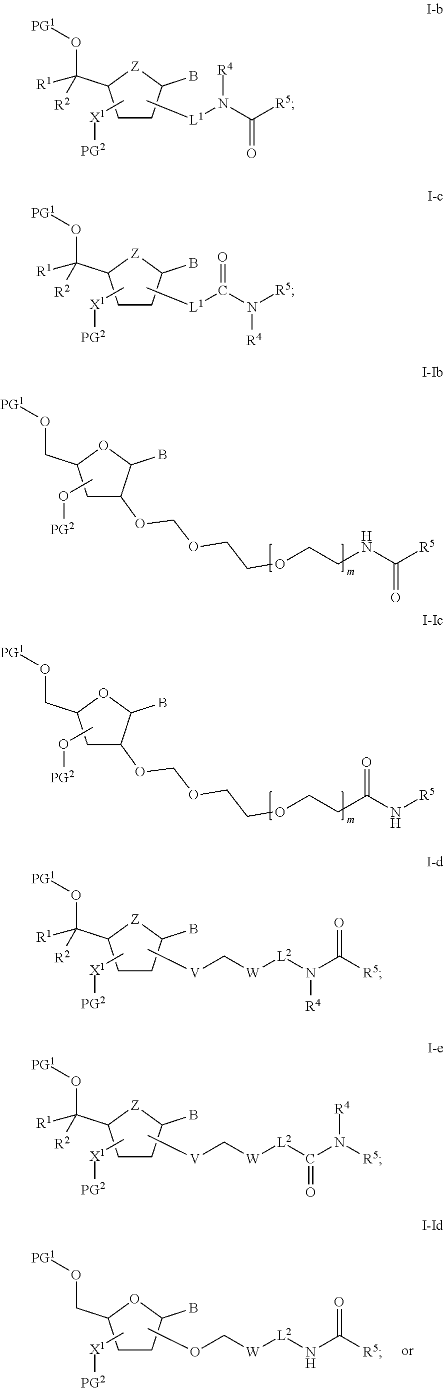

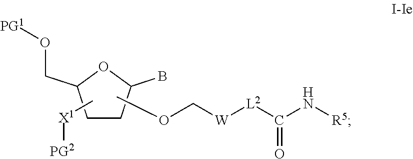

or a pharmaceutically acceptable salt thereof, wherein each variable is as defined and described herein.

In another aspect, the present disclosure presents oligonucleotide-ligand conjugates represented by formula II or II-a:

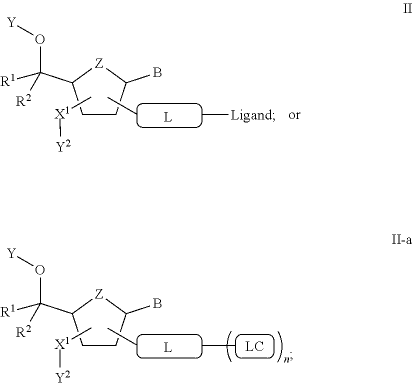

or a pharmaceutically acceptable salt thereof, wherein each variable is as defined and described herein.

In some embodiments, the oligonucleotide-lipid conjugates are represented by formula II-b, II-c, II-Ib, II-Ic, II-d, II-e, II-Id or II-Ie:

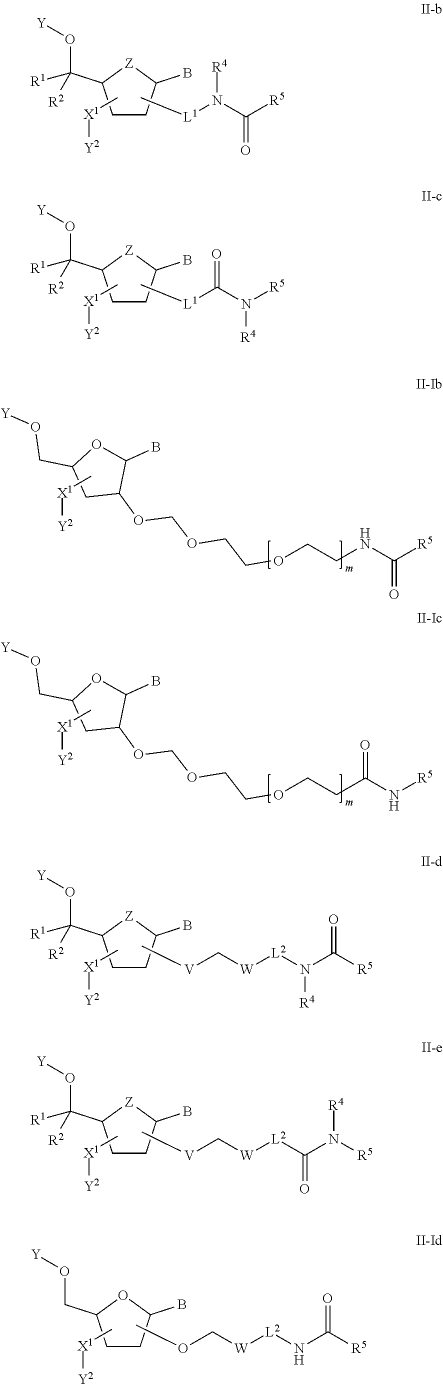

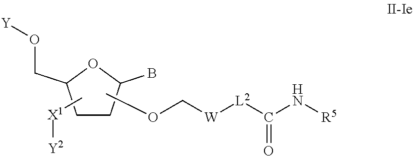

or a pharmaceutically acceptable salt thereof, wherein each variable is as defined and described herein.

Oligonucleotide-ligand conjugates of the present disclosure comprise one or more nucleic acid-ligand conjugate units represented by any of the formula I, I-a, I-b, I-c, I-Ib, II-Ic, I-d, I-e, I-Id, I-Ie, II, II-a, II-b, II-c, II-Ib, II-Ic, II-d, II-e, II-Id or II-Ie.

Nucleic acid-ligand conjugates and oligonucleotide-ligand conjugates of the present disclosure, and pharmaceutically acceptable compositions thereof, are useful for treating a variety of diseases, disorders, or conditions, associated with regulation of intracellular RNA levels. Such diseases, disorders, or conditions include those described herein. Methods of making and methods of delivering these nucleic acid-ligand conjugates and oligonucleotide-lipid conjugates are disclosed herein.

Nucleic acid-ligand conjugates and oligonucleotide-ligand conjugates provided by this disclosure are also useful for the study of gene expression in biological and pathological phenomena; the study of RNA levels in bodily tissues; and the comparative evaluation of new RNA interference agents, in vitro or in vivo. Nucleic acid-ligand conjugates and oligonucleotide-ligand conjugates disclosed herein are useful in reducing expression of a target gene

### BRIEF DESCRIPTION OF THE DRAWINGS

FIG. 1 shows the gene silencing of ALDH2 mRNA in different tissues at day 5 after a single 15 mg/kg intravenous injection of GalXC lipid conjugates.

FIG. 2 shows the dose-response effect of gene silencing of ALDH2 mRNA in extrahepatic tissues by a single intravenous injection of Duplex 1c (C22), at day 6 and day 14 after dosing

FIG. 3 shows the durable ALDH2 silencing activity of Duplex 1c (C22) in different tissues following one single subcutaneous dosing of 15 mg/kg.

FIG. 4 shows the gene silencing activity of GalXC diacyl lipid conjugates Duplex 1h (diacyl C16), 1i (diacyl C18:1), 1j (PEG2K-diacyl C18) and mono lipid conjugate Duplex 1b (C18) in extrahepatic tissues following one single subcutaneous dosing of 15 mg/kg.

FIG. 5 shows the gene silencing activity of GalXC long-lipid conjugates Duplex 1d (C24), 1e (C26), 1g (C24:1) and adamantane conjugate Duplex 5b (3Xacetyladamantane) in different tissues at day 7 and day 14 after a single subcutaneous dosing of 15 mg/kg.

FIG. 6 shows the gene silencing of ALDH2 mRNA level in different tissues at day 7 and day 14 after a single subcutaneous dosing of 15 mg/kg of these GalXC lipid conjugates.

### DETAILED DESCRIPTION OF CERTAIN EMBODIMENTS

### 1. Nucleic Acid-Ligand Conjugates:

The disclosed novel nucleic acid-ligand conjugates elicit broad extrahepatic RNAi activity. Incorporation of the lipid moiety facilitates systemic delivery of the nucleic acids or analogues thereof into several tissues, for example the CNS, muscle, adipose, and adrenal gland.

Nucleic acid-ligand conjugates thereof of the present disclosure, and compositions thereof, are useful as RNA interference agents. In some embodiments, a provided nucleic acid-ligand conjugate or analogue thereof inhibits gene expression in a cell.

In a first embodiment, the present disclosure provides a nucleic acid-lipid conjugate represented by formula I:

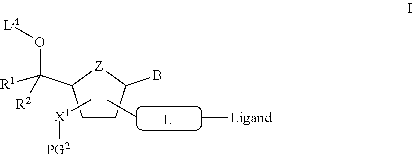

or a pharmaceutically acceptable salt thereof, wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃, or:
   R¹ and R² on the same carbon are taken together with their intervening atoms to form a 3-membered saturated or partially unsaturated ring having 0-3 heteroatoms, independently selected from nitrogen, oxygen, and sulfur;
each R^{A} is independently an optionally substituted group selected from C1-6 aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
   L^{A} is independently PG¹, or -L-ligand;
   PG¹ is hydrogen or a suitable hydroxyl protecting group;
   each ligand is independently -(LC)ₙ, or an adamantyl group;
each LC is independently a lipid conjugate moiety comprising a saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, or —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, adamantanenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —N(R)—C(O)—, —S—, —C(O)—, —S(O)—, —S(O)₂—, —P(O)OR—, —P(S)OR—, —V¹CR²W¹— or

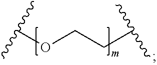

m is 1-50;
X¹, V¹ and W¹ are independently —C(R)₂—, —OR, —O—, —S—, —Se—, or —NR—;
Z is —O—, —S—, —NR—, or —CR₂—; and
PG² is hydrogen, a phosphoramidite analogue, or a suitable protecting group.

In a second embodiment, the nucleic acid-ligand conjugate of the first embodiment is represented by formula I-a:

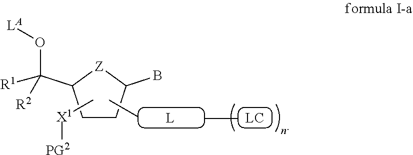

In a third embodiment, the nucleic acid-ligand conjugate of the first embodiment is represented by formula I-b or I-c:

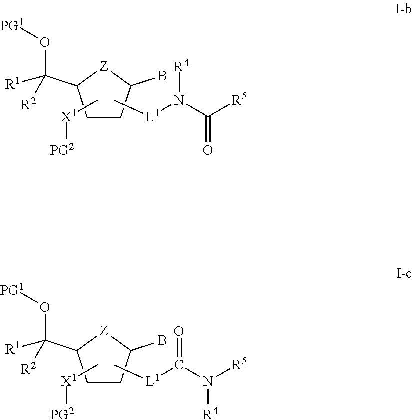

or a pharmaceutically acceptable salt thereof, wherein
L¹ is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, —P(S)OR—, or

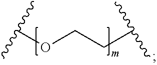

R⁴ is hydrogen, R^{A}, or a suitable amine protection group; and
R⁵ is adamantyl, or a saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —C(O)O—, —S(O)—, —S(O)₂—, —P(O)OR—, or —P(S)OR—.

In a fourth embodiment, the nucleic acid-ligand conjugate is represented by formula I-d or I-e:

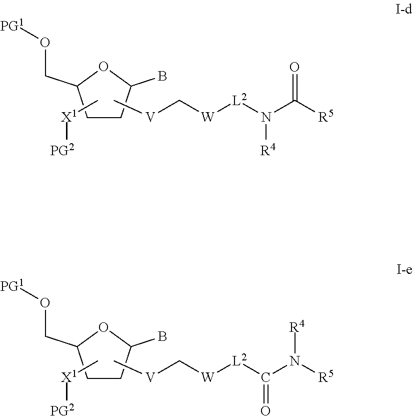

or a pharmaceutically acceptable salt thereof, wherein
B is a nucleobase or hydrogen;
PG¹ and PG² are independently a hydrogen, a phosphoramidite analogue, or a suitable protecting group; and
R⁵ is adamantyl, or a saturated or unsaturated, straight or branched C₁₋₅₀ hydrocarbon chain, wherein 0-10 methylene units of the hydrocarbon chain are independently replaced by —O—, —NR—, —S—, —C(O)—, —C(O)O—, —S(O)—, —S(O)₂—, —P(O)OR—, or —P(S)OR—.
V is a bivalent group selected from —O—, —S—, and —NR—;
W is a bivalent group selected from —O—, —S—, —NR—, —C(O)NR—, —OC(O)NR—, —SC(O)NR—,

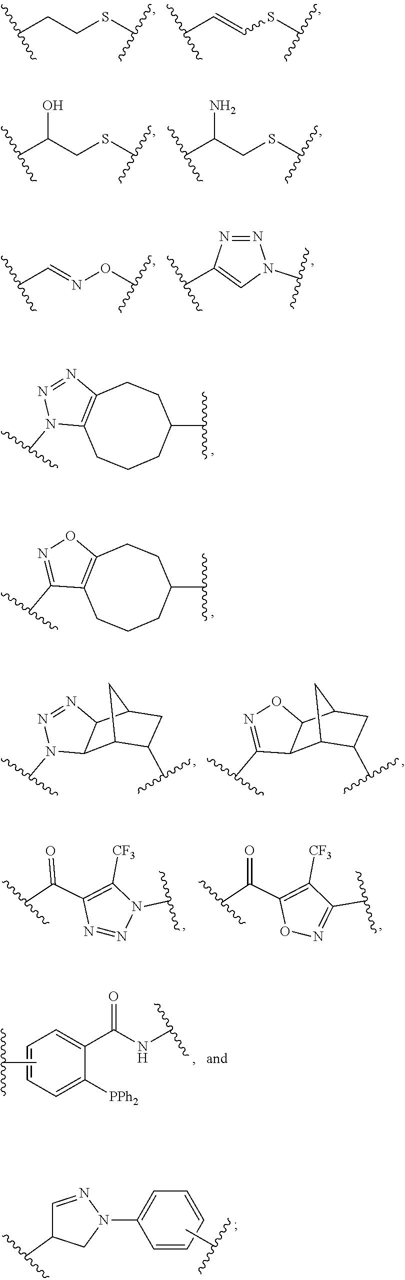

L² is a covalent bond or a bivalent saturated or unsaturated, straight or branched C₁₋₅₀ hydrocarbon chain, wherein 0-10 methylene units of the hydrocarbon chain are independently replaced by —O—, —NR—, —S—, —C(O)—, —S(O)—, —S(O)₂—, —P(O)OR—, —P(S)OR—, or

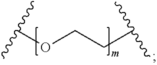

m is 1-50;
X¹ is —C(R)₂—, —OR, —O—, —S—, —Se—, or —NR—;
R⁴ is hydrogen, R^{A}, or a suitable amine protection group; and
R⁵ is adamantyl, or a saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In a fifth embodiment, the nucleic acid-ligand conjugate of the fourth embodiment, wherein:
V is —O—;
L² is a covalent bond or a bivalent saturated or unsaturated, straight or branched C₁₋₅₀ hydrocarbon chain, wherein 0-10 methylene units of the hydrocarbon chain are independently replaced by —O—, —C(O)—,

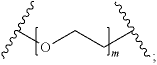

R⁴ is hydrogen;
w is —O—, —NR—, —C(O)NR—, —OC(O)NR

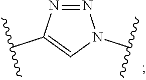 [SMILES: *c1cn(*)nn1]

and
R⁵ is a saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, or —C(O)O—.

In a sixth embodiment, a nucleic acid-ligand conjugate is represented by formula I-Ib or I-Ic:

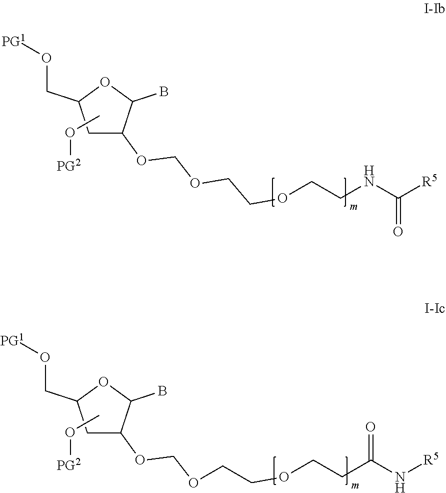

or a pharmaceutically acceptable salt thereof, wherein
B is a nucleobase or hydrogen;
m is 1-50;
PG¹ and PG² are independently a hydrogen, a phosphoramidite analogue, or a suitable protecting group; and
R⁵ is adamantyl, or a saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—; and
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In a seventh embodiment, the nucleic acid-ligand of the sixth embodiment, wherein the R⁵ is selected from

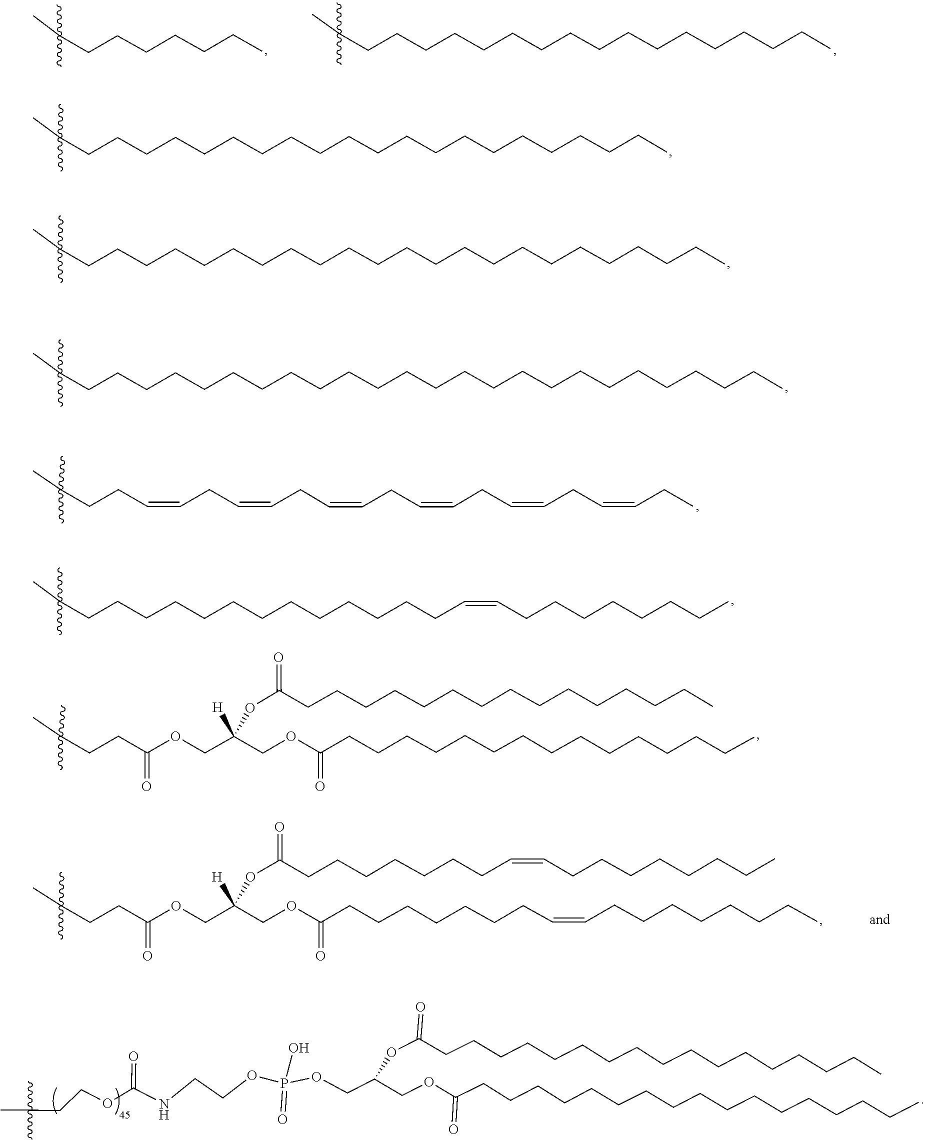

In some embodiments, the oligonucleotide-ligand conjugates comprise one or more nucleic acid-conjugate units of any one of the above disclosed embodiments one to seven represented by any one of the formula I, I-a, I-b, I-c, I-d, I-e, I-Ib or I-Ic.

In some embodiments, the oligonucleotide-ligand conjugate of the present disclosure comprises 1, 2, 3, 4, 5, 6, 7, 8, 9 or 10 nucleic acid-ligand conjugate units. In some embodiments, the conjugate comprises 1 nucleic acid-ligand conjugate unit. In some embodiments, the conjugate comprises 2 nucleic acid-ligand conjugate units. In some embodiments, the conjugate comprises 3 nucleic acid-ligand conjugate units.

### 2. Oligonucleotide-Ligand Conjugates

The disclosed novel oligonucleotide-ligand conjugates elicit broad extrahepatic RNase H activity. Incorporation of the hydrophobic moiety e.g. adamntyl or the lipid moiety facilitates systemic delivery of the oligonucleotides or analogues thereof into several tissues, for example the CNS, muscle, adipose, and adrenal gland.

Oligonucleotide-ligand conjugates thereof of the present disclosure, and compositions thereof, are useful as RNA interference agents. In some embodiments, a provided oligonucleotide-ligand conjugate or analogue thereof inhibits gene expression in a cell.

Another aspect of the present disclosure provides an oligonucleotide comprising nucleic acid-ligand conjugates, in which the oligonucleotides comprise an antisense strand of 15 to 30 nucleotides in length and one or more ligand moieties. In some embodiments, the ligand moiety is independently adamantyl or a lipid moiety. In some embodiments, the antisense strand has a region of complementarity to a target gene sequence. In some embodiments, the region of complementarity is at least 15, at least 16, at least 17, at least 18, at least 19, at least 20, or at least 21 contiguous nucleotides in length. In some embodiments, the antisense strand is 19 to 27 nucleotides in length. In some embodiments, the antisense strand is 21 to 27 nucleotides in length.

In some embodiments, the oligonucleotide further comprises a sense strand of 10 to 53 nucleotides in length, in which the sense strand forms a duplex region with the antisense strand and the lipid moiety is attached to sense strand. In some embodiments, the sense strand is 12 to 40 nucleotides in length. In some embodiments, the sense strand is 15 to 40 nucleotides in length. In some embodiments, the duplex region is at least 15, at least 16, at least 17, at least 18, at least 19, at least 20, or at least 21 nucleotides in length. In some embodiments, the region of complementarity to the target sequence is at least 19 contiguous nucleotides in length. In some embodiments, the sense strand comprises at its 3′-end a stem-loop set forth as: S1-L-S2, in which S1 is complementary to S2, and in which L forms a loop between S1 and S2 of 3 to 5 nucleotides in length. In some embodiments, the lipid moiety is attached to the loop L.

An eighth embodiment of the present disclosure discloses an oligonucleotide-ligand conjugate comprising one or more nucleic acid-ligand conjugates represented by formula II:

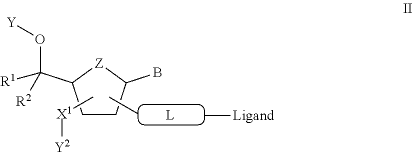

or a pharmaceutically acceptable salt thereof, wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃; or
   R¹ and R² on the same carbon are taken together with their intervening atoms to form a 3-7 membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur; or
   two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
ligand is independently -(LC)ₙ, or an adamantyl group;
each LC is independently a lipid conjugate moiety comprising a saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —N(R)—C(O)—, —S—, —C(O)—, —S(O)—, —S(O)₂—, —P(O)OR—, —P(S)OR—, —V¹CR²W¹—, or

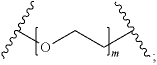

m is 1-50;
X¹, V¹ and W¹ are independently —C(R)₂—, —OR, —O—, —S—, —Se—, or —NR—;
Y is hydrogen, a suitable hydroxyl protecting group,

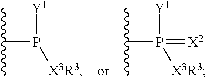

R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, S, or NR;
X³ is —O—, —S—, —BH₂—, or a covalent bond;
Y¹ is a linking group attaching to the 2′- or 3′-terminal of a nucleoside, a nucleotide, or an oligonucleotide;
Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support; and
Z is —O—, —S—, —NR—, or —CR₂—.

A ninth embodiment discloses the oligonucleotide-ligand conjugate, wherein the conjugate of the eighth embodiment is represented by formula II-a or II-a-1

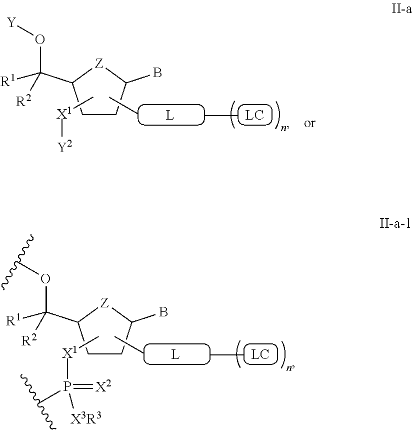

or a pharmaceutically acceptable salt thereof, wherein:
each of B, R¹, R², Y, L, LC, n, and Z is as defined above.

Some embodiments disclose the oligonucleotide-ligand conjugate, wherein X¹ is —O—, Y² is phosphoramidite

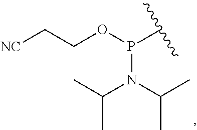 [SMILES: *P(OCCC#N)N(C(C)C)C(C)C]

and the connectivity and stereochemistry is as shown in formula II-a1:

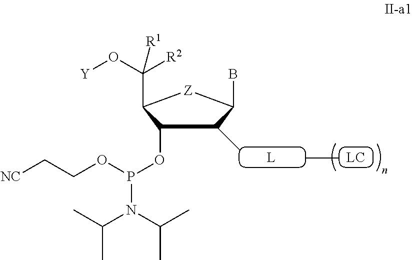

or a pharmaceutically acceptable salt thereof, wherein:
each of B, R¹, R², Y, L, LC, n, and Z is as defined above.

Some embodiments disclose the oligonucleotide-ligand conjugate, wherein X¹ is —O—, Y² is a phosphate interlinking group, and the connectivity and stereochemistry are as shown in formula II-a2:

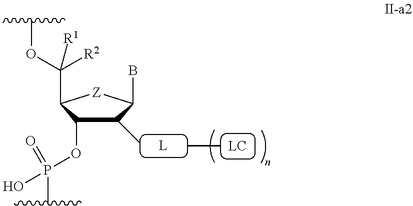

or a pharmaceutically acceptable salt thereof, wherein:
each of B, R¹, R², Y, L, LC, n, and Z is as defined above.

A tenth embodiment discloses the oligonucleotide-ligand conjugate of the any one of the above disclosed oligonucleotide-ligand conjugate embodiments, wherein the conjugate is represented by formula II-b or II-c:

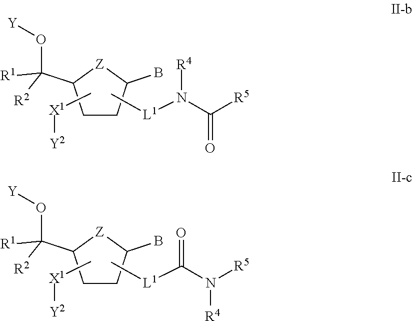

or a pharmaceutically acceptable salt thereof, wherein:
L¹ is a covalent bond, a monovalent or a bivalent saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, —P(S)OR—, or

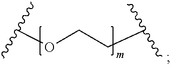

R⁴ is hydrogen, R^{A}, or a suitable amine protection group; and
R⁵ is adamantyl, or a saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, or —P(S)OR—.

An eleventh embodiment discloses the oligonucleotide-ligand conjugate of the eighth embodiment, wherein the conjugate is represented by formula II-d or II-e:

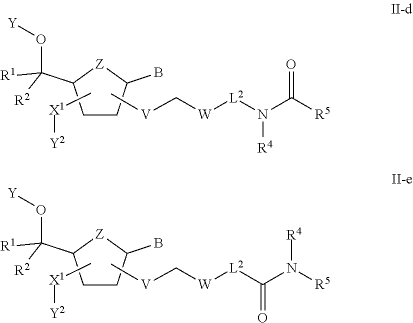

or a pharmaceutically acceptable salt thereof;
V is a bivalent group selected from —O—, —S—, and —NR—;
W is a bivalent group selected from —O—, —S—, —NR—, —C(O)NR—, —OC(O)NR—, —SC(O)NR—,

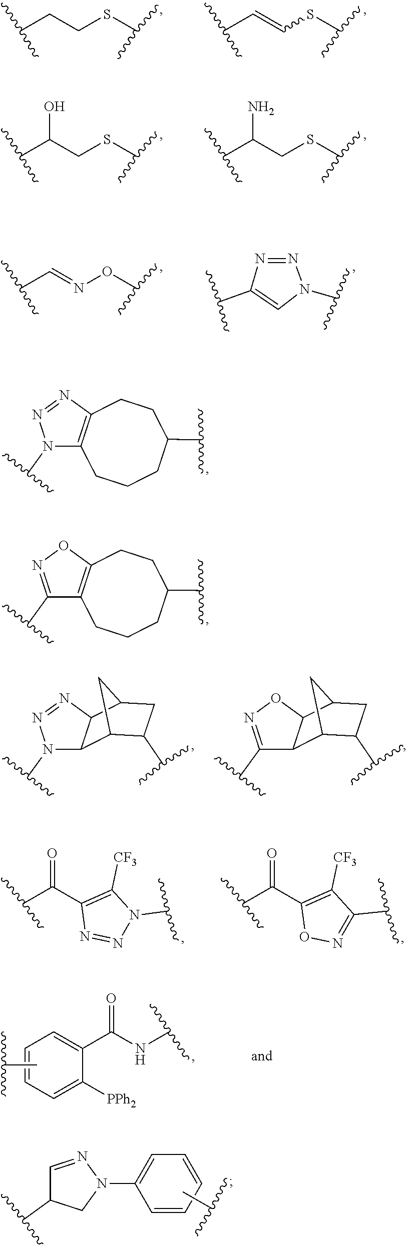

L² is a covalent bond or a bivalent saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, —P(S)OR—, or

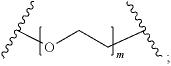

R⁴ is hydrogen, R^{A}, or a suitable amine protection group; and
R⁵ is a saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, or —P(S)OR—.

A twelfth embodiment discloses an oligonucleotide-ligand conjugate represented by formula II-Id or II-Ie:

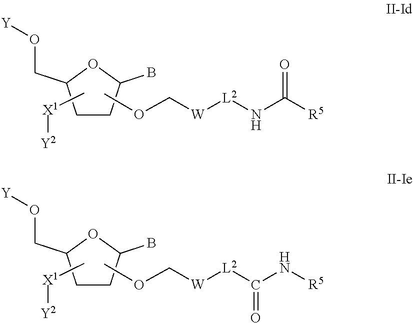

or a pharmaceutically acceptable salt thereof; wherein:
m is 1-50;
B is H, or a nucleobase;
X¹ is —C(R)₂—, —OR, —O—, —S—, or —NR—;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
w is a bivalent group selected from —O—, —S—, —NR—, —C(O)NR—, —OC(O)NR—,

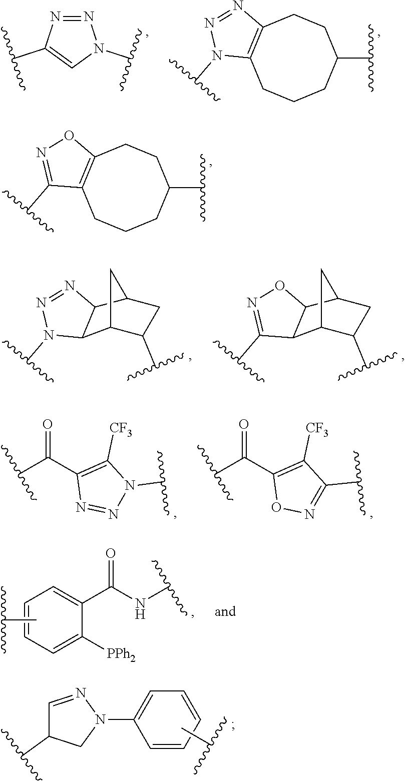

L² is a covalent bond or a bivalent saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, —P(S)OR—, or

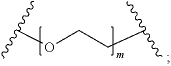

Y is hydrogen,

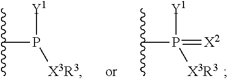

R³ is hydrogen, or a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, or S;
X³ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support; and
R⁵ is adamantyl, or a saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—.

A thirteenth embodiment discloses the oligonucleotide-ligand conjugate of the eleventh embodiment, wherein:
R⁵ is selected from

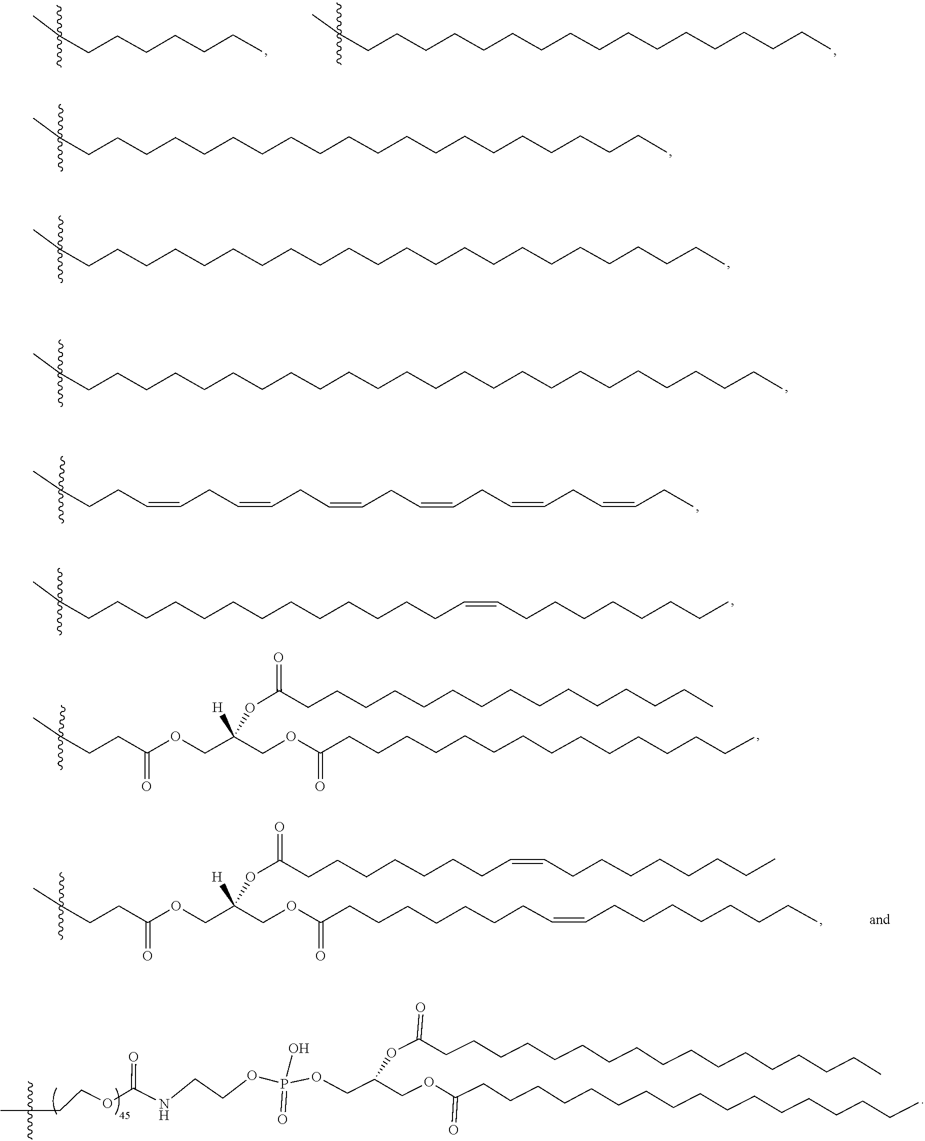

A fourteenth embodiment discloses an oligonucleotide-ligand conjugate represented by formula II-Ib or II-Ic:

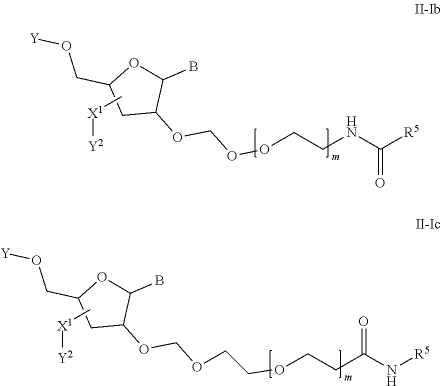

or a pharmaceutically acceptable salt thereof; wherein
B is a nucleobase or hydrogen;
m is 1-50;
X¹ is —O—, or —S—;
Y is hydrogen,

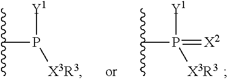

R³ is hydrogen, or a suitable protecting group;
X² is O, or S;
X³ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support;
R⁵ is adamantyl, or a saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—; and
R is hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

A fifteenth embodiment discloses the oligonucleotide-ligand conjugate of the fourteenth embodiment, wherein:
R⁵ is selected from

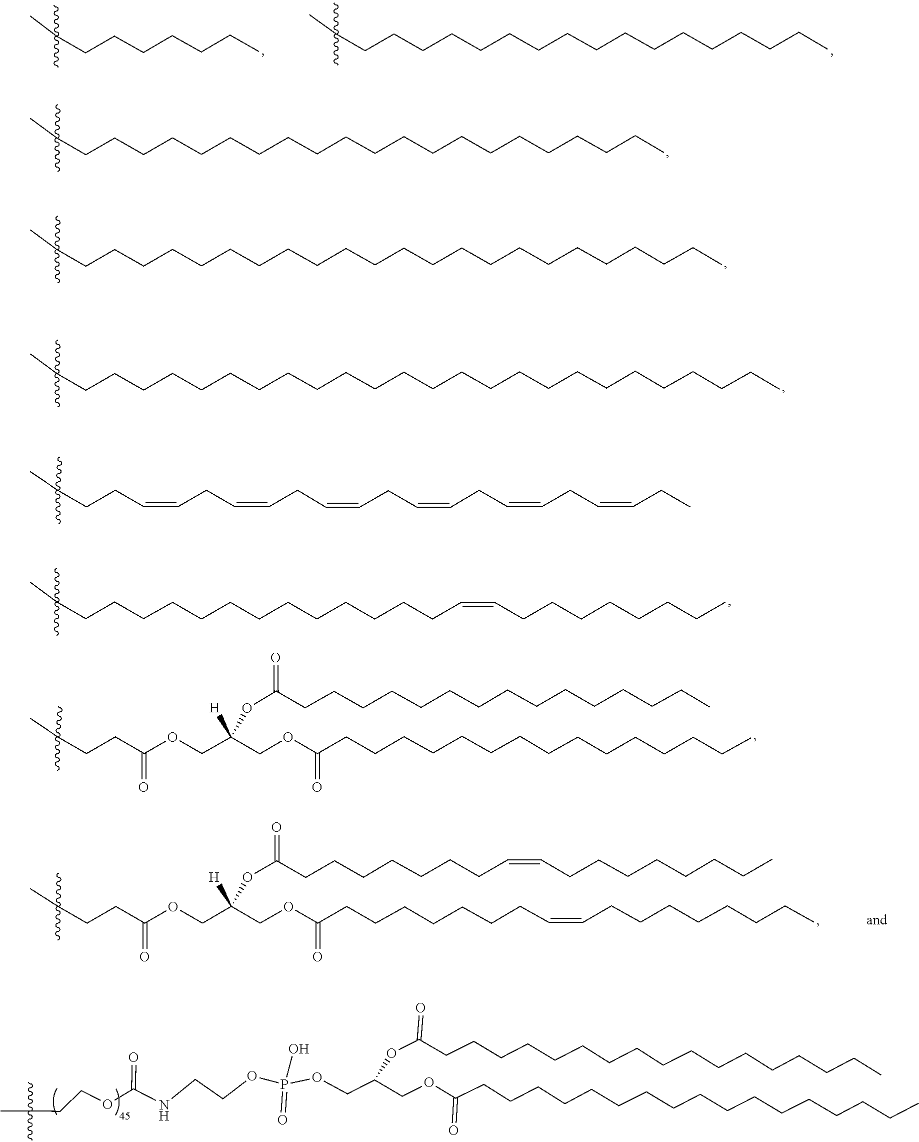

In some embodiments, X¹ is —O—, Y² is phosphoramidite

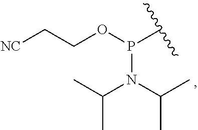 [SMILES: *P(OCCC#N)N(C(C)C)C(C)C]

and the connectivity and stereochemistry are as shown in formula II-b-1 or II-c-1:

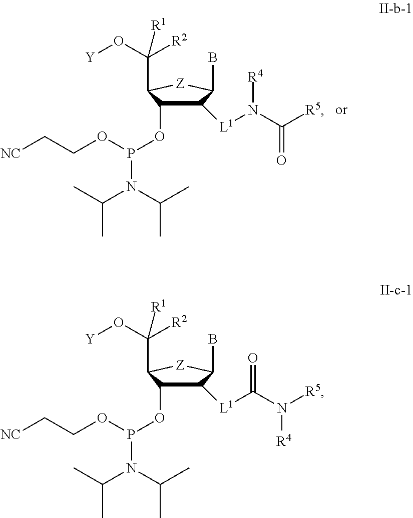

or a pharmaceutically acceptable salt thereof, wherein:
each of B, R¹, R², R³, R⁴, Y, L¹, and Z is as defined above.

In some oligonucleotide-ligand conjugate embodiments, X¹ is —O—, Y² is a phosphate interlinking group, and the connectivity and stereochemistry is as shown in formula II-b-2 or II-c-2:

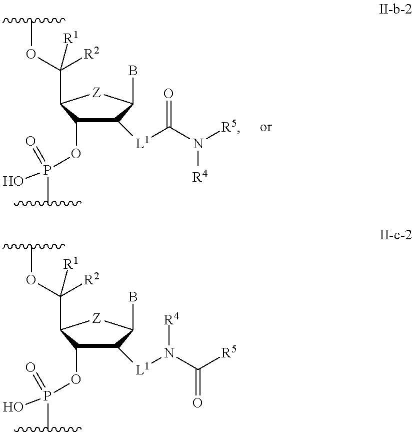

or a pharmaceutically acceptable salt thereof, wherein:
each of B, R¹, R², R³, R⁴, L¹, and Z is as defined above.

In some embodiments, the oligonucleotide-ligand conjugate of the eighth embodiment, wherein the conjugate is represented by formula II-d or II-e:

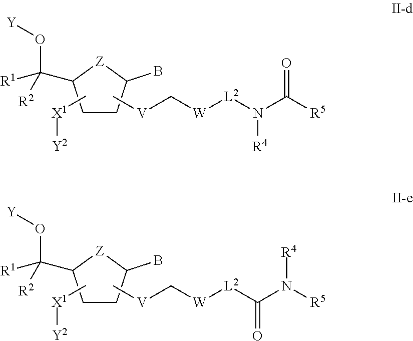

or a pharmaceutically acceptable salt thereof;
V is a bivalent group selected from —O—, —S—, and —NR—;
W is a bivalent group selected from —O—, —S—, —NR—, —C(O)NR—, —OC(O)NR—, —SC(O)NR—,
L² is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, —P(S)OR—, or

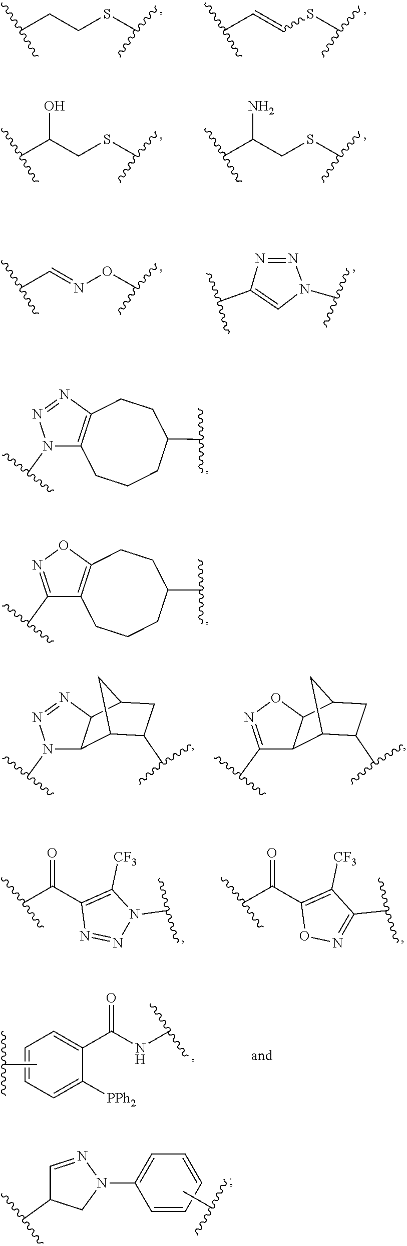

L² is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, —P(S)OR—, or

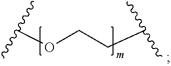

R⁴ is hydrogen, R^{A}, or a suitable amine protection group; and
R⁵ is a saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, or —P(S)OR—.

In some oligonucleotide-ligand conjugate embodiments, wherein X¹ is —O—, Y² is phosphoramidite

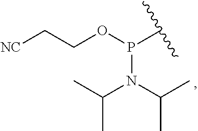 [SMILES: *P(OCCC#N)N(C(C)C)C(C)C]

and the connectivity and stereochemistry is as shown in formula II-d-1 or II-e-1:

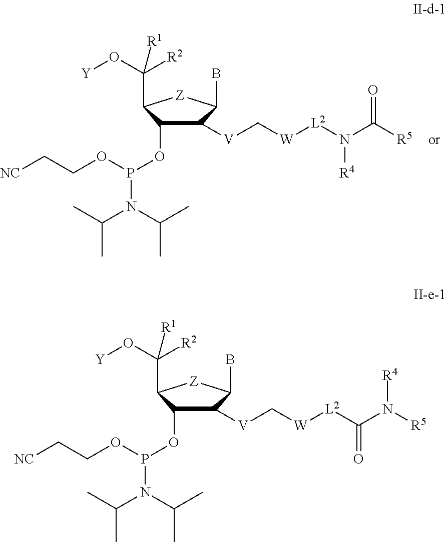

or a pharmaceutically acceptable salt thereof, wherein:
each of B, R¹, R², R³, R⁴, Y, L², V, W, and Z is as defined above.

In some oligonucleotide-ligand conjugate embodiments, wherein X¹ is —O—, Y² is a phosphate interlinking group, and the connectivity and stereochemistry is as shown, thereby forming an oligonucleotide-ligand conjugate comprising a unit of formula II-d-2 or II-e-2:

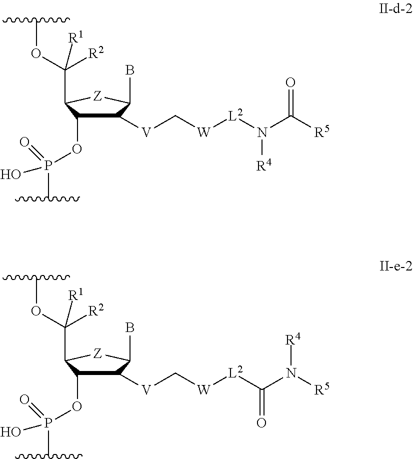

or a pharmaceutically acceptable salt thereof, wherein:
each of B, R¹, R², R³, R⁴, Y, L², V, W, and Z is as defined above.

In a sixteenth embodiment, the oligonucleotide-ligand conjugate of any one of eighth to fifteenth embodiments, wherein the conjugate comprises 1 to 10 nucleic acid-ligand conjugate units. In some embodiments, the conjugate comprises 1, 2, 3, 4, 5, 6, 7, 8, 9 or 10 nucleic acid-ligand conjugate units. In some embodiments, the conjugate comprises 1 nucleic acid-ligand conjugate unit. In some embodiments, the conjugate comprises 2 nucleic acid-ligand conjugate units. In some embodiments, the conjugate comprises 3 nucleic acid-ligand conjugate units.

In certain embodiments of any of the above disclosed aspects or embodiments, each LC is a fatty acid selected from C8:0, C10:0, C11:0, C12:0, C14:0, C16:0, C17:0, C18:0, C22:0, C24:0, C26:0, C22:6, C24:1, diacyl C16:0, diacyl C18:1, and adamantane carboxylic acid. In certain embodiments, the adamantane carboxylic acid is Adamantane acetic acid.

In certain embodiments of any of the above disclosed aspects or embodiments, n is 1 or 2.

In some embodiments, B is a nucleobase or hydrogen. In some embodiments, B is a nucleobase. In some embodiments, B is a nucleobase analogue. In some embodiments, B is a modified nucleobase. In some embodiments, B is a universal nucleobase. In some embodiments, B is a hydrogen.

In some embodiments, B is selected from guanine (G), cytosine (C), adenine (A), thymine (T), uracil (U),

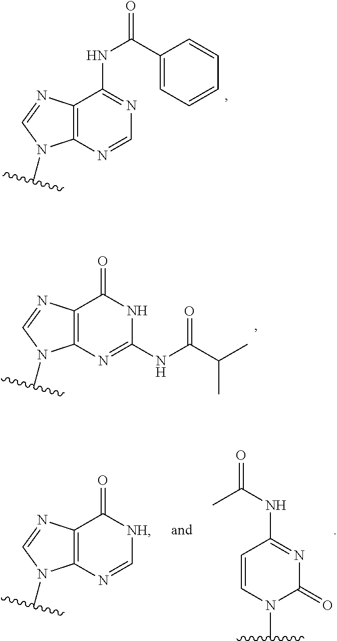

In some embodiments, B is selected from those depicted in Table 1.

As defined above and described herein, R¹ and R² are independently hydrogen, halogen, R^{A}, —CN, —S(O)R, —S(O)₂R, —Si(OR)₂R, —Si(OR)R₂, or —SiR₃, or R¹ and R² on the same carbon are taken together with their intervening atoms to form a 3-7 membered saturated or partially unsaturated ring having 0-3 heteroatoms, independently selected from nitrogen, oxygen, and sulfur.

In some embodiments, R¹ and R² are independently hydrogen, deuterium, or halogen. In some embodiments, R¹ and R² are independently R^{A}, —CN, —S(O)R or —S(O)₂R. In some embodiments, R¹ and R² are independently —Si(OR)₂R, —Si(OR)R₂ or —SiR₃. In some embodiments, R and R² on the same carbon are taken together with their intervening atoms to form a 3-7 membered saturated or partially unsaturated ring having 0-3 heteroatoms, independently selected from nitrogen, oxygen, and sulfur.

In some embodiments, R is methyl and R² is hydrogen.

In some embodiments, R¹ and R² are selected from those depicted in Table 1.

As defined above and described herein, each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur.

In some embodiments, R is a suitable protecting group. In some embodiments, R is hydrogen, C₁₋₆ aliphatic or an optionally substituted phenyl. In some embodiments, R is an optionally substituted 4-7 membered saturated or partially unsaturated heterocyclic having 1-2 heteroatoms independently selected from nitrogen, oxygen, and sulfur, or R is an optionally substituted 5-6 membered heteroaryl ring having 1-4 heteroatoms independently selected from nitrogen, oxygen, and sulfur. In some embodiments, two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur.

In some embodiments, R is hydrogen. In some embodiments, R is selected from those depicted in Table 1, below.

As defined above and described herein, each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R^{A} is an optionally substituted C₁₋₆ aliphatic, or an optionally substituted phenyl. In some embodiments, R^{A} is an optionally substituted 4-7 membered saturated or partially unsaturated heterocyclic ring having 1-2 heteroatoms independently selected from nitrogen, oxygen, and sulfur, or an optionally substituted 5-6 membered heteroaryl ring having 1-4 heteroatoms independently selected from nitrogen, oxygen, and sulfur.

In some embodiments, R^{A} is selected from those depicted in Table 1, below.

As defined above and described herein, each ligand is independently hydrogen, or a hydrophobic moiety selected from adamantyl group and lipid moiety.

As defined above and described herein, each LC is independently a lipid conjugate moiety comprising a saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, or —P(S)OR—.

In some embodiments, LC is a lipid conjugate moiety comprising a saturated or partially unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, or —P(S)OR—.

As used herein, the lipid conjugate moiety is formed from the coupling of a nucleic acid or analogue thereof described herein with a lipophilic compound. In some embodiments, LC is a lipid conjugate moiety comprising an esterified or amidated saturated straight-chain fatty acid. In some embodiments, LC is —OC(O)CH₃ or —NHC(O)CH₃. In some embodiments, LC is —OC(O)C₂H₅ or —NHC(O)C₂H₅. In some embodiments, LC is —OC(O)C₃H₇ or —NHC(O)C₃H₇. In some embodiments, LC is —OC(O)C₄H₉ or —NHC(O)C₄H₉. In some embodiments, LC is —OC(O)C₅H₁₁ or —NHC(O)C₅H₁₁. In some embodiments, LC is —OC(O)C₆H₁₃ or —NHC(O)C₆H₁₃. In some embodiments, LC is —OC(O)C₇H₁₅ or —NHC(O)C₇H₁₅. In some embodiments, LC is —OC(O)C₅H₁₇ or —NHC(O)C₅H₁₇. In some embodiments, LC is —OC(O)C₉H₁₉ or —NHC(O)C₉H₁₉. In some embodiments, LC is —OC(O)C₁₀H₂₁ or —NHC(O)C₁₀H₂₁. In some embodiments, LC is —OC(O)C₁₁H₂₃ or —NHC(O)C₁₁H₂₃. In some embodiments, LC is —OC(O)C₁₂H₂₅ or —NHC(O)C₁₂H₂₅. In some embodiments, LC is —OC(O)C₁₃H₂₇ or —NHC(O)C₁₃H₂₇. In some embodiments, LC is —OC(O)C₁₄H₂₉ or —NHC(O)C₁₄H₂₉. In some embodiments, LC is —OC(O)C₁₅H₃₁ or —NHC(O)C₁₅H₃₁. In some embodiments, LC is —OC(O)C₁₆H₃₃ or —NHC(O)C₁₆H₃₃. In some embodiments, LC is —OC(O)C₁₇H₃₅ or —NHC(O)C₁₇H₃₅. In some embodiments, LC is —OC(O)C₁₈H₃₇ or —NHC(O)C₁₈H₃₇. In some embodiments, LC is —OC(O)C₁₉H₃₉ or —NHC(O)C₁₉H₃₉. In some embodiments, LC is —OC(O)C₂₀H₄₁ or —NHC(O)C₂₀H₄₁. In some embodiments, LC is —OC(O)C₂₁H₄₃ or —NHC(O)C₂₁H₄₃. In some embodiments, LC is —OC(O)C₂₂H₄₅ or —NHC(O)C₂₂H₄₅. In some embodiments, LC is —OC(O)C₂₃H₄₇ or —NHC(O)C₂₃H₄₇. In some embodiments, LC is —OC(O)C₂₄H₂₉ or —NHC(O)C₂₄H₂₉. In some embodiments, LC is —OC(O)C₂₅H₅₁ or —NHC(O)C₂₅H₅₁. In some embodiments, LC is —OC(O)C₂₆H₅₃ or —NHC(O)C₂₆H₅₃. In some embodiments, LC is —OC(O)C₂₇H₅₅ or —NHC(O)C₂₇H₅₅. In some embodiments, LC is —OC(O)C₂₈H₅₇ or —NHC(O)C₂₈H₅₇. In some embodiments, LC is —OC(O)C₂₉H₅₉ or —NHC(O)C₂₉H₅₉. In some embodiments, LC is —OC(O)C₃₀H₆₁ or —NHC(O)C₃₀H₆₁.

In some embodiments, LC is a lipid conjugate moiety comprising an esterified or amidated partially unsaturated straight-chain fatty acid. In some embodiments, LC is esterified or amidated myristoleic acid. In some embodiments, LC is esterified or amidated palmitoleic acid. In some embodiments, LC is esterified or amidated sapienic acid. In some embodiments, LC is esterified or amidated oleic acid, i.e.,

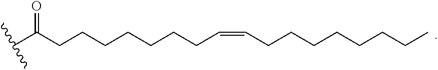 [SMILES: *C(=O)CCCCCCC/C=C\CCCCCCCC]

In some embodiments, LC is esterified or amidated elaidic acid. In some embodiments, LC is esterified or amidated vaccenic acid. In some embodiments, LC is esterified or amidated linoleic acid. In some embodiments, LC is esterified or amidated limoelaidic acid. In some embodiments, LC is esterified or amidated α-linolenic acid, i.e.,

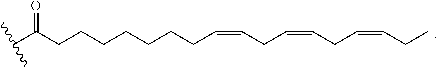 [SMILES: *C(=O)CCCCCCC/C=C\C/C=C\C/C=C\CC]

In some embodiments, LC is esterified or amidated arachidonic acid. In some embodiments, LC is esterified or amidated eicosapentaenoic acid, i.e.,

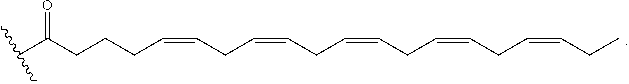 [SMILES: *C(=O)CCC/C=C\C/C=C\C/C=C\C/C=C\C/C=C\CC]

In some embodiments, LC is esterified or amidated erucic acid. In some embodiments, LC is esterified or amidated docosahexaenoic acid, i.e.,

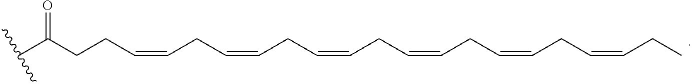 [SMILES: *C(=O)CC/C=C\C/C=C\C/C=C\C/C=C\C/C=C\C/C=C\CC]

In some embodiments, LC is esterified or amidated adamantanecarboxylic acid. In some embodiments, LC is esterified or amidated adamantaneacetic acid. In some embodiments, R⁵ is —C(O)(CH₂)₁₋₁₀adamantane.

In some embodiments, LC is selected from those depicted in Table 1, below.

As defined above and described herein, n is 1, 2, 3, 4, or 5. In some embodiments, n is 1, or 2. In some embodiments, n is selected from those depicted in Table 1, below.

As defined above and described herein, L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, —P(S)OR—, or

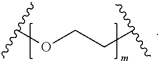

In some embodiments, L is a covalent bond. In some embodiments, L is

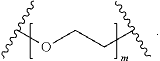

In some embodiments, L is selected from those depicted in Table 1, below.

As defined above and described herein, L¹ is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, —P(S)OR—, or

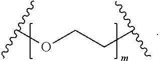

In some embodiments, L¹ is a covalent bond. In some embodiments, L¹ is

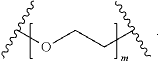

In some embodiments, L¹ is selected from those depicted in Table 1, below.

As defined above and described herein, L² is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, —P(S)OR—, or

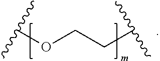

In some embodiments, L² is a covalent bond. In some embodiments, L² is

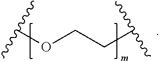

In some embodiments, L² is selected from those depicted in Table 1, below.

As defined above and described herein, m is 1-50.

In some embodiments, m is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

In some embodiments, m is selected from those depicted in Table 1, below.

As defined above and described herein, R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R³ is hydrogen, or a suitable protecting group. In some embodiments, R³ is a suitable prodrug. In some embodiments, R³ is a suitable phosphate/phosphonate prodrug, which is a glutathione-sensitive moiety. In some embodiments, R³ is a glutathione-sensitive moiety selected from those as described in International Patent Application No. PCT/US2017/048239, which is hereby incorporated by reference in its entirety.

In some embodiments, R³ is an optionally substituted C₁₋₆ aliphatic, an optionally substituted phenyl, an optionally substituted 4-7 membered saturated or partially unsaturated heterocyclic having 1-2 heteroatoms, or an optionally substituted 5-6 membered heteroaryl ring having 1-4 heteroatoms, wherein the heteroatoms are independently selected from nitrogen, oxygen, and sulfur.

In some embodiments, R³ is methyl, or ethyl. In some embodiments, R³ is

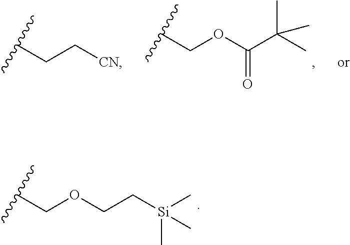

In some embodiments, R³ is selected from those depicted in Table 1, below.

As defined above and described herein, R⁴ is hydrogen, R^{A}, or a suitable amine protection group.

In some embodiments, R⁴ is hydrogen. In some embodiments, R⁴ is R^{A}. In some embodiments, R⁴ is a suitable amine protecting group.

Suitable amine protecting groups and the reagents and reaction conditions appropriate for using them to protect and deprotect amine groups are well known in the art and include those described in detail in PROTECTING GROUPS IN ORGANIC SYNTHESIS, (T. W. Greene and P. G. M. Wuts, 3^{rd} edition, John Wiley & Sons, 1999), the entirety of which is incorporated herein by reference. Suitable amine protecting groups, taken with the nitrogen to which it is attached, include, but are not limited to, aralkyl amines, carbamates, allyl amines, amides, and the like. Examples of amine protecting groups of the compounds of the formulae described herein include tert-butyloxycarbonyl (Boc), ethyloxycarbonyl, methyloxycarbonyl, trichloroethyloxycarbonyl, allyloxycarbonyl (Alloc), benzyloxocarbonyl (Cbz), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like.

In some embodiments, R⁴ is selected from those depicted in Table 1, below.

As defined above and described herein, each R⁵ is a saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, or —P(S)OR—.

In some embodiments, R⁵ is —CH₃. In some embodiments, R⁵ is —C₂H₅. In some embodiments, R⁵ is —C₃H₇. In some embodiments, R⁵ is —C₄H₉. In some embodiments, R⁵ is C₅H₁₁. In some embodiments, R⁵ is —C₆H₁₃. In some embodiments, R⁵ is —C₇H₁₅. In some embodiments, R⁵ is —C₅H₁₇. In some embodiments, R⁵ is —C₉H₁₉. In some embodiments, R⁵ is —C₁₀H₂₁. In some embodiments, R⁵ is —C₁₁H₂₃. In some embodiments, R⁵ is —C₁₂H₂₅. In some embodiments, R⁵ is —C₁₃H₂₇. In some embodiments, R⁵ is —C₁₄H₂₉. In some embodiments, R⁵ is —C₁₅H₃₁. In some embodiments, R⁵ is —C₁₆H₃₃. In some embodiments, R⁵ is —C₁₇H₃₅. In some embodiments, R⁵ is —C₁₈H₃₇. In some embodiments, R⁵ is —C₁₉H₃₉. In some embodiments, R⁵ is —C₂₀H₄₁. In some embodiments, R⁵ is —C₂₁H₄₃. In some embodiments, R⁵ is —C₂₂H₄₅. In some embodiments, R⁵ is —C₂₃H₄₇. In some embodiments, R⁵ is —C₂₄H₂₉. In some embodiments, R⁵ is —C₂₅H₅₁. In some embodiments, R⁵ is —C₂₆H₅₃. In some embodiments, R⁵ is —C₂₇H₅₅. In some embodiments, R⁵ is —C₂₈H₅₇. In some embodiments, R⁵ is —C₂₉H₅₉. In some embodiments, R⁵ is —C₃₀H₆₁.

In some embodiments, R⁵ is a partially unsaturated straight-chain C₁₋₅₀ hydrocarbon. In some embodiments, R⁵ is —C₁₃H₂₅. In some embodiments, R⁵ is —C₁₅H₂₉. In some embodiments, R⁵ is —C₁₇H₃₃. In some embodiments, R⁵ is —C₁₉H₃₇. In some embodiments, R⁵ is —C₂₁H₄₁. In some embodiments, R⁵ is —C₁₇H₃₁. In some embodiments, R⁵ is —C₁₇H₂₉. In some embodiments, R⁵ is —C₁₉H₃₁. In some embodiments, R⁵ is —C₁₉H₂₉. In some embodiments, R⁵ is —C₂₁H₄₁. In some embodiments, R⁵ is —C₂₁H₃₁.

In some embodiments, R⁵ is -adamantane. In some embodiments, R⁵ is —CH₂adamantane. In some embodiments, R⁵ is —(CH₂)₁₋₁₀adamantane.

In some embodiments, R⁵ is

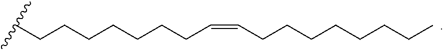 [SMILES: *CCCCCCC/C=C\CCCCCCCC]

In some embodiments, R⁵ is

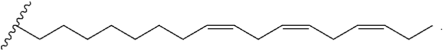 [SMILES: *CCCCCCC/C=C\C/C=C\C/C=C\CC]

In some embodiments, R⁵ is

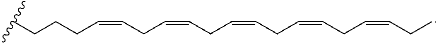 [SMILES: *CCC/C=C\C/C=C\C/C=C\C/C=C\C/C=C\CC]

In some embodiments, R⁵ is

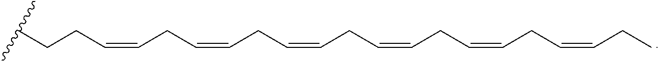 [SMILES: *CC/C=C\C/C=C\C/C=C\C/C=C\C/C=C\C/C=C\CC]

In some embodiments, R⁵ is selected from those depicted in Table 1, below.

As defined above and described herein, V is a bivalent group selected from —O—, —S—, and —NR—.

In some embodiments, V is —O—. In some embodiments, V is —S—. In some embodiments, V is —NR—.

In some embodiments, V is selected from those depicted in Table 1, below.

As defined above and described herein, W is a bivalent group selected from —O—, —S—, —NR—, —C(O)NR—, —OC(O)NR—, —SC(O)NR—,

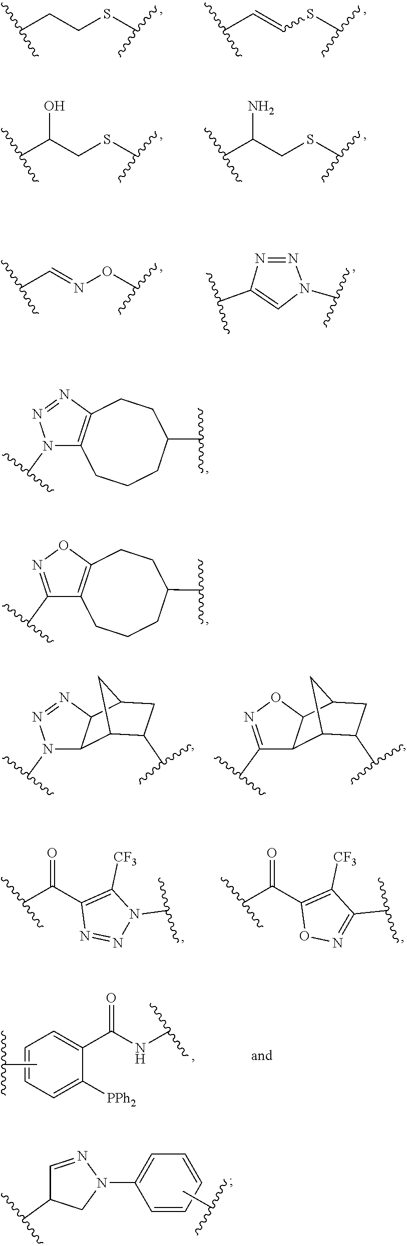

Without being limited to the current disclosure, the assembly of the nucleic acid or analogue thereof comprising lipid conjugates of the current disclosure can be facilitated using a range of cross-linking technologies. It is within the purview of those having ordinary skill in the art that W above or the coupling of lipophilic compounds to nucleic acids or analogue thereof described herein could be facilitated by suitable coupling moieties that react with each other to covalently link. Exemplary cross-linking technologies envisioned for use in the current disclosure also include those listed in Table A.

TABLE A
Exemplary Cross-linking Technologies

| Reaction | |
|---|---|
| Type | Reaction Summary |
| Thiol-yne | 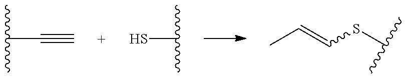 |
| NHS ester | 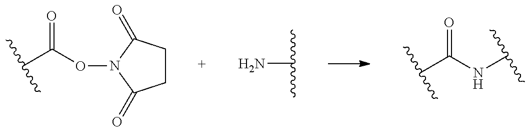 |
| Thiol-ene | 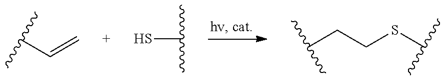 |
| Isocyanate | 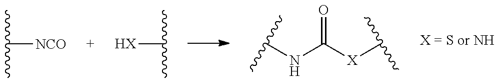 |
| Epoxide or aziridine | 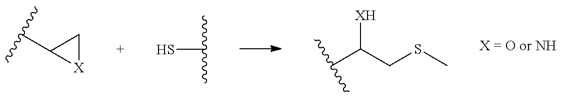 |
| Aldehyde- aminoxy | 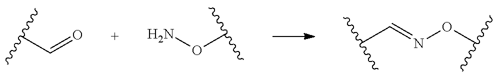 |
| Cu- catalyzed- azide-alkyne cyclo- addition | 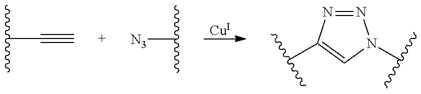 |
| Strain- promoted cyclo- addition | 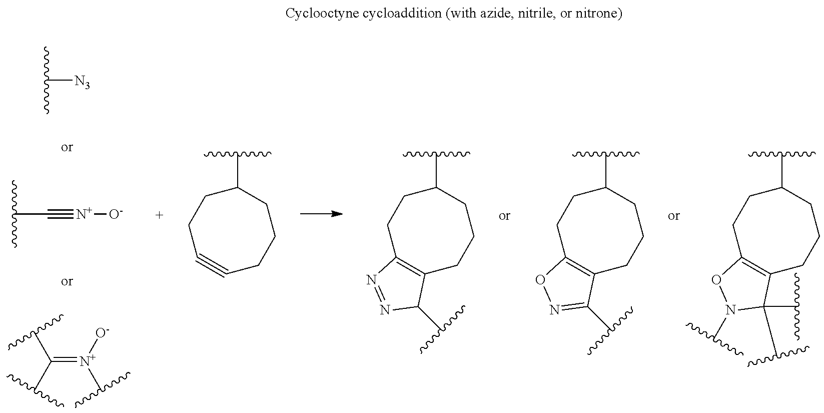 |
| | 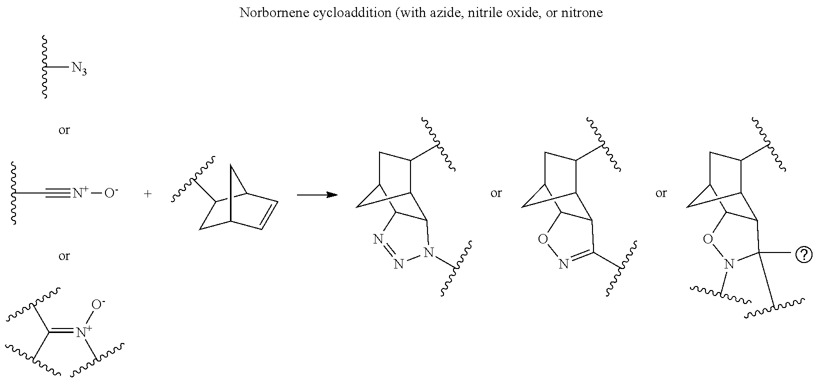 |
| | 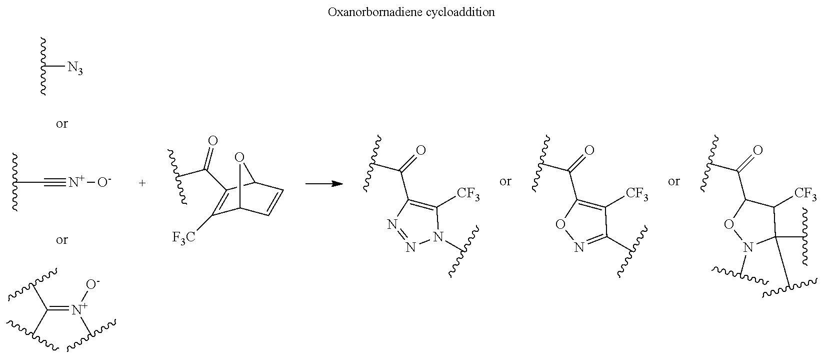 |
| Staudinger ligation | 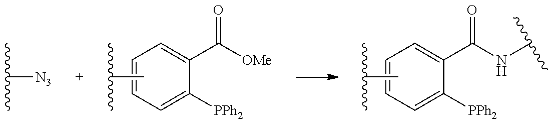 |
| Tetrazine ligation | 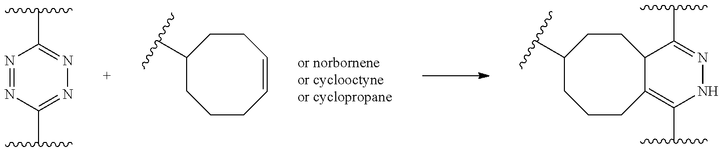 [SMILES: *c1nnc(*)nn1 ; *C1CC/C=C\CCC1 ; *C1=NNC(*)=C2CCCC(*)CCC12] |
| Photo- induced tetrazole- alkene cyclo- addition | 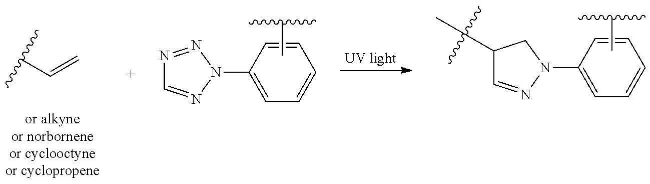 |
| [4 + 1] cyclo- addition | 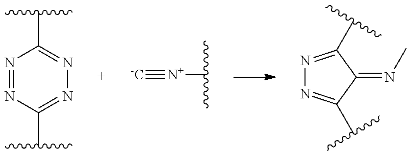 |
| Quadri- cyclane ligation | 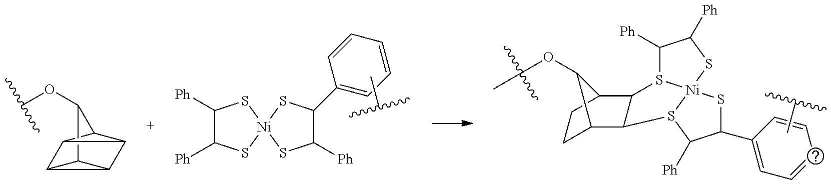 |

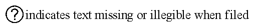

In some embodiments, W is —O—. In some embodiments, W is —S—, —NR—. In some embodiments, W is —C(O)NR—. In some embodiments, W is —OC(O)NR—. In some embodiments, W is —SC(O)NR—. In some embodiments, W is

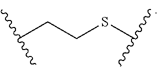 [SMILES: *CCS*]

In some embodiments, W is

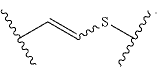 [SMILES: *C=CS*]

In some embodiments, W is

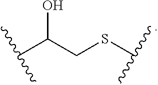 [SMILES: *SCC(*)O]

In some embodiments, W is

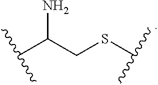 [SMILES: *SCC(*)N]

In some embodiments, W is

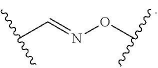 [SMILES: */C=N/O*]

In some embodiments, W is

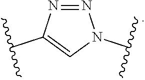 [SMILES: *c1cn(*)nn1]

In some embodiments, W is

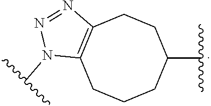 [SMILES: *C1CCCc2c(nnn2*)CC1]

In some embodiments, W is

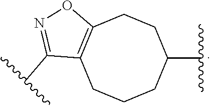 [SMILES: *c1noc2c1CCCC(*)CC2]

In some embodiments, W is

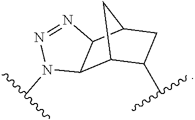 [SMILES: *C1CC2CC1C1C2N=NN1*]

In some embodiments, W is

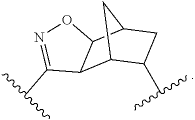 [SMILES: *C1=NOC2C3CC(*)C(C3)C12]

In some embodiments, W is

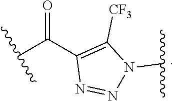 [SMILES: *C(=O)c1nnn(*)c1C(F)(F)F]

In some embodiments, W is

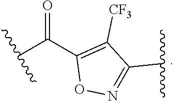 [SMILES: *C(=O)c1onc(*)c1C(F)(F)F]

In some embodiments, W is

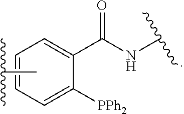

In some embodiments, W is

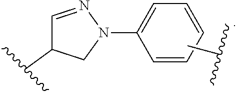

In some embodiments, W is selected from those depicted in Table 1, below.

As defined below and described herein, X is hydrogen, a suitable protecting group or a cross-linking group.

In some embodiments, X is hydrogen. In some embodiments, X is a suitable protecting group. In some embodiments, X is a cross-linking group. In some embodiments, the cross-linking group is —OH, —SH, —NHR, —COH, —CO₂H, —N₃, alkyne, alkene, including any of the cross-linking groups mentioned in Table A.

In some embodiments, X is selected from those depicted in Table 1, below.

As defined above and described herein, X¹ is —O—, —S—, —Se—, or —NR—.

In some embodiments, X¹ is —O—. In some embodiments, X¹ is —S—. In some embodiments, X¹ is —Se—. In some embodiments, X¹ is —NR—.

In some embodiments, X¹ is selected from those depicted in Table 1, below.

As defined above and described herein, X² is O, S, or NR.

In some embodiments, X² is O. In some embodiments, X² is S. In some embodiments, X² is NR.

In some embodiments, X² is selected from those depicted in Table 1, below.

As defined above and described herein, X³ is —O—, —S—, —BH₂—, or a covalent bond.

In some embodiments, X³ is —O—. In some embodiments, X³ is —S—. In some embodiments, X³ is —BH₂—. In some embodiments, X³ and R⁴ form —BH₃. In some embodiments, X³ is a covalent bond. In some embodiments, X³ is a covalent bond that constitutes a boranophosphate backbone.

In some embodiments, X³ is selected from those depicted in Table 1, below.

As defined above and described herein, Y is hydrogen, a suitable hydroxyl protecting group

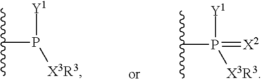

In some embodiments, Y is hydrogen. In some embodiments, Y is a suitable hydroxyl protecting group. In some embodiments, Y is

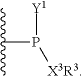

In some embodiments, Y is

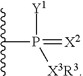

In some embodiments, Y is selected from those depicted in Table 1, below.

As defined above and described herein, Y¹ is a linking group attaching to the 2′- or 3′-terminal of a nucleoside, a nucleotide, or an oligonucleotide.

In some embodiments, Y¹ is a linking group attaching to the 2′-terminal of a nucleoside, a nucleotide, or an oligonucleotide. In some embodiments, Y¹ is a linking group attaching to the 3′-terminal of a nucleoside, a nucleotide, or an oligonucleotide.

In some embodiments, Y¹ is

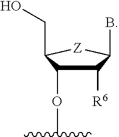

In some embodiments, Y¹ is

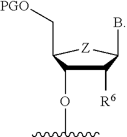

In some embodiments, Y¹ is

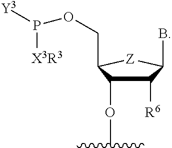

In some embodiments, Y¹ is

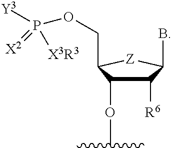

In some embodiments, Y¹ is selected from those depicted in Table 1, below.

As defined above and described herein, Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support.

In some embodiments, Y² is hydrogen. In some embodiments, Y² is a suitable protecting group. In some embodiments, Y² is a phosphoramidite analogue. In some embodiments, Y² is a phosphoramidite analogue of formula:

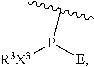

wherein each of R³ and X³ are independently as described herein, and E is a halogen or —NR₂. In some embodiments, Y² is an internucleotide linking group attaching to the 5′-terminal of a nucleoside, a nucleotide, or an oligonucleotide. In some embodiments, Y² is a linking group attaching to a solid support.

In some embodiments, Y² is benzoyl. In some embodiments, Y² is t-butyldimethylsilyl. In some embodiments, Y² is

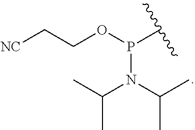 [SMILES: *P(OCCC#N)N(C(C)C)C(C)C]

In some embodiments, Y² is

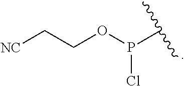 [SMILES: *P(Cl)OCCC#N]

In some embodiments, Y² is

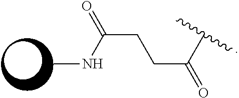 [SMILES: *C(=O)CCC(=O)NC]

In some embodiments, Y² is

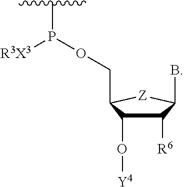

In some embodiments, Y² is

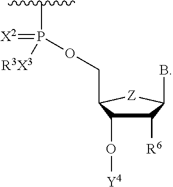

In some embodiments, Y² is selected from those depicted in Table 1, below.

As shown above in an embodiment, E is a halogen or —NR₂.

In some embodiments, E is a halogen. In some embodiments, E is —NR₂. In some embodiments, E is a chloro. In some embodiments, E is —N(iPr)₂.

In some embodiments, E is selected from those depicted in Table 1, below.

As shown above in some embodiments of Y¹, Y³ is a linking group attaching to the 2′- or 3′-terminal of a nucleoside, a nucleotide, or an oligonucleotide.

In some embodiments, Y³ is a linking group attaching to the 2′-terminal of a nucleoside, a nucleotide, or an oligonucleotide. In some embodiments, Y³ is a linking group attaching to the 3′-terminal of a nucleoside, a nucleotide, or an oligonucleotide.

In some embodiments, Y³ is selected from those depicted in Table 1, below.

As shown above in some embodiments of Y², Y⁴ is hydrogen, a protecting group, a phosphoramidite analogue, an internucleotide linking group attaching to the 4′- or 5′-terminal of a nucleoside, a nucleotide, or an oligonucleotide, or a linking group attaching to a solid support.

In some embodiments, Y⁴ is hydrogen. In some embodiments, Y⁴ is a protecting group. In some embodiments, Y⁴ is a phosphoramidite analogue. In some embodiments, Y⁴ is a phosphoramidite analogue of formula:

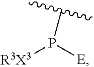

wherein each of R³, X³, and E is independently as described herein. In some embodiments, Y⁴ is an internucleotide linking group attaching to the 4′-terminal of a nucleoside, a nucleotide, or an oligonucleotide. In some embodiments, Y⁴ is an internucleotide linking group attaching to the 5′-terminal of a nucleoside, a nucleotide, or an oligonucleotide. In some embodiments, Y⁴ is a linking group attaching to a solid support.

In some embodiments, Y⁴ is benzoyl. In some embodiments, Y⁴ is t-butyldimethylsilyl. In some embodiments, Y⁴ is

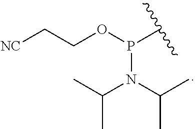 [SMILES: *P(OCCC#N)N(C(C)C)C(C)C]

In some embodiments, Y⁴ is

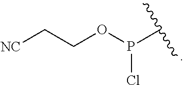 [SMILES: *P(Cl)OCCC#N]

In some embodiments, Y⁴ is

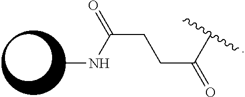 [SMILES: *C(=O)CCC(=O)NC]

In some embodiments, Y⁴ is selected from those depicted in Table 1, below.

As defined above and described herein, each R⁶ is independently hydrogen, a suitable prodrug, R^{A}, halogen, —CN, —NO₂, —OR, —SR, —NR₂, —Si(OR)₂R, —Si(OR)R₂, —S(O)₂R, —S(O)₂NR₂, —S(O)R, —C(O)R, —C(O)OR, —C(O)NR₂, —C(O)N(R)OR, —OC(O)R, —OC(O)NR₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —N(R)P(O)R₂, —N(R)P(O)(OR)₂, —N(R)P(O)(OR)NR₂, —N(R)P(O)(NR₂)₂, —N(R)S(O)₂R, —Si(OR)₂R, —Si(OR)R₂, or —SiR₃.

In some embodiments, R⁶ is hydrogen. In some embodiments, R⁶ is deuterium. In some embodiments, R⁶ is a suitable prodrug. In some embodiments, R⁶ is a suitable phosphate/phosphonate prodrug, which is a glutathione-sensitive moiety. In some embodiments, R⁶ is a glutathione-sensitive moiety selected from those as described in International Patent Application No. PCT/US2013/072536, which is hereby incorporated by reference in its entirety. In some embodiments, R⁶ is R^{A}. In some embodiments, R⁶ is halogen. In some embodiments, R⁶ is —CN. In some embodiments, R⁶ is —NO₂. In some embodiments, R⁶ is —OR. In some embodiments, R⁶ is —SR. In some embodiments, R⁶ is —NR₂. In some embodiments, R⁶ is —S(O)₂R. In some embodiments, R⁶ is —S(O)₂NR₂. In some embodiments, R⁶ is —S(O)R. In some embodiments, R⁶ is —C(O)R. In some embodiments, R⁶ is —C(O)OR. In some embodiments, R⁶ is —C(O)NR₂. In some embodiments, R⁶ is —C(O)N(R)OR. In some embodiments, R⁶ is —C(R)₂N(R)C(O)R. In some embodiments, R⁶ is —C(R)₂N(R)C(O)NR₂. In some embodiments, R⁶ is —OC(O)R. In some embodiments, R⁶ is —OC(O)NR₂. In some embodiments, R⁶ is —OP(O)R₂. In some embodiments, R⁶ is —OP(O)(OR)₂. In some embodiments, R⁶ is —OP(O)(OR)NR₂. In some embodiments, R⁶ is —OP(O)(NR₂)₂—. In some embodiments, R⁶ is —N(R)C(O)OR. In some embodiments, R⁶ is —N(R)C(O)R. In some embodiments, R⁶ is —N(R)C(O)NR₂. In some embodiments, R⁶ is —N(R)P(O)R₂. In some embodiments, R⁶ is —N(R)P(O)(OR)₂. In some embodiments, R⁶ is —N(R)P(O)(OR)NR₂. In some embodiments, R⁶ is —N(R)P(O)(NR₂)₂. In some embodiments, R⁶ is —N(R)S(O)₂R. In some embodiments, R⁶ is —Si(OR)₂R. In some embodiments, R⁶ is —Si(OR)R₂. In some embodiments, R⁶ is —SiR₃.

In some embodiments, R⁶ is hydroxyl. In some embodiments, R⁶ is fluoro. In some embodiments, R⁶ is methoxy. In some embodiments, R⁶ is

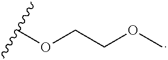 [SMILES: *OCCOC]

In some embodiments, R⁶ is selected from those depicted in Table 1.

As defined above and described herein, E is a halogen or —NR₂.

In some embodiments, E is a halogen. In some embodiments, E is —NR₂.

In some embodiments, E is selected from those depicted in Table 1, below.

As defined above and described herein, Z is —O—, —S—, —NR—, or —CR₂—.

In some embodiments, Z is —O—. In some embodiments, Z is —S—. In some embodiments, Z is —NR—. In some embodiments, Z is —CR₂—.

In some embodiments, Z is selected from those depicted in Table 1, below.

As defined above and described herein, PG¹ is hydrogen or a suitable hydroxyl protecting group.

In some embodiments, PG¹ is hydrogen. In some embodiments, PG¹ is a suitable hydroxyl protecting group.

As defined above and described herein, PG² is hydrogen, a phosphoramidite analogue, or a suitable protecting group.

In some embodiments, PG² is hydrogen. In some embodiments, PG¹ is a phosphoramidite analogue. In some embodiments, PG² is a hydroxyl protecting group.

In some embodiments, each of PG¹ and PG², taken with the oxygen atom to which it is bound, is independently selected from the suitable hydroxyl protecting groups described above for Y. In some embodiments, PG¹ and PG² are taken together with their intervening atoms to form a cyclic diol protecting group, such as a cyclic acetal or ketal. Such groups include methylene, ethylidene, benzylidene, isopropylidene, cyclohexylidene, and cyclopentylidene, silylene derivatives such as di-t-butylsilylene and 1,1,3,3-tetraisopropylidisiloxanylidene, a cyclic carbonate, a cyclic boronate, and cyclic monophosphate derivatives based on cyclic adenosine monophosphate (i.e., cAMP). In some embodiments, the cyclic diol protection group is 1,1,3,3-tetraisopropylidisiloxanylidene.

In some embodiments, PG¹ and PG² are selected from those depicted in Table 1, below.

As defined above and described herein, PG³ is hydrogen or a suitable amine protecting group.

In some embodiments, PG³ is hydrogen. In some embodiments, PG³ is a suitable amine protecting group. In some embodiments, PG³ and R⁴ for a cyclic amine protecting group (e.g., phthalimide).

In some embodiments, PG³ are selected from those depicted in Table 1, below.

As defined above and described herein, PG⁴ is hydrogen or a suitable hydroxyl protecting group.

In some embodiments, PG⁴ is hydrogen. In some embodiments, PG⁴ is a suitable hydroxyl protecting group.

In some embodiments, PG⁴ are selected from those depicted in Table 1, below.

TABLE 1
Exemplary Nucleic Acid-lipid conjugates

| | |
|---|---|
| 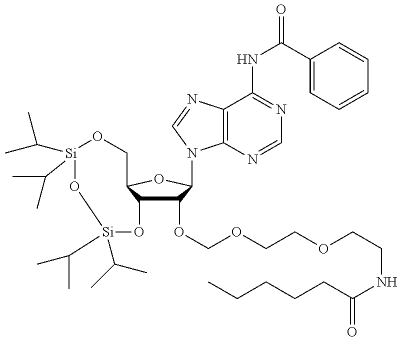 | 2- 1a |
| 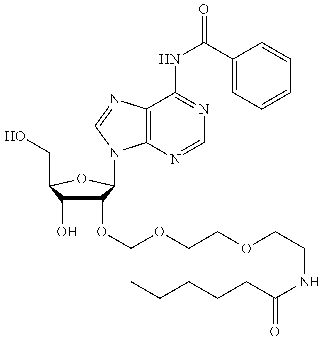 | 2- 2a |
| 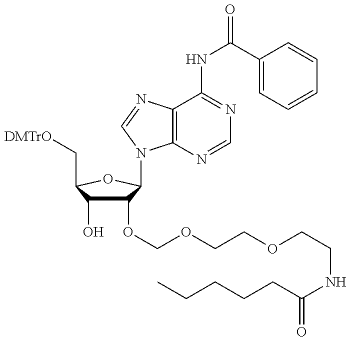 | 2- 3a |
| 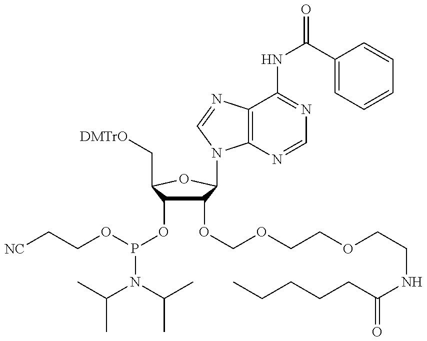 | 2- 4a |
| 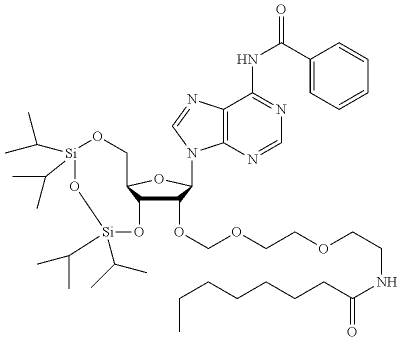 | 2- 1b |
| 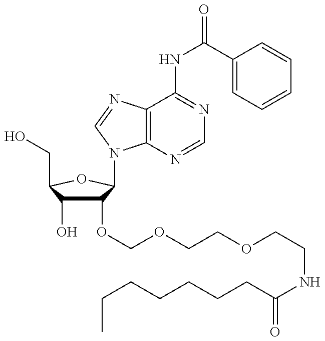 | 2- 2b |
| 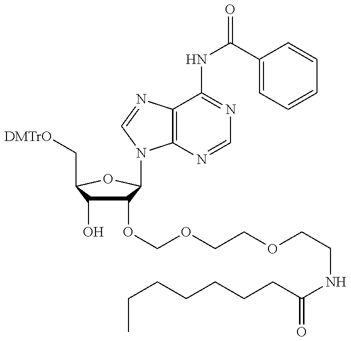 | 2- 3b |
| 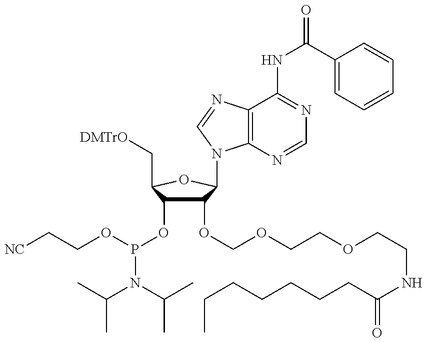 | 2- 4b |
| 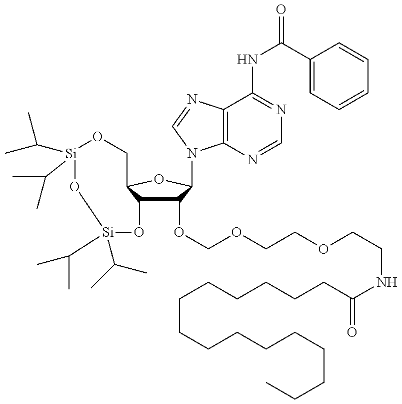 | 2- 1c |
| 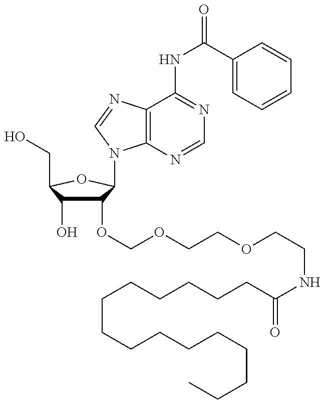 | 2- 2c |
| 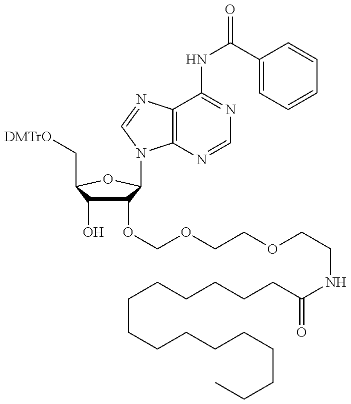 | 2- 3c |
| 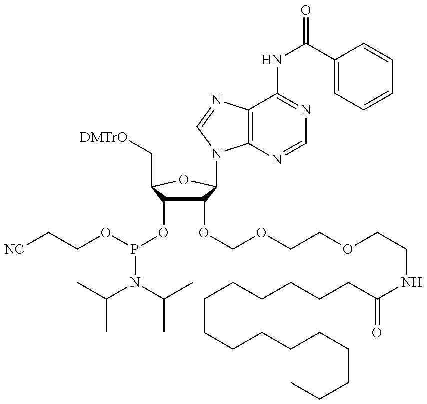 | 2- 4c |
| 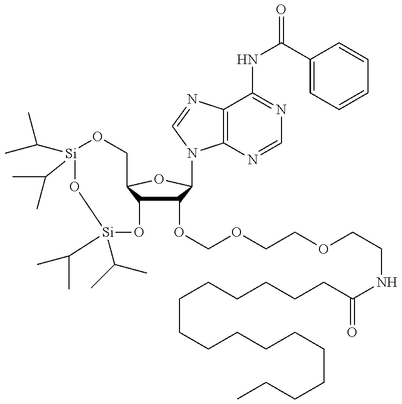 | 2- 1d |
| 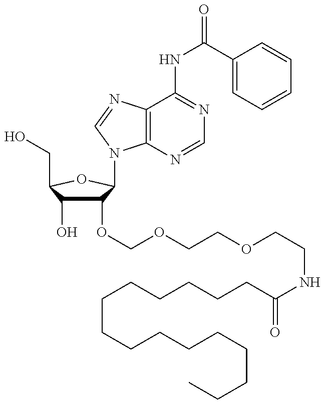 | 2- 2d |
| 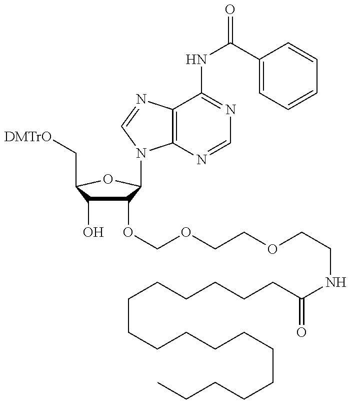 | 2- 3d |
| 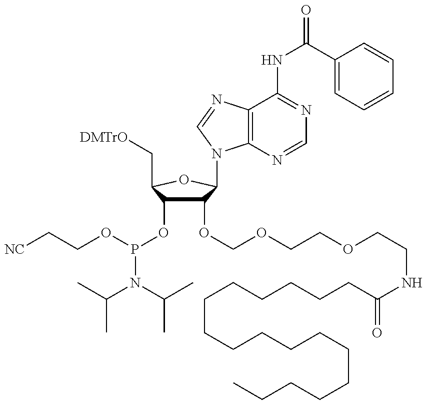 | 2- 4d |
| 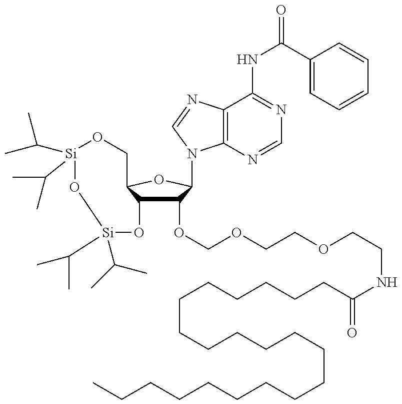 | 2- 1e |
| 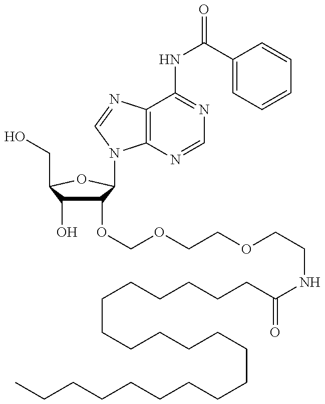 | 2- 2e |
| 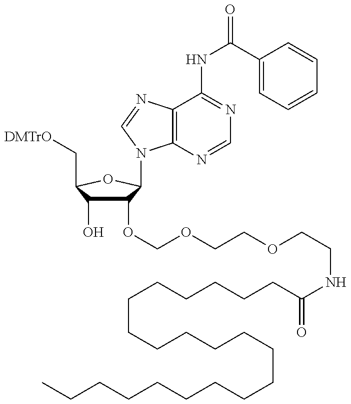 | 2- 3e |
| 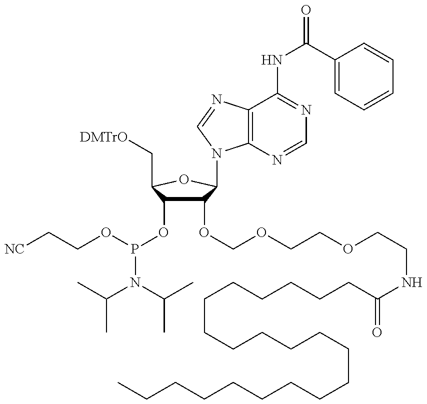 | 2- 4e |

In some embodiments, the present disclosure provides a nucleic acid or analogue thereof comprising a lipid conjugate of the disclosure set forth in Table 1, above, or a pharmaceutically acceptable salt thereof.

In some embodiments, the present disclosure provides an oligonucleotide-ligand conjugate comprising one or more nucleic acid-lipid conjugates of the disclosure, as described in the examples, or a pharmaceutically acceptable salt thereof.

### 3. Definitions

Compounds of the present disclosure (i.e., nucleic acid-ligand conjugates, oligonucleotide-ligand conjugates and analogues thereof) include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl. A carbocyclyl group may be monocyclic, bicyclic, bridged bicyclic, spirocyclic, or adamantane.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally, or alternatively, any substitutable nitrogen of a bridged bicyclic group is optionally substituted. Exemplary bridged bicyclics include:

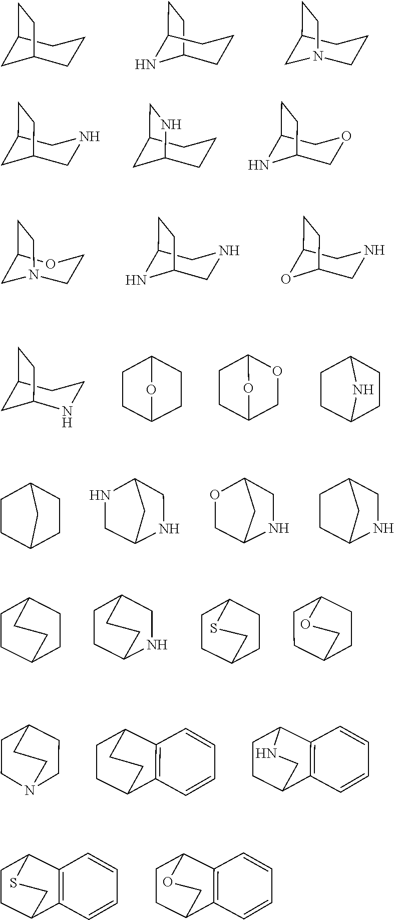

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

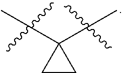 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 □ electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be monocyclic, bicyclic, bridged bicyclic, or spirocyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR—, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘} taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR′₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the disclosure. Unless otherwise stated, all tautomeric forms of the compounds of the disclosure are within the scope of the disclosure. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this disclosure. Such compounds are useful, for example, as analytical tools, as probes in biological assays, or as therapeutic agents in accordance with the present disclosure

As used herein, the singular forms “a,” “an,” and “the” include plural references unless the context clearly dictates otherwise. For example, a reference to “a method” includes one or more methods, and/or steps of the type described herein and/or which will become apparent to those persons skilled in the art upon reading this disclosure and so forth.

As used herein, the term “and/or” is used in this disclosure to mean either “and” or “or” unless indicated otherwise.

### 4. Oligonucleotide-Ligand Conjugates for Reducing Gene Expression

Another aspect discloses an oligonucleotide-ligand conjugate for reducing expression of a target gene, wherein the nucleic acid-conjugate unit is represented by formula II:

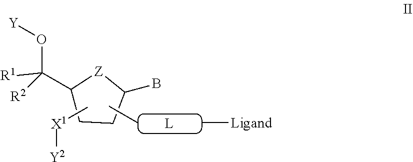

or a pharmaceutically acceptable salt thereof, wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃; or
   R¹ and R² on the same carbon are taken together with their intervening atoms to form a 3-7 membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur; or two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
ligand is independently -(LC)ₙ, or an adamantyl group;
each LC is independently a lipid conjugate moiety comprising a saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —N(R)—C(O)—, —S—, —C(O)—, —S(O)—, —S(O)₂—, —P(O)OR—, —P(S)OR—, —V¹CR²W¹—, or

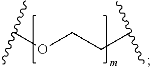

m is 1-50;
X¹, V¹ and W¹ are independently —C(R)₂—, —OR, —O—, —S—, —Se—, or —NR—;
Y is hydrogen, a suitable hydroxyl protecting group,

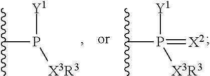

R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, S, or NR;
X³ is —O—, —S—, —BH₂—, or a covalent bond;
Y¹ is a linking group attaching to the 2′- or 3′-terminal of a nucleoside, a nucleotide, or an oligonucleotide;
Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support;
Z is —O—, —S—, —NR—, or —CR₂—; and
wherein the oligonucleotide comprises a sense strand of 15-53 nucleotides in length and an antisense strand of 19-53 nucleotides in length, wherein the antisense oligonucleotide strand has sequence complementary to at least 15 consecutive nucleotides of a target gene sequence;
and wherein the antisense strand and the sense strand form a duplex structure but are not covalently linked.

In some embodiments, the oligonucleotide-ligand conjugate of any one of the above mentioned aspects or embodiments comprises one or more nucleic acid-ligand conjugate unit selected from the formula I, I-a, I-b, I-c, I-d, I-e, I-Ia, I-Ib, I-Ic, I-Id, I-Ie, II, II-a, II-b, II-c, II-d, II-e, II-Ia, II-Ib, II-Ic, II-Id and IT-Ie, or a pharmaceutically acceptable salt thereof/

In certain embodiments, the oligonucleotide-ligand conjugate of any one of the above mentioned aspects or embodiments, the oligonucleotide comprises a sense strand of 15-53 nucleotides in length and an antisense strand of 19-53 nucleotides in length, wherein the antisense oligonucleotide strand has sequence complementary to at least 15 consecutive nucleotides of a target gene sequence and reduces the gene expression when the oligonucleotide-conjugate is introduced into a mammalian cell.

In some embodiments, the region of complementarity is fully complementary to at least 15, at least 16, at least 17, at least 18, at least 19, at least 20, or at least 21 contiguous nucleotides of the target mRNA. In some embodiments, L is a tetraloop. In some embodiments, L is 4 nucleotides in length. In some embodiments, L comprises a sequence set forth as GAAA. In some embodiments, the antisense strand is 21 to 27 nucleotides in length and the sense strand is 12, 15, 20 or 25 nucleotides in length. In some embodiments, the antisense strand and sense strand form a duplex region of 25 nucleotides in length. In some embodiments, the duplex has blunt ends. In certain embodiments, the duplex has a tetraloop.

In certain oligonucleotide-ligand conjugate embodiments, the nucleic acid-ligand conjugate units are present in the sense strand.

In some oligonucleotide-ligand conjugate embodiments, the antisense strand is 19 to 27 nucleotides in length.

In some oligonucleotide-ligand conjugate embodiments, the sense strand is 12 to 40 nucleotides in length.

In some oligonucleotide-ligand conjugate embodiments, the sense strand forms a duplex region with the antisense strand. In certain embodiments, the duplex has blunt ends. In some embodiments, the sense strand is truncated.

In some oligonucleotide-ligand conjugate embodiments, the region of complementarity is fully complementary to the target sequence.

In certain embodiments, the sense strand has a sequence

5′ GGUGGAUGAAACUCAGUUUAGCAGCCGAAAGGCUGC.

In certain embodiments, the antisense strand has a sequence

3′ GGCCACCUACUUUGAGUCAAAU.

In some oligonucleotide-ligand conjugate embodiments, wherein the sense strand comprises at its 3′-end a stem-loop set forth as: S₁-L-S₂, wherein S₁ is complementary to S₂, and wherein L forms a loop between S₁ and S₂ of 3 to 5 nucleotides in length.

In some oligonucleotide-ligand conjugate embodiments, L is a tetraloop. In certain embodiments, L comprises a sequence set forth as GAAA

In some oligonucleotide-ligand conjugate embodiments, the conjugate further comprises a 3′-overhang sequence on the antisense strand of two nucleotides in length. In certain embodiments, the oligonucleotide further comprises a 3′-overhang sequence of one or more nucleotides in length, wherein the 3′-overhang sequence is present on the antisense strand, the sense strand, or the antisense strand and sense strand.

In some oligonucleotide-ligand conjugate embodiments, the oligonucleotide comprises at least one modified nucleotide. In certain embodiments, the modified nucleotide comprises a 2′-modification. In some embodiments, the 2′-modification is a modification selected from: 2′-aminoethyl, 2′-fluoro, 2′-O-methyl, 2′-O-methoxyethyl, and 2′-deoxy-2′-fluoro-β-d-arabinonucleic acid.

In some oligonucleotide-ligand conjugate embodiments, all the nucleotides of the oligonucleotide are modified.

In some oligonucleotide-ligand conjugate embodiments, the oligonucleotide comprises at least one modified internucleotide linkage. In certain embodiments, the at least one modified internucleotide linkage is a phosphorothioate linkage.

In some oligonucleotide-ligand conjugate embodiments, the 4′-carbon of the sugar of the 5′-nucleotide of the antisense strand comprises a phosphate analog. In certain embodiments, the phosphate analog is oxymethylphosphonate, vinylphosphonate, or malonylphosphonate.

As used herein, the term “4′-O-methylene phosphonate” refers all substituted methylene analogues (e.g., methylene substituted with methyl, dimethyl, ethyl, fluoro, cyclopropyl, etc.) and all phosphonate analogues (e.g., phosphorothioate, phosphorodithioate, phosphodiester etc.) described herein.

As used herein, the term “5′-terminal nucleotide” refers to the nucleotide located at the 5′-end of an oligonucleotide. The 5′-terminal nucleotide may also be referred to as the “N₁ nucleotide” in this application.

As used herein, the term, “alcoholism” refers to repeated use of ethanol by an individual despite recurrent adverse consequences, which may or may not be combined with tolerance, withdrawal, and/or an uncontrollable drive to consume alcohol. Alcoholism may be classified as alcohol abuse, alcohol use disorder or alcohol dependence. A variety of approaches may be used to identify an individual suffering from alcoholism. For example, the World Health Organization has established the Alcohol Use Disorders Identification Test (AUDIT) as a tool for identifying potential alcohol misuse, including dependence and other similar tests have been developed, including the Michigan Alcohol Screening Test (MAST). Laboratory tests may be used to evaluate blood markers for detecting chronic use and/or relapse in alcohol drinking, including tests to detect levels of gamma-glutamyl transferase (GGT), mean corpuscular volume (red blood cell size), aspartate aminotransferase (AST), alanine aminotransferase (ALT), carbohydrate-deficient transferring (CDT), ethyl glucuronide (EtG), ethyl sulfate (EtS), and/or phosphatidylethanol (PEth).

Animal models (e.g., mouse models) of alcoholism have been established (see, e.g., Rijk H, Crabbe J C, Rigter H., A Mouse Model of Alcoholism, PHYSIOL BEHAV. (1982) November; 29(5):833-39; Elizabeth Brandon-Warner, et al., Rodent Models of Alcoholic Liver Disease: of Mice and Men, ALCOHOL. 2012 December; 46(8): 715-25 (2012 December; 46(8)): and, Adeline Bertola, et al., Mouse Model of Chronic and Binge Ethanol Feeding (the NIAAA model). NATURE PROTOCOLS 8, 627-37 (2013))

As used herein, the term, “ALDH2” refers to the aldehyde dehydrogenase 2 family (mitochondrial) gene. ALDH2 encodes proteins that belong to the aldehyde dehydrogenase family of proteins and that function as the second enzyme of the oxidative pathway of alcohol metabolism that synthesizes acetate (acetic acid) from ethanol. Homologs of ALDH2 are conserved across a range of species, including human, mouse, rat, non-human primate species, and others (see, e.g., NCBI HomoloGene:55480). ALDH2 also has homology with other aldehyde dehydrogenase encoding genes, including, for example, ALDH1A1. In humans, ALDH2 encodes at least two transcripts, namely NM_000690.3 (variant 1) and NM_001204889.1 (variant 2), each encoding a different isoform, NP_000681.2 (isoform 1) and NP_001 191818.1 (isoform 2), respectively. Transcript variant 2 lacks an in-frame exon in the 5′ coding region, compared to transcript variant 1, and encodes a shorter isoform (2), compared to isoform 1. Polymorphisms in ALDH2 have been identified (see, e.g., Chang J S, Hsiao J R, Chen C H., ALDH2 polymorphism and alcohol-related cancers in Asians: a public health perspective, J BIOMED SCI. (2017 Mar. 3); 24(1): 19 Review).

As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%1, 16%%, 15%, 14%, 13%, %12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the terms “administering” or “administration” means to provide a substance (e.g., an oligonucleotide) to a subject in a manner that is pharmacologically useful (e.g., to treat a condition in the subject).

As used herein, the term “Asialoglycoprotein receptor” or “ASGPR” refers to a bipartite C-type lectin formed by a major 48 kDa (ASGPR-l) and minor 40 kDa subunit (ASGPR-2). ASGPR is primarily expressed on the sinusoidal surface of hepatocyte cells and has a major role in binding, internalization, and subsequent clearance of circulating glycoproteins that contain terminal galactose or N-acetylgalactosamine residues (asialoglycoproteins).

As used herein, the term “aptamer” refers to an oligonucleotide that has binding affinity for a specific target including a nucleic acid, a protein, a specific whole cell or a particular tissue. Aptamers may be obtained using methods known in the art, for example, by in vitro selection from a large random sequence pool of nucleic acids. Lee et al., NUCLEIC ACID RES., 2004, 32:D95-D100.

As used herein, the term “antagomir” refers to an oligonucleotide that has binding affinity for a specific target including the guide strand of an exogenous RNAi inhibitor molecule or natural miRNA (Krutzfeldt et al., NATURE 2005, 438(7068):685-89).

A double stranded RNAi inhibitor molecule comprises two oligonucleotide strands: an antisense strand and a sense strand. The antisense strand or a region thereof is partially, substantially or fully complementary to a corresponding region of a target nucleic acid. In addition, the antisense strand of the double stranded RNAi inhibitor molecule or a region thereof is partially, substantially or fully complementary to the sense strand of the double stranded RNAi inhibitor molecule or a region thereof. In certain embodiments, the antisense strand may also contain nucleotides that are non-complementary to the target nucleic acid sequence. The non-complementary nucleotides may be on either side of the complementary sequence or may be on both sides of the complementary sequence. In certain embodiments, where the antisense strand or a region thereof is partially or substantially complementary to the sense strand or a region thereof, the non-complementary nucleotides may be located between one or more regions of complementarity (e.g., one or more mismatches). The antisense strand of a double stranded RNAi inhibitor molecule is also referred to as the guide strand.

As used herein, the term “canonical RNA inhibitor molecule” refers to two strands of nucleic acids, each 21 nucleotides long with a central region of complementarity that is 19 base-pairs long for the formation of a double stranded nucleic acid and two nucleotide overhands at each of the 3′-ends.

As used herein, the term “complementary” refers to a structural relationship between two nucleotides (e.g., on two opposing nucleic acids or on opposing regions of a single nucleic acid strand) that permits the two nucleotides to form base pairs with one another. For example, a purine nucleotide of one nucleic acid that is complementary to a pyrimidine nucleotide of an opposing nucleic acid may base pair together by forming hydrogen bonds with one another. In some embodiments, complementary nucleotides can base pair in the Watson-Crick manner or in any other manner that allows for the formation of stable duplexes. “Fully complementarity” or 100% complementarity refers to the situation in which each nucleotide monomer of a first oligonucleotide strand or of a segment of a first oligonucleotide strand can form a base pair with each nucleotide monomer of a second oligonucleotide strand or of a segment of a second oligonucleotide strand. Less than 100% complementarity refers to the situation in which some, but not all, nucleotide monomers of two oligonucleotide strands (or two segments of two oligonucleotide strands) can form base pairs with each other. “Substantial complementarity” refers to two oligonucleotide strands (or segments of two oligonucleotide strands) exhibiting 90% or greater complementarity to each other. “Sufficiently complementary” refers to complementarity between a target mRNA and a nucleic acid inhibitor molecule, such that there is a reduction in the amount of protein encoded by a target mRNA.

As used herein, the term “complementary strand” refers to a strand of a double stranded nucleic acid inhibitor molecule that is partially, substantially or fully complementary to the other strand.

As used herein, the term “conventional antisense oligonucleotide” refers to single stranded oligonucleotides that inhibit the expression of a targeted gene by one of the following mechanisms: (1) Steric hindrance, e.g., the antisense oligonucleotide interferes with some step in the sequence of events involved in gene expression and/or production of the encoded protein by directly interfering with, for example, transcription of the gene, splicing of the pre-mRNA and translation of the mRNA; (2) Induction of enzymatic digestion of the RNA transcripts of the targeted gene by RNase H; (3) Induction of enzymatic digestion of the RNA transcripts of the targeted gene by RNase L; (4) Induction of enzymatic digestion of the RNA transcripts of the targeted gene by RNase P: (5) Induction of enzymatic digestion of the RNA transcripts of the targeted gene by double stranded RNase; and (6) Combined steric hindrance and induction of enzymatic digestion activity in the same antisense oligo. Conventional antisense oligonucleotides do not have an RNAi mechanism of action like RNAi inhibitor molecules. RNAi inhibitor molecules can be distinguished from conventional antisense oligonucleotides in several ways including the requirement for Ago2 that combines with an RNAi antisense strand such that the antisense strand directs the Ago2 protein to the intended target(s) and where Ago2 is required for silencing of the target.

Clustered Regularly Interspaced Short Palindromic Repeats (“CRISPR”) is a microbial nuclease system involved in defense against invading phages and plasmids. Wright et al., Cell, 2016, 164:29-44. This prokaryotic system has been adapted for use in editing target nucleic acid sequences of interest in the genome of eukaryotic cells. Cong et al., SCIENCE, 2013, 339:819-23; Mali et al., SCIENCE, 2013, 339:823-26; Woo Cho et al., NAT. BIOTECHNOLOGY, 2013, 31(3):230-232. As used herein, the term “CRISPR RNA” refers to a nucleic acid comprising a “CRISPR” RNA (crRNA) portion and/or a trans activating crRNA (tracrRNA) portion, wherein the CRISPR portion has a first sequence that is partially, substantially or fully complementary to a target nucleic acid and a second sequence (also called the tracer mate sequence) that is sufficiently complementary to the tracrRNA portion, such that the tracer mate sequence and tracrRNA portion hybridize to form a guide RNA. The guide RNA forms a complex with an endonuclease, such as a Cas endonuclease (e.g., Cas9) and directs the nuclease to mediate cleavage of the target nucleic acid. In certain embodiments, the crRNA portion is fused to the tracrRNA portion to form a chimeric guide RNA. Jinek et al., SCIENCE, 2012, 337:816-21. In certain embodiments, the first sequence of the crRNA portion includes between about 16 to about 24 nucleotides, preferably about 20 nucleotides, which hybridize to the target nucleic acid. In certain embodiments, the guide RNA is about 10-500 nucleotides. In other embodiments, the guide RNA is about 20-100 nucleotides.

As used herein, the term “delivery agent” refers to a transfection agent or a ligand that is complexed with or bound to an oligonucleotide and which mediates its entry into cells. The term encompasses cationic liposomes, for example, which have a net positive charge that binds to the oligonucleotide's negative charge. This term also encompasses the conjugates as described herein, such as GalNAc and cholesterol, which can be covalently attached to an oligonucleotide to direct delivery to certain tissues. Further specific suitable delivery agents are also described herein.

As used herein, the term “deoxyribonucleotide” refers to a nucleotide which has a hydrogen group at the 2′-position of the sugar moiety. A modified deoxyribonucleotide is a deoxyribonucleotide having one or more modifications or substitutions of atoms other than at the 2′ position, including modifications or substitutions in or of the sugar, phosphate group or base.

As used herein, the term “disulfide” refers to a chemical compound containing the group

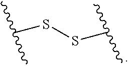 [SMILES: *SS*]

Typically, each sulfur atom is covalently bound to a hydrocarbon group. In certain embodiments, at least one sulfur atom is covalently bound to a group other than a hydrocarbon. The linkage is also called an SS-bond or a disulfide bridge.

As used herein, the term “double-stranded oligonucleotide” or “double stranded nucleic acid (dsNA)” refers to an oligonucleotide that is substantially in a duplex form. In some embodiments, complementary base-pairing of duplex region(s) of a double-stranded oligonucleotide is formed between antiparallel sequences of nucleotides of covalently separate nucleic acid strands. In some embodiments, complementary base-pairing of duplex region(s) of a double-stranded oligonucleotide is formed between antiparallel sequences of nucleotides of nucleic acid strands that are covalently linked. In some embodiments, complementary base pairing of duplex region(s) of a double-stranded oligonucleotide is formed from a single nucleic acid strand that is folded (e.g., via a hairpin loop) to provide complementary antiparallel sequences of nucleotides that base pair together. In some embodiments, a double-stranded oligonucleotide comprises two covalently separate nucleic acid strands that are fully duplexed with one another. However, in some embodiments, a double-stranded oligonucleotide comprises two covalently separate nucleic acid strands that are partially duplexed, e.g., having overhangs at one or both ends. In some embodiments, a double-stranded oligonucleotide comprises antiparallel sequences of nucleotides that are partially complementary, and thus, may have one or more mismatches, which may include internal mismatches or end mismatches.

As used herein, the term “duplex” is used in reference to nucleic acids (e.g., oligonucleotides), and specifically refers to a double helical structure formed through complementary base pairing of two antiparallel sequences of nucleotides.

As used herein, the term “excipient” refers to a non-therapeutic agent that may be included in a composition, for example to provide or contribute to a desired consistency or stabilizing effect.

As used herein, the term “furanose” refers to a carbohydrate having a five-membered ring structure, where the ring structure has 4 carbon atoms and one oxygen atom represented by

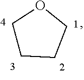

wherein the numbers represent the positions of the 4 carbon atoms in the five-membered ring structure.

As used herein, the term “hepatocyte” or “hepatocytes” refers to cells of the parenchymal tissues of the liver. These cells make up approximately 70-85% of the liver's mass and manufacture serum albumin, fibrinogen, and the prothrombin group of clotting factors (except for Factors 3 and 4). Markers for hepatocyte lineage cells may include but are not limited to: transthyretin (Ttr), glutamine synthetase (Glul), hepatocyte nuclear factor 1a (Hnfla), and hepatocyte nuclear factor 4a (Hnf4a). Markers for mature hepatocytes may include but are not limited to: cytochrome P450 (Cyp3al 1), fumarylacetoacetate hydrolase (Fah), glucose 6-phosphate (G6p), albumin (Alb), and OC2-2F8. See, e.g., Huch et al., (2013), NATURE, 494(7436): 247-50, the contents of which relating to hepatocyte markers is incorporated herein by reference.

As used herein, the term “glutathione” (GSH) refers to a tripeptide having structure

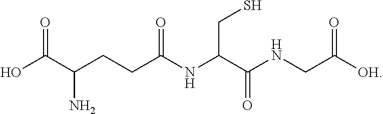 [SMILES: NC(CCC(=O)NC(CS)C(=O)NCC(=O)O)C(=O)O]

GSH is present in cells at a concentration of approximately 1-10 mM. GSH reduces glutathione-sensitive bonds, including disulfide bonds. In the process, glutathione is converted to its oxidized form, glutathione disulfide (GSSG). Once oxidized, glutathione can be reduced back by glutathione reductase, using NADPH as an electron donor.

As used herein, the terms “glutathione-sensitive compound”, or “glutathione-sensitive moiety”, are used interchangeably and refers to any chemical compound (e.g., oligonucleotide, nucleotide, or nucleoside) or moiety containing at least one glutathione-sensitive bond, such as a disulfide bridge or a sulfonyl group. As used herein, a “glutathione-sensitive oligonucleotide” is an oligonucleotide containing at least one nucleotide containing a glutathione-sensitive bond. A glutathione-sensitive moiety can be located at the 2′-carbon or 3′-carbon of the sugar moiety and comprises a sulfonyl group or a disulfide bridge. In certain embodiment, a glutathione-sensitive moiety is compatible with phosphoramidite oligonucleotide synthesis methods, as described, for example, in International Patent Application No. PCT/US2017/048239, which is hereby incorporated by reference in its entirety. A glutathione-sensitive moiety can also be located at the phosphorous containing internucleotide linkage. In certain embodiment, a glutathione-sensitive moiety is selected from those as described in PCT/US2013/072536, which is hereby incorporated by reference in its entirety.

As used herein, the term “internucleotide linking group” or “internucleotide linkage” refers to a chemical group capable of covalently linking two nucleoside moieties. Typically, the chemical group is a phosphorus-containing linkage group containing a phospho or phosphite group. Phospho linking groups are meant to include a phosphodiester linkage, a phosphorodithioate linkage, a phosphorothioate linkage, a phosphotriester linkage, a thionoalkylphosphonate linkage, a thionalkylphosphotriester linkage, a phosphoramidite linkage, a phosphonate linkage and/or a boranophosphate linkage. Many phosphorus-containing linkages are well known in the art, as disclosed, for example, in U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050. In other embodiments, the oligonucleotide contains one or more internucleotide linking groups that do not contain a phosphorous atom, such short chain alkyl or cycloalkyl internucleotide linkages, mixed heteroatom and alkyl or cycloalkyl internucleotide linkages, or one or more short chain heteroaromatic or heterocyclic internucleotide linkages, including, but not limited to, those having siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; and amide backbones. Non-phosphorous containing linkages are well known in the art, as disclosed, for example, in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

As used herein, the term “loop” refers to a structure formed by a single strand of a nucleic acid, in which complementary regions that flank a particular single stranded nucleotide region hybridize in a way that the single stranded nucleotide region between the complementary regions is excluded from duplex formation or Watson-Crick base pairing. A loop is a single stranded nucleotide region of any length. Examples of loops include the unpaired nucleotides present in such structures as hairpins and tetraloops.

As used herein, the terms “microRNA” “mature microRNA” “miRNA” and “miR” are interchangeable and refer to non-coding RNA molecules encoded in the genomes of plants and animals. Typically, mature microRNA are about 18-25 nucleotides in length. In certain instances, highly conserved, endogenously expressed microRNAs regulate the expression of genes by binding to the 3′-untranslated regions (3′-UTR) of specific mRNAs. Certain mature microRNAs appear to originate from long endogenous primary microRNA transcripts (also known as pre-microRNAs, pri-microRNAs, pri-mirs, pri-miRs or pri-pre-microRNAs) that are often hundreds of nucleotides in length (Lee, et al., EMBO 1, 2002, 21(17), 4663-70).

As used herein, the term “modified nucleoside” refers to a nucleoside containing one or more of a modified or universal nucleobase or a modified sugar. The modified or universal nucleobases (also referred to herein as base analogs) are generally located at the 1′-position of a nucleoside sugar moiety and refer to nucleobases other than adenine, guanine, cytosine, thymine and uracil at the 1′-position. In certain embodiments, the modified or universal nucleobase is a nitrogenous base. In certain embodiments, the modified nucleobase does not contain nitrogen atom. See e.g., U.S. Published Patent Application No. 20080274462. In certain embodiments, the modified nucleotide does not contain a nucleobase (abasic). A modified sugar (also referred herein to a sugar analog) includes modified deoxyribose or ribose moieties, e.g., where the modification occurs at the 2′, 3′-, 4′, or 5′-carbon position of the sugar. The modified sugar may also include non-natural alternative carbon structures such as those present in locked nucleic acids (“LNA”) (see, e.g., Koshkin et al. (1998), TETRAHEDRON, 54, 3607-30); bridged nucleic acids (“BNA”) (see, e.g., U.S. Pat. No. 7,427,672 and Mitsuoka et al. (2009), NUCLEIC ACIDS RES., 37(4):1225-38); and unlocked nucleic acids (“UNA”) (see, e.g., Snead et al. (2013), MOLECULAR THERAPY—NUCLEIC ACIDS, 2). Suitable modified or universal nucleobases or modified sugars in the context of the present disclosure are described herein.

As used herein, the term “modified nucleotide” refers to a nucleotide containing one or more of a modified or universal nucleobase, a modified sugar, or a modified phosphate. The modified or universal nucleobases (also referred to generally herein as nucleobase) are generally located at the 1′-position of a nucleoside sugar moiety and refer to nucleobases other than adenine, guanine, cytosine, thymine and uracil at the 1′-position. In certain embodiments, the modified or universal nucleobase is a nitrogenous base. In certain embodiments, the modified nucleobase does not contain nitrogen atom. See e.g., U.S. Published Patent Application No. 20080274462. In certain embodiments, the modified nucleotide does not contain a nucleobase (abasic). A modified sugar (also referred herein to a sugar analog) includes modified deoxyribose or ribose moieties, e.g., where the modification occurs at the 2′-, 3′-, 4′-, or 5′-carbon position of the sugar. The modified sugar may also include non-natural alternative carbon structures such as those present in locked nucleic acids (“LNA”) (see, e.g., Koshkin et al. (1998), TETRAHEDRON, 54, 3607-3630), bridged nucleic acids (“BNA”) (see, e.g., U.S. Pat. No. 7,427,672 and Mitsuoka et al. (2009), NUCLEIC ACIDS RES., 37(4):1225-38); and unlocked nucleic acids (“UNA”) (see, e.g., Snead et al. (2013), MOLECULAR THERAPY—NUCLEIC ACIDS, 2). Modified phosphate groups refer to a modification of the phosphate group that does not occur in natural nucleotides and includes non-naturally occurring phosphate mimics as described herein. Modified phosphate groups also include non-naturally occurring internucleotide linking groups, including both phosphorous containing internucleotide linking groups and non-phosphorous containing linking groups, as described herein. Suitable modified or universal nucleobases, modified sugars, or modified phosphates in the context of the present disclosure are described herein.

As used herein, the term “modified internucleotide linkage” refers to an internucleotide linkage having one or more chemical modifications compared with a reference internucleotide linkage comprising a phosphodiester bond. In some embodiments, a modified nucleotide is a non-naturally occurring linkage. Typically, a modified internucleotide linkage confers one or more desirable properties to a nucleic acid in which the modified internucleotide linkage is present. For example, a modified nucleotide may improve thermal stability, resistance to degradation, nuclease resistance, solubility, bioavailability, bioactivity, reduced immunogenicity, etc.

As used herein, the term “naked nucleic acid” refers to a nucleic acid that is not formulated in a protective lipid nanoparticle or other protective formulation and is thus exposed to the blood and endosomal/lysosomal compartments when administered in vivo.

As used herein, the term “natural nucleoside” refers to a heterocyclic nitrogenous base in N-glycosidic linkage with a sugar (e.g., deoxyribose or ribose or analog thereof). The natural heterocyclic nitrogenous bases include adenine, guanine, cytosine, uracil and thymine.

As used herein, the term “natural nucleotide” refers to a heterocyclic nitrogenous base in N-glycosidic linkage with a sugar (e.g., ribose or deoxyribose or analog thereof) that is linked to a phosphate group. The natural heterocyclic nitrogenous bases include adenine, guanine, cytosine, uracil and thymine.

A “nicked tetraloop structure” is a structure of a RNAi oligonucleotide characterized by the presence of separate sense (passenger) and antisense (guide) strands, in which the sense strand has a region of complementarity to the antisense strand such that the two strands form a duplex, and in which at least one of the strands, generally the sense strand, extends from the duplex in which the extension contains a tetraloop and two self-complementary sequences forming a stem region adjacent to the tetraloop, in which the tetraloop is configured to stabilize the adjacent stem region formed by the self-complementary sequences of the at least one strand.

As used herein, the term “nucleic acid or analogue thereof” refers to any natural or modified nucleotide, nucleoside, oligonucleotide, conventional antisense oligonucleotide, ribonucleotide, deoxyribonucleotide, ribozyme, RNAi inhibitor molecule, antisense oligo (ASO), short interfering RNA (siRNA), canonical RNA inhibitor molecule, aptamer, antagomir, exon skipping or splice altering oligos, mRNA, miRNA, or CRISPR nuclease systems comprising one or more of the lipid conjugates described herein. In certain embodiments, the provided nucleic acids or analogues thereof are used in antisense oligonucleotides, siRNA, and dicer substrate siRNA, including those described in U.S. 2010/331389, U.S. Pat. Nos. 8,513,207, 10,131,912, 8,927,705, CA 2,738,625, EP 2,379,083, and EP 3,234,132, the entirety of each of which is herein incorporated by reference.

As used herein, the term “nucleic acid inhibitor molecule” refers to an oligonucleotide molecule that reduces or eliminates the expression of a target gene wherein the oligonucleotide molecule contains a region that specifically targets a sequence in the target gene mRNA. Typically, the targeting region of the nucleic acid inhibitor molecule comprises a sequence that is sufficiently complementary to a sequence on the target gene mRNA to direct the effect of the nucleic acid inhibitor molecule to the specified target gene. The nucleic acid inhibitor molecule may include ribonucleotides, deoxyribonucleotides, and/or modified nucleotides.

As used herein, the term “nucleobase” refers to a natural nucleobase, a modified nucleobase, or a universal nucleobase. The nucleobase is the heterocyclic moiety which is located at the 1′ position of a nucleotide sugar moiety in a modified nucleotide that can be incorporated into a nucleic acid duplex (or the equivalent position in a nucleotide sugar moiety substitution that can be incorporated into a nucleic acid duplex). Accordingly, the present disclosure provides a nucleic acid and analogue thereof comprising a lipid conjugate, wherein the lipid conjugate is represented by formula I or II where the nucleobase is generally either a purine or pyrimidine base. In some embodiments, the nucleobase can also include the common bases guanine (G), cytosine (C), adenine (A), thymine (T), or uracil (U), or derivatives thereof, such as protected derivatives suitable for use in the preparation of oligonucleotides. In some embodiments, each of nucleobases G, A, and C independently comprises a protecting group selected from isobutyryl, acetyl, difluoroacetyl, trifluoroacetyl, phenoxyacetyl, isopropylphenoxyacetyl, benzoyl, 9-fluorenylmethoxycarbonyl, phenoxyacetyl, dimethylformamidine, dibutylforamidine and N,N-diphenylcarbamate. Nucleobase analogs can duplex with other bases or base analogs in dsRNAs. Nucleobase analogs include those useful in the nucleic acids and analogues thereof and methods of the disclosure, e.g., those disclosed in U.S. Pat. Nos. 5,432,272 and 6,001,983 to Benner and U.S. Patent Publication No. 20080213891 to Manoharan, which are herein incorporated by reference. Non-limiting examples of nucleobases include hypoxanthine (I), xanthine (X), 30-D-ribofuranosyl-(2,6-diaminopyrimidine) (K), 3-β-D-ribofuranosyl-(1-methyl-pyrazolo[4,3-d]pyrimidine-5,7(4H,6H)-dione) (P), iso-cytosine (iso-C), iso-guanine (iso-G), 1-β-D-ribofuranosyl-(5-nitroindole), 1-β-D-ribofuranosyl-(3-nitropyrrole), 5-bromouracil, 2-aminopurine, 4-thio-dT, 7-(2-thienyl)-imidazo[4,5-b]pyridine (Ds) and pyrrole-2-carbaldehyde (Pa), 2-amino-6-(2-thienyl)purine (S), 2-oxopyridine (Y), difluorotolyl, 4-fluoro-6-methylbenzimidazole, 4-methylbenzimidazole, 3-methyl isocarbostyrilyl, 5-methyl isocarbostyrilyl, and 3-methyl-7-propynyl isocarbostyrilyl, 7-azaindolyl, 6-methyl-7-azaindolyl, imidizopyridinyl, 9-methyl-imidizopyridinyl, pyrrolopyrizinyl, isocarbostyrilyl, 7-propynyl isocarbostyrilyl, propynyl-7-azaindolyl, 2,4,5-trimethylphenyl, 4-methylindolyl, 4,6-dimethylindolyl, phenyl, napthalenyl, anthracenyl, phenanthracenyl, pyrenyl, stilbenzyl, tetracenyl, pentacenyl, and structural derivatives thereof (Schweitzer et al., J. ORG. CHEM., 59:7238-7242 (1994); Berger et al., NUCLEIC ACIDS RESEARCH, 28(15):2911-2914 (2000); Moran et al., J. AM. CHEM. SOC., 119:2056-2057 (1997); Morales et al., J. AM. CHEM. SOC., 121:2323-2324 (1999); Guckian et al., J. AM. CHEM. SOC., 118:8182-8183 (1996); Morales et al., J. AM. CHEM. SOC., 122(6):1001-1007 (2000); McMinn et al., J. AM. CHEM. SOC., 121:11585-11586 (1999); Guckian et al., J. ORG. CHEM., 63:9652-9656 (1998); Moran et al., PROC. NATL. ACAD. SCI., 94:10506-10511 (1997); Das et al., J. CHEM. SOC., PERKIN TRANS., 1:197-206 (2002); Shibata et al., J. CHEM. SOC., Perkin Trans., 1: 1605-1611 (2001); Wu et al., J. AM. CHEM. SOC., 122(32):7621-7632 (2000); O'Neill et al., J. ORG. CHEM., 67:5869-5875 (2002); Chaudhuri et al., J. AM. CHEM. SOC., 117:10434-10442 (1995); and U.S. Pat. No. 6,218,108.). Base analogs may also be a universal base.

As used herein, the term “nucleoside” refers to a natural nucleoside or a modified nucleoside.

As used herein, the term “nucleotide” refers to a natural nucleotide or a modified nucleotide.

As used herein, the term “nucleotide position” refers to a position of a nucleotide in an oligonucleotide as counted from the nucleotide at the 5′-terminus. For example, nucleotide position 1 refers to the 5′-terminal nucleotide of an oligonucleotide.

As used herein, the term “oligonucleotide” as used herein refers to a polymeric form of nucleotides ranging from 2 to 2500 nucleotides. Oligonucleotides may be single-stranded or double-stranded. In certain embodiments, the oligonucleotide has 500-1500 nucleotides, typically, for example, where the oligonucleotide is used in gene therapy. In certain embodiments, the oligonucleotide is single or double stranded and has 7-100 nucleotides. In certain embodiments, the oligonucleotide is single or double stranded and has 15-100 nucleotides. In another embodiment, the oligonucleotide is single or double stranded has 15-50 nucleotides, typically, for example, where the oligonucleotide is a nucleic acid inhibitor molecule. In another embodiment, the oligonucleotide is single or double stranded has 25-40 nucleotides, typically, for example, where the oligonucleotide is a nucleic acid inhibitor molecule. In yet another embodiment, the oligonucleotide is single or double stranded and has 19-40 or 19-25 nucleotides, typically, for example, where the oligonucleotide is a double-stranded nucleic acid inhibitor molecule and forms a duplex of at least 18-25 base pairs. In other embodiments, the oligonucleotide is single stranded and has 15-25 nucleotides, typically, for example, where the oligonucleotide nucleotide is a single stranded RNAi inhibitor molecule. Typically, the oligonucleotide contains one or more phosphorous containing internucleotide linking groups, as described herein. In other embodiments, the internucleotide linking group is a non-phosphorus containing linkage, as described herein. An oligonucleotide can comprise ribonucleotides, deoxyribonucleotides, and/or modified nucleotides including, for example, modified ribonucleotides. An oligonucleotide may be single-stranded or double-stranded. An oligonucleotide may or may not have duplex regions. As a set of non-limiting examples, an oligonucleotide may be, but is not limited to, a small interfering RNA (siRNA), microRNA (miRNA), short hairpin RNA (shRNA), dicer substrate interfering RNA (dsiRNA), antisense oligonucleotide, short siRNA, or single-stranded siRNA. In some embodiments, a double-stranded oligonucleotide is an RNAi oligonucleotide.

As used herein, the term “overhang” refers to terminal non-base pairing nucleotide(s) at either end of either strand of a double-stranded nucleic acid inhibitor molecule. In certain embodiments, the overhang results from one strand or region extending beyond the terminus of the complementary strand to which the first strand or region forms a duplex. One or both of two oligonucleotide regions that are capable of forming a duplex through hydrogen bonding of base pairs may have a 5′- and/or 3′-end that extends beyond the 3′- and/or 5′-end of complementarity shared by the two polynucleotides or regions. The single-stranded region extending beyond the 3′- and/or 5′-end of the duplex is referred to as an overhang.

As used herein, the term “pharmaceutical composition” comprises a pharmacologically effective amount of a phosphate analog-modified oligonucleotide and a pharmaceutically acceptable excipient. As used herein, “pharmacologically effective amount” “therapeutically effective amount” or “effective amount” refers to that amount of a phosphate analog-modified oligonucleotide of the present disclosure effective to produce the intended pharmacological, therapeutic or preventive result.

As used herein, the term “pharmaceutically acceptable excipient”, means that the excipient is suitable for use with humans and/or animals without undue adverse side effects (such as toxicity, irritation, and allergic response) commensurate with a reasonable benefit/risk ratio.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in, J. PHARMACEUTICAL SCIENCES, 1977, (66); 1-19, incorporated herein by reference. Pharmaceutically acceptable salts of the nucleic acids and analogues thereof of this disclosure include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

As used herein, the term “phosphate analog” refers to a chemical moiety that mimics the electrostatic and/or steric properties of a phosphate group. In some embodiments, a phosphate analog is positioned at the 5′ terminal nucleotide of an oligonucleotide in place of a 5′-phosphate, which is often susceptible to enzymatic removal. In some embodiments, a 5′ phosphate analog contains a phosphatase-resistant linkage. Examples of phosphate analogs include 5′ phosphonates, such as 5′ methylenephosphonate (5′-MP) and 5′-(E)-vinylphosphonate (5′-VP). In some embodiments, an oligonucleotide has a phosphate analog at a 4′-carbon position of the sugar (referred to as a “4′-phosphate analog”) at a 5′-terminal nucleotide. An example of a 4′-phosphate analog is oxymethylphosphonate, in which the oxygen atom of the oxymethyl group is bound to the sugar moiety (e.g., at its 4′-carbon) or analog thereof. See, for example, International Patent Application PCT/US2017/049909, filed on Sep. 1, 2017, U.S. Provisional Application Nos. 62/383,207, filed on Sep. 2, 2016, and 62/393,401, filed on Sep. 12, 2016, the contents of each of which relating to phosphate analogs are incorporated herein by reference. Other modifications have been developed for the 5′ end of oligonucleotides (see, e.g., WO 2011/133871; U.S. Pat. No. 8,927,513; and Prakash et al. (2015), NUCLEIC ACIDS RES., 43(6):2993-3011, the contents of each of which relating to phosphate analogs are incorporated herein by reference).

As used herein, the term “reduced expression” of a gene refers to a decrease in the amount of RNA transcript or protein encoded by the gene and/or a decrease in the amount of activity of the gene in a cell or subject, as compared to an appropriate reference cell or subject. For example, the act of treating a cell with a double-stranded oligonucleotide (e.g., one having an antisense strand that is complementary to ALDH2 mRNA sequence) may result in a decrease in the amount of RNA transcript, protein and/or enzymatic activity (e.g., encoded by the ALDH2 gene) compared to a cell that is not treated with the double-stranded oligonucleotide. Similarly, “reducing expression” as used herein refers to an act that results in reduced expression of a gene (e.g., ALDH2).

As used herein, the term “region of complementarity” refers to a sequence of nucleotides of a nucleic acid (e.g., a double-stranded oligonucleotide) that is sufficiently complementary to an antiparallel sequence of nucleotides (e.g., a target nucleotide sequence within an mRNA) to permit hybridization between the two sequences of nucleotides under appropriate hybridization conditions, e.g., in a phosphate buffer, in a cell, etc. A region of complementarity may be fully complementary to a nucleotide sequence (e.g., a target nucleotide sequence present within an mRNA or portion thereof). For example, a region of complementary that is fully complementary to a nucleotide sequence present in an mRNA has a contiguous sequence of nucleotides that is complementary, without any mismatches or gaps, to a corresponding sequence in the mRNA. Alternatively, a region of complementarity may be partially complementary to a nucleotide sequence (e.g., a nucleotide sequence present in an mRNA or portion thereof). For example, a region of complementary that is partially complementary to a nucleotide sequence present in an mRNA has a contiguous sequence of nucleotides that is complementary to a corresponding sequence in the mRNA but that contains one or more mismatches or gaps (e.g., 1, 2, 3, or more mismatches or gaps) compared with the corresponding sequence in the mRNA, provided that the region of complementarity remains capable of hybridizing with the mRNA under appropriate hybridization conditions. In some embodiments, the region of complementarity is at least 12, at least 13, at least 14, at least 15, at least 16, at least 17, at least 18, at least 19, at least 20, at least 21, at least 22, at least 23, at least 24, at least 25 nucleotides in length.

As used herein, the term “strand” refers to a single contiguous sequence of nucleotides linked together through internucleotide linkages (e.g., phosphodiester linkages, phosphorothioate linkages). In some embodiments, a strand has two free ends, e.g., a 5′-end and a 3′-end.

As used herein, the term “subject” means any mammal, including mice, rabbits, and humans. In one embodiment, the subject is a human or non-human primate. The terms “individual” or “patient” may be used interchangeably with “subject.”

As used herein, the term “synthetic” refers to a nucleic acid or other molecule that is artificially synthesized (e.g., using a machine (e.g., a solid state nucleic acid synthesizer)) or that is otherwise not derived from a natural source (e.g., a cell or organism) that normally produces the molecule.

As used herein, the term “suitable prodrug” is meant to indicate a compound that may be converted under physiological conditions or by solvolysis to a biologically active nucleic acid or analogue thereof described herein. Thus, the term “prodrug” refers to a precursor of a biologically active nucleic acid or analogue thereof that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgard, H., DESIGN OF PRODRUGS (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in BIOREVERSIBLE CARRIERS IN DRUG DESIGN, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of suitable prodrugs include, but are not limited to glutathione, acyloxy, thioacyloxy, 2-carboalkoxyethyl, disulfide, thiaminal, and enol ester derivatives of a phosphorus atom-modified nucleic acid. The term “pro-oligonucleotide” or “pronucleotide” or “nucleic acid prodrug” refers to an oligonucleotide which has been modified to be a prodrug of the oligonucleotide. Phosphonate and phosphate prodrugs can be found, for example, in Wiener et al., “Prodrugs or phosphonates and phosphates: crossing the membrane” TOP. CURR. CHEM. 2015, 360:115-160, the entirety of which is herein incorporated by reference.

As used herein, the phrase “suitable hydroxyl protecting group” are well known in the art and when taken with the oxygen atom to which it is bound, is independently selected from esters, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl) ethoxymethyl, and tetrahydropyranyl ethers. Examples of arylalkyl ethers include benzyl, p-methoxybenzyl, 3,4-dimethoxybenzyl, O-nitrobenzyl, p-nitrobenzyl, p-halobenzyl, 2,6-dichlorobenzyl, p-cyanobenzyl, and 2- and 4-picolyl. In some embodiments, the suitable hydroxyl protecting group is an acid labile group such as trityl, 4-methyoxytrityl, 4,4′-dimethyoxytrityl (DMTr), 4,4′,4″-trimethyoxytrityl, 9-phenyl-xanthen-9-yl, 9-(p-tolyl)-xanthen-9-yl, pixyl, 2,7-dimethylpixyl, and the like, suitable for deprotection during both solution-phase and solid-phase synthesis of acid-sensitive oligonucleotides using for example, dichloroacetic acid, trichloroacetic acid, trifluoroacetic acid, or acetic acid. The t-butyldimethylsilyl group is stable under the acidic conditions used to remove the DMTr group during synthesis but can be removed after cleavage and deprotection of the RNA oligomer with a fluoride source, e.g., tetrabutylammonium fluoride or pyridine hydrofluoride.

As used herein, the phrase “suitable amino protecting group” are well known in the art and when taken with the nitrogen to which it is attached, include, but are not limited to, aralkylamines, carbamates, allyl amines, amides, and the like. Examples of mono-protection groups for amines include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like. Examples of di-protection groups for amines include amines that are substituted with two substituents independently selected from those described above as mono-protection groups, and further include cyclic imides, such as phthalimide, maleimide, succinimide, 2,2,5,5-tetramethyl-1,2,5-azadisilolidine, azide, and the like. It will be appreciated that upon acid hydrolysis of an amino protecting groups, a salt compound thereof is formed. For example, when an amino protecting group is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms are contemplated.

As used herein, the term “phosphoramidite” refers to a nitrogen containing trivalent phosphorus derivative. Examples of suitable phosphoramidites are described herein.

As used herein, “potency” refers to the amount of an oligonucleotide or other drug that must be administered in vivo or in vitro to obtain a particular level of activity against an intended target in cells. For example, an oligonucleotide that suppresses the expression of its target by 90% in a subject at a dosage of 1 mg/kg has a greater potency than an oligonucleotide that suppresses the expression of its target by 90% in a subject at a dosage of 100 mg/kg.

As used herein, the term “protecting group” is used in the conventional chemical sense as a group which reversibly renders unreactive a functional group under certain conditions of a desired reaction. After the desired reaction, protecting groups may be removed to deprotect the protected functional group. All protecting groups should be removable under conditions which do not degrade a substantial proportion of the molecules being synthesized.

As used herein, the term “provided nucleic acid” refers to any genus, subgenus, and/or species set forth herein.

As used herein, the term “ribonucleotide” refers to a nucleotide having a ribose as its pentose sugar, which contains a hydroxyl group at its 2′ position. A modified ribonucleotide is a ribonucleotide having one or more modifications or substitutions of atoms other than at the 2′ position, including modifications or substitutions in or of the ribose, phosphate group or base.

As used herein, the term “ribozyme” refers to a catalytic nucleic acid molecule that specifically recognizes and cleaves a distinct target nucleic acid sequence, which can be either DNA or RNA. Each ribozyme has a catalytic component (also referred to as a “catalytic domain”) and a target sequence-binding component consisting of two binding domains, one on either side of the catalytic domain.

As used herein, the term “RNAi inhibitor molecule” refers to either (a) a double stranded nucleic acid inhibitor molecule (“dsRNAi inhibitor molecule”) having a sense strand (passenger) and antisense strand (guide), where the antisense strand or part of the antisense strand is used by the Argonaute 2 (Ago2) endonuclease in the cleavage of a target mRNA or (b) a single stranded nucleic acid inhibitor molecule (“ssRNAi inhibitor molecule”) having a single antisense strand, where that antisense strand (or part of that antisense strand) is used by the Ago2 endonuclease in the cleavage of a target mRNA.

A double stranded RNAi inhibitor molecule comprises two oligonucleotide strands: an antisense strand and a sense strand. The sense strand or a region thereof is partially, substantially or fully complementary to the antisense strand of the double stranded RNAi inhibitor molecule or a region thereof. In certain embodiments, the sense strand may also contain nucleotides that are non-complementary to the antisense strand. The non-complementary nucleotides may be on either side of the complementary sequence or may be on both sides of the complementary sequence. In certain embodiments, where the sense strand or a region thereof is partially or substantially complementary to the antisense strand or a region thereof, the non-complementary nucleotides may be located between one or more regions of complementarity (e.g., one or more mismatches). The sense strand is also called the passenger strand.

As used herein, the term “systemic administration” refers to in vivo systemic absorption or accumulation of drugs in the blood stream followed by distribution throughout the entire body.

As used herein, the term “target site” “target sequence,” “target nucleic acid”, “target region,” “target gene” are used interchangeably and refer to a RNA or DNA sequence that is “targeted,” e.g., for cleavage mediated by an RNAi inhibitor molecule that contains a sequence within its guide/antisense region that is partially, substantially, or perfectly or sufficiently complementary to that target sequence.

As used herein, the term “targeting ligand” refers to a molecule (e.g., a carbohydrate, amino sugar, cholesterol, polypeptide or lipid) that selectively binds to a cognate molecule (e.g., a receptor) of a tissue or cell of interest and that can be conjugated to another substance for purposes of targeting the other substance to the tissue or cell of interest. For example, in some embodiments, a targeting ligand may be conjugated to an oligonucleotide for purposes of targeting the oligonucleotide to a specific tissue or cell of interest. In some embodiments, a targeting ligand selectively binds to a cell surface receptor. Accordingly, in some embodiments, a targeting ligand when conjugated to an oligonucleotide facilitates delivery of the oligonucleotide into a particular cell through selective binding to a receptor expressed on the surface of the cell and endosomal internalization by the cell of the complex comprising the oligonucleotide, targeting ligand and receptor. In some embodiments, a targeting ligand is conjugated to an oligonucleotide via a linker that is cleaved following or during cellular internalization such that the oligonucleotide is released from the targeting ligand in the cell.

As used herein, the term “treat” refers to the act of providing care to a subject in need thereof, e.g., through the administration a therapeutic agent (e.g., an oligonucleotide) to the subject, for purposes of improving the health and/or well-being of the subject with respect to an existing condition (e.g., a disease, disorder) or to prevent or decrease the likelihood of the occurrence of a condition. In some embodiments, treatment involves reducing the frequency or severity of at least one sign, symptom or contributing factor of a condition (e.g., disease, disorder) experienced by a subject.

As used herein, the term “tetraloop” refers to a loop (a single stranded region) that forms a stable secondary structure that contributes to the stability of an adjacent Watson-Crick hybridized nucleotides. Without being limited to theory, a tetraloop may stabilize an adjacent Watson-Crick base pair by stacking interactions. In addition, interactions among the nucleotides in a tetraloop include but are not limited to non-Watson-Crick base pairing, stacking interactions, hydrogen bonding, and contact interactions (Cheong et al., NATURE 1990; 346(6285):680-2; Heus and Pardi, SCIENCE 1991; 253(5016):191-4). A tetraloop confers an increase in the melting temperature (Tm) of an adjacent duplex that is higher than expected from a simple model loop sequence consisting of random bases. For example, a tetraloop can confer a melting temperature of at least 50° C., at least 55° C., at least 56° C., at least 58° C., at least 60° C., at least 65° C. or at least 75° C. in 10 mM NaHPO₄ to a hairpin comprising a duplex of at least 2 base pairs in length. A tetraloop may contain ribonucleotides, deoxyribonucleotides, modified nucleotides, and combinations thereof. In certain embodiments, a tetraloop consists of four nucleotides. In certain embodiments, a tetraloop consists of five nucleotides.

Examples of RNA tetraloops include the UNCG family of tetraloops (e.g., UUCG), the GNRA family of tetraloops (e.g., GAAA), and the CUUG tetraloop. (Woese et al., PNAS, 1990, 87(21):8467-71; Antao et al., NUCLEIC ACIDS RES., 1991, 19(21):5901-5). Examples of DNA tetraloops include the d(GNNA) family of tetraloops (e.g., d(GTTA), the d(GNRA)) family of tetraloops, the d(GNAB) family of tetraloops, the d(CNNG) family of tetraloops, and the d(TNCG) family of tetraloops (e.g., d(TTCG)). (Nakano et al., BIOCHEMISTRY, 2002, 41(48):14281-14292. Shinji et al., NIPPON KAGAKKAI KOEN YOKOSHU, 2000, 78(2):731), which are incorporated by reference herein for their relevant disclosures. In some embodiments, the tetraloop is contained within a nicked tetraloop structure.

As used herein, “universal base” refers to a heterocyclic moiety located at the 1′ position of a nucleotide sugar moiety in a modified nucleotide, or the equivalent position in a nucleotide sugar moiety substitution, that, when present in a nucleic acid duplex, can be positioned opposite more than one type of base without altering the double helical structure (e.g., the structure of the phosphate backbone). Additionally, the universal base does not destroy the ability of the single stranded nucleic acid in which it resides to duplex to a target nucleic acid. The ability of a single stranded nucleic acid containing a universal base to duplex a target nucleic can be assayed by methods apparent to one in the art (e.g., UV absorbance, circular dichroism, gel shift, single stranded nuclease sensitivity, etc.). Additionally, conditions under which duplex formation is observed may be varied to determine duplex stability or formation, e.g., temperature, as melting temperature (Tm) correlates with the stability of nucleic acid duplexes. Compared to a reference single stranded nucleic acid that is exactly complementary to a target nucleic acid, the single stranded nucleic acid containing a universal base forms a duplex with the target nucleic acid that has a lower Tm than a duplex formed with the complementary nucleic acid. However, compared to a reference single stranded nucleic acid in which the universal base has been replaced with a base to generate a single mismatch, the single stranded nucleic acid containing the universal base forms a duplex with the target nucleic acid that has a higher Tm than a duplex formed with the nucleic acid having the mismatched base.

Some universal bases are capable of base pairing by forming hydrogen bonds between the universal base and all of the bases guanine (G), cytosine (C), adenine (A), thymine (T), and uracil (U) under base pair forming conditions. A universal base is not a base that forms a base pair with only one single complementary base. In a duplex, a universal base may form no hydrogen bonds, one hydrogen bond, or more than one hydrogen bond with each of G, C, A, T, and U opposite to it on the opposite strand of a duplex. Preferably, the universal bases do not interact with the base opposite to it on the opposite strand of a duplex. In a duplex, base pairing between a universal base occurs without altering the double helical structure of the phosphate backbone. A universal base may also interact with bases in adjacent nucleotides on the same nucleic acid strand by stacking interactions. Such stacking interactions stabilize the duplex, especially in situations where the universal base does not form any hydrogen bonds with the base positioned opposite to it on the opposite strand of the duplex. Non-limiting examples of universal-binding nucleotides include inosine, 1-β-D-ribo furanosyl-5-nitroindole, and/or 1-β-D-ribofuranosyl-3-nitropyrrole (US Pat. Appl. Publ. No. 20070254362 to Quay et al.; Van Aerschot et al., An acyclic 5-nitroindazole nucleoside analogue as ambiguous nucleoside, NUCLEIC ACIDS RES. 1995 Nov. 11; 23(21):4363-70; Loakes et al., 3-Nitropyrrole and 5-nitroindole as universal bases in primers for DNA sequencing and PCR, NUCLEIC ACIDS RES. 1995 Jul. 11; 23(13):2361-66; Loakes and Brown, 5-Nitroindole as a universal base analogue, NUCLEIC ACIDS RES. 1994 Oct. 11; 22(20):4039-43).

The disclosed nucleic acids or analogs thereof comprising one or more lipid conjugate can be incorporated into multiple different oligonucleotide structures (or formats). For example, in some embodiments, the disclosed nucleic acids can be incorporated into oligonucleotides that comprise sense and antisense strands that are both in the range of 17 to 36 nucleotides in length. In some embodiments, oligonucleotides incorporating the disclosed nucleic acids are provided that have a tetraloop structure within a 3′ extension of their sense strand, and two terminal overhang nucleotides at the 3′ end of its antisense strand. In some embodiments, the two terminal overhang nucleotides are GG. Typically, one or both of the two terminal GG nucleotides of the antisense strand is or are not complementary to the target.

In some embodiments, oligonucleotides incorporating the disclosed nucleic acids or analogs thereof comprising one or more lipid conjugate are provided that have sense and antisense strands that are both in the range of 21 to 23 nucleotides in length. In some embodiments, a 3′ overhang is provided on the sense, antisense, or both sense and antisense strands that is 1 or 2 nucleotides in length. In some embodiments, an oligonucleotide has a guide strand of 23 nucleotides and a passenger strand of 21 nucleotides, in which the 3′-end of passenger strand and 5′-end of guide strand form a blunt end and where the guide strand has a two nucleotide 3′ overhang.

In some embodiments, the oligonucleotide-ligand conjugate is a duplex structure with blunt ends. In some embodiments, the conjugate has truncated passenger/sense strand.

In some embodiments, 1 or more (e.g., 1, 2, 3, 4, 5 or 6) nucleotides of an oligonucleotide comprise a lipid conjugate. In some embodiments, 2 to 4 nucleotides of a provided oligonucleotide are each conjugated to a separate lipid conjugate. In some embodiments, 2 to 4 nucleotides comprise lipid conjugates at either ends of the sense or antisense strand (e.g., lipids are conjugated to a 2 to 4 nucleotide overhang or extension on the 5′- or 3′-end of the sense or antisense strand) such that the lipid moieties resemble bristles of a toothbrush and the oligonucleotide resembles a toothbrush. For example, a provided oligonucleotide may comprise a stem-loop at either the 5′- or 3′-end of the sense strand and 1, 2, 3 or 4 nucleotides of the loop of the stem may be individually lipid conjugated.

In some embodiments, a provided oligonucleotide is conjugated to a monovalent lipid conjugate. In some embodiments, the oligonucleotide is conjugated to more than one monovalent lipid conjugate (i.e., is conjugated to 2, 3, or 4 monovalent lipid conjugates, and is typically conjugated to 3 or 4 monovalent lipid conjugates). In some embodiments, a provided oligonucleotide is conjugated to one or more bivalent lipid conjugate, trivalent lipid conjugate, or tetravalent lipid conjugate moieties.

In some embodiments, a provided oligonucleotide is conjugated to an adamantyl or a lipid moiety at 2′ or 3′ position of the nucleotide. In some embodiments, a provided oligonucleotide is conjugated to an adamantyl or a lipid moeity at the 5′ end of the nucleotide.

In some embodiments, 1 or more (e.g., 1, 2, 3, 4, 5 or 6) nucleotides of a provided oligonucleotide are each conjugated to one or more lipid conjugates. In some embodiments, 2 to 4 nucleotides of the loop of the stem-loop are each conjugated to a separate lipid conjugate. In some embodiments, lipids are conjugated to 2 to 4 nucleotides at either ends of the sense or antisense strand (e.g., lipids are conjugated to a 2 to 4 nucleotide overhang or extension on the 5′ or 3′ end of the sense or antisense strand) such that the lipid moieties resemble bristles of a toothbrush and the oligonucleotide resembles a toothbrush. For example, an oligonucleotide may comprise a stem-loop at either the 5′- or 3′-end of the sense strand and 1, 2, 3 or 4 nucleotides of the loop of the stem may be individually conjugated to a lipid moiety. In some embodiments, lipid moieties are conjugated to a nucleotide of the sense strand. For example, four lipid moieties can be conjugated to nucleotides in the tetraloop of the sense strand, where each lipid moiety is conjugated to one nucleotide.

i. Oligonucleotide Structures

There are a variety of structures of oligonucleotides that are useful for targeting RNA in the methods of the present disclosure, including RNAi, miRNA, etc. An oligonucleotide comprising one or more lipid conjugate described herein may be used as a framework to incorporate or target an RNA sequence. Double-stranded oligonucleotides for targeting RNA expression (e.g., via the RNAi pathway) generally have a sense strand and an antisense strand that form a duplex with one another. In some embodiments, the sense and antisense strands are not covalently linked. However, in some embodiments, the sense and antisense strands are covalently linked.

In some embodiments, a double-stranded oligonucleotides is provided for reducing the expression of RNA expression engage RNA interference (RNAi). For example, RNAi oligonucleotides have been developed with each strand having sizes of 19-25 nucleotides with at least one 3′ overhang of 1 to 5 nucleotides (see, e.g., U.S. Pat. No. 8,372,968). Longer oligonucleotides have also been developed that are processed by Dicer to generate active RNAi products (see, e.g., U.S. Pat. No. 8,883,996). Further work produced extended double-stranded oligonucleotides where at least one end of at least one strand is extended beyond a duplex targeting region, including structures where one of the strands includes a thermodynamically-stabilizing tetraloop structure (see, e.g., U.S. Pat. Nos. 8,513,207 and 8,927,705, as well as WO 2010/033225, which are incorporated by reference herein for their disclosure of these oligonucleotides). Such structures may include single-stranded extensions (on one or both sides of the molecule) as well as double-stranded extensions.

In some embodiments, a provided oligonucleotide may be in the range of 21 to 23 nucleotides in length. In some embodiments, a provided oligonucleotide may have an overhang (e.g., of 1, 2, or 3 nucleotides in length) in the 3′ end of the sense and/or antisense strands. In some embodiments, a provided oligonucleotide (e.g., siRNA) may comprise a 21 nucleotide guide strand that is antisense to a target RNA and a complementary passenger strand, in which both strands anneal to form a 19-bp duplex and 2 nucleotide overhangs at either or both 3′ ends. See, for example, U.S. Pat. Nos. 9,012,138, 9,012,621, and 9,193,753, the contents of each of which are incorporated herein for their relevant disclosures.

In some embodiments, a provided oligonucleotide has a 36 nucleotide sense strand that comprises an region extending beyond the antisense-sense duplex, where the extension region has a stem-tetraloop structure where the stem is a six base pair duplex and where the tetraloop has four nucleotides. In certain of those embodiments, in addition to one or more lipid conjugates, one or more of the tetraloop nucleotides are each conjugated to a monovalent GalNac ligand.

In some embodiments, a provided oligonucleotide comprises a 12-25 nucleotide sense strand and a 19-27 nucleotide antisense strand that when acted upon by a dicer enzyme results in an antisense strand that is incorporated into the mature RISC.

In some embodiments, a provided oligonucleotide comprises a 25 nucleotide sense strand and a 27 nucleotide antisense strand that when acted upon by a dicer enzyme results in an antisense strand that is incorporated into the mature RISC.

Other oligonucleotides design for use with the compositions and methods disclosed herein include: 16-mer siRNAs (see, e.g., NUCLEIC ACIDS IN CHEMISTRY AND BIOLOGY. Blackburn (ed.), ROYAL SOCIETY OF CHEMISTRY, 2006), shRNAs (e.g., having 19 bp or shorter stems; see, e.g., Moore et al. METHODS MOL. BIOL. 2010; 629:141-58), blunt siRNAs (e.g., of 19 bps in length; see: e.g., Kraynack and Baker, RNA Vol. 12, r163-176 (2006)), asymmetrical siRNAs (aiRNA; see, e.g., Sun et al., NAT. BIOTECHNOL. 26, 1379-1382 (2008)), asymmetric shorter-duplex siRNA (see, e.g., Chang et al, MOL THER. 2009 April; 17(4): 725-32), fork siRNAs (see, e.g., Hohjoh, FEBS LETTERS, Vol 557, issues 1-3; (January 2004), p 193-98), single-stranded siRNAs (Elsner; NATURE BIOTECHNOLOGY 30, 1063 (2012)), dumbbell-shaped circular siRNAs (see, e.g., Abe et al. J AM CHEM SOC 129: 15108-15109 (2007)), and small internally segmented interfering RNA (sisiRNA; see, e.g., Bramsen et al., NUCLEIC ACIDS RES. 2007 September; 35(17): 5886-97). Each of the foregoing references is incorporated by reference in its entirety for the related disclosures therein. Further non-limiting examples of an oligonucleotide structures that may be used in some embodiments to reduce or inhibit gene expression are microRNA (miRNA), short hairpin RNA (shRNA), and short siRNA (see, e.g., Hamilton et al, EMBO J., 2002, 21(17): 4671-4679; see also U.S. Application No. 20090099115).

As has been shown in the instant disclosure is that siRNAs acting via RNA interference mechanisms are useful in the recognition and degradation of targeted mRNA sequences. A chief difficulty in the prior art has been the low efficiency of siRNA delivery to target cells outside the liver and the degradation of siRNAs by nucleases in various biological fluids, these difficulties have been sufficient to prevent useful systemic delivery of siRNA to various tissues. According to the current invention, however, various conjugates can also be used in association with the chemical structures provided here to enhance and enable delivery to various organ systems and tissues within a mammalian host. Such conjugates have, according to the prior, have taken the form cationic lipid solutions, polymers, and nanoparticles. According to the current invention the structures provided herein can be conjugated to include various biogenic molecules. Such molecules include, and are not limited to, small lipophilic molecules or chains, antibodies, aptamers, ligands, peptides, or polymers each of various sizes. Such conjugates are preferred since they do not need a positive charge to form complexes, have limited toxicity and are less immunogenic.

Such conjugates may also have a variety of positions and clustering patterns on the passenger strand and/or guide strand. Such positioning can assist in contributing to the efficiency and capacity of siRNAs to degrade target mRNAs. As is known, siRNAs are polyanions and thus are unable to penetrate directly through the hydrophobic cell membrane and can enter the cell only by endocytosis or pinocytosis. Likewise, the chemical modifications as described herein may impact the properties of the siRNA molecules of the current invention including: their sensitivity to ribonucleases, recognition by the RNAi system, hydrophobicity, toxicity, duplex melting temperature, and conformation of the RNA helix. Typically, modifications can be divided into modifications of ribose, phosphates, and nucleobases. It is assumed that the total melting point of the duplex can contribute to the efficiency of siRNA interfering activity (Park and Shin, 2015). Thus, according to the current invention conjugates positioned at different locations of the hairpin other than the stem loop will also have impact on the effectiveness of the siRNA molecules. The use of multiple conjugates that are attached to the siRNA hairpin molecule can either be focused on one section or end of the dsRNA or spread out over the length of the oligonucleotide strand. Such multiple conjugates will typically be short aliphatic chains and lead to molecules with significantly shortened passenger strands.

In another embodiment of such oligonucleotide modifications bicyclic derivatives (LNA) can be added to keep shorter passenger strands stable with significant increases to the melting temperature of the resulting siRNA. In the case of LNA, affinity for the complementary strand is increased by 2-8° C. per nucleotide due to the extra cycle between 2′ and 4′ carbon, which fixes the 3′ endo ribose conformation (Julien et al., 2008). However, the introduction of this modification into siRNA strongly affects its interfering activity and the antisense strand is especially sensitive to this modification;

Since thermal asymmetry of the duplex makes a primary contribution to “guide” strand selection, modifications stabilizing the duplex formed by the 3′ end of the antisense strand and 5′ end of the sense strand and, conversely, modifications destabilizing the duplex formed by the 3′ end of the sense strand and 5′ end of the antisense strand can increase the efficiency of RNAi by providing favorable duplex thermal asymmetry. Thus, the introduction of LNA, UNA, or GNA at different ends of the duplex can lead to an increase in siRNA efficiency by increasing the probability of incorporation of the antisense strand into the RISC (Vaish et al., 2011). The use of conjugation as a method of delivering siRNA to cells involves forming siRNA conjugates with various molecules in old in the art. Such conjugations have included the use of folate or cholesterol (Thomas et al., 2009; and Letsinger et al., 1989), antibodies (Dassie et al., 2009) aptamers (Aronin, 2006), small peptides (Cesarone et al., 2007) and carbohydrates (Nair et al., 2014). Such references are incorporated herein by reference. According to the current invention conjugation molecules are used to aid in the delivery of molecules to target cells and penetrate the cell by known physiological transport mechanisms (ex: cholesterol (Lorenz et al., 2004)). Such short chains conjugates, even ethyl or propyl conjugates will change the behavior of the oligonucleotide of the invention if there are more than one of them.

a. Antisense Strands

In some embodiments, an oligonucleotide comprising one or more lipid conjugate is provided for targeting RNA comprises an antisense strand. In some embodiments, a provided oligonucleotide comprises an antisense strand comprising or consisting of at least 12 (e.g., at least 12, at least 13, at least 14, at least 15, at least 16, at least 17, at least 18, at least 19, at least 20, at least 21, at least 22, or at least 23) contiguous nucleotides of a sequence.

In some embodiments, a provided double-stranded oligonucleotide may have an antisense strand of up to 40 nucleotides in length (e.g., up to 40, up to 35, up to 30, up to 27, up to 25, up to 21, up to 19, up to 17, or up to 12 nucleotides in length). In some embodiments, a provided oligonucleotide may have an antisense strand of at least 12 nucleotides in length (e.g., at least 12, at least 15, at least 19, at least 21, at least 25, at least 27, at least 30, at least 35, or at least 38 nucleotides in length). In some embodiments, a provided oligonucleotide may have an antisense strand in a range of 12 to 40 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40, or 32 to 40) nucleotides in length. In some embodiments, a provided oligonucleotide may have an antisense strand of 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, an antisense strand of an oligonucleotide may be referred to as a “guide strand.” For example, if an antisense strand can engage with RNA-induced silencing complex (RISC) and bind to an Argonaut protein, or engage with or bind to one or more similar factors, and direct silencing of a target gene, it may be referred to as a guide strand. In some embodiments, a sense strand complementary to a guide strand may be referred to as a “passenger strand.”

b. Sense Strands

In some embodiments, an oligonucleotide comprising one or more lipid conjugate is provided for targeting RNA comprises a sense strand. In some embodiments, a provided oligonucleotide has a sense strand that comprises or consists of at least 12 (e.g., at least 13, at least 14, at least 15, at least 16, at least 17, at least 18, at least 19, at least 20, at least 21, at least 22, or at least 23) contiguous nucleotides of a sequence.

In some embodiments, a provided oligonucleotide may have a sense strand (or passenger strand) of up to 40 nucleotides in length (e.g., up to 40, up to 35, up to 30, up to 27, up to 25, up to 21, up to 19, up to 17, or up to 12 nucleotides in length). In some embodiments, a provided oligonucleotide may have a sense strand of at least 12 nucleotides in length (e.g., at least 12, at least 15, at least 19, at least 21, at least 25, at least 27, at least 30, at least 35, or at least 38 nucleotides in length). In some embodiments, a provided oligonucleotide may have a sense strand in a range of 12 to 40 (e.g., 12 to 40, 12 to 36, 12 to 32, 12 to 28, 15 to 40, 15 to 36, 15 to 32, 15 to 28, 17 to 21, 17 to 25, 19 to 27, 19 to 30, 20 to 40, 22 to 40, 25 to 40, or 32 to 40) nucleotides in length. In some embodiments, a provided oligonucleotide may have a sense strand of 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, a provided sense strand comprises a stem-loop structure at its 3′-end. In some embodiments, a provided sense strand comprises a stem-loop structure at its 5′-end. In some embodiments, a provided stem is a duplex of 2, 3, 4, 5, 6, 7, 8, 9, 10, 11, 12, 13, or 14 nucleotides in length. In some embodiments, a provided stem-loop provides the molecule better protection against degradation (e.g., enzymatic degradation) and facilitates targeting characteristics for delivery to a target cell. For example, in some embodiments, the loop provides added nucleotides on which modification can be made without substantially affecting the gene expression inhibition activity of an oligonucleotide. In certain embodiments, an oligonucleotide is provided herein in which the sense strand comprises (e.g., at its 3′-end) a stem-loop set forth as: S₁-L-S₂, in which S₁ is complementary to S₂, and in which L forms a loop between S₁ and S₂ of up to 10 nucleotides in length (e.g., 3, 4, 5, 6, 7, 8, 9, or 10 nucleotides in length).

In some embodiments, a provided loop of a stem-loop is a tetraloop (e.g., within a nicked tetraloop structure). A tetraloop may contain ribonucleotides, deoxyribonucleotides, modified nucleotides, and combinations thereof. Typically, a tetraloop has 4 to 5 nucleotides.

c. Duplex Length

In some embodiments, a duplex formed between a sense and antisense strand is at least 12 (e.g., at least 15, at least 16, at least 17, at least 18, at least 19, at least 20, or at least 21) nucleotides in length. In some embodiments, a duplex formed between a sense and antisense strand is in the range of 12-30 nucleotides in length (e.g., 12 to 30, 12 to 27, 12 to 22, 15 to 25, 18 to 30, 18 to 22, 18 to 25, 18 to 27, 18 to 30, 19 to 30 or 21 to 30 nucleotides in length). In some embodiments, a duplex formed between a sense and antisense strand is 12, 13, 14, 15, 16, 17, 18, 19, 20, 21, 22, 23, 24, 25, 26, 27, 28, 29, or 30 nucleotides in length. In some embodiments a duplex formed between a sense and antisense strand does not span the entire length of the sense strand and/or antisense strand. In some embodiments, a duplex between a sense and antisense strand spans the entire length of either the sense or antisense strands. In certain embodiments, a duplex between a sense and antisense strand spans the entire length of both the sense strand and the antisense strand.

d. Oligonucleotide Ends

In some embodiments, an oligonucleotide comprising one or more lipid conjugate described herein comprises sense and antisense strands, such that there is a 3′-overhang on either the sense strand or the antisense strand, or both the sense and antisense strand. In some embodiments, oligonucleotides provided herein have one 5′end that is thermodynamically less stable compared to the other 5′ end. In some embodiments, an asymmetric oligonucleotide is provided that includes a blunt end at the 3′ end of a sense strand and an overhang at the 3′ end of an antisense strand. In some embodiments, a 3′ overhang on an antisense strand is 1-8 nucleotides in length (e.g., 1, 2, 3, 4, 5, 6, 7 or 8 nucleotides in length).

Typically, a provided oligonucleotide for RNAi has a two-nucleotide overhang on the 3′ end of the antisense (guide) strand. However, other overhangs are possible. In some embodiments, an overhang is a 3′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides. However, in some embodiments, the overhang is a 5′ overhang comprising a length of between one and six nucleotides, optionally one to five, one to four, one to three, one to two, two to six, two to five, two to four, two to three, three to six, three to five, three to four, four to six, four to five, five to six nucleotides, or one, two, three, four, five or six nucleotides.

In some embodiments, one or more (e.g., 2, 3, 4) terminal nucleotides of the 3′ end or 5′ end of a sense and/or antisense strand are modified. For example, in some embodiments, one or two terminal nucleotides of the 3′ end of an antisense strand are modified. In some embodiments, the last nucleotide at the 3′ end of an antisense strand is modified, e.g., comprises 2′-modification, e.g., a 2′-O-methoxyethyl. In some embodiments, the last one or two terminal nucleotides at the 3′ end of an antisense strand are complementary to the target. In some embodiments, the last one or two nucleotides at the 3′ end of the antisense strand are not complementary to the target. In some embodiments, the 5′ end and/or the 3′ end of a sense or antisense strand has an inverted cap nucleotide.

e. Mismatches

In some embodiments, there is one or more (e.g., 1, 2, 3, 4, 5) mismatches between a sense and antisense strand. If there is more than one mismatch between a sense and antisense strand, they may be positioned consecutively (e.g., 2, 3 or more in a row), or interspersed throughout the region of complementarity. In some embodiments, the 3′-terminus of the sense strand contains one or more mismatches. In one embodiment, two mismatches are incorporated at the 3′-terminus of the sense strand. In some embodiments, base mismatches or destabilization of segments at the 3′-end of the sense strand of the oligonucleotide improved the potency of synthetic duplexes in RNAi, possibly through facilitating processing by Dicer.

ii. Single-Stranded Oligonucleotides

In some embodiments, a provided oligonucleotide for reducing RNA expression comprising a lipid conjugate is single-stranded. Such structures may include, but are not limited to, single-stranded RNAi oligonucleotides. Recent efforts have demonstrated the activity of single-stranded RNAi oligonucleotides (see, e.g., Matsui et al. (May 2016), MOLECULAR THERAPY, Vol. 24(5), 946-55). However, in some embodiments, oligonucleotides provided herein are antisense oligonucleotides (ASOs). An antisense oligonucleotide is a single-stranded oligonucleotide that has a nucleobase sequence which, when written in the 5′ to 3′ direction, comprises the reverse complement of a targeted segment of a particular nucleic acid and is suitably modified (e.g., as a gapmer) so as to induce RNaseH mediated cleavage of its target RNA in cells or (e.g., as a mixmer) so as to inhibit translation of the target mRNA in cells. Antisense oligonucleotides for use in the instant disclosure may be modified in any suitable manner known in the art including, for example, as shown in U.S. Pat. No. 9,567,587, which is incorporated by reference herein for its disclosure regarding modification of antisense oligonucleotides (including, e.g., length, sugar moieties of the nucleobase (pyrimidine, purine), and alterations of the heterocyclic portion of the nucleobase). Further, antisense molecules have been used for decades to reduce expression of specific target genes (see, e.g., Bennett et al.; Pharmacology of Antisense Drugs, Annual Review of Pharmacology and Toxicology, Vol. 57: 81-105).

iii. Oligonucleotide Modifications

The provided oligonucleotides comprising a lipid conjugate may be modified in various ways to improve or control specificity, stability, delivery, bioavailability, resistance from nuclease degradation, immunogenicity, base-paring properties, RNA distribution and cellular uptake and other features relevant to therapeutic or research use. See, e.g., Bramsen et al., NUCLEIC ACIDS RES., 2009, 37, 2867-81; Bramsen and Kjems (FRONTIERS IN GENETICS, 3 (2012): 1-22). Accordingly, in some embodiments, oligonucleotides of the present disclosure may include one or more suitable modifications. In some embodiments, a modified nucleotide has a modification in its base (or nucleobase), the sugar (e.g., ribose, deoxyribose), or the phosphate group.

The number of modifications on an oligonucleotide and the positions of those nucleotide modifications may influence the properties of an oligonucleotide. For example, oligonucleotides may be delivered in vivo by encompassing them in a lipid nanoparticle (LNP) or similar carrier. However, when an oligonucleotide is not protected by an LNP or similar carrier (e.g., “naked delivery”), it may be advantageous for at least some of the its nucleotides to be modified. Accordingly, in certain embodiments of any of the oligonucleotides provided herein, all or substantially all of the nucleotides of an oligonucleotide are modified. In certain embodiments, more than half of the nucleotides are modified. In certain embodiments, less than half of the nucleotides are modified. Typically, with naked delivery, every sugar is modified at the 2′-position. These modifications may be reversible or irreversible. In some embodiments, a provided oligonucleotide has a number and type of modified nucleotides sufficient to cause the desired characteristic (e.g., protection from enzymatic degradation, capacity to target a desired cell after in vivo administration, and/or thermodynamic stability).

a. Sugar Modifications

In some embodiments, a modified sugar (also referred to herein as a sugar analog) includes a modified deoxyribose or ribose moiety, e.g., in which one or more modifications occur at the 2′, 3′, 4′, and/or 5′ carbon position of the sugar. In some embodiments, a modified sugar may also include non-natural alternative carbon structures such as those present in locked nucleic acids (“LNA”) (see, e.g., Koshkin el al. (1998), TETRAHEDRON 54, 3607-3630), unlocked nucleic acids (“UNA”) (see, e.g., Snead et al. (2013), MOLECULAR THERAPY—NUCLEIC ACIDS, 2, e103), and bridged nucleic acids (“BNA”) (see, e.g., Imanishi and Obika (2002), THE ROYAL SOCIETY OF CHEMISTRY, CHEM. COMMUN., 1653-1659). Koshkin et al, Snead et al, and Imanishi and Obika are incorporated by reference herein for their disclosures relating to sugar modifications.

In some embodiments, a nucleotide modification in a sugar comprises a 2′-modification. In certain embodiments, the 2′-modification may be 2′-aminoethyl, 2′-fluoro, 2′-O-methyl, 2′-O-methoxyethyl, or 2′-deoxy-2′-fluoro-β-d-arabinonucleic acid. Typically, the modification is 2′-fluoro, 2-O-methyl, or 2′-O-methoxyethyl. However, a large variety of 2′ position modifications that have been developed for use in oligonucleotides can be employed in oligonucleotides disclosed herein. See, e.g., Bramsen et al., NUCLEIC ACIDS RES., 2009, 37, 2867-2881. In some embodiments, a modification in a sugar comprises a modification of the sugar ring, which may comprise modification of one or more carbons of the sugar ring. For example, a modification of a sugar of a nucleotide may comprise a linkage between the 2′-carbon and a 1′-carbon or 4′-carbon of the sugar. For example, the linkage may comprise an ethylene or methylene bridge. In some embodiments, a modified nucleotide has an acyclic sugar that lacks a 2′-carbon to 3′-carbon bond. In some embodiments, a modified nucleotide has a thiol group, e.g., in the 4′-position of the sugar.

In some embodiments, the terminal 3′-end group (e.g., a 3′-hydroxyl) is a phosphate group or other group, which can be used, for example, to attach linkers, adapters or labels or for the direct ligation of an oligonucleotide to another nucleic acid.

b. 5′-Terminal Phosphates

5′-Terminal phosphate groups of oligonucleotides may or in some circumstances enhance the interaction with Argonaute 2. However, oligonucleotides comprising a 5′-phosphate group may be susceptible to degradation via phosphatases or other enzymes, which can limit their bioavailability in vivo. In some embodiments, a provided oligonucleotide includes analogs of 5′-phosphates that are resistant to such degradation. In some embodiments, a phosphate analog may be oxymethylphosphonate, vinylphosphonate, or malonylphosphonate. In certain embodiments, the 5′-end of an oligonucleotide strand is attached to a chemical moiety that mimics the electrostatic and steric properties of a natural 5′-phosphate group (“phosphate mimic”) (see, e.g., Prakash et al. (2015), NUCLEIC ACIDS RES., March 31; 43(6): 2993-3011, the contents of which relating to phosphate analogs are incorporated herein by reference). Many phosphate mimics have been developed that can be attached to the 5′-end (see, e.g., U.S. Pat. No. 8,927,513, the contents of which relating to phosphate analogs are incorporated herein by reference). Other modifications have been developed for the 5′ end of oligonucleotides (see, e.g., WO 2011/133871, the contents of which relating to phosphate analogs are incorporated herein by reference). In certain embodiments, a hydroxyl group is attached to the 5′-end of the oligonucleotide.

In some embodiments, a provided oligonucleotide has a phosphate analog at a 4′-carbon position of the sugar (referred to as a “4′-phosphate analog”). See, for example, WO 2018/045317 and US 2019/177729, the contents of each of which relating to phosphate analogs are incorporated herein by reference. In some embodiments, an oligonucleotide provided herein comprises a 4′-phosphate analog at a 5′-terminal nucleotide. In some embodiments, the phosphate analog is an oxymethylphosphonate, in which the oxygen atom of the oxymethyl group is bound to the sugar moiety (e.g., at its 4′-carbon) or analog thereof. In other embodiments, the 4′-phosphate analog is a thiomethylphosphonate or an aminomethylphosphonate, in which the sulfur atom of the thiomethyl group or the nitrogen atom of the aminomethyl group is bound to the 4′-carbon of the sugar moiety or analog thereof. In certain embodiments, the 4′-phosphate analog is an oxymethylphosphonate. In some embodiments, an oxymethylphosphonate is represented by the formula —O—CH₂—PO(OH)₂ or —O—CH₂—PO(OR)₂, in which R is independently selected from H, —CH₃, an alkyl group, —CH₂CH₂CN, —CH₂OCOC(CH₃)₃, —CH₂OCH₂CH₂Si(CH), or a protecting group. In certain embodiments, the alkyl group is —CH₂CH₃. More typically, R is independently selected from H, —CH₃, or —CH₂CH₃.

c. Modified Internucleoside Linkages

In some embodiments, a provided oligonucleotide may comprise a modified internucleoside linkage. In some embodiments, phosphate modifications or substitutions may result in an oligonucleotide that comprises at least one (e.g., at least 1, at least 2, at least 3 or at least 5) modified internucleotide linkage. In some embodiments, any one of the oligonucleotides disclosed herein comprises 1 to 10 (e.g., 1 to 10, 2 to 8, 4 to 6, 3 to 10, 5 to 10, 1 to 5, 1 to 3 or 1 to 2) modified internucleotide linkages. In some embodiments, any one of the oligonucleotides disclosed herein comprises 1, 2, 3, 4, 5, 6, 7, 8, 9, or 10 modified internucleotide linkages.

A modified internucleotide linkage may be a phosphorodithioate linkage, a phosphorothioate linkage, a phosphotriester linkage, a thionoalkylphosphonate linkage, a thionoalkylphosphotriester linkage, a phosphoramidite linkage, a phosphonate linkage or a boranophosphate linkage. In some embodiments, at least one modified internucleotide linkage of any one of the oligonucleotides as disclosed herein is an oxymethylphosphonate, or phosphorothioate linkage.

d. Base Modifications

In some embodiments, oligonucleotides provided herein have one or more modified nucleobases. In some embodiments, modified nucleobases (also referred to herein as base analogs) are linked at the 1′-position of a nucleotide sugar moiety. In certain embodiments, a modified nucleobase is a nitrogenous base. In certain embodiments, a modified nucleobase does not contain a nitrogen atom. See e.g., US 2008/274462. In some embodiments, a modified nucleotide comprises a universal base. In some embodiments, a universal base is a heterocyclic moiety located at the 1-position of a nucleotide sugar moiety in a modified nucleotide, or the equivalent position in a nucleotide sugar moiety substitution that, when present in a duplex, can be positioned opposite more than one type of base without substantially altering the structure of the duplex. In some embodiments, compared to a reference single-stranded nucleic acid (e.g., oligonucleotide) that is fully complementary to a target nucleic acid, a single-stranded nucleic acid containing a universal base forms a duplex with the target nucleic acid that has a lower Tₘ than a duplex formed with the complementary nucleic acid. However, in some embodiments, compared to a reference single-stranded nucleic acid in which the universal base has been replaced with a base to generate a single mismatch, the single-stranded nucleic acid containing the universal base forms a duplex with the target nucleic acid that has a higher Tₘ than a duplex formed with the nucleic acid comprising the mismatched base.

Non-limiting examples of universal-binding nucleotides include inosine, 1-β-D-ribofuranosyl-5-nitroindole, and/or 1-β-D-ribofuranosyl-3-nitropyrrole. See e.g., US 2007/254362; Van Aerschot et al., NUCLEIC ACIDS RES. 1995 Nov. 11; 23(21):4363-70; Loakes et al., NUCLEIC ACIDS RES. 1995 Jul. 11; 23(13):2361-6; and Loakes and Brown, NUCLEIC ACIDS RES. 1994 Oct. 11; 22(20):4039-43, the entity of each of which is hereby incorporated by reference.

e. Reversible Modifications

While certain modifications to protect an oligonucleotide from the in vivo environment before reaching target cells can be made, they can reduce the potency or activity of the oligonucleotide once it reaches the cytosol of the target cell. Reversible modifications can be made such that the molecule retains desirable properties outside of the cell, which are then removed upon entering the cytosolic environment of the cell. Reversible modification can be removed, for example, by the action of an intracellular enzyme or by the chemical conditions inside of a cell (e.g., through reduction by intracellular glutathione).

In some embodiments, a reversibly modified nucleotide comprises a glutathione-sensitive moiety. Typically, nucleic acid molecules have been chemically modified with cyclic disulfide moieties to mask the negative charge created by the internucleotide diphosphate linkages and improve cellular uptake and nuclease resistance. See US 2011/0294869, WO 2015/188197, Meade et al., NATURE BIOTECHNOLOGY, 2014, 32:1256-63, and WO 2014/088920, the entity of each of which is hereby incorporated by reference for their disclosures of such modifications. This reversible modification of the internucleotide diphosphate linkages is designed to be cleaved intracellularly by the reducing environment of the cytosol (e.g. glutathione). Earlier examples include neutralizing phosphotriester modifications that were reported to be cleavable inside cells (Dellinger et al. J. AM. CHEM. SOC. 2003, 125:940-950).

In some embodiments, such a reversible modification allows protection during in vivo administration (e.g., transit through the blood and/or lysosomal/endosomal compartments of a cell) where the oligonucleotide will be exposed to nucleases and other harsh environmental conditions (e.g., pH). When released into the cytosol of a cell where the levels of glutathione are higher compared to extracellular space, the modification is reversed and the result is a cleaved oligonucleotide. Using reversible, glutathione sensitive moieties, it is possible to introduce sterically larger chemical groups into the oligonucleotide of interest as compared to the options available using irreversible chemical modifications. This is because these larger chemical groups will be removed in the cytosol and, therefore, should not interfere with the biological activity of the oligonucleotides inside the cytosol of a cell. As a result, these larger chemical groups can be engineered to confer various advantages to the nucleotide or oligonucleotide, such as nuclease resistance, lipophilicity, charge, thermal stability, specificity, and reduced immunogenicity. In some embodiments, the structure of the glutathione-sensitive moiety can be engineered to modify the kinetics of its release.

In some embodiments, a glutathione-sensitive moiety is attached to the sugar of the nucleotide. In some embodiments, a glutathione-sensitive moiety is attached to the 2′-carbon of the sugar of a modified nucleotide. In some embodiments, the glutathione-sensitive moiety is located at the 5′-carbon of a sugar, particularly when the modified nucleotide is the 5′-terminal nucleotide of the oligonucleotide. In some embodiments, the glutathione-sensitive moiety is located at the 3′-carbon of a sugar, particularly when the modified nucleotide is the 3′-terminal nucleotide of the oligonucleotide. In some embodiments, the glutathione-sensitive moiety comprises a sulfonyl group. See, e.g., WO 2018/039364, the entity of which is hereby incorporated by reference

v. Targeting Ligands

In some embodiments, a provided oligonucleotide comprising a lipid conjugate targets one or more cells or one or more organs. Such a targeting strategy may help to avoid undesirable effects in other organs, or may avoid undue loss of the oligonucleotide to cells, tissue or organs that would not benefit for the oligonucleotide. Accordingly, in some embodiments, a provided oligonucleotide may be further modified to facilitate improved targeting of a tissue, cell, or organ. In certain embodiments, oligonucleotides disclosed herein may facilitate delivery of the oligonucleotide to a broad range of tissues, e.g., CNS, muscle, adipose, or adrenal gland. In some embodiments, a provided oligonucleotide comprises a nucleotide that is conjugated to one or more targeting ligands. A targeting ligand may comprise a carbohydrate, amino sugar, cholesterol, peptide, polypeptide, protein or part of a protein (e.g., an antibody or antibody fragment). In some embodiments, a targeting ligand is an aptamer. For example, a targeting ligand may be an RGD peptide that is used to target tumor vasculature or glioma cells, CREKA peptide to target tumor vasculature or stoma, transferrin, lactoferrin, or an aptamer to target transferrin receptors expressed on CNS vasculature, or an anti-EGFR antibody to target EGFR on glioma cells.

Appropriate methods or chemistry (e.g., click chemistry) can be used to link a targeting ligand to a nucleotide. In some embodiments, a targeting ligand is conjugated to a nucleotide using a click linker. In some embodiments, an acetal-based linker is used to conjugate a targeting ligand to a nucleotide of any one of the oligonucleotides described herein. Acetal-based linkers are disclosed, for example, in WO 2016/100401, the entity of which is hereby incorporated by reference. In some embodiments, the linker is a labile linker. However, in other embodiments, the linker is stable. In some embodiments, a duplex extension (up to 3, 4, 5, or 6 base pairs in length) is provided between a targeting ligand and a double-stranded oligonucleotide.

In some embodiments, the oligonucleotide comprises 1, 2, 3, or 4 units formula II-b-2. In some embodiments, the oligonucleotide comprises one or more units of formula II-b-2 wherein B is guanine (G) or adenine (A). In some embodiments, the oligonucleotide comprises a GAAA tetraloop comprising 1, 2, 3, or 4 units formula II-b-2

Exemplary nucleic acid-ligand conjugates thereof comprising a lipid conjugate of the disclosure are set forth in Table 1.

Exemplary oligonucleotide-ligand conjugates or analogues thereof comprising one or more adamntyl or lipid moiety are disclosed in Table 2:

TABLE 2
Exemplary oligonucleotide-ligand conjugates
Exemplary Oligonucleotide-ligand conjugate duplexes

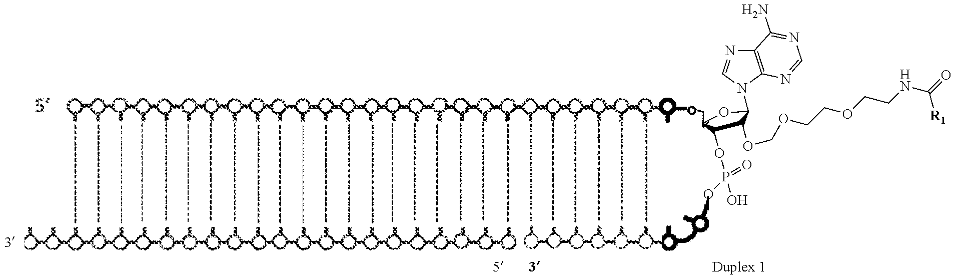

R₁COOH group represents fatty acid C8:0, C10:0, C11:0, C12:0, C14:0, C16:0, C17:0, C18:0, C22:0,
C24:0, C26:0, C22:6, C24:1, diacyl C16:0 or diacyl C18:1

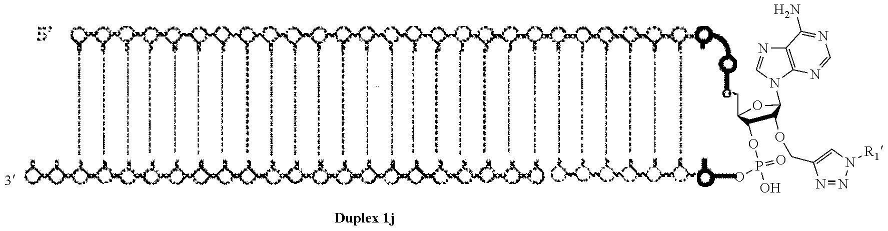

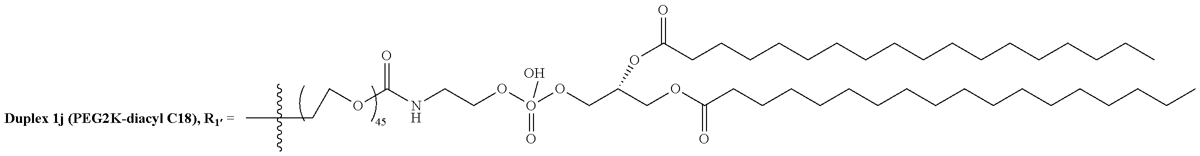

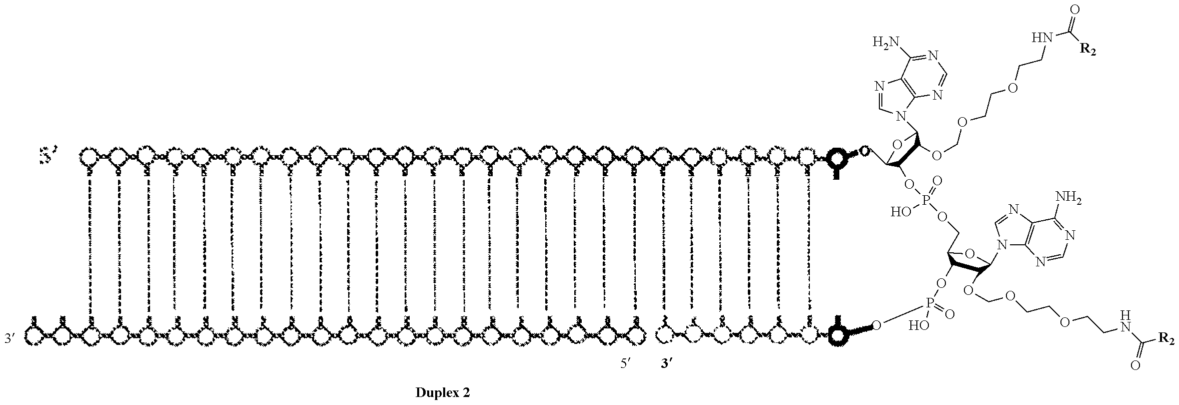

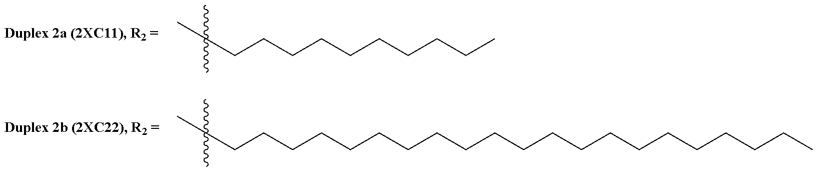 [SMILES: *CCCCCCCCCC ; *CCCCCCCCCCCCCCCCCCCCC]

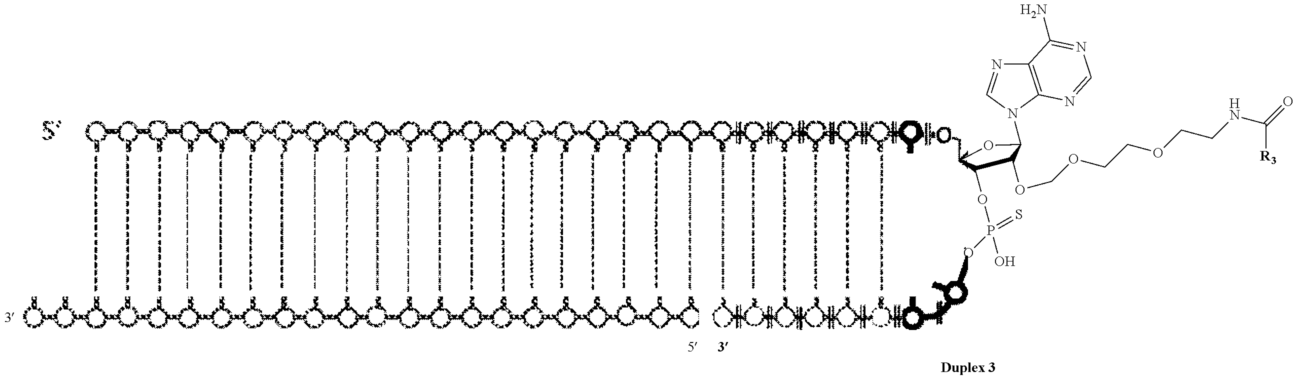

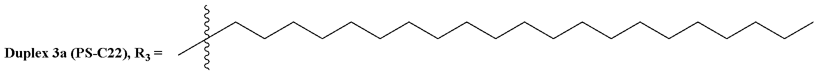 [SMILES: *CCCCCCCCCCCCCCCCCCCCC]

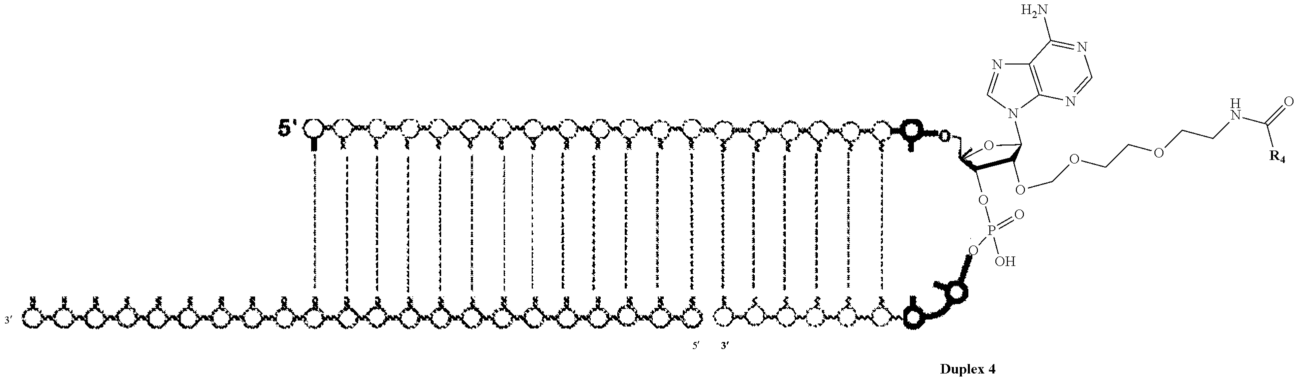

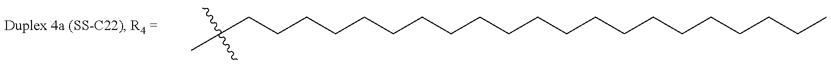 [SMILES: *CCCCCCCCCCCCCCCCCCCCC]

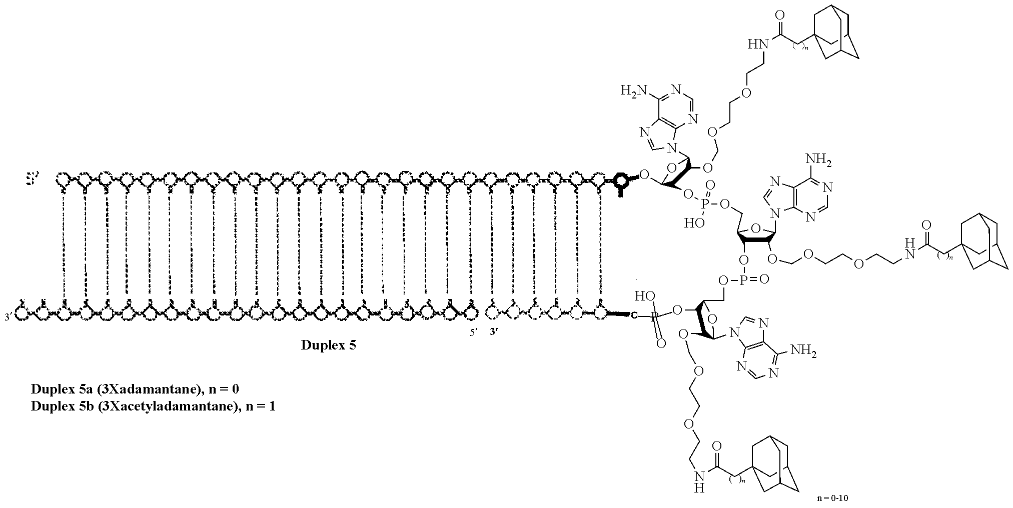

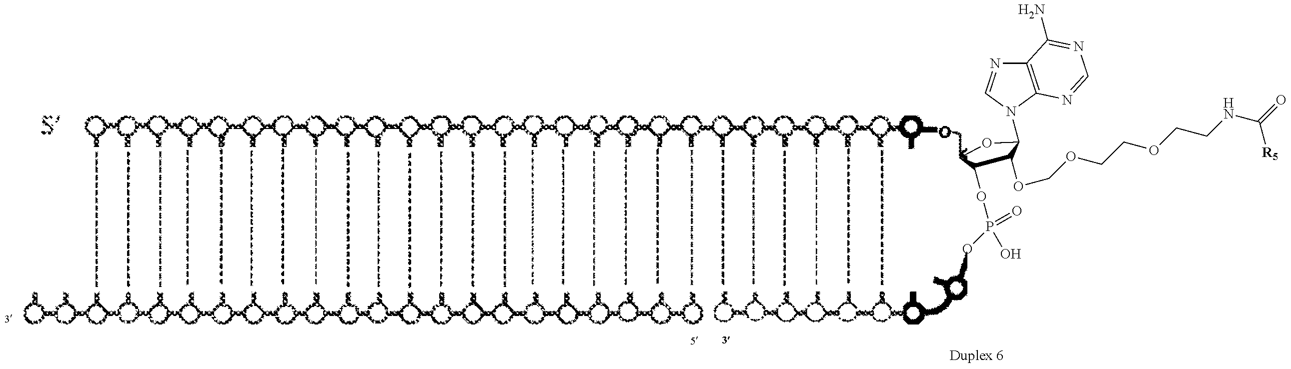

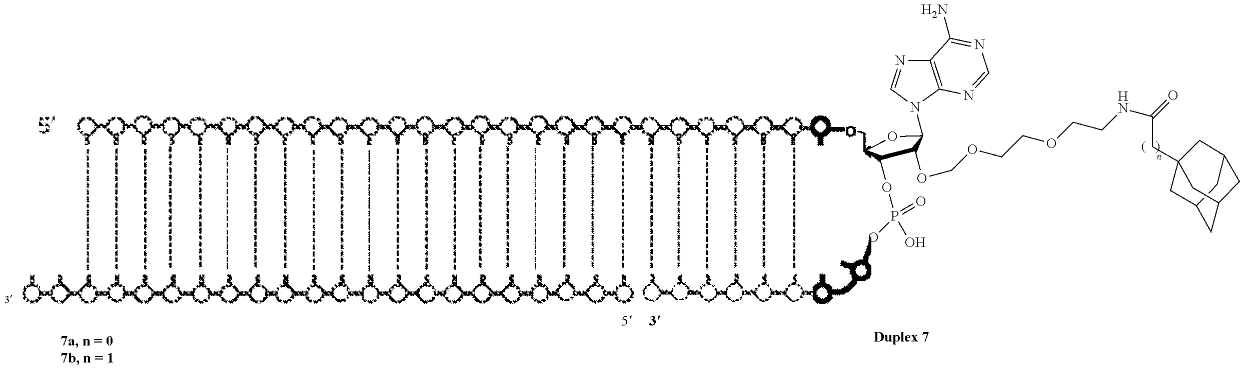

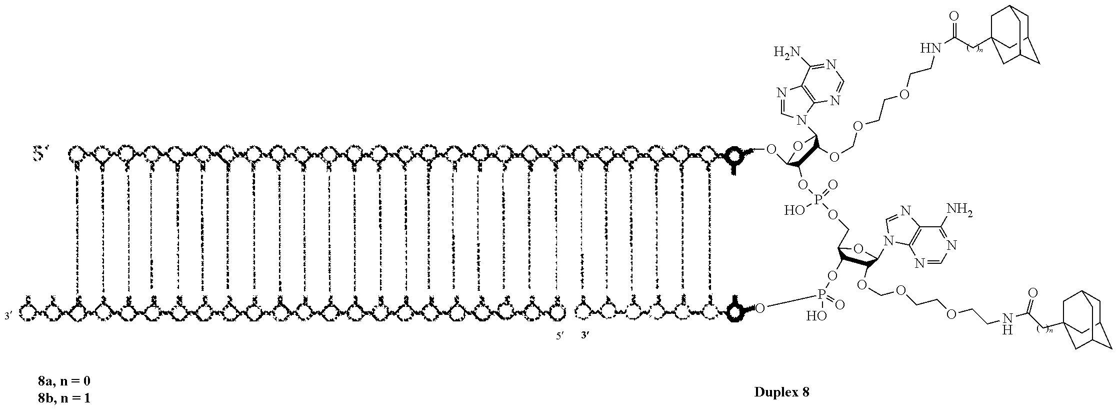

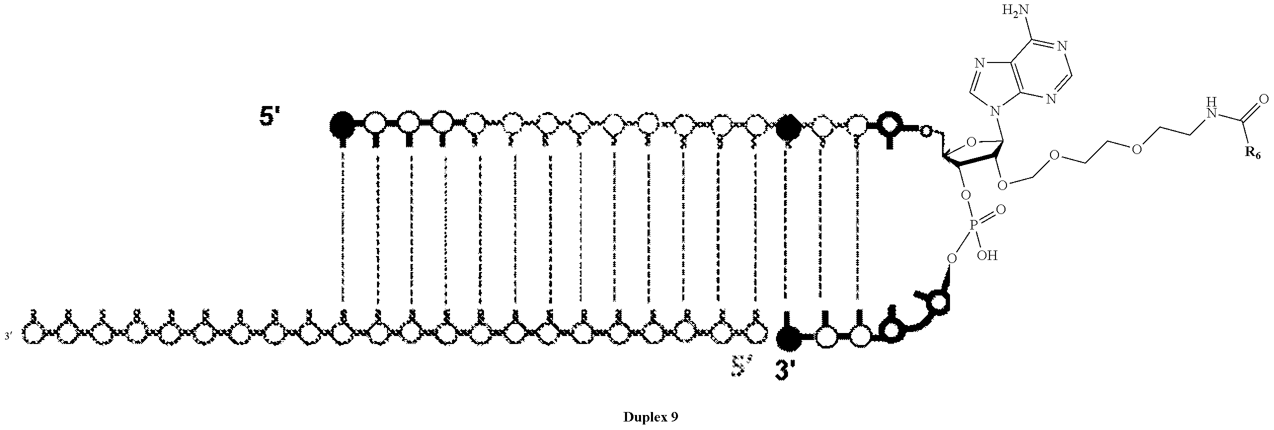

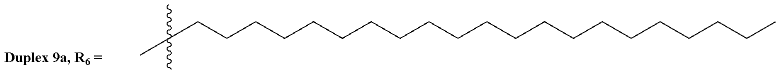 [SMILES: *CCCCCCCCCCCCCCCCCCCCC]

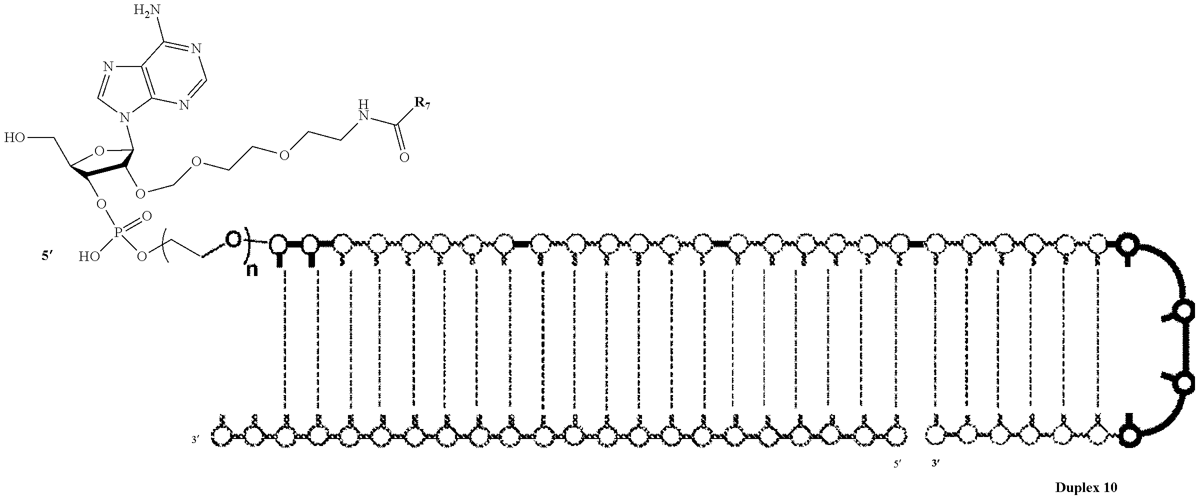

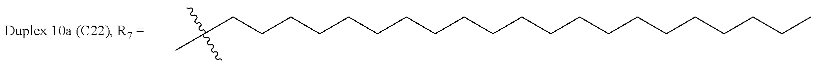 [SMILES: *CCCCCCCCCCCCCCCCCCCCC]

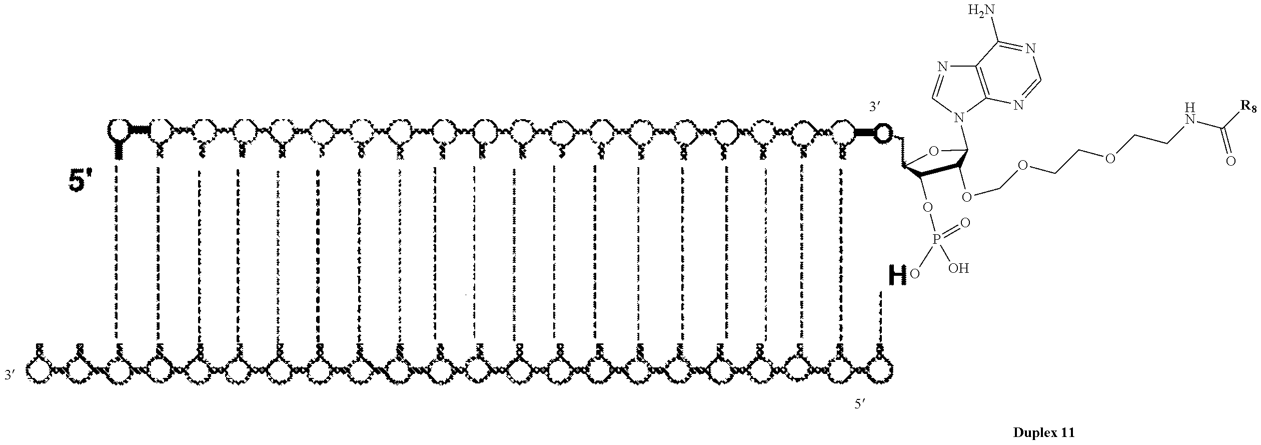

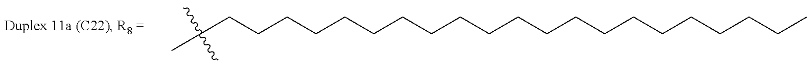 [SMILES: *CCCCCCCCCCCCCCCCCCCCC]

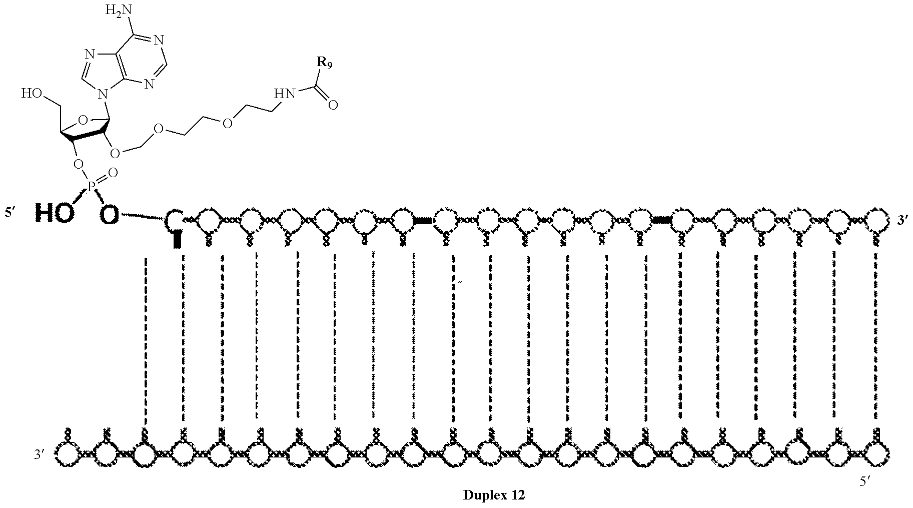

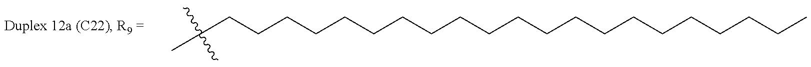 [SMILES: *CCCCCCCCCCCCCCCCCCCCC]

In some embodiments, the present disclosure provides an oligonucleotide-ligand conjugate comprising one or more adamantyl or lipid moieties, as described in table 2, in the description and the examples, or a pharmaceutically acceptable salt thereof.

In some embodiments, the present disclosure provides a double stranded oligonucleotide comprising one or more ligand conjugates of the disclosure, as in table 2, in the description and the examples, or a pharmaceutically acceptable salt thereof.

### 5. General Methods of Providing the Nucleic Acids and Analogues Thereof

The nucleic acids and analogues thereof comprising lipid conjugate described herein can be made using a variety of synthetic methods known in the art, including standard phosphoramidite methods. Any phosphoramidite synthesis method can be used to synthesize the provided nucleic acids of this disclosure. In certain embodiments, phosphoramidites are used in a solid phase synthesis method to yield reactive intermediate phosphite compounds, which are subsequently oxidized using known methods to produce phosphonate-modified oligonucleotides, typically with a phosphodiester or phosphorothioate internucleotide linkages. The oligonucleotide synthesis of the present disclosure can be performed in either direction: from 5′ to 3′ or from 3′ to 5′ using art known methods.

In certain embodiments, the method for synthesizing a provided nucleic acid comprises (a) attaching a nucleoside or analogue thereof to a solid support via a covalent linkage; (b) coupling a nucleoside phosphoramidite or analogue thereof to a reactive hydroxyl group on the nucleoside or analogue thereof of step (a) to form an internucleotide bond there between, wherein any uncoupled nucleoside or analogue thereof on the solid support is capped with a capping reagent; (c) oxidizing said internucleotide bond with an oxidizing agent; and (d) repeating steps (b) to (c) iteratively with subsequent nucleoside phosphoramidites or analogue thereof to form a nucleic acid or analogue thereof, wherein at least the nucleoside or analogue thereof of step (a), the nucleoside phosphoramidite or analogue thereof of step (b) or at least one of the subsequent nucleoside phosphoramidites or analogues thereof of step (d) comprises a lipid conjugate moiety as described herein. Typically, the coupling, capping/oxidizing steps and optionally, deprotecting steps, are repeated until the oligonucleotide reaches the desired length and/or sequence, after which it is cleaved from the solid support. In certain embodiments, an oligonucleotide is prepared comprising 1-3 nucleic acid or analogues thereof comprising lipid conjugates units on a tetraloop.

In Scheme A below, where a particular protecting group, leaving group, or transformation condition is depicted, one of ordinary skill in the art will appreciate that other protecting groups, leaving groups, and transformation conditions are also suitable and are contemplated. Certain reactive functional groups (e.g., —N(H)—, —OH, etc.) envisioned in the genera in Scheme A requiring additional protection group strategies are also contemplated and is appreciated by those having ordinary skill in the art. Such groups and transformations are described in detail in March's Advanced Organic Chemistry: Reactions, Mechanisms, and Structure, M. B. Smith and J. March, 5^{th} Edition, John Wiley & Sons, 2001, COMPREHENSIVE ORGANIC TRANSFORMATIONS, (R. C. Larock, 2^{nd} Edition, John Wiley & Sons, 1999), and PROTECTING GROUPS IN ORGANIC SYNTHESIS, (T. W. Greene and P. G. M. Wuts, 3^{rd} edition, John Wiley & Sons, 1999), the entirety of each of which is hereby incorporated herein by reference.

In certain embodiments, nucleic acids and analogues thereof of the present disclosure are generally prepared according to Scheme A, Scheme A1 and Scheme B set forth below:

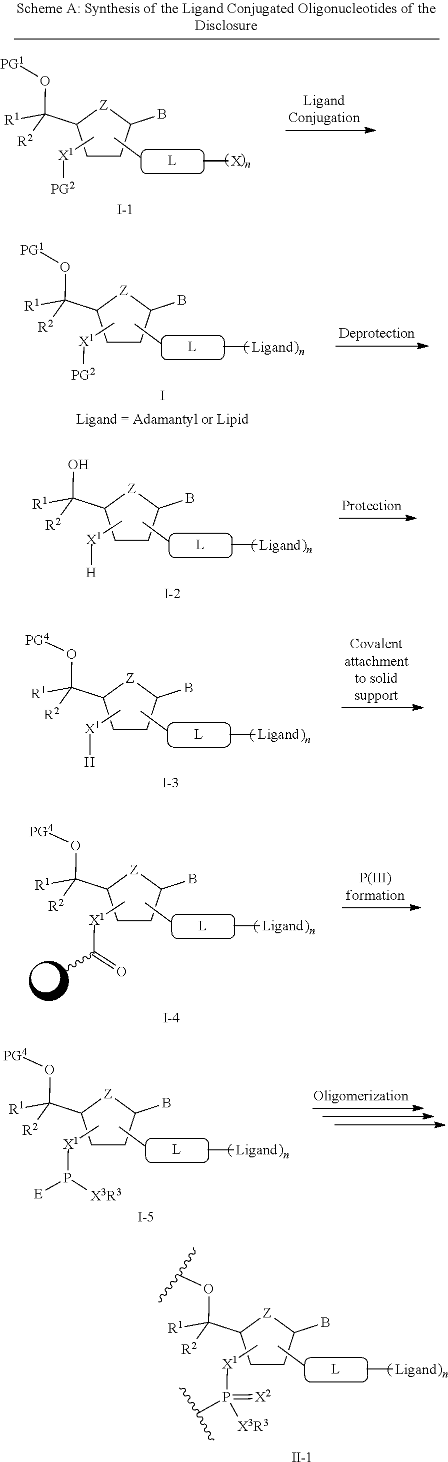

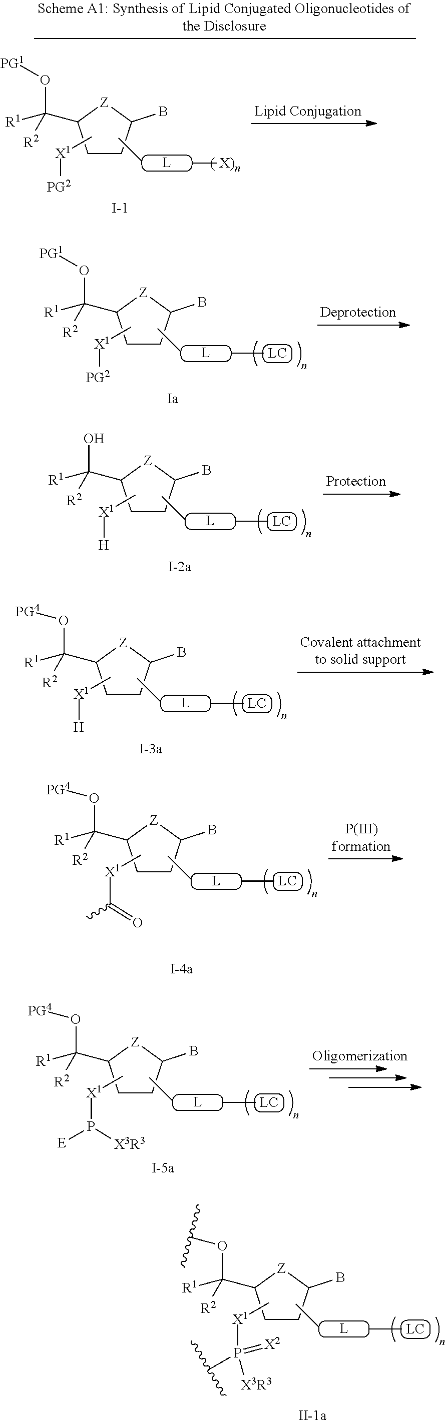

As depicted in Scheme A and Scheme A1 above, a nucleic acid or analogue thereof of formula I-1 is conjugated with one or more ligand/lipophilic compound to form a compound of formula I or Ia comprising one more ligand/lipid conjugates. Typically, conjugation is performed through an esterification or amidation reaction between a nucleic acid or analogue thereof of formula I-1 or I-1a and one or more adamantyl and/or lipophilic compound (e.g., fatty acid) in series or in parallel by known techniques in the art. Nucleic acid or analogue thereof of formula I or Ia can then be deprotected to form a compound of formula 1-2 or I-2a and protected with a suitable hydroxyl protecting group (e.g., DMTr) to form a compound of formula I-3 or I-3a. In one aspect, nucleic acid-ligand conjugates of formula 1-3 or I-3a can be covalently attached to a solid support (e.g., through a succinic acid linking group) to form a solid support nucleic acid-ligand conjugate or analogue thereof of formula 1-4 or I-4a comprising one or more adamantyl and/or lipid conjugate. In another aspect, a nucleic acid-ligand conjugates of formula I-3 or I-3a can react with a P(III) forming reagent (e.g., 2-cyanoethyl N,N-diisopropylchlorophosphoramidite) to form a nucleic acid or analogue thereof of formula I-5 or I-5a comprising a P(III) group. A nucleic acid-ligand conjugate or analogue thereof of formula I-5 or I-5a can then be subjected to oligomerization forming conditions preformed using known and commonly applied processes to prepare oligonucleotides in the art. For example, the compound of formula I-5 or I-5a is coupled to a solid supported nucleic acid-ligand conjugate or analogue thereof bearing a 5′-hydroxyl group. Further steps can comprise one or more deprotections, couplings, phosphite oxidation, and/or cleavage from the solid support to provide an oligonucleotide of various nucleotide lengths, including one or more lipid conjugate nucleotide units represented by a compound of formula II-1 or II-Ia. Each of B, E, L, ligand, LC, n, PG¹, PG², PG⁴, R¹, R², R³, X, X¹, X², X³, and Z is as defined above and described herein.

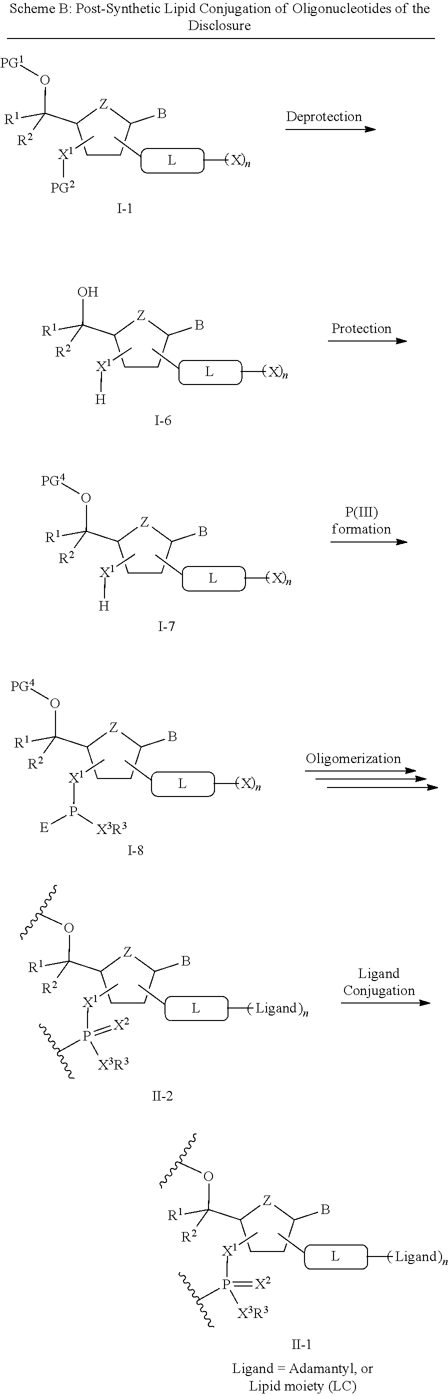

As depicted in Scheme B above, a nucleic acid or analogue thereof of formula I-1 can be deprotected to form a compound of formula I-6, protected with a suitable hydroxyl protecting group (e.g., DMTr) to form a compound of formula I-7, and reacted with a P(III) forming reagent (e.g., 2-cyanoethyl N,N-diisopropylchlorophosphoramidite) to form a nucleic acid or analogue thereof of formula I-8 comprising a P(III) group. Next, a nucleic acid or analogue thereof of formula I-8 is subjected to oligomerization forming conditions preformed using known and commonly applied processes to prepare oligonucleotides in the art. For example, the compound of formula I-8 is coupled to a solid supported nucleic acid or analogue thereof bearing a 5′-hydroxyl group. Further steps can comprise one or more deprotections, couplings, phosphite oxidation, and/or cleavage from the solid support to provide an oligonucleotide of various nucleotide lengths represented by a compound of formula II-2. An oligonucleotide of formula II-2 can then be conjugated with one or more ligands e.g. adamntyl, or lipophilic compound (e.g., fatty acid) to form a compound of formula II-1 comprising one or more ligand conjugates. Typically, conjugation is performed through an esterification or amidation reaction between a nucleic acid or analogue thereof of formula II-2 and one or more adamantyl or fatty acid in series or in parallel by known techniques in the art. Each of B, E, L, ligand, LC, n, PG¹, PG², PG⁴, R¹, R², R³, X, X¹, X², X³, and Z is as defined above and described herein.

In certain embodiments, nucleic acids and analogues thereof of the present disclosure are prepared according to Scheme C and Scheme D set forth below:

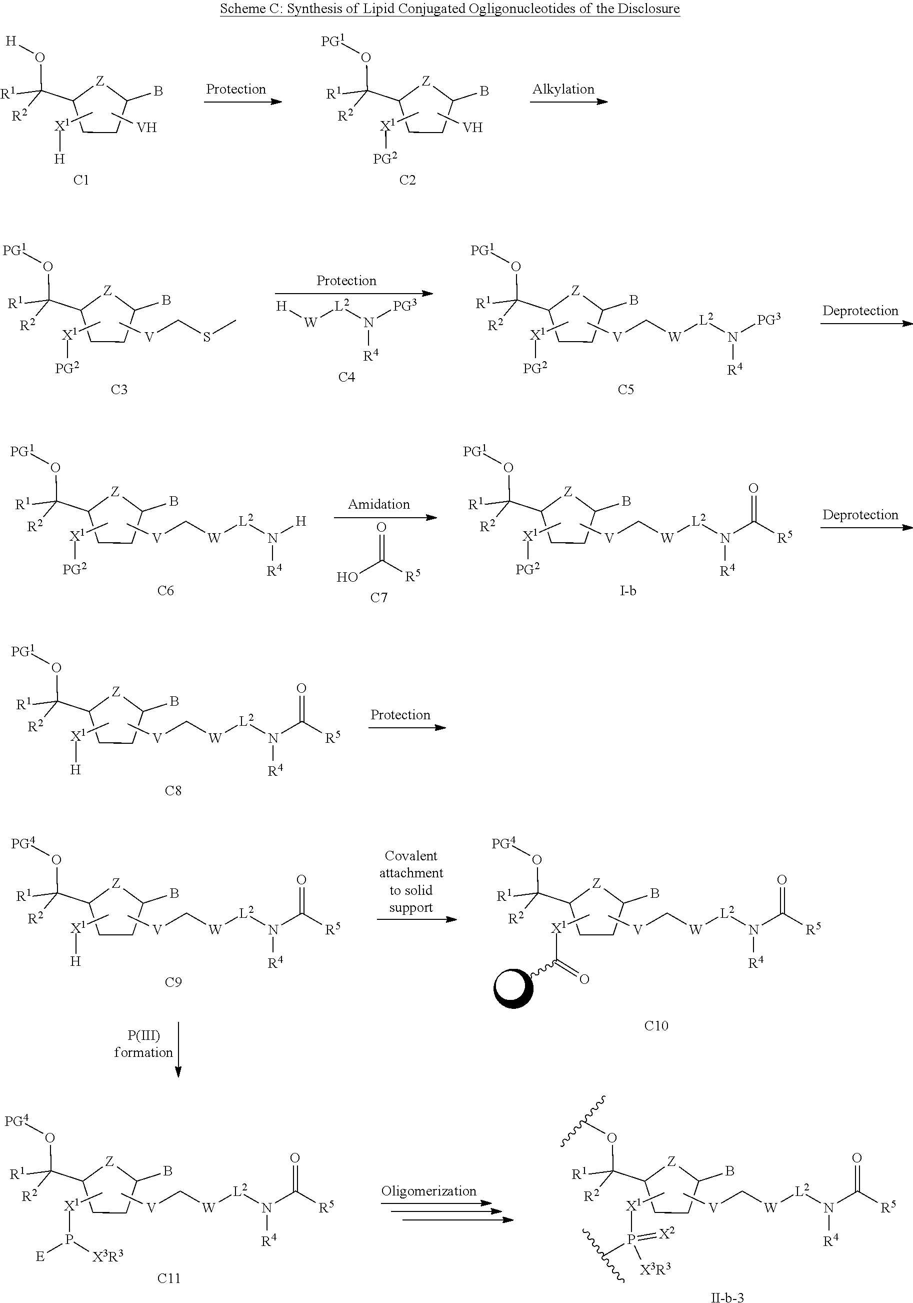

As depicted in Scheme C above, a nucleic acid or analogue thereof of formula C1 is protected to form a compound of formula C2. Nucleic acid or analogue thereof of formula C2 is then alkylated (e.g., using DMSO and acetic acid via the Pummerer rearrangement) to form a monothioacetal compound of formula C3. Next, nucleic acid or analogue thereof of formula C3 is coupled with C4 under appropriate conditions (e.g., mild oxidizing conditions) to form a nucleic acid or analogue thereof of formula C5. Nucleic acid or analogue thereof of formula C5 can then be deprotected to form a compound of formula C6 and coupled with a ligand (adamntyl or lipophilic compound(e.g., a fatty acid)) of formula C7 under appropriate amide forming conditions (e.g., HATU, DIPEA), to form a nucleic acid-ligand conjugate or analogue thereof of formula I-b comprising a lipid conjugate of the disclosure. Nucleic acid-ligand conjugate or analogue thereof of formula I-b can then be deprotected to form a compound of formula C8 and protected with a suitable hydroxyl protecting group (e.g., DMTr) to form a compound of formula C9. In one aspect, nucleic acid or analogue thereof of formula C9 can be covalently attached to a solid support (e.g., through a succinic acid linking group) to form a solid support nucleic acid-ligand conjugate or analogue thereof of formula C10 comprising a ligand conjugate (adamntyl or lipid moiety) of the disclosure. In another aspect, a nucleic acid-ligand conjugate or analogue thereof of formula C9 can reacted with a P(III) forming reagent (e.g., 2-cyanoethyl N,N-diisopropylchlorophosphoramidite) to form a nucleic acid-ligand conjugate or analogue thereof of formula C11 comprising a P(III) group. A nucleic acid-ligand conjugate or analogue thereof of formula C11 can then be subjected to oligomerization forming conditions preformed using known and commonly applied processes to prepare oligonucleotides in the art. For example, the compound of formula C11 is coupled to a solid supported nucleic acid-ligand conjugate or analogue thereof bearing a 5′-hydroxyl group. Further steps can comprise one or more deprotections, couplings, phosphite oxidation, and/or cleavage from the solid support to provide an oligonucleotide of various nucleotide lengths, including one or more adamantyl and/or lipid conjugate nucleotide units represented by a compound of formula II-b-3. Each of B, E, L², PG¹, PG², PG³, PG⁴, R¹, R², R³, R⁴, R⁵, X¹, X², X³, V, W, and Z is as defined above and described herein.

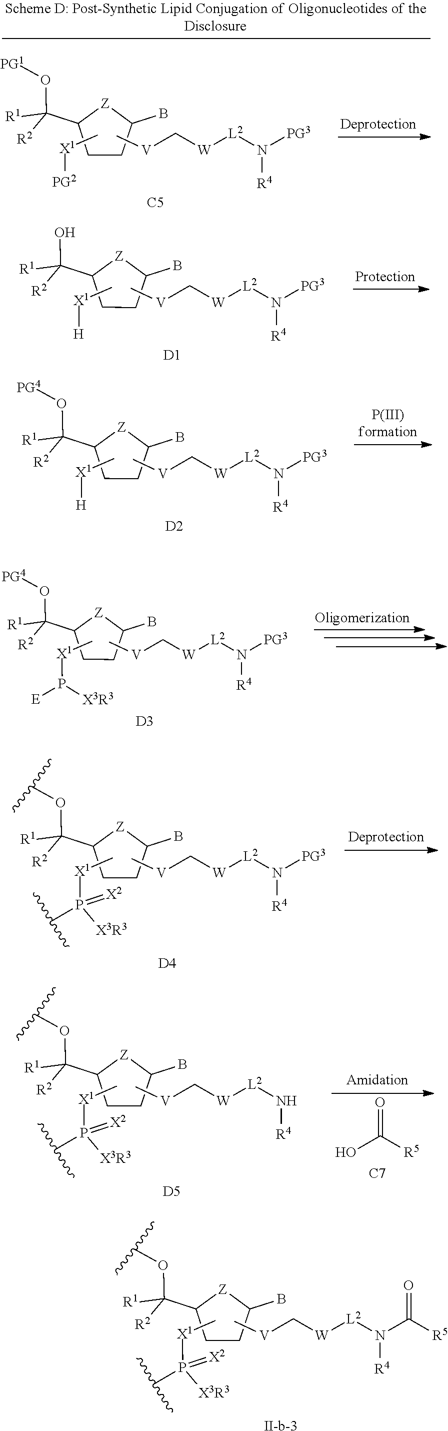

Each of B, E, L², PG¹, PG², PG³, PG⁴, R¹, R², R², R⁴, R¹, X¹, X², X³, V, W, and Z is as defined above and described herein. As depicted in Scheme D above, a nucleic acid or analogue thereof of formula C5 can be selectively deprotected to form a compound of formula D1, protected with a suitable hydroxyl protecting group (e.g., DMTr) to form a compound of formula D2, and reacted with a P(III) forming reagent (e.g., 2-cyanoethyl N,N-diisopropylchlorophosphoramidite) to form a nucleic acid or analogue thereof of formula D3. Next, a nucleic acid or analogue thereof of formula D3 is subjected to oligomerization forming conditions preformed using known and commonly applied processes to prepare oligonucleotides in the art. For example, the compound of formula D3 is coupled to a solid supported nucleic acid or analogue thereof bearing a 5′-hydroxyl group. Further steps can comprise one or more deprotections, couplings, phosphite oxidation, and/or cleavage from the solid support to provide an oligonucleotide of various nucleotide lengths, represented by a compound of formula D4. A oligonucleotide of formula D4 can then be deprotected to form a compound of formula D5 and coupled with a hydrophobic ligand (e.g. adamantyl or a lipophilic moiety) to form a compound of formula C7 (e.g., adamantyl or a fatty acid) under appropriate amide forming conditions (e.g., HATU, DIPEA), to form an oligonucleotide of formula II-b-3 comprising a ligand (e.g. adamantyl or a fatty acid) conjugate of the disclosure.

One of skill in the art will appreciate that various functional groups present in the nucleic acid or analogues thereof of the disclosure such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. See for example, “MARCH'S ADVANCED ORGANIC CHEMISTRY”, (5^{th} Ed., Ed.: Smith, M. B. and March, J., John Wiley & Sons, New York: 2001), the entirety of each of which is herein incorporated by reference. Such interconversions may require one or more of the aforementioned techniques, and certain methods for synthesizing the provided nucleic acids of the disclosure are described below in the Exemplification.

In some embodiments, the present disclosure provides a method for preparing an oligonucleotide comprising one or more lipid conjugate, said lipid conjugate unit represent by formula II-a-1:

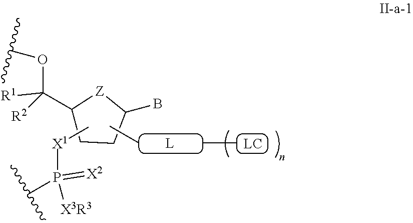

or a pharmaceutically acceptable salt thereof, comprising the steps of:
(a) providing a nucleic acid or analogue thereof of formula I-5a:

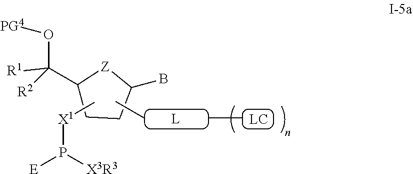

or salt thereof, and
(b) oligomerizing said compound of formula I-5a to form a compound of formula II-1a, wherein each of B, E, L, LC, n, PG⁴, R¹, R², R³, X, X¹, X², X³, E, and Z is as defined above and described herein.

In step (b) above, oligomerizing refers to preforming oligomerization forming conditions using known and commonly applied processes to prepare oligonucleotides in the art. For example, the compound of formula I-5a is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula II-1a comprising a lipid conjugate of the disclosure.

In some embodiments, the present disclosure provides a method for preparing an oligonucleotide comprising one or more lipid conjugate, further comprising preparing a nucleic acid or analogue thereof of formula I-5a:

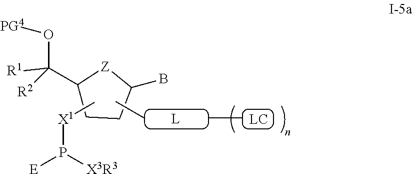

or a salt thereof, comprising the steps of:
(a) providing a nucleic acid or analogue thereof of formula Ia:

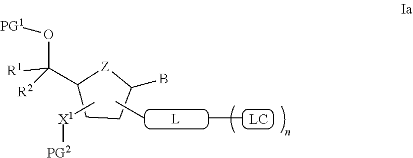

or salt thereof,
(b) deprotecting said nucleic acid or analogue thereof of formula Ia to form a compound of formula I-2a:

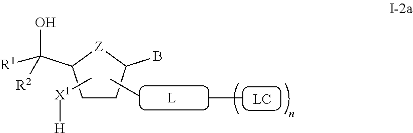

or salt thereof,
(c) protecting said nucleic acid or analogue thereof of formula I-2 to form a compound of formula I-3a:

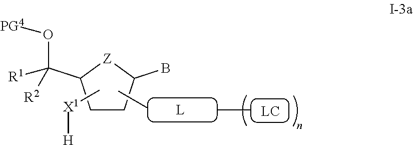

or salt thereof, and
(d) treating said nucleic acid or analogue thereof of formula I-3a with a P(III) forming reagent to form a nucleic acid or analogue thereof of formula I-5a, wherein each of B, E, L, LC, n, PG⁴, R¹, R², R³, X, X¹, X², X³, E, and Z is as defined above and described herein.

In step (b) above, PG¹ and PG² of a compound of formula Ia comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula I-2a is protected with a suitable hydroxyl protecting group. In certain embodiments, the protecting group PG⁴ used for protection of the 5′-hydroxyl group of a compound of formula I-2a includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula I-3a is treated with a P(III) forming reagent to afford a compound of formula I-5a. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula I-3a is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide comprising one or more lipid conjugates, further comprising preparing a nucleic acid-lipid conjugate or analogue thereof of formula Ia:

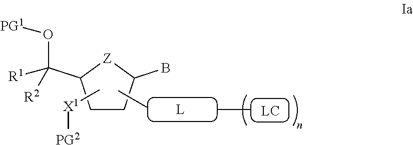

or a salt thereof, comprising the steps of:
(a) providing a nucleic acid or analogue thereof of formula I-1:

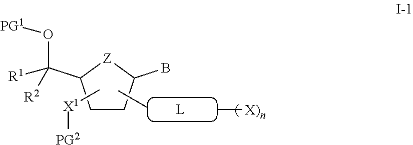

or salt thereof, and,
(b) conjugating one or more lipophilic compounds to a nucleic acid or analogue thereof of formula I-1 to form a nucleic acid or analogue thereof of formula Ia comprising one or more lipid conjugates, wherein: each of B, E, L, LC, n, PG¹, PG², R¹, R², X, X¹, and Z is as defined above and described herein.

In step (b) above, a nucleic acid or analogue thereof of formula I-Ia is conjugated with one or more lipophilic compounds to form a compound of formula Ia comprising one more lipid conjugates of the disclosure. Typically, conjugation is performed through an esterification or amidation reaction between a nucleic acid or analogue thereof of formula I-Ia and one or more fatty acids in series or in parallel by known techniques in the art. In certain embodiments, conjugation is performed under suitable amide forming conditions to afford a compound of formula I comprising one more lipid conjugates. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. Alternatively, conjugation of a lipophilic compound can be accomplished by any one of the cross-coupling technologies described in Table A herein.

In some embodiments, the present disclosure provides a method for preparing an oligonucleotide comprising one or more lipid conjugate, said lipid conjugate unit represent by formula II-1:

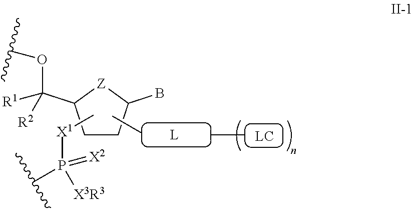

or a pharmaceutically acceptable salt thereof, comprising the steps of:
(a) providing an oligonucleotide of formula II-2:

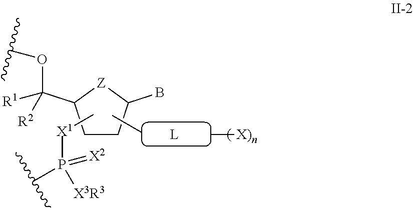

or salt thereof, and,
(b) conjugating one or more lipophilic compounds to an oligonucleotide of formula II-2 to form an oligonucleotide of formula II-1 comprising one or more lipid conjugates. In step (b) above, an oligonucleotide of formula II-2 is conjugated with one or more lipophilic compounds to form an oligonucleotide of formula II-1 comprising one more lipid conjugates of the disclosure. Typically, conjugation is performed through an esterification or amidation reaction between an oligonucleotide of formula II-2 and one or more fatty acids in series or in parallel by known techniques in the art. In certain embodiments, conjugation is performed under suitable amide forming conditions to afford an oligonucleotide of formula II-1 comprising one more lipid conjugates. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. Alternatively, conjugation of a lipophilic compound can be accomplished by any one of the cross-coupling technologies described in Table A herein.

In some embodiments, the present disclosure provides a method for preparing an oligonucleotide comprising a unit represent by formula II-2:

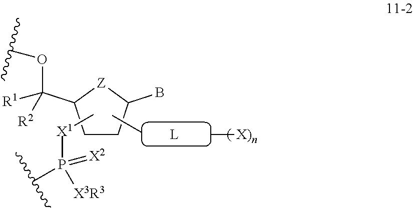

or a pharmaceutically acceptable salt thereof, comprising the steps of:
(a) providing a nucleic acid or analogue thereof of formula I-8:

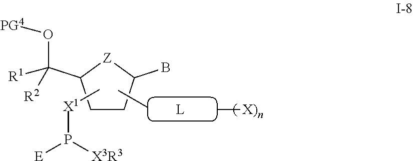

or salt thereof, and
(b) oligomerizing said compound of formula I-8 to form a compound of formula II-2.

In step (b) above, oligomerizing refers to preforming oligomerization forming conditions using known and commonly applied processes to prepare oligonucleotides in the art. For example, the compound of formula I-8 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula II-2.

In some embodiments, the present disclosure provides a method for preparing a nucleic acid or analogue thereof comprising one or more lipid conjugate, further comprising preparing a nucleic acid or analogue thereof of formula I-8:

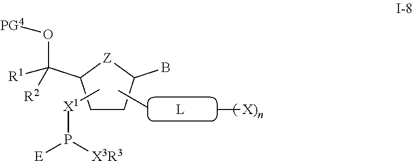

or a salt thereof, comprising the steps of:
(a) providing a nucleic acid or analogue thereof of formula I-1:

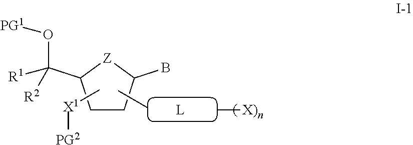

or salt thereof,
(b) deprotecting said nucleic acid or analogue thereof of formula I-1 to form a compound of formula I-6:

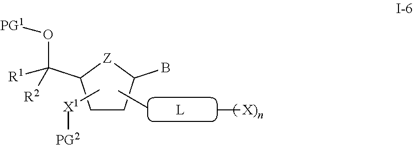

or salt thereof,
(c) protecting said nucleic acid or analogue thereof of formula I-6 to form a compound of formula I-7:

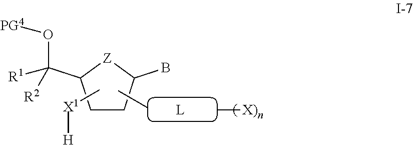

or salt thereof, and
(d) treating said nucleic acid or analogue thereof of formula I-7 with a P(III) forming reagent to form a nucleic acid or analogue thereof of formula I-8, In step (b) above, PG¹ and PG² of a compound of formula I-1 comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula I-6 is protected with a suitable hydroxyl protecting group. In certain embodiments, the protecting group PG⁴ used for protection of the 5′-hydroxyl group of a compound of formula I-6 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula I-7 is treated with a P(III) forming reagent to afford a compound of formula I-8. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula 1-7 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide-ligand conjugate comprising one or more adamantyl and/or lipid moieties, said conjugate unit represented by formula II-b-3:

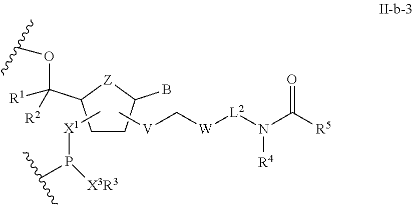

or a pharmaceutically acceptable salt thereof, comprising the steps of:
(a) providing a nucleic acid-ligand conjugate or analogue thereof of formula C11:

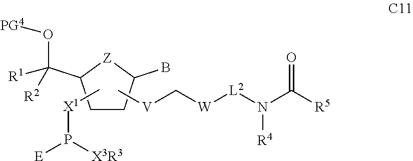

or salt thereof, and
(b) oligomerizing said compound of formula C11 to form a compound of formula II-b-3, In step (b) above, oligomerizing refers to preforming oligomerization forming conditions using known and commonly applied processes to prepare oligonucleotides in the art. For example, the compound of formula C11 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide-ligand conjugate of various nucleotide lengths, with one or more nucleic acid-ligand conjugate units, wherein each unit is represented by a compound of formula II-b-3 comprising an adamantyl or lipid moiety of the disclosure.

In some embodiments, the method for preparing an oligonucleotide of formula II-b-3 comprising one or more lipid conjugate, further comprises preparing a nucleic acid-ligand conjugate or analogue thereof of formula C11:

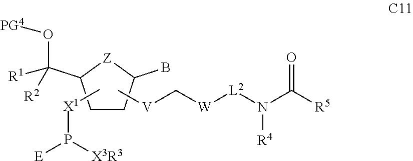

or a salt thereof, comprising the steps of:
(a) providing a nucleic acid-ligand conjugate or analogue thereof of formula I-b:

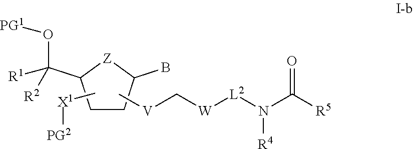

or salt thereof,
(b) deprotecting said nucleic acid-ligand conjugate or analogue thereof of formula I-b to form a compound of formula C8:

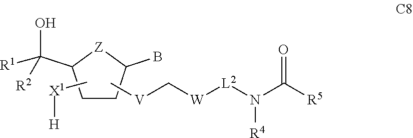

or salt thereof,
(c) protecting said nucleic acid-ligand conjugate or analogue thereof of formula C8 to form a compound of formula C9:

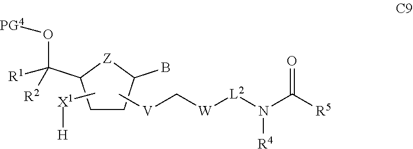

or salt thereof, and
(d) treating said nucleic acid-ligand conjugate or analogue thereof of formula C9 with a P(III) forming reagent to form a nucleic acid or analogue thereof of formula C11, In step (b) above, PG¹ and PG² of a compound of formula I-b comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a compound of formula C8 is protected with a suitable hydroxyl protecting group. In certain embodiments, the protecting group PG⁴ used for protection of the 5′-hydroxyl group of a compound of formula C8 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a compound of formula C9 is treated with a P(III) forming reagent to afford a compound of formula C11. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula C9 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

In some embodiments, the present disclosure provides a method for preparing an oligonucleotide-ligand conjugate of formula II-b-3 comprising one or more nucleic acid-ligand conjugate units each comprising one or more adamantyl or lipid moieties, further comprising preparing a nucleic acid-ligand conjugate or analogue thereof of formula I-b:

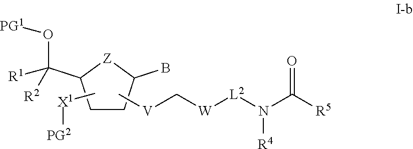

or a salt thereof, comprising the steps of:
(a) providing a nucleic acid-ligand conjugate or analogue thereof of formula C6:

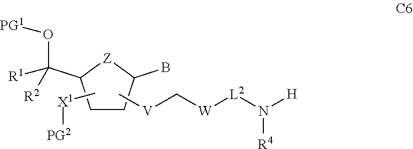

or salt thereof, and,
(b) conjugating a lipophilic compound to a nucleic acid or analogue thereof of formula C6 to form a nucleic acid-ligand conjugate or analogue thereof of formula I-b comprising one or more adamantyl and/or lipid conjugates, In step (b) above, conjugation is performed under suitable amide forming conditions to afford a compound of formula I-b comprising an adamantyl and/or lipid conjugate. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the amide forming conditions comprise HATU and DIPEA or TEA.

In certain embodiments, a nucleic acid-ligand conjugate or analogue thereof of formula C6 is provided in salt form (e.g., a fumarate salt) and is first converted to the free base (e.g., using sodium bicarbonate) before preforming the conjugation step.

In some embodiments, the present disclosure provides a method for preparing an oligonucleotide-ligand conjugate of formula II-b-3 comprising one or more nucleic acid-ligand conjugate units, further comprises preparing a nucleic acid-ligand conjugate or analogue thereof of formula C6:

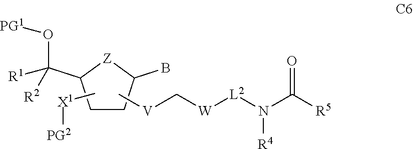

or a salt thereof, comprising the steps of:
(a) providing a nucleic acid or analogue thereof of formula C1:

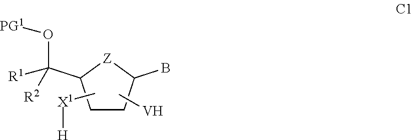

or salt thereof, and,
(b) protecting said nucleic acid or analogue thereof of formula C1 to form a compound of formula C2:

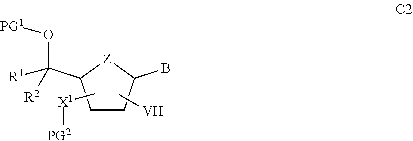

or salt thereof,
(c) alkylating said nucleic acid or analogue thereof of formula C2 to form a compound of formula C3:

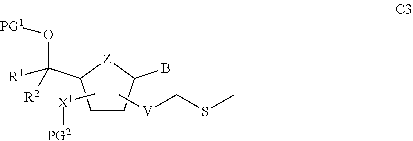

or salt thereof,
(d) substituting said nucleic acid or analogue thereof of formula C3 with a compound of formula C4:

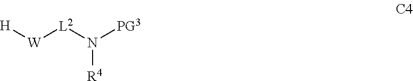

or salt thereof, to form a compound of formula C5:

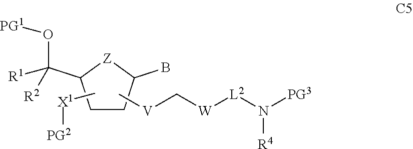

or salt thereof,
(e) deprotecting said nucleic acid or analogue thereof of formula C5 to form a nucleic acid-ligand conjugate or analogue thereof of formula C6. In step (b) above, PG¹ and PG² groups of formula C2 are taken together with their intervening atoms to form a cyclic diol protecting group, such as a cyclic acetal or ketal. Such groups include methylene, ethylidene, benzylidene, isopropylidene, cyclohexylidene, and cyclopentylidene, silylene derivatives such as di-t-butylsilylene and 1,1,3,3-tetraisopropylidisiloxanylidene, a cyclic carbonate, a cyclic boronate, and cyclic monophosphate derivatives based on cyclic adenosine monophosphate (i.e., cAMP). In certain embodiments, the cyclic diol protection group is 1,1,3,3-tetraisopropylidisiloxanylidene prepared from the reaction of a diol of formula C1 and 1,3-dichloro-1,1,3,3-tetraisopropyldisiloxane under basic conditions.

In step (c) above, a nucleic acid or analogue thereof of formula C2 is alkylated with a mixture of DMSO and acetic anhydride under acidic conditions. In certain embodiments, when -V-H is a hydroxyl group, the mixture of DMSO and acetic anhydride in the presence of acetic acid forms (methylthio)methyl acetate in situ via the Pummerer rearrangement which then reacts with the hydroxyl group of the nucleic acid or analogue thereof of formula C2 to provide a monothioacetal functionalized fragment nucleic acid or analogue thereof of formula C3.

In step (d) above, substitution of the thiomethyl group of a nucleic acid or analogue thereof of formula C3 using a nucleic acid or analogue thereof of formula C4 affords a nucleic acid or analogue thereof of formula C4. In certain embodiments, substitution occurs under mild oxidizing and/or acidic conditions. In some embodiments, V is oxygen. In some embodiments, the mild oxidation reagent includes a mixture of elemental iodine and hydrogen peroxide, urea hydrogen peroxide complex, silver nitrate/silver sulfate, sodium bromate, ammonium peroxodisulfate, tetrabutylammonium peroxydisulfate, Oxone®, Chloramine T, Selectfluor®, Selectfluor® II, sodium hypochlorite, or potassium iodate/sodium periodiate. In certain embodiments, the mild oxidizing agent includes N-iodosuccinimide, N-bromosuccinimide, N-chlorosuccinimide, 1,3-diiodo-5,5-dimethylhydantion, pyridinium tribromide, iodine monochloride or complexes thereof, etc. Acids that are typically used under mild oxidizing condition include sulfuric acid, p-toluenesulfonic acid, trifluoromethanesulfonic acid, methanesulfonic acid, and trifluoroacetic acid. In certain embodiments, the mild oxidation reagent includes a mixture of N-iodosuccinimide and trifluoromethanesulfonic acid.

In step (e) above, removal of PG³ and optionally R⁴ (when R⁴ is a suitable amine protecting group) of a nucleic acid-ligand conjugate or analogue thereof of formula C5 affords a nucleic acid-ligand conjugate or analogue thereof of formula C6 or a salt thereof. In some embodiments, PG³ and/or R⁴ comprise carbamate derivatives that can be removed under acidic or basic conditions. In certain embodiments, the protecting groups (e.g., both PG³ and R⁴ or either of PG³ or R⁴ independently) of a nucleic acid-ligand conjugate or analogue thereof of formula C5 are removed by acid hydrolysis. It will be appreciated that upon acid hydrolysis of the protecting groups of a nucleic acid-ligand conjugate or analogue thereof of formula C5, a salt of formula C6 thereof is formed. For example, when an acid-labile protecting group of a nucleic acid-ligand conjugate or analogue thereof of formula C5 is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a nucleic acid or analogue thereof of formula C6 are contemplated.

In other embodiments, the protecting groups (e.g., both PG³ and R⁴ or either of PG³ or R⁴ independently) of a nucleic acid or analogue thereof of formula C5 are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-diazabicyclo[5.4.0]undec-7-ene (DBU). In certain embodiments, a nucleic acid-ligand conjugate or analogue thereof of formula C5 is deprotected under basic conditions followed by treating with an acid to form a salt of formula C6. In certain embodiments, the acid is fumaric acid the salt of formula C6 is the fumarate.

In some embodiments, the present disclosure provides a method for preparing an oligonucleotide-ligand conjugate comprising one or more nucleic acid-ligand conjugate, said nucleic acid-ligand conjugate unit represented by formula II-b-3:

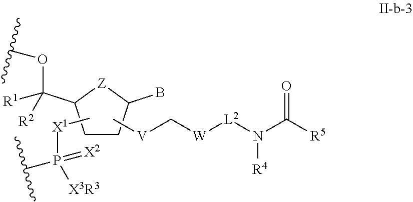

or a pharmaceutically acceptable salt thereof, comprising the steps of:
(a) providing an oligonucleotide of formula D5:

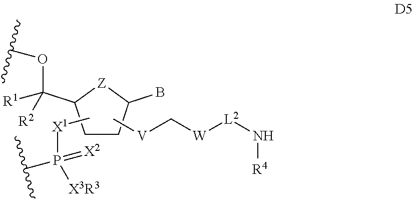

or salt thereof, and,
(b) conjugating one or more adamantyl or lipophilic compounds to an oligonucleotide of formula D5 to form an oligonucleotide-ligand conjugate of formula II-b-3 comprising one or more nucleic acid-ligand conjugate units, In step (b) above, conjugation is performed under suitable amide forming conditions to afford a compound of formula D5 comprising an adamantyl or lipid conjugate. Suitable amide forming conditions can include the use of an amide coupling reagent known in the art such as, but not limited to HATU, PyBOP, DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU. In certain embodiments, the amide forming conditions comprise HATU and DIPEA or TEA.

In some embodiments, the present disclosure provides a method for preparing an oligonucleotide-ligand conjugate comprising a unit represent by formula D5:

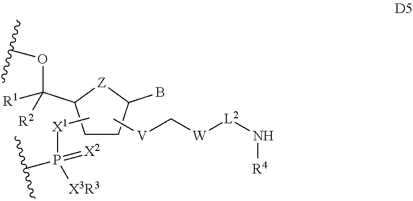

or a salt thereof, comprising the steps of:
(a) providing a nucleic acid-ligand conjugate or analogue thereof of formula D4:

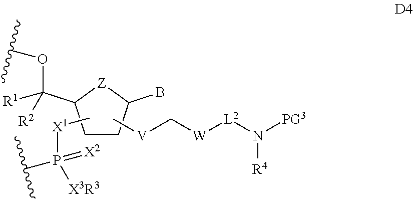

or salt thereof, and
(b) deprotecting said compound of formula D4 to form a compound of formula D5, In step (b) above, removal of PG³ and optionally R⁴ (when R⁴ is a suitable amine protecting group) of an oligonucleotide of formula D4 affords an oligonucleotide-ligand conjugate of formula D5 or a salt thereof. In some embodiments, PG³ and/or R⁴ comprise carbamate derivatives that can be removed under acidic or basic conditions. In certain embodiments, the protecting groups (e.g., both PG³ and R⁴ or either of PG³ or R⁴ independently) of an oligonucleotide-ligand conjugate of formula D4 are removed by acid hydrolysis. It will be appreciated that upon acid hydrolysis of the protecting groups of an oligonucleotide-ligand conjugate of formula D4, a salt of formula D5 thereof is formed. For example, when an acid-labile protecting group of an oligonucleotide of formula D4 is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms of a nucleic acid-ligand conjugate unit or analogue thereof of formula D5 are contemplated.

In other embodiments, the protecting groups (e.g., both PG³ and R⁴ or either of PG³ or R⁴ independently) of an oligonucleotide-ligand conjugate of formula D4 are removed by base hydrolysis. For example, Fmoc and trifluoroacetyl protecting groups can be removed by treatment with base. One of ordinary skill in the art would recognize that a wide variety of bases are useful for removing amino protecting groups that are base-labile. In some embodiments, a base is piperidine. In some embodiments, a base is 1,8-diazabicyclo[5.4.0]undec-7-ene (DBU).

In some embodiments, the present disclosure provides a method for preparing an oligonucleotide-ligand conjugate comprising one or more nucleic acid-ligand conjugate unit with one or more adamantyl and/or lipid moiety, said conjugate unit represented by formula D4:

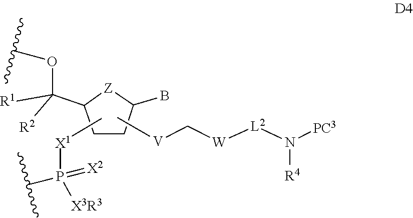

or a pharmaceutically acceptable salt thereof, comprising the steps of:
(a) providing a nucleic acid or analogue thereof of formula D3:

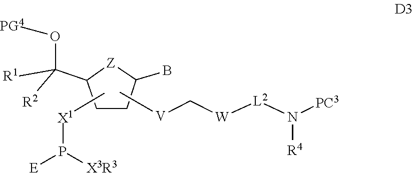

or salt thereof, and
(b) oligomerizing said compound of formula D3 to form a compound of formula D4,

In step (b) above, oligomerizing refers to preforming oligomerization forming conditions using known and commonly applied processes to prepare oligonucleotides in the art. For example, the nucleic acid or analogue thereof of formula D3 is coupled to a solid supported nucleic acid or analogue thereof bearing a 5′-hydroxyl group. Further steps can comprise one or more deprotections, couplings, phosphite oxidation, and cleavage from the solid support to provide an oligonucleotide of various nucleotide lengths, represented by a compound of formula D4 comprising an adamantyl or lipid conjugate of the disclosure.

In some embodiments, the present disclosure provides a method for preparing a nucleic acid or analogue thereof comprising one or more lipid conjugate, further comprising preparing a nucleic acid or analogue thereof of formula D3:

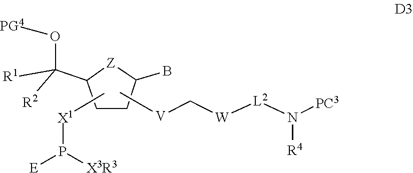

or a salt thereof, comprising the steps of:
(a) providing a nucleic acid or analogue thereof of formula C5:

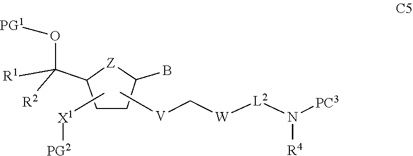

or salt thereof,
(b) deprotecting said nucleic acid or analogue thereof of formula C5 to form a compound of formula D1:

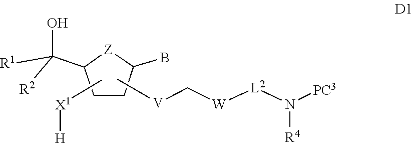

or salt thereof,
(c) protecting said nucleic acid or analogue thereof of formula D1 to form a nucleic acid or analogue thereof of formula D2:

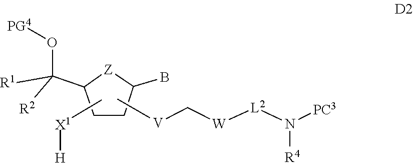

or salt thereof, and
(d) treating said nucleic acid or analogue thereof of formula D2 with a P(III) forming reagent to form a nucleic acid or analogue thereof of formula D3, In step (b) above, PG¹ and PG² of a nucleic acid or analogue thereof of formula C5 comprise silyl ethers or cyclic silylene derivatives that can be removed under acidic conditions or with fluoride anion. Examples of reagents providing fluoride anion for the removal of silicon-based protecting groups include hydrofluoric acid, hydrogen fluoride pyridine, triethylamine trihydrofluoride, tetra-N-butylammonium fluoride, and the like.

In step (c) above, a nucleic acid or analogue thereof of formula D1 is protected with a suitable hydroxyl protecting group. In certain embodiments, the protecting group PG⁴ used for protection of the 5′-hydroxyl group of a compound of formula D1 includes an acid labile protecting group such as trityl, 4-methyoxytrityl, 4,4′-dimethyoxytrityl, 4,4′,4″-trimethyoxytrityl, 9-phenyl-xanthen-9-yl, 9-(p-tolyl)-xanthen-9-yl, pixyl, 2,7-dimethylpixyl, and the like. In certain embodiments, the acid labile protecting group is suitable for deprotection during both solution-phase and solid-phase synthesis of acid-sensitive nucleic acids or analogues thereof using for example, dichloroacetic acid or trichloroacetic acid.

In step (d) above, a nucleic acid or analogue thereof of formula D2 is treated with a P(III) forming reagent to afford a compound of formula D3. In the context of the present disclosure, a P(III) forming reagent is a phosphorus reagent that is reacted to for a phosphorus (III) compound. In some embodiments, the P(III) forming reagent is 2-cyanoethyl N,N-diisopropylchlorophosphoramidite or 2-cyanoethyl phosphorodichloridate. In certain embodiments, the P(III) forming reagent is 2-cyanoethyl N,N-diisopropylchlorophosphoramidite. One of ordinary skill would recognize that the displacement of a leaving group in a P(III) forming reagent by X¹ of a compound of formula D2 is achieved either with or without the presence of a suitable base. Such suitable bases are well known in the art and include organic and inorganic bases. In certain embodiments, the base is a tertiary amine such as triethylamine or diisopropylethylamine. In other embodiments, step (d) above is preformed using N,N-dimethylphosphoramic dichloride as a P(V) forming reagent.

### 6. Uses, Formulation and Administration

Pharmaceutically Acceptable Compositions

According to another embodiment, the disclosure provides a composition comprising a, nucleic acid-ligand conjugate or analogue thereof. In another embodiment, the disclosure provides oligonucleotide-ligand conjugate comprising one or more nucleic acid-ligand conjugate units with adamantyl or lipid group as a ligand and a pharmaceutically acceptable carrier, adjuvant, or vehicle. The amount of an oligonucleotide-ligand conjugate in the compositions of this disclosure is effective to measurably modulate the expression of a target gene in a biological sample or in a patient. In certain embodiments, a composition of this disclosure is formulated for administration to a patient in need of such composition. In some embodiments, a composition of this disclosure is formulated for parenteral or oral administration to a patient. In some embodiments, the composition comprises a pharmaceutically acceptable carrier, adjuvant, or vehicle, and a nucleic acid inhibitor molecule, wherein the nucleic acid inhibitor molecule comprises at least one nucleotide comprising a lipid conjugate, as described herein.

The term “patient,” as used herein, means an animal, preferably a mammal, and most preferably a human.

The term “pharmaceutically acceptable carrier, adjuvant, or vehicle” refers to anon-toxic carrier, adjuvant, or vehicle that does not destroy the pharmacological activity of a provided nucleic acid with which it is formulated. Pharmaceutically acceptable carriers, adjuvants or vehicles that may be used in the compositions of this disclosure include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a provided nucleic acid of this disclosure that, upon administration to a recipient, is capable of providing, either directly or indirectly, a provided nucleic acid of this disclosure or an inhibitory active metabolite or residue thereof.

As used herein, the term “inhibitory active metabolite or residue thereof” means that a metabolite or residue thereof is also useful to modulate the expression of a target gene in a biological sample or in a patient.

Compositions of the present disclosure may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrastemal, intrathecal, intrahepatic, intralesional and intracranial injection or infusion techniques. Preferably, the compositions are formulated in liquid form for parenteral administration, for example, by subcutaneous, intramuscular, intravenous or epidural injection. Dosage forms suitable for parenteral administration typically comprise one or more suitable vehicles for parenteral administration including, by way of example, sterile aqueous solutions, saline, low molecular weight alcohols such as propylene glycol, polyethylene glycol, vegetable oils, gelatin, fatty acid esters such as ethyl oleate, and the like. The parenteral formulations may contain sugars, alcohols, antioxidants, buffers, bacteriostats, solutes which render the formulation isotonic with the blood of the intended recipient or suspending or thickening agents. Proper fluidity can be maintained, for example, by the use of surfactants. Liquid formulations can be lyophilized and stored for later use upon reconstitution with a sterile injectable solution.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this disclosure may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. Compositions of this disclosure formulated for oral administration may be administered with or without food. In some embodiments, pharmaceutically acceptable compositions of this disclosure are administered without food. In other embodiments, pharmaceutically acceptable compositions of this disclosure are administered with food.

Alternatively, pharmaceutically acceptable compositions of this disclosure may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this disclosure may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be affected in a rectal suppository formulation (see above) or in a suitable enema formulation. Topically transdermal patches may also be used.

For topical applications, provided pharmaceutically acceptable compositions may be formulated in a suitable ointment containing the active component suspended or dissolved in one or more carriers. Carriers for topical administration of nucleic acid or analogues thereof of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this disclosure may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In certain embodiments, a provided nucleic acid-ligand conjugate or an oligonucleotide-ligand conjugate (e.g., nucleic acid inhibitor molecule) may be admixed, encapsulated, conjugated or otherwise associated with other molecules, molecule structures or mixtures of compounds, including, for example, liposomes and lipids such as those disclosed in U.S. Pat. Nos. 6,815,432, 6,586,410, 6,858,225, 7,811,602, 7,244,448 and 8,158,601; polymeric materials such as those disclosed in U.S. Pat. Nos. 6,835,393, 7,374,778, 7,737,108, 7,718,193, 8,137,695 and U.S. Published Patent Application Nos. 2011/0143434, 2011/0129921, 2011/0123636, 2011/0143435, 2011/0142951, 2012/0021514, 2011/0281934, 2011/0286957 and 2008/0152661; capsids, capsoids, or receptor targeted molecules for assisting in uptake, distribution or absorption, the entirety of each of which is herein incorporated by reference.

In certain embodiments, a provided nucleic acid-ligand conjugate or an oligonucleotide-ligand conjugate (e.g., nucleic acid inhibitor molecule) is formulated in a lipid nanoparticle (LNP). Lipid-nucleic acid nanoparticles (e.g. lipid-oligonucleotide-ligand conjugate nanoparticles) typically form spontaneously upon mixing lipids with nucleic acid to form a complex. Depending on the desired particle size distribution, the resultant nanoparticle mixture can be optionally extruded through a polycarbonate membrane (e.g., 100 nm cut-off) using, for example, a thermobarrel extruder, such as LIPEX® Extruder (Northern Lipids, Inc). To prepare a lipid nanoparticle for therapeutic use, it may desirable to remove solvent (e.g., ethanol) used to form the nanoparticle and/or exchange buffer, which can be accomplished by, for example, dialysis or tangential flow filtration. Methods of making lipid nanoparticles containing nucleic acid inhibitor molecules are known in the art, as disclosed, for example in U.S. Published Patent Application Nos. 2015/0374842 and 2014/0107178, the entirety of each of which is herein incorporated by reference.

In certain embodiments, the LNP comprises a lipid core comprising a cationic liposome and a pegylated lipid. The LNP can further comprise one or more envelope lipids, such as a cationic lipid, a structural or neutral lipid, a sterol, a pegylated lipid, or mixtures thereof.

In certain embodiments, a provided nucleic acid is covalently conjugated to a ligand that directs delivery of the nucleic acid to a tissue of interest. Many such ligands have been explored. See, e.g., Winkler, THER. DELIV., 2013, 4(7): 791-809. For example, a provided nucleic acid can be conjugated to multiple sugar ligand moieties (e.g., N-acetylgalactosamine (GalNAc)) to direct uptake of the nucleic acid into the liver. See, e.g., WO 2016/100401. Other ligands that can be used include, but are not limited to, mannose-6-phosphate, cholesterol, folate, transferrin, and galactose (for other specific exemplary ligands see, e.g., WO 2012/089352). Typically, when a provided nucleic acid is conjugated to a ligand, the nucleic acid is administered as a naked nucleic acid, wherein the oligonucleotide is not also formulated in an LNP or other protective coating. In certain embodiments, each nucleotide within the naked nucleic acid is modified at the 2′-position of the sugar moiety, typically with 2′-F or 2′-OMe.

These pharmaceutical compositions may be sterilized by conventional sterilization techniques or may be sterile filtered. The resulting aqueous solutions may be packaged for use as is, or lyophilized, the lyophilized preparation being combined with a sterile aqueous excipient prior to administration. The pH of the preparations typically will be between 3 and 11, more preferably between 5 and 9 or between 6 and 8, and most preferably between 7 and 8, such as 7 to 7.5. The pharmaceutical compositions in solid form may be packaged in multiple single dose units, each containing a fixed amount of the above-mentioned agent or agents, such as in a sealed package of tablets or capsules. The pharmaceutical compositions in solid form can also be packaged in a container for a flexible quantity, such as in a squeezable tube designed for a topically applicable cream or ointment.

The amount of nucleic acid-ligand conjugate, oligonucleotide-ligand conjugate or analogue thereof of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the nucleic acid or analogue thereof can be administered to a patient receiving these compositions.

It should also be understood that a specific dosage and treatment regimen for any particular patient will depend upon a variety of factors, including the activity of the specific nucleic acid or analogue thereof employed, the age, body weight, general health, sex, diet, time of administration, rate of excretion, drug combination, and the judgment of the treating physician and the severity of the particular disease being treated. The amount of a nucleic acid or analogue thereof of the present disclosure in the composition will also depend upon the particular nucleic acid or analogue thereof in the composition.

Uses of Nucleic Acids and Analogues Thereof and Pharmaceutically Acceptable Compositions

Nucleic acid-ligand conjugates, oligonucleotide-ligand conjugate and analogues thereof and compositions described herein are generally useful for modulation of intracellular RNA levels. A provided nucleic acid-ligand conjugate or an oligonucleotide-ligand conjugate or analogue thereof can be used in a method of modulating the expression of a target gene in a cell. Typically, such methods comprise introducing a provided nucleic acid inhibitor molecule (e.g. oligonucleotide-ligand conjugate) into a cell in an amount sufficient to modulate the expression of a target gene. In certain embodiments, the method is carried out in vivo. The method can also be carried out in vitro or ex vivo. In certain embodiments, the cell is a mammalian cell, including, but not limited to, a human cell.

In certain embodiments, a provided nucleic acid-ligand conjugate or an oligonucleotide-ligand conjugate or analogue thereof (e.g., nucleic acid inhibitor molecule) can be used in a method of treating a patient in need thereof. Typically, such methods comprise administering a therapeutically effective amount of a pharmaceutical composition comprising a provided nucleic acid inhibitor molecule, as described herein, to a patient in need thereof.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

In certain embodiments, the pharmaceutical compositions disclosed herein may be useful for the treatment or prevention of symptoms related to a viral infection in a patient in need thereof. One embodiment is directed to a method of treating a viral infection, comprising administering to a subject a pharmaceutical composition comprising a therapeutically effective amount of a provided nucleic acid comprising a lipid conjugate or analogue thereof (e.g., nucleic acid inhibitor molecule), as described herein. Non-limiting examples of such viral infections include HCV, HBV, HPV, HSV, HDV, HEV or HIV infection.

In certain embodiments, the pharmaceutical compositions disclosed herein may be useful for the treatment or prevention of symptoms related to cancer in a patient in need thereof. One embodiment is directed to a method of treating cancer, comprising administering to a subject a pharmaceutical composition comprising a therapeutically effective amount of a provided nucleic acid-ligand conjugate or an oligonucleotide-ligand conjugate (e.g. nucleic acid inhibitor molecule), as described herein. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymocytic leukemia (ALL), acute myeloid leukemia (AML), chronic lymphocytic (CLL), chronic myeloid (CML), chronic myelomonocytic (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T-cell lymphomas, non-Hodgkin lymphoma, precursor T-lymphoblastic lymphoma/leukemia, peripheral T-cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Typically, the present disclosure features methods of treating liver cancer, liver carcinoma, hepatoma, hepatocellular carcinoma, cholangiocarcinoma and hepatoblastoma by administering a therapeutically effective amount of a pharmaceutical composition as described herein.

In certain embodiments the pharmaceutical compositions disclosed herein may be useful for treatment or prevention of symptoms related to proliferative, inflammatory, autoimmune, neurologic, ocular, respiratory, metabolic, dermatological, auditory, liver, kidney, or infectious diseases. One embodiment is directed to a method of treating a proliferative, inflammatory, autoimmune, neurologic, ocular, respiratory, metabolic, dermatological, auditory, liver, kidney, or infectious disease, comprising administering to a subject a pharmaceutical composition comprising a therapeutically effective amount of a provided nucleic acid-ligand conjugate or an oligonucleotide-ligand conjugate (e.g. a nucleic acid inhibitor molecule), as described herein. Typically, the disease or condition is disease of the liver.

In some embodiments, the present disclosure provides a method for reducing expression of a target gene in a subject comprising administering a pharmaceutical composition to a subject in need thereof in an amount sufficient to reduce expression of the target gene, wherein the pharmaceutical composition comprises a provided nucleic acid-ligand conjugate or an oligonucleotide-ligand conjugate (e.g. a nucleic acid inhibitor molecule), as described herein and a pharmaceutically acceptable excipient as also described herein.

In some embodiments, a provided nucleic acid-ligand conjugate or an oligonucleotide-ligand conjugate (e.g. a nucleic acid inhibitor molecule) is an RNAi inhibitor molecule as described herein, including a dsRNAi inhibitor molecule or an ssRNAi inhibitor molecule.

The target gene may be a target gene from any mammal, such as a human target gene. Any gene may be silenced according to the instant method. Exemplary target genes include, but are not limited to, Factor VII, Eg5, PCSK9, TPX2, apoB, SAA, TTR, HBV, HCV, RSV, PDGF beta gene, Erb-B gene, Src gene, CRK gene, GRB2 gene, RAS gene, MEKK gene, JNK gene, RAF gene, Erk1/2 gene, PCNA(p21) gene, MYB gene, JUN gene, FOS gene, BCL-2 gene, Cyclin D gene, VEGF gene, EGFR gene, Cyclin A gene, Cyclin E gene, WNT-1 gene, beta-catenin gene, c-MET gene, PKC gene, NFKB gene, STAT3 gene, survivin gene, Her2/Neu gene, topoisomerase I gene, topoisomerase II alpha gene, p73 gene, p21(WAF1/CIP1) gene, p27(KIP1) gene, PPM1D gene, RAS gene, caveolin I gene, MIB I gene, MTAI gene, M68 gene, mutations in tumor suppressor genes, p53 tumor suppressor gene, LDHA, and combinations thereof.

In some embodiments, a provided nucleic acid-ligand conjugate or an oligonucleotide-ligand conjugate (e.g. a nucleic acid inhibitor molecule), silences a target gene and thus can be used to treat a subject having or at risk for a disorder characterized by unwanted expression of the target gene. For example, in some embodiments, the provided nucleic acid-ligand conjugate or an oligonucleotide-ligand conjugate (e.g. a nucleic acid inhibitor molecule) silences the beta-catenin gene, and thus can be used to treat a subject having or at risk for a disorder characterized by unwanted beta-catenin expression, e.g., adenocarcinoma or hepatocellular carcinoma.

Typically, a provided nucleic acid-ligand conjugate or an oligonucleotide-ligand conjugate (e.g. a nucleic acid inhibitor molecule) of the disclosure is administered intravenously or subcutaneously. However, the pharmaceutical compositions disclosed herein may also be administered by any method known in the art, including, for example, oral, buccal, sublingual, rectal, vaginal, intraurethral, topical, intraocular, intranasal, and/or intra-auricular, which administration may include tablets, capsules, granules, aqueous suspensions, gels, sprays, suppositories, salves, ointments, or the like.

In certain embodiments, the pharmaceutical composition is delivered via systemic administration (such as via intravenous or subcutaneous administration) to relevant tissues or cells in a subject or organism, such as the liver. In other embodiments, the pharmaceutical composition is delivered via local administration or systemic administration. In certain embodiments, the pharmaceutical composition is delivered via local administration to relevant tissues or cells, such as lung cells and tissues, such as via pulmonary delivery.

The therapeutically effective amount of the nucleic acid-ligand conjugate or an oligonucleotide-ligand conjugate disclosed herein may depend on the route of administration and the physical characteristics of the patient, such as the size and weight of the subject, the extent of the disease progression or penetration, the age, health, and sex of the subject.

In certain embodiments, a provided nucleic acid-ligand conjugate or an oligonucleotide-ligand conjugate, as described herein, is administered at a dosage of 20 micrograms to 10 milligrams per kilogram body weight of the recipient per day, 100 micrograms to 5 milligrams per kilogram body weight of the recipient per day, or 0.5 to 2.0 milligrams per kilogram body weight of the recipient per day.

A pharmaceutical composition of the instant disclosure may be administered every day or intermittently. For example, intermittent administration of a nucleic acid-ligand conjugate or an oligonucleotide-ligand conjugate of the instant disclosure may be one to six days per week, one to six days per month, once weekly, once every other week, once monthly, once every other month, or once or twice per year or divided into multiple yearly, monthly, weekly, or daily doses. In some embodiments, intermittent dosing may mean administration in cycles (e.g. daily administration for one day, one week or two to eight consecutive weeks, then a rest period with no administration for up to one week, up to one month, up to two months, up to three months or up to six months or more) or it may mean administration on alternate days, weeks, months or years.

In any of the methods of treatment of the disclosure, the nucleic acid-ligand conjugate or an oligonucleotide-ligand conjugate or analogues thereof may be administered to the subject alone as a monotherapy or in combination with additional therapies known in the art.

### EXEMPLIFICATION

### Abbreviations

Ac: acetyl
AcOH: acetic acid
ACN: acetonitrile
Ad: adamantly
AIBN: 2,2′-azo bisisobutyronitrile
Anhyd: anhydrous
Aq: aqueous
B₂Pin₂: bis (pinacolato)diboron-4,4,4′,4′,5,5,5′,5′-octamethyl-2,2′-bi(1,3,2-dioxaborolane)
BINAP: 2,2′-bis(diphenylphosphino)-1,1′-binaphthyl
BH₃: Borane
Bn: benzyl
Boc: tert-butoxycarbonyl
Boc₂O: di-tert-butyl dicarbonate
BPO: benzoyl peroxide
ⁿBuOH: n-butanol
CDI: carbonyldiimidazole
COD: cyclooctadiene
d: days
DABCO: 1,4-diazobicyclo[2.2.2]octane
DAST: diethylaminosulfur trifluoride
dba: dibenzylideneacetone
DBU: 1,8-diazobicyclo[5.4.0]undec-7-ene
DCE: 1,2-dichloroethane
DCM: dichloromethane
DEA: diethylamine
DHP: dihydropyran
DIBAL-H: diisobutylaluminum hydride
DIPA: diisopropylamine
DIPEA or DIEA: N,N-diisopropylethylamine
DMA: N,N-dimethylacetamide
DME: 1,2-dimethoxyethane
DMAP: 4-dimethylaminopyridine
DMF: N,N-dimethylformamide
DMP: Dess-Martin periodinane
DMSO-dimethyl sulfoxide
DMTr: 4,4′-dimethyoxytrityl
DPPA: diphenylphosphoryl azide
dppf: 1,1′-bis(diphenylphosphino)ferrocene
EDC or EDCI: 1-(3-dimethylaminopropyl)-3-ethylcarbodiimide hydrochloride
ee: enantiomeric excess
ESI: electrospray ionization
EA: ethyl acetate
EtOAc: ethyl acetate
EtOH: ethanol
FA: formic acid
h or hrs: hours
HATU: N,N,N′,N′-tetramethyl-O-(7-azabenzotriazol-1-yl)uronium hexafluorophosphate
HCl: hydrochloric acid
HPLC: high performance liquid chromatography
HOAc: acetic acid
IBX: 2-iodoxybenzoic acid
IPA: isopropyl alcohol
KHMDS: potassium hexamethyldisilazide
K₂CO₃: potassium carbonate
LAH: lithium aluminum hydride
LDA: lithium diisopropylamide
L-DBTA: dibenzoyl-L-tartaric acid
m-CPBA: meta-chloroperbenzoic acid
M: molar
MeCN: acetonitrile
MeOH: methanol
Me₂S: dimethyl sulfide
MeONa: sodium methylate
MeI: iodomethane
min: minutes
mL: milliliters
mM: millimolar
mmol: millimoles
MPa: mega pascal
MOMCl: methyl chloromethyl ether
MsCl: methanesulfonyl chloride
MTBE: methyl tert-butyl ether
nBuLi: n-butyllithium
NaNO₂: sodium nitrite
NaOH: sodium hydroxide
Na₂SO₄: sodium sulfate
NBS: N-bromosuccinimide
NCS: N-chlorosuccinimide
NFSI: N-Fluorobenzenesulfonimide
NMO: N-methvlnorpholine N-oxide
NMP: N-methylpyrrolidine
NMR: Nuclear Magnetic Resonance
° C.: degrees Celsius
Pd/C: Palladium on Carbon
Pd(OAc)₂: Palladium Acetate
PBS: phosphate buffered saline
PE: petroleum ether
POCl₃: phosphorus oxychloride
PPh₃: triphenylphosphine
PyBOP: (Benzotriazol-1-yloxy)tripyrrolidinophosphonium hexafluorophosphate
Rel: relative
R.T. or rt: room temperature
s or sec: second
sat: saturated
SEMCl: chloromethyl-2-trimethylsilylethyl ether
SFC: supercritical fluid chromatography
SOCl₂: sulfur dichloride
tBuOK: potassium tert-butoxide
TBAB: tetrabutylammonium bromide
TBAF: tetrabutylammmonium fluoride
TBAI: tetrabutylammonium iodide
TEA: triethylamine
Tf: trifluoromethanesulfonate
TfAA, TFMSA or Tf₂O: trifluoromethanesulfonic anhydride
TFA: trifluoroacetic acid
TIBSCl: 2,4,6-triisopropylbenzenesulfonyl chloride
TIPS: triisopropylsilyl
THF: tetrahydrofuran
THP: tetrahydropyran
TLC: thin layer chromatography
TMEDA: tetramethylethylenediamine
pTSA: para-toluenesulfonic acid
UPLC: Ultra Performance Liquid Chromatography
wt: weight
Xantphos: 4,5-bis(diphenylphosphino)-9,9-dimethylxanthene

### General Synthetic Methods

The following examples are intended to illustrate the disclosure and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the nucleic acid or analogues thereof of the present disclosure are either commercially available or can be produced by organic synthesis methods known to one of ordinary skill in the art (METHODS OF ORGANIC SYNTHESIS, Thieme, Volume 21 (Houben-Weyl 4th Ed. 1952)). Further, the nucleic acid or analogues thereof of the present disclosure can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain nucleic acid or analogues thereof disclosed herein, one or more ¹H shifts overlap with residual proteo solvent signals; these signals have not been reported in the experimental provided hereinafter.

As depicted in the Examples below, in certain exemplary embodiments, the nucleic acid or analogues thereof were prepared according to the following general procedures. It will be appreciated that, although the general methods depict the synthesis of certain nucleic acid or analogues thereof of the present disclosure, the following general methods, and other methods known to one of ordinary skill in the art, can be applied to all nucleic acid or analogues thereof and subclasses and species of each of these nucleic acid or analogues thereof, as described herein.

### Example 1. Synthesis of 2-(2-((((6aR,8R,9R,9aR)-8-(6-benzamido-9H-purin-9-yl)-2,2,4,4-tetraisopropyltetrahydro-6H-furo[3,2-f][1,3,5,2,4]trioxadisilocin-9-yl)oxy)methoxy)ethoxy) ethan-1-ammonium formate (1-6)

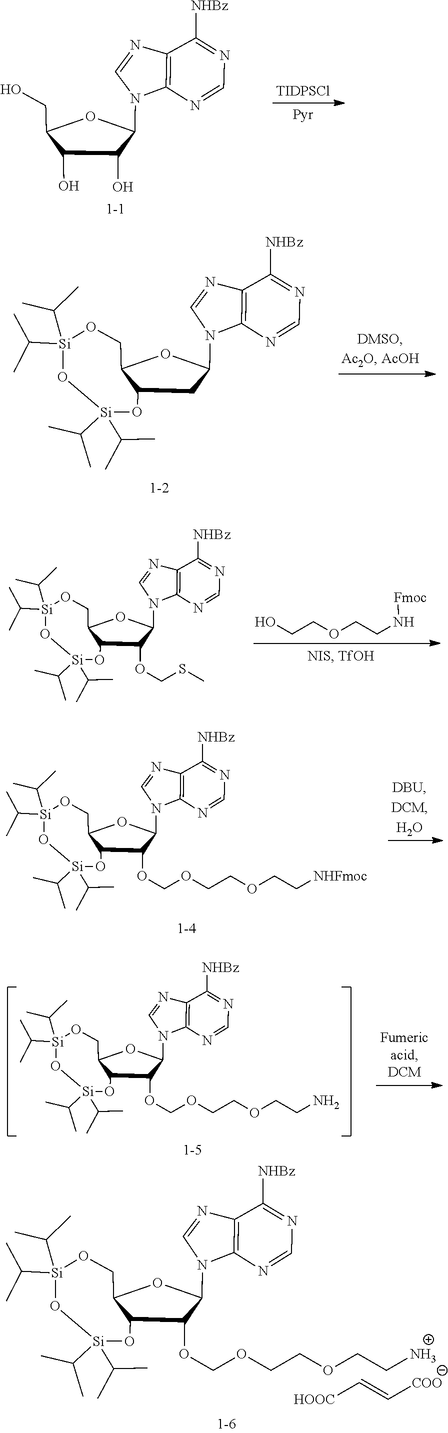

A solution of compound 1-1 (25.00 g, 67.38 mmol) in 20 mL of DMF was treated with pyridine (11 mL, 134.67 mmol) and tetraisopropyldisiloxane dichloride (22.63 mL, 70.75 mmol) at 10° C. The resulting mixture was stirred at 25° C. for 3 h and quenched with 20% citric acid (50 mL). The aqueous layer was extracted with EtOAc (3×50 mL) and the combined organic layers were concentrated in vacuo. The crude residue was recrystallized from a mixture of MTBE and n-heptane (1:15, 320 mL) to afford compound 1-2 (37.20 g, 90%) as a white oily solid.

A solution of compound 1-2 (37.00 g, 60.33 mmol) in 20 mL of DMSO was treated with AcOH (20 mL, 317.20 mmol) and Ac₂O (15 mL, 156.68 mmol). The mixture was stirred at 25° C. for 15 h. The reaction was diluted with EtOAc (100 mL) and quenched with sat. K₂CO₃ (50 mL). The aqueous layer was extracted with EtOAc (3×50 mL). The combined organic layers were concentrated and recrystallized with ACN (30 mL) to afford compound 1-3 (15.65 g, 38.4%) as a white solid.

A solution of compound 1-3 (20.00 g, 29.72 mmol) in 120 mL of DCM was treated with Fmoc-amino-ethoxy ethanol (11.67 g, 35.66 mmol) at 25° C. The mixture was stirred to afford a clear solution and then treated with 4 Å molecular sieves (20.0 g), N-iodosuccinimide (8.02 g, 35.66 mmol), and TfOH (5.25 mL, 59.44 mmol). The mixture was stirred at 30° C. until the HPLC analysis indicated >95% consumption of compound 1-3. The reaction was quenched with TEA (6 mL) and filtered. The filtrate was diluted with EtOAc, washed with sat. NaHCO₃(2×100 mL), sat. Na₂SO₃ (2×100 mL), and water (2×100 mL) and concentrated in vacuo to afford crude compound 1-4 (26.34 g, 93.9%) as a yellow solid, which was used directly for the next step without further purification.

A solution of compound 1-4 (26.34 g, 27.62 mmol) in a mixture of DCM/water (10:7, 170 mL) was treated with DBU (7.00 mL, 45.08 mmol) at 5° C. The mixture was stirred at 5-25° C. for 1 h. The organic layer was then separated, washed with water (100 mL), and diluted with DCM (130 mL). The solution was treated with fumaric acid (7.05 g, 60.76 mmol) and 4 Å molecular sieves (26.34 g) in four portions. The mixture was stirred for 1 h, concentrated, and recrystallized from a mixture of MTBE and DCM (5:1) to afford compound 1-6 (14.74 g, 62.9%) as a white solid: ¹H NMR (400 MHz, d₆-DMSO) 8.73 (s, 1H), 8.58 (s, 1H), 8.15-8.02 (m, 2H), 7.65-7.60 (m, 1H), 7.59-7.51 (m, 2H), 6.52 (s, 2H), 6.15 (s, 1H), 5.08-4.90 (m, 3H), 4.83-4.78 (m, 1H), 4.15-3.90 (m, 3H), 3.79-3.65 (m, 2H), 2.98-2.85 (m, 6H), 1.20-0.95 (m, 28H).

### Example 2. Synthesis of (2R,3R,4R,5R)-5-(6-benzamido-9H-purin-9-yl)-2-((bis(4-methoxyphenyl)(phenyl)methoxy)methyl)-4-((2-(2-[lipid]-amidoethoxy)ethoxy)methoxy) tetrahydrofuran-3-yl (2-cyanoethyl) diisopropylphosphoramidite (2-4a to 2-4e)

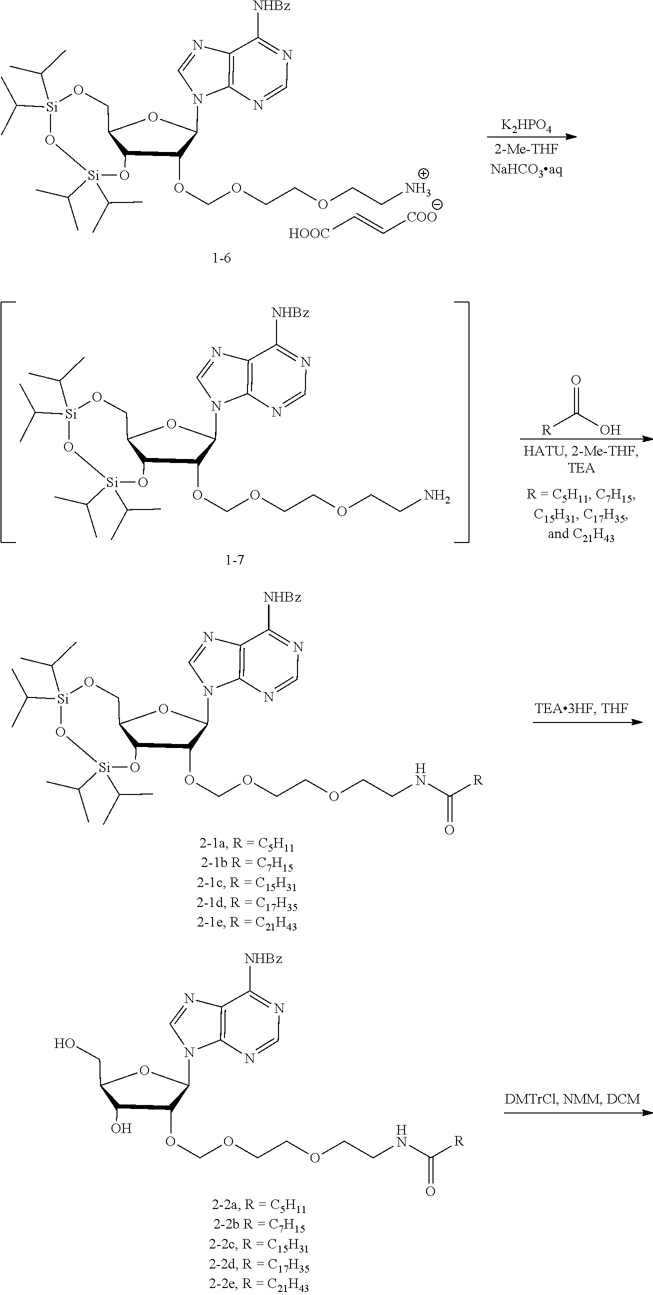

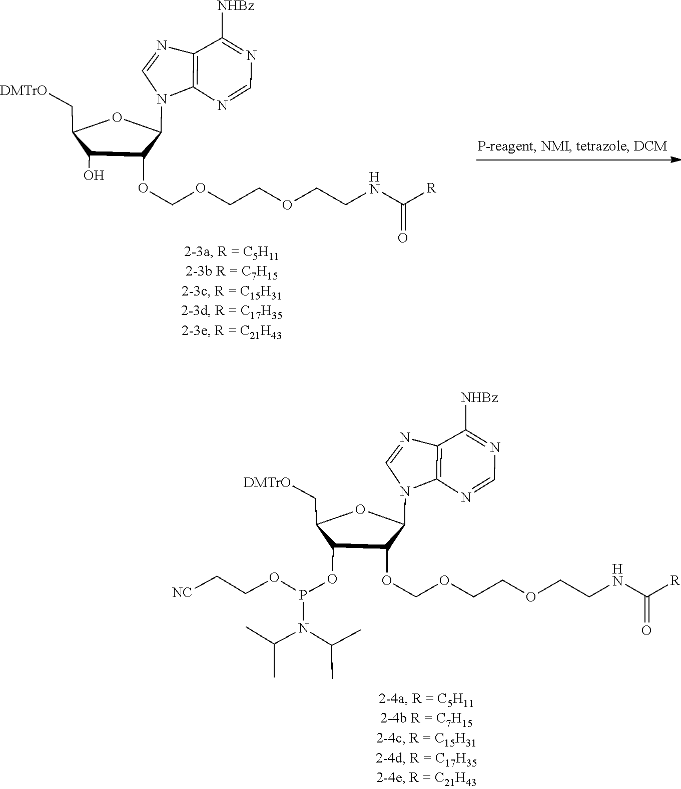

A solution of compound 1-6 (50.00 g, 59.01 mmol) in 150 mL of 2-methyltetrahydrofuran was washed with ice cold aqueous K₂HPO₄ (6%, 100 mL) and brine (20%, 2×100 mL). The organic layer was separated and treated with hexanoic acid (10.33 mL, 82.61 mmol), HATU (33.66 g, 88.52 mmol), and DMAP (10.81 g, 147.52 mmol) at 0° C. The resulting mixture was warmed to 25° C. and stirred for 1 h. The solution was washed with water (2×100 mL), brine (100 mL), and concentrated in vacuo to afford a crude residue. Flash chromatography on silica gel (1:1 hexanes/acetone) gave compound 2-1a (34.95 g, 71.5%) as a white solid.

A mixture of compound 2-1a (34.95 g, 42.19 mmol) and TEA (9.28 mL, 126.58 mmol) in 80 mL of THF was treated with triethylamine trihydrofluoride (20.61 mL, 126.58 mmol) dropwise at 10° C. The mixture was warmed to 25° C. and stirred for 2 h. The reaction was concentrated, dissolved in DCM (100 mL), and washed with sat. NaHCO₃(5×20 mL) and brine (50 mL). The organic layer was concentrated in vacuo to afford crude compound 2-2a (24.72 g, 99%), which was used directly for the next step without further purification.

A solution of compound 2-2a (24.72 g, 42.18 mmol) in 50 mL of DCM was treated with N-methylmorpholine (18.54 mL, 168.67 mmol) and DMTr-Cl (15.69 g, 46.38 mmol). The mixture was stirred at 25° C. for 2 h and quenched with sat. NaHCO₃(50 mL). The organic layer was separated, washed with water, concentrated to afford a slurry crude. Flash chromatography on silica gel (1:1 hexanes/acetone) gave compound 2-3a (30.05 g, 33.8 mmol, 79.9%) as a white solid.

A solution of compound 2-3a (25.00 g, 28.17 mmol) in 50 mL of DCM was treated with N-methylmorpholine (3.10 mL, 28.17 mmol) and tetrazole (0.67 mL, 14.09 mmol) under nitrogen atmosphere. Bis(diisopropylamino) chlorophosphine (9.02 g, 33.80 mmol) was added to the solution dropwise and the resulting mixture was stirred at 25° C. for 4 h. The reaction was quenched with water (15 mL), and the aqueous layer was extracted with DCM (3×50 mL). The combined organic layers were washed with sat. NaHCO₃(50 mL), concentrated to afford a crude solid that was recrystallized from a mixture of DCM/MTBE/n-hexane (1:4:40) to afford compound 2-4a (25.52 g, 83.4%) as a white solid: ¹H NMR (400 MHz, d₆-DMSO) 11.25 (s, 1H), 8.65-8.60 (m, 2H), 8.09-8.02 (m, 2H), 7.71 (s, 1H), 7.67-7.60 (m, 1H), 7.59-7.51 (m, 2H), 7.38-7.34 (m, 2H), 7.30-7.25 (m, 7H), 6.85-6.79 (m, 4H), 6.23-6.20 (m, 1H), 5.23-5.14 (m, 1H), 4.80-4.69 (m, 3H), 4.33-4.23 (m, 2H), 3.90-3.78 (m, 1H), 3.75 (s, 6H), 3.74-3.52 (m, 3H), 3.50-3.20 (m, 6H), 3.14-3.09 (m, 2H), 3.09 (s, 1H), 2.82-2.80 (m, 1H), 2.65-2.60 (m, 1H), 2.05-1.96 (m, 2H), 1.50-1.39 (m, 2H), 1.31-1.10 (m, 14H), 1.08-1.05 (m, 2H), 0.85-0.79 (m, 3H); ³¹P NMR (162 MHz, d₆-DMSO) 149.43, 149.18.

Compound 2-4b, 2-4c, 2-4d, and 2-4e were prepared using similar procedures described above for compound 2-4a. Compound 2-4b was obtained (25.50 g, 85.4%) as a white solid: ¹H NMR (400 MHz, d₆-DMSO) 11.23 (s, 1H), 8.65-8.60 (m, 2H), 8.05-8.02 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.97 (m, 2H), 1.50-1.38 (m, 2H), 1.31-1.10 (m, 18H), 1.08-1.05 (m, 2H), 0.85-0.78 (m, 3H); ³¹P NMR (162 MHz, d₆-DMSO) 149.43, 149.19.

Compound 2-4c was obtained (36.60 g, 66.3%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.25-5.17 (m, 1H), 4.80-4.69 (m, 3H), 4.40-4.21 (m, 2H), 3.91-3.80 (m, 1H), 3.74 (s, 6H), 3.74-3.50 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.33-1.12 (m, 38H), 1.08-1.05 (m, 2H), 0.86-0.80 (m, 3H); ³¹P NMR (162 MHz, d₆-DMSO) 149.42, 149.17.

Compound 2-4d was obtained (26.60 g, 72.9%) as an off-white solid: ¹H NMR (400 MHz, d₆-DMSO) 11.22 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.33 (m, 2H), 7.30-7.25 (m, 7H), 6.89-6.80 (m, 4H), 6.21-6.15 (m, 1H), 5.22-5.17 (m, 1H), 4.80-4.69 (m, 3H), 4.40-4.21 (m, 2H), 3.91-3.80 (m, 1H), 3.74 (s, 6H), 3.74-3.52 (m, 3H), 3.50-3.20 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.08 (m, 38H), 1.08-1.05 (m, 2H), 0.85-0.79 (m, 3H); ³¹P NMR (162 MHz, d₆-DMSO) 149.47, 149.22.

Compound 2-4e was obtained (38.10 g, 54.0%) as a white solid: ¹H NMR (400 MHz, d₆-DMSO) 11.21 (s, 1H), 8.64-8.59 (m, 2H), 8.05-8.00 (m, 2H), 7.73-7.70 (m, 1H), 7.67-7.60 (m, 1H), 7.59-7.51 (m, 2H), 7.38-7.34 (m, 2H), 7.30-7.25 (m, 7H), 6.89-6.80 (m, 4H), 6.21-6.15 (m, 1H), 5.23-5.17 (m, 1H), 4.80-4.69 (m, 3H), 4.40-4.21 (m, 2H), 3.91-3.80 (m, 1H), 3.73 (s, 6H), 3.74-3.52 (m, 3H), 3.47-3.22 (m, 6H), 3.14-3.09 (m, 2H), 3.09 (s, 1H), 2.83-2.79 (m, 1H), 2.68-2.62 (m, 1H), 2.05-1.99 (m, 2H), 1.50-1.38 (m, 2H), 1.35-1.06 (m, 46H), 1.08-1.06 (m, 2H), 0.85-0.77 (m, 3H); ³¹P NMR (162 MHz, d₆-DMSO) 149.41, 149.15.

### Example 3. Synthesis of Lipid GalXC Conjugates

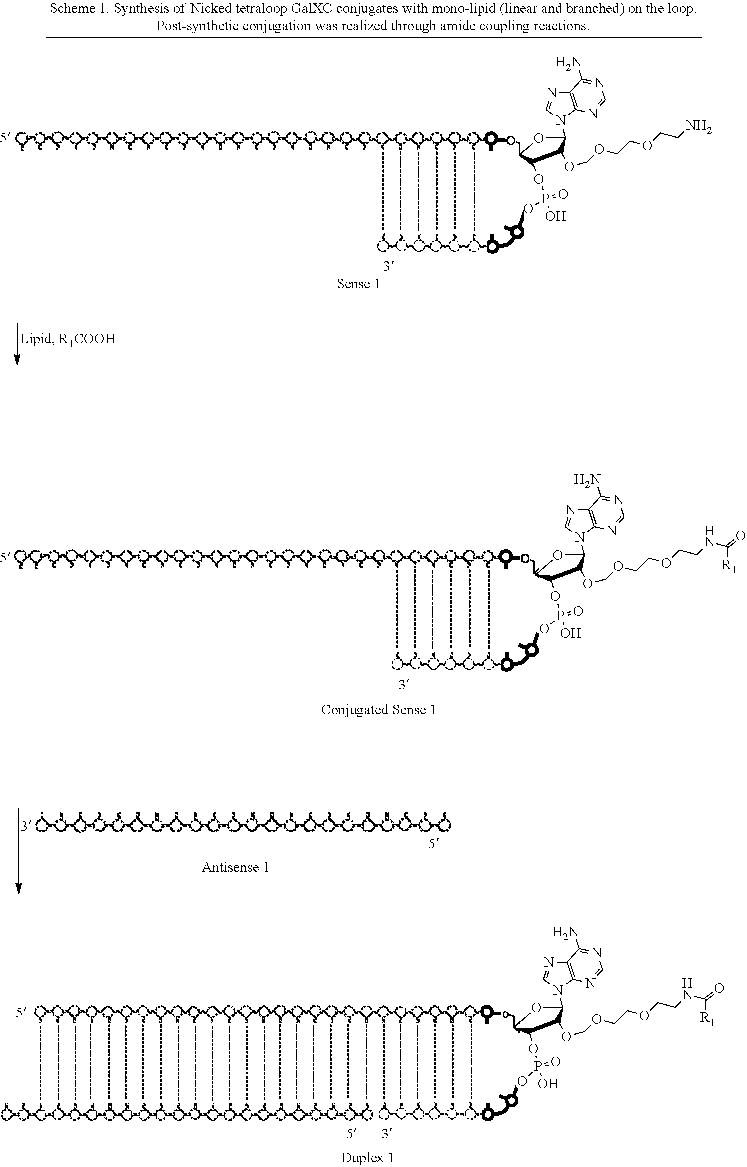 [0800] R₁COOH group represents fatty acid C8:0, C10:0, C11:0, C12:0, C14:0, C16:0, C17:0, C18:0, C18:1, C18:2, C22:5, C22:0, C24:0, C26:0, C22:6, C24:1 diacyl C16:0 or diacyl C18:1

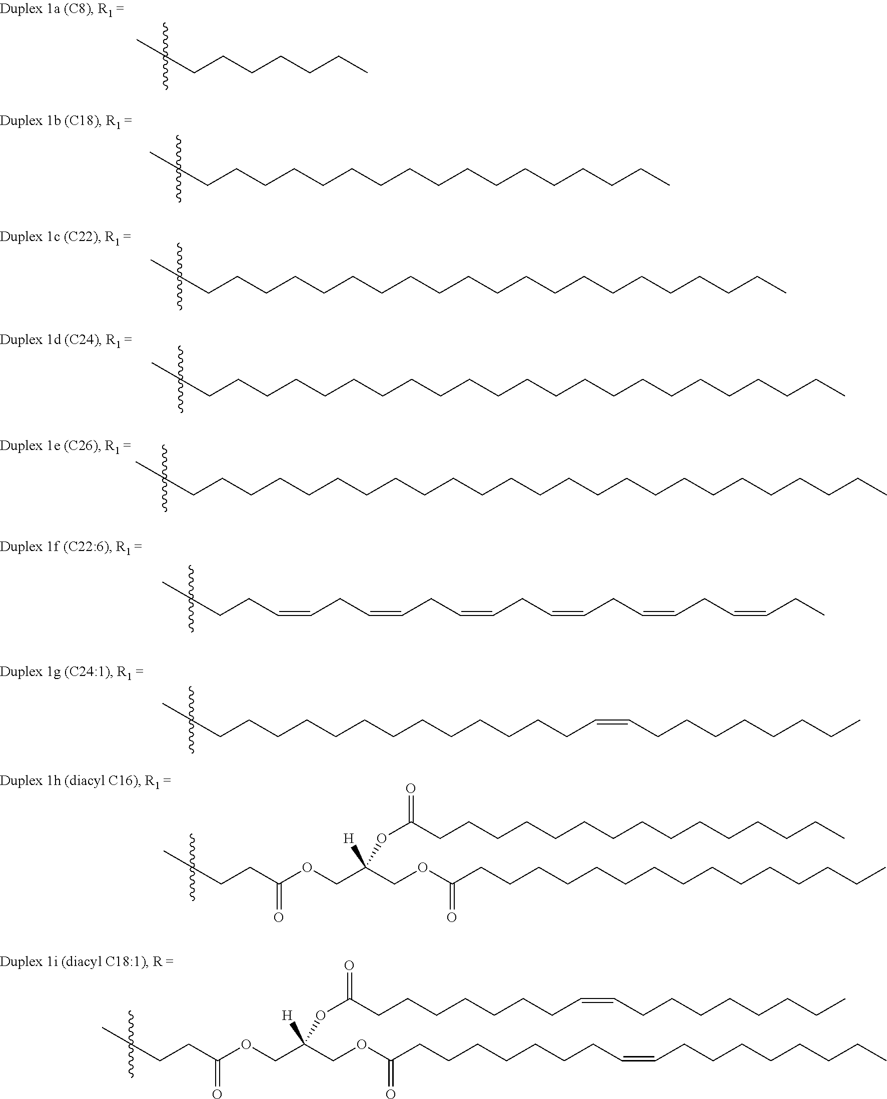

Synthesis Sense 1 and Antisense 1 were prepared by solid-phase synthesis.

### Synthesis of Conjugated Sense 1a-1i

Conjugated Sense 1a was synthesized through post-syntenic conjugation approach. In Eppendorf tube 1, a solution of octanoic acid (0.58 mg, 4 umol) in DMA (0.75 mL) was treated with HATU (1.52 mg, 4 umol) at rt. In Eppendorf tube 2, a solution of oligo Sense 1 (10.00 mg, 0.8 umol) in H₂O (0.25 mL) was treated with DIPEA (1.39 uL, 8 umol). The solution in Eppendorf tube 1 was added to the Eppendorf tube 2 and mixed using ThermoMixer at rt. After the reaction was completed indicated by LC-MS analysis, the reaction mixture was diluted with 5 mL of water and purified by revers phase XBridge C18 column using a 5-95% gradient of 100 mM TEAA in ACN and H₂O. The product fractions were concentrated under reduced pressure using Genevac. The combined residual solvent was dialyzed against water (1 X), saline (1 X), and water (3 X) using Amicon® Ultra-15 Centrifugal (3K). The Amicon membrane was washed with water (3×2 mL) and the combined solvents were then lyophilized to afford an amorphous white solid of Conjugated Sense 1a (6.43 mg, 64% yield).

Conjugated Sense 1b-1i were prepared using similar procedures as described for the synthesis of Conjugated Sense 1a and obtained in 42%-69% yields.

Annealing of Duplex 1a-1j.

Conjugated Sense 1a (10 mg, measured by weight) was dissolved in 0.5 mL deionized water to prepare a 20 mg/mL solution. Antisense 1 (10 mg, measured by OD) was dissolved in 0.5 mL deionized water to prepare a 20 mg/mL solution, which was used for the titration of the conjugated sense and quantification of the duplex amount. Based on the calculation of molar amounts of both conjugated sense and antisense, a proportion of required Antisense 1 was added to the Conjugated Sense 1a solution. The resulting mixture was stirred at 95° C. for 5 min and allowed to cool down to rt. The annealing progress was monitored by ion-exchange HPLC. Based on the annealing progress, several proportions of Antisense 1 were further added to complete the annealing with >95% purity. The solution was lyophilized to afford Duplex 1a (C8) and its amount was calculated based on the molar amount of the antisense consumed in the annealing.

Duplex 1b-1i were prepared using the same procedures as described for the annealing of Duplex 1a (C8).

The following Scheme 1-2 depicts the synthesis of Nicked tetraloop GalXC conjugates with mono-lipid on the loop. Post-synthetic conjugation was realized through Cu-catalyzed alkyne-azide cycloaddition reaction.

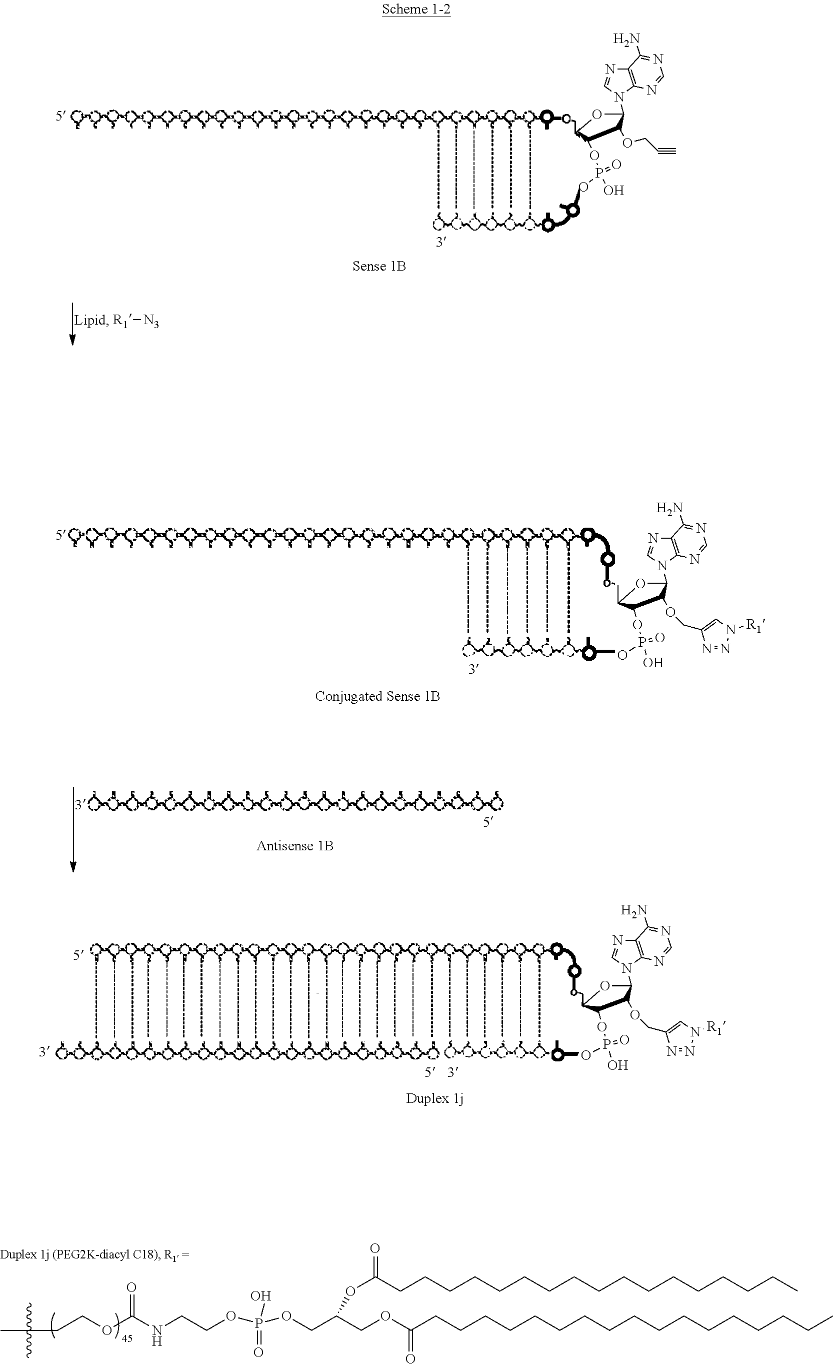

Sense 1B and Antisense 1B were prepared by solid-phase synthesis. Synthesis of Conjugated Sense 1j.

In Eppendorf tube 1, a solution of oligo (10.00 mg, 0.8 umol) in a 3:1 mixture of DMA/H₂O (0.5 mL) was treated with the lipid linker azide (11.26 mg, 4 umol). In Eppendorf tube 2, CuBr dimethyl sulfide (1.64 mg, 8 umol) was dissolved in ACN (0.5 mL). Both solutions were degassed for 10 min by bubbling N₂ through them. The ACN solution of CuBrSMe₂ was then added into tube 1 and the resulting mixture was stirred at 40° C. After the reaction was completed indicated by LC-MS analysis, the reaction mixture was diluted with 0.5 M EDTA (2 mL) and dialyzed against water (2×) using a Amicon® Ultra-15 Centrifugal (3K). The reaction crude was purified by revers phase XBridge C18 column using a 5-95% gradient of 100 mM TEAA in ACN (with 30% IPA spiked in) and H₂O. The product fractions were concentrated under reduced pressure using Genevac. The combined residual solvent was dialyzed against water (1×), saline (1×), and water (3×) using Amicon® Ultra-15 Centrifugal (3K). The Amicon membrane was washed with water (3×2 mL) and the combined solvents were lyophilized to afford an amorphous white solid of Conjugated Sense 1j (6.90 mg, 57% yield).

Duplex 1j (PEG2K-diacyl C18) was prepared using the same procedures as described for the annealing of Duplex 1a (C8).

The following Scheme 1-3 depicts the synthesis of Nicked tetraloop GalXC conjugates with di-lipid on the loop using post-synthetic conjugation approach.

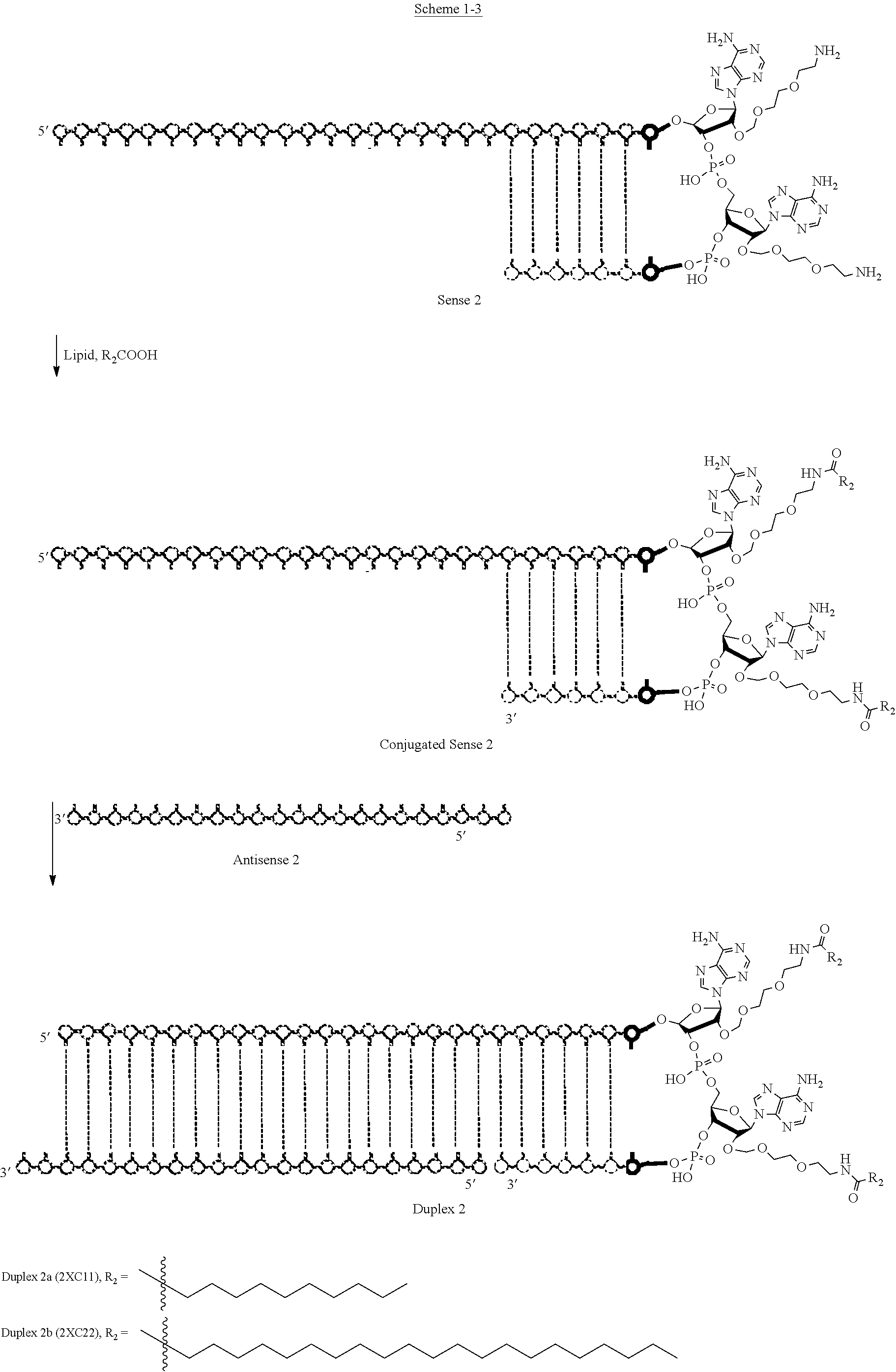

Sense 2 and Antisense 2 were prepared by solid-phase synthesis.

Conjugated Sense 2a and 2b were prepared using similar procedures as described for the synthesis of Conjugated Sense 1a but with 10 eq of lipid, 10 eq of HATU, and 20 eq of DIPEA.

Duplex 2a (2XC11) and 2b (2XC22) were prepared using the same procedures as described for the annealing of Duplex 1a (C8).

The following Scheme 1-4 depicts the synthesis of GalXC of fully phosphorothioated stem-loop conjugated with mono-lipid using post-synthetic conjugation approach.

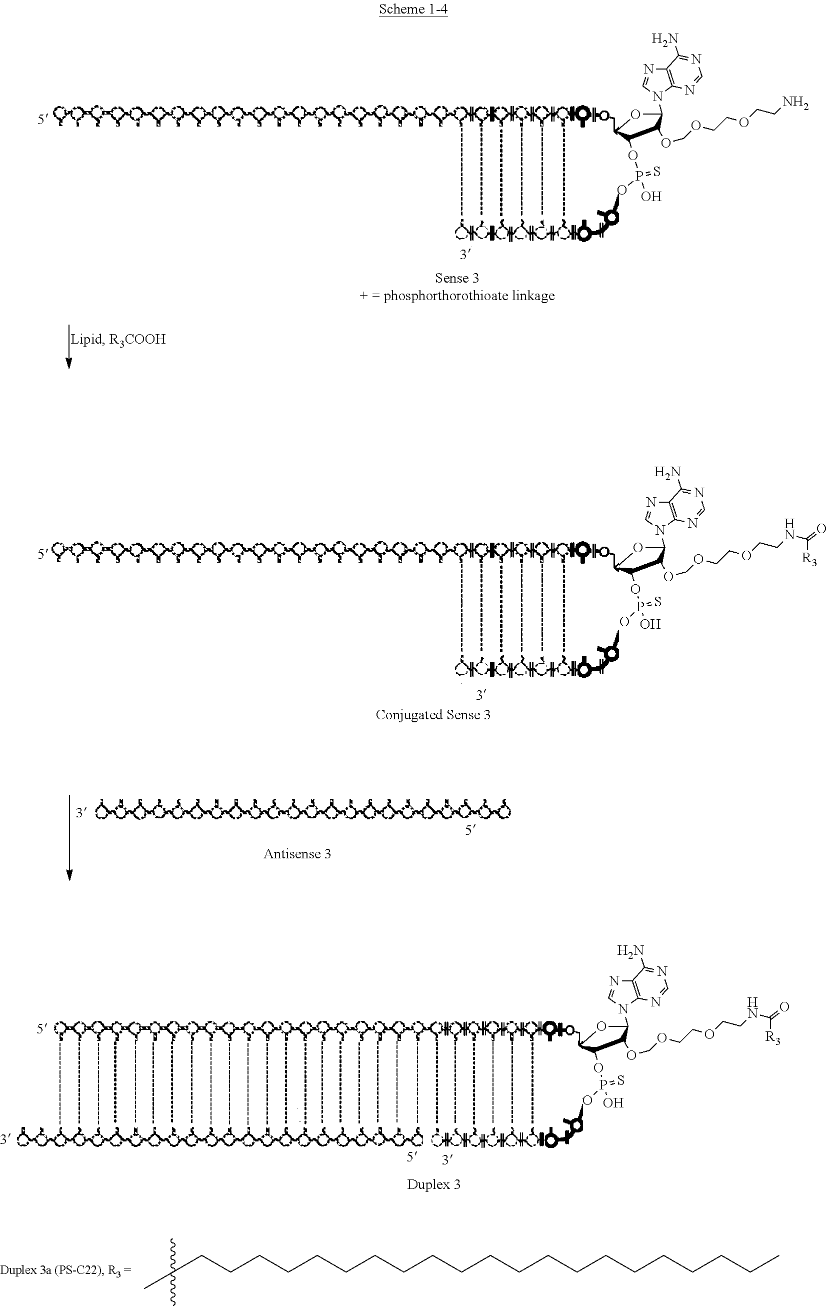

Sense 3 and Antisense 3 were prepared by solid-phase synthesis.

Conjugated Sense 3a was prepared using similar procedures as described for the synthesis of Conjugated Sense 1a and obtained in a 65% yield.

Duplex 3a (PS-C22) was prepared using the same procedures as described for the annealing of Duplex 1a (C8).

The following Scheme1-5 depicts the synthesis of GalXC of short sense conjugated with mono-lipid using post-synthetic conjugation approach.

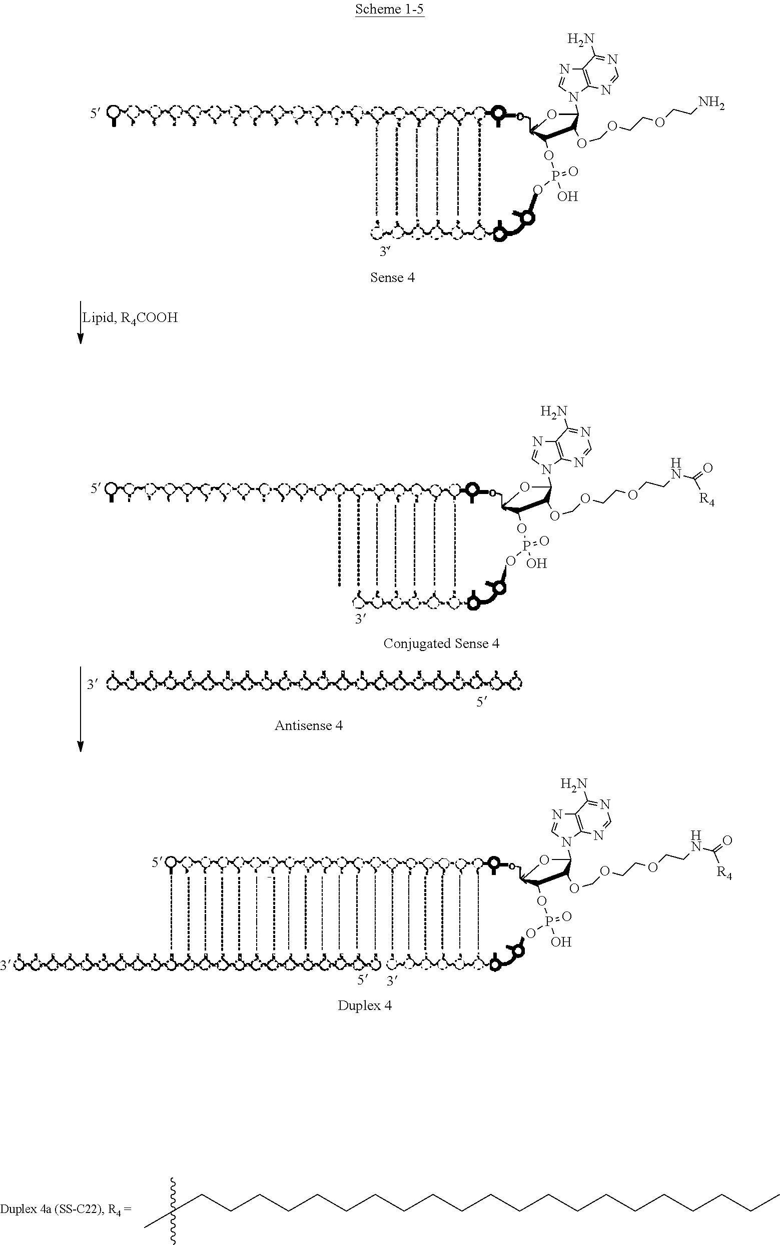

Sense 4 and Antisense 4 were prepared by solid-phase synthesis.

Conjugated Sense 4a was prepared using similar procedures as described for the synthesis of Conjugated Sense 1a and obtained in a 74% yield.

Duplex 4a (SS-C22) was prepared using the same procedures as described for the annealing of Duplex 1a (C8).

The following Scheme 1-6 depicts the synthesis of Nicked tetraloop GalXC conjugated with tri-adamantane moiety on the loop using post-synthetic conjugation approach.

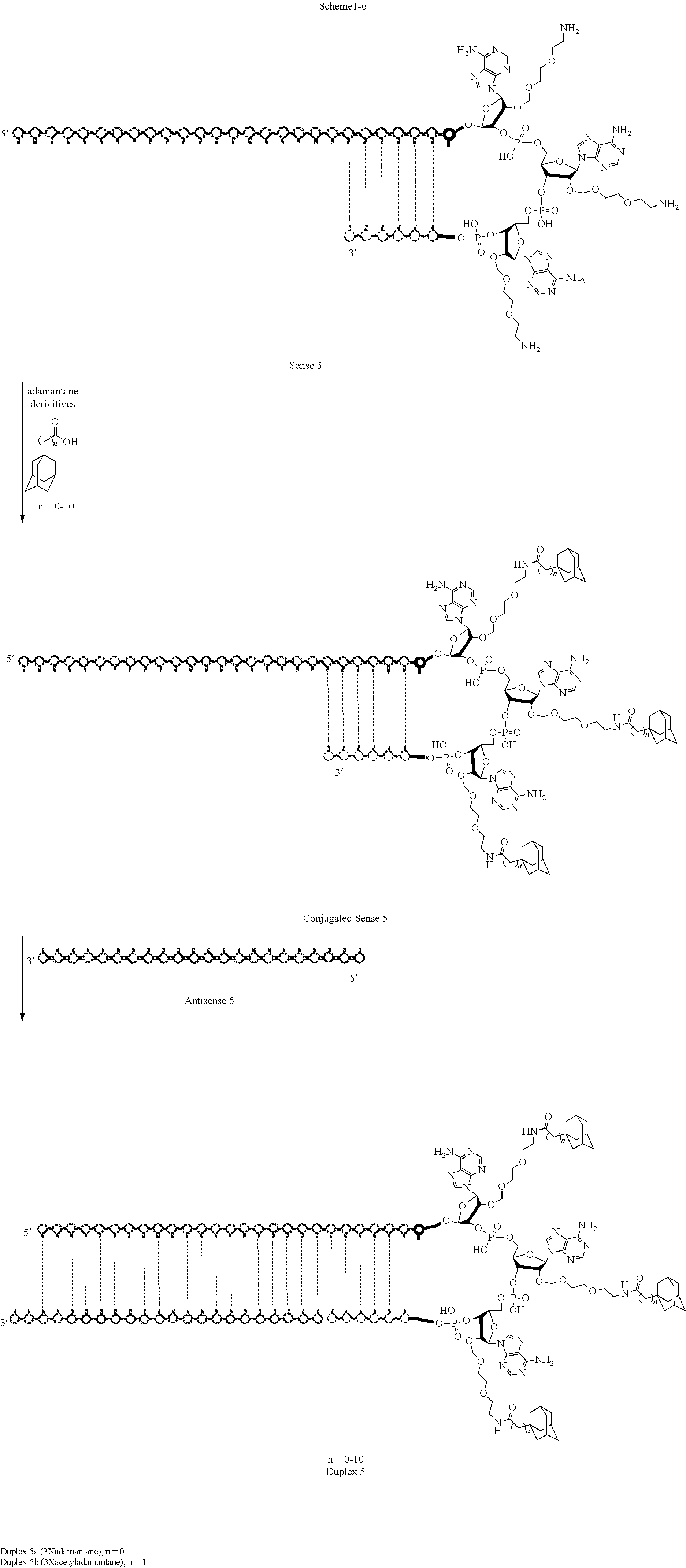

Sense 5 and Antisense 5 were prepared by solid-phase synthesis.

Conjugated Sense 5a and 5b were prepared using similar procedures as described for the synthesis of Conjugated Sense 1a and obtained in 42%-73% yields.

Duplex 5a (3Xadamantane) and Duplex 5b (3Xacetyladamantane) were prepared using the same procedures as described for the annealing of Duplex 1a (C8).

The following scheme 1-7 depicts an example of solid phase synthesis of Nicked tetraloop GalXC conjugated with lipid(s) on the loop.

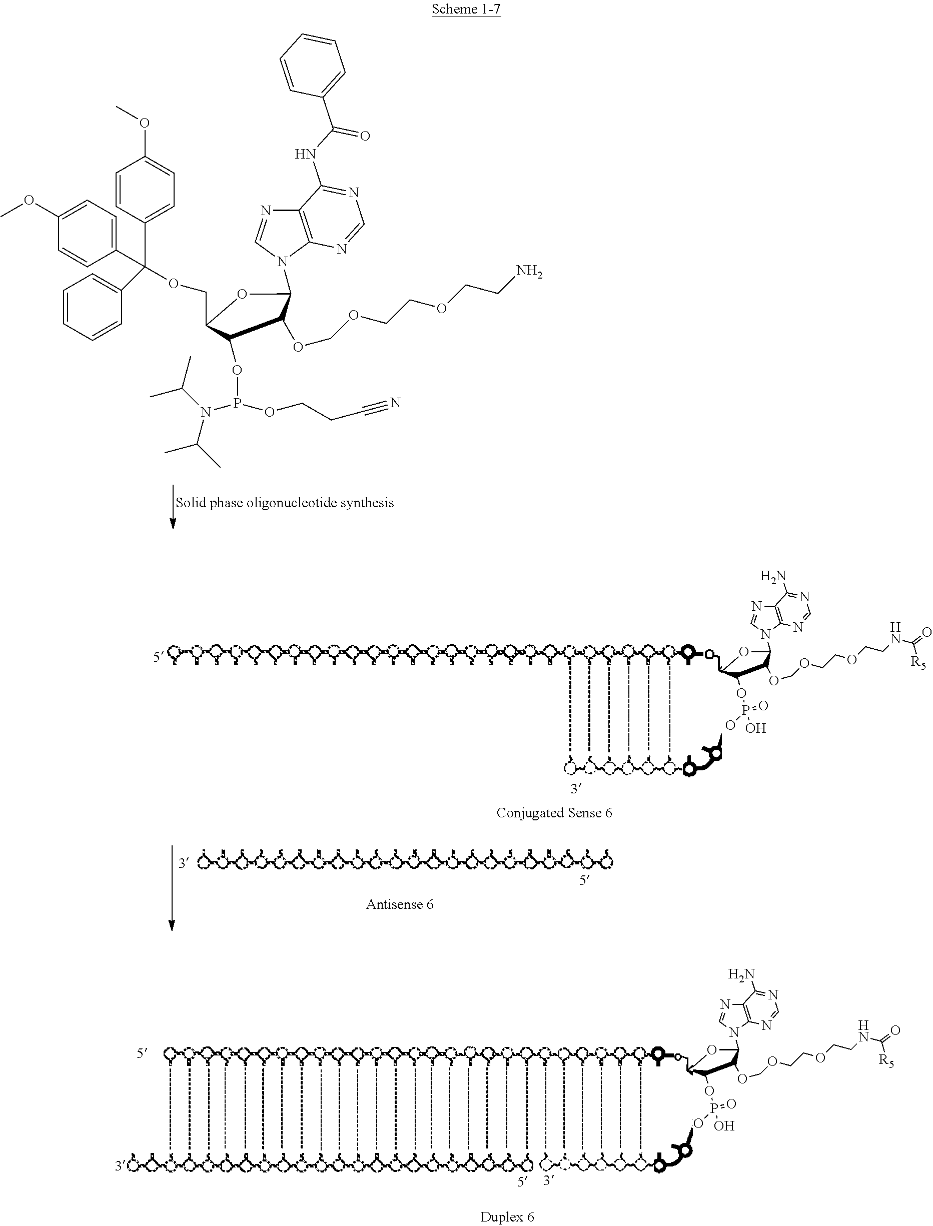

### Synthesis of Conjugated Sense 6

Conjugated Sense 6 was prepared by solid-phase synthesis using a commercial oligo synthesizer. The oligonucleotides were synthesized using 2′-modified nucleoside phosphoramidites, such as 2′-F or 2′-OMe, and 2′-diethoxymethanol linked fatty acid amide nucleoside phosphoramidites. Oligonucleotide synthesis was conducted on a solid support in the 3′ to 5′direction using a standard oligonucleotide synthesis protocol. 5-ethylthio-1H-tetrazole (ETT) was used as an activator for the coupling reaction. Iodine solution was used for phosphite triester oxidation. 3-(Dimethylaminomethylidene)amino-3H-1,2,4-dithiazole-3-thione (DDTT) was used for the formation of phosphorothioate linkages. Synthesized oligonucleotides were treated with concentrated aqueous ammonium for 10 h. The ammonia was removed from the suspension and the solid support residues were removed by filtration. The crude oligonucleotide was treated with TEAA, analyzed and purified by strong anion exchange high performance liquid chromatography (SAX-HPLC). The fractions were combined and dialyzed against water (3×), saline (1×), and water (3×) using Amicon® Ultra-15 Centrifugal (3K). The remaining solvent was then lyophilized to afford the desired Conjugated Sense 6.

Duplex 6 was prepared using the same procedures as described for the annealing of Duplex 1a (C8).

Scheme 8. Synthesis of Nicked tetraloop GalXC conjugated with one adamantane unit on the loop via a post-synthetic conjugation approach.

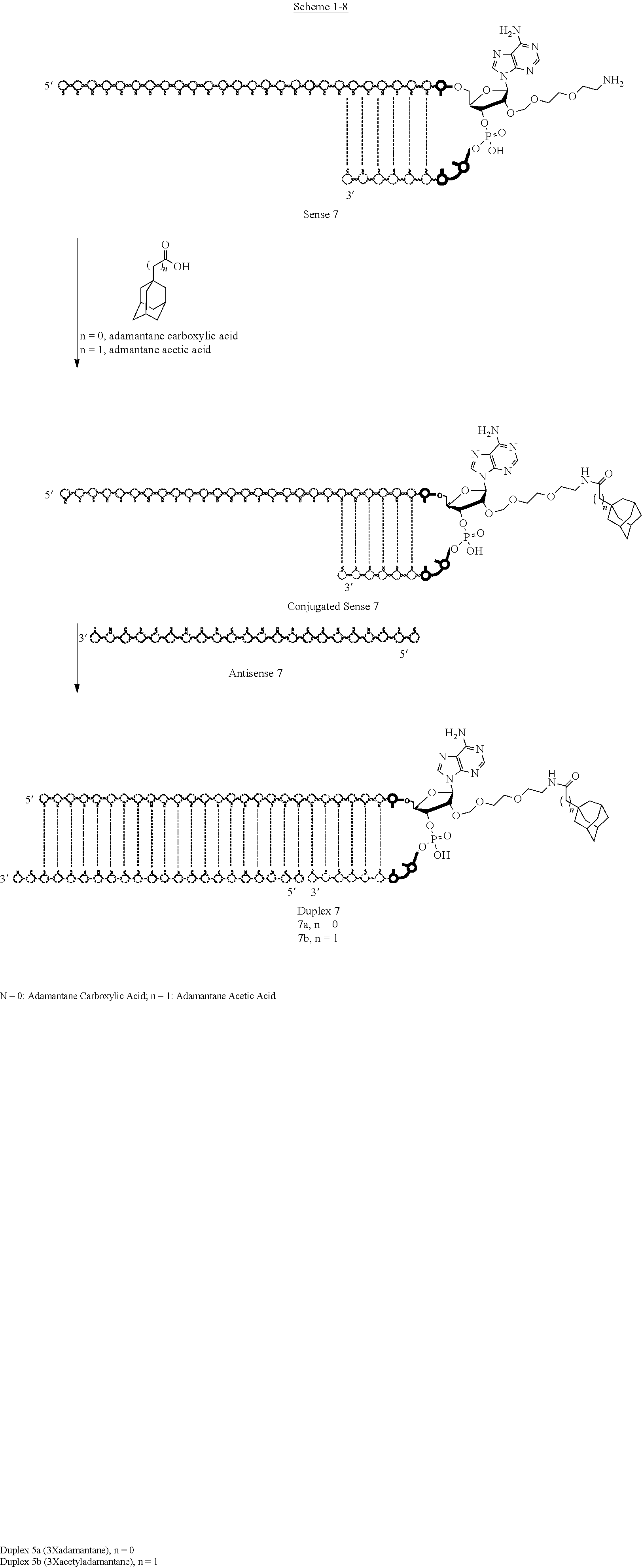

### Synthesis of Conjugated Sense 7a and 7b

Conjugated Sense 7a and Sense 7b were obtained using the same method or a substantially similar method to the synthesis of Conjugated Sense 5.

### Synthesis Example of Duplex 7a and 7b

Duplex 7a and Duplex 7b were obtained using the same method or a substantially similar method to the synthesis of Duplex 5.

Scheme 9. Synthesis of nicked tetraloop GalXC conjugated with two adamantane units on the loop via a post-synthetic conjugation approach.

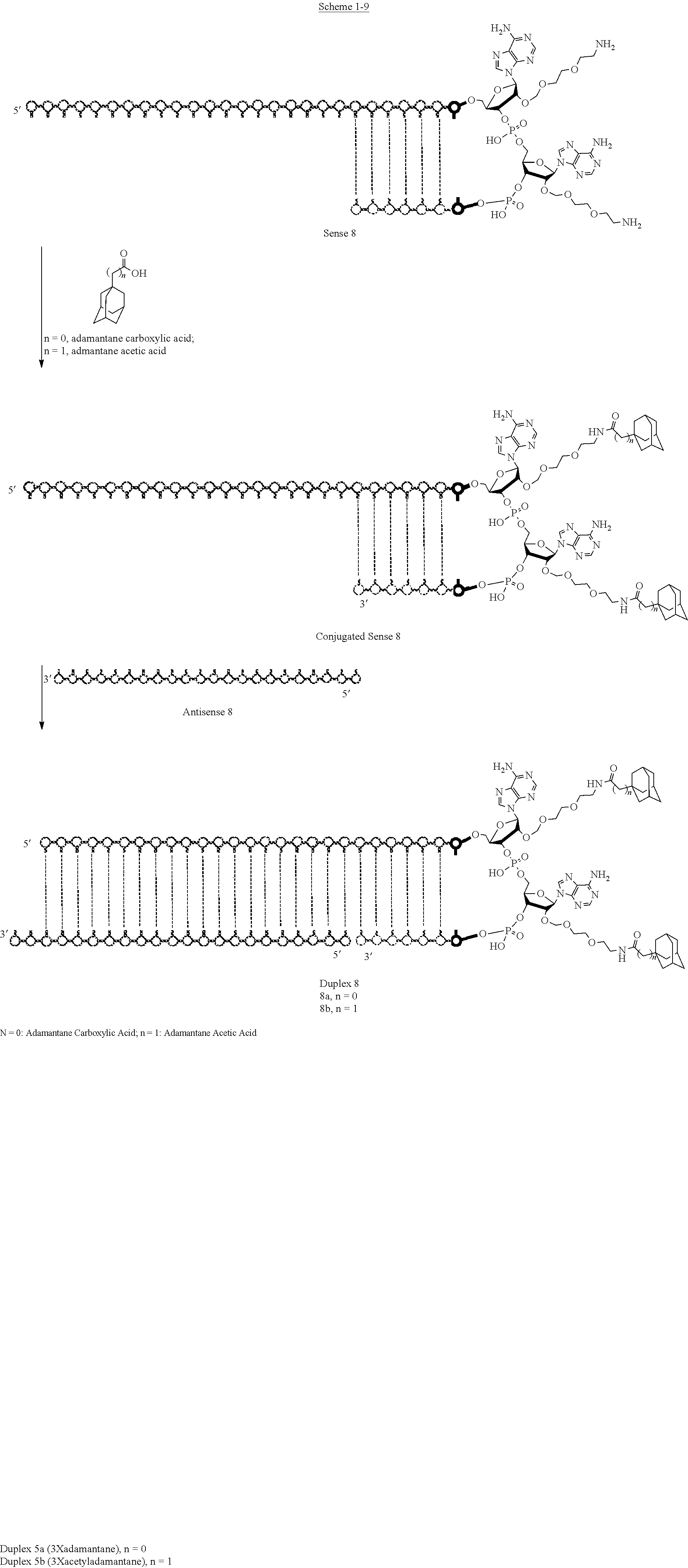

### Synthesis of Conjugated Sense 8a and 8b

Conjugated Sense 8a and Sense 8b were obtained using the same method or a substantially similar method to the synthesis of Conjugated Sense 5.

### Synthesis Example of Duplex 8a and 8b

Duplex 8a and Duplex 8b were obtained using the same method or a substantially similar method to the synthesis of Duplex 5.

The following Scheme1-10 depicts the synthesis of GalXC of short sense and short stem loop conjugated with mono-lipid using post-synthetic conjugation approach.

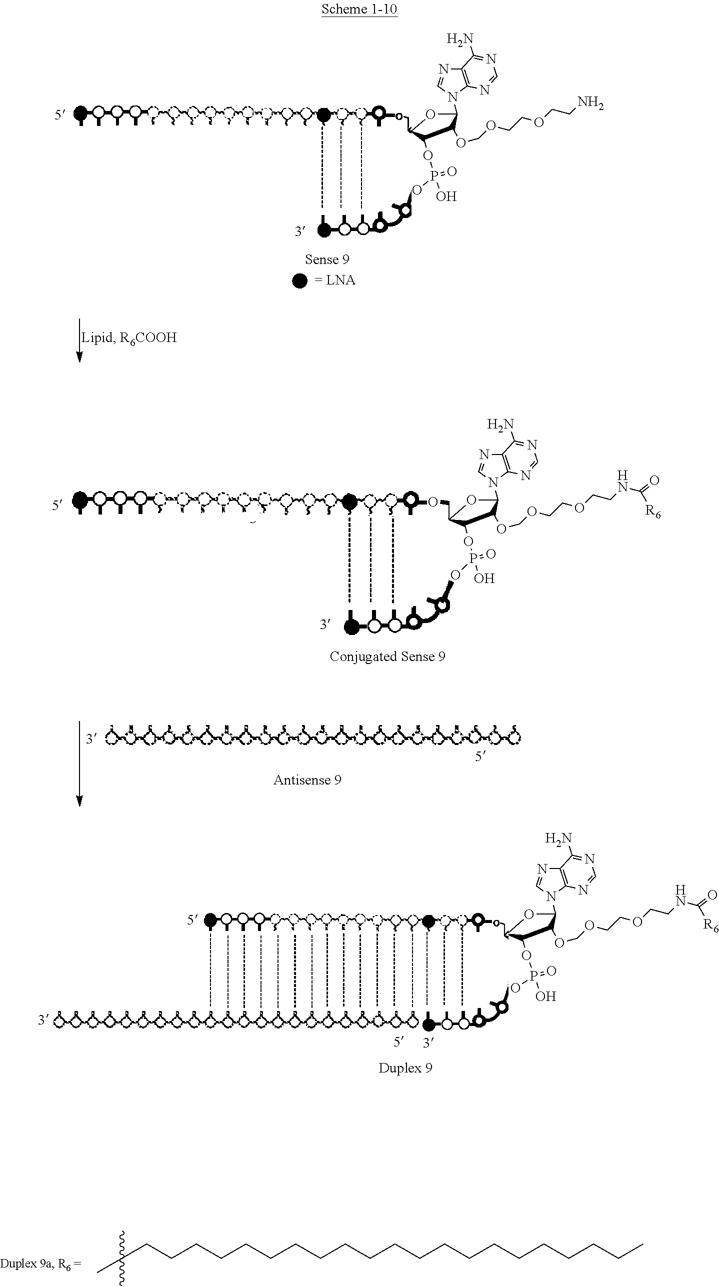

### Synthesis of Sense 9a

Conjugated Sense 9a was obtained using the same method or a substantially similar method to the synthesis of Conjugated Sense 5.

### Synthesis Example of Duplex 9a

Duplex 9a was obtained using the same method or a substantially similar method to the synthesis of Duplex 5.

The following Scheme1-11 depicts the synthesis of GalXC conjugated with mono-lipid at 5′-end using post-synthetic conjugation approach.

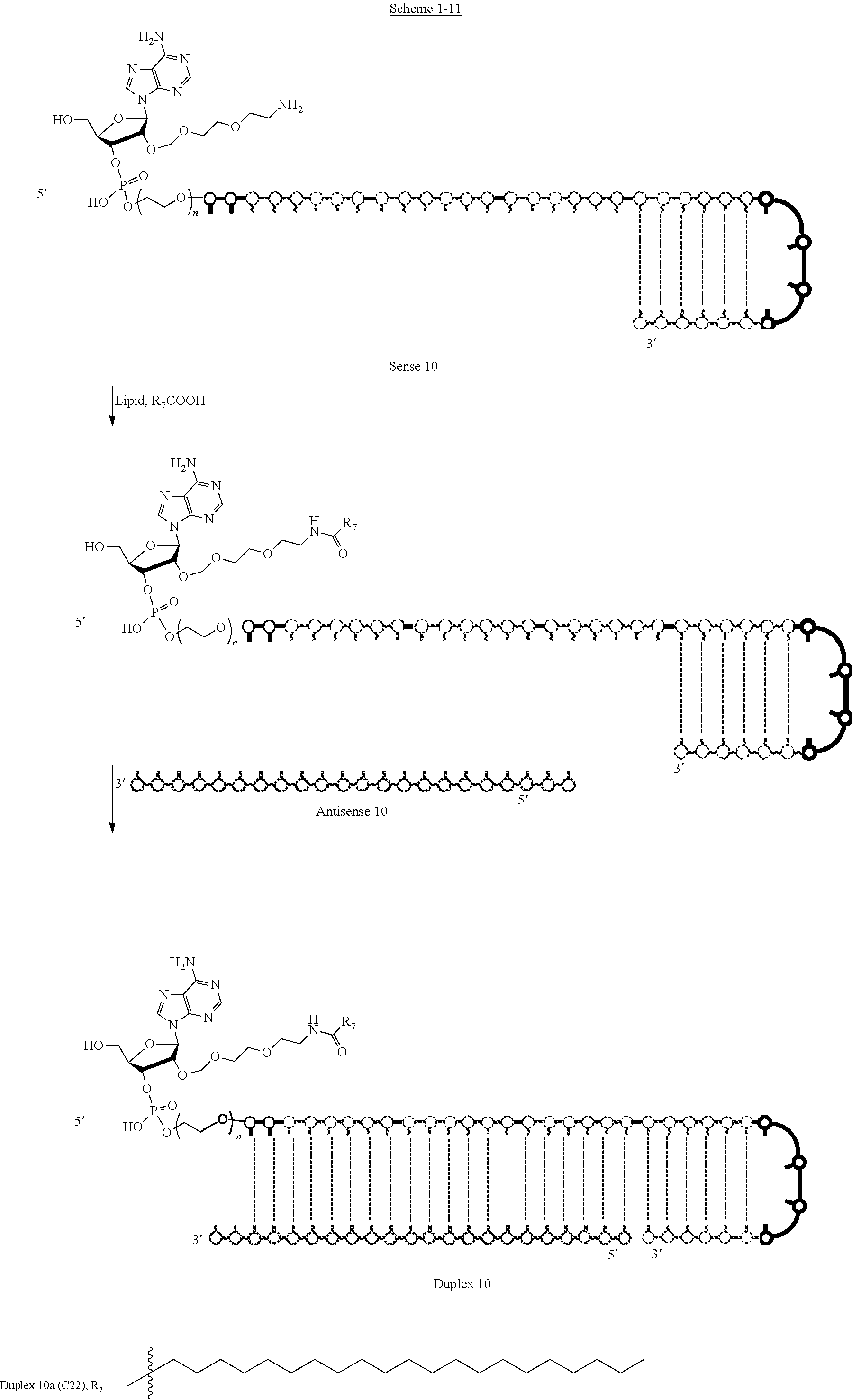

### Synthesis of Conjugated Sense 10a

Conjugated Sense 10a was obtained using the same method or a substantially similar method to the synthesis of Conjugated Sense 5.

### Synthesis Example of Duplex 10a

Duplex 10a was obtained using the same method or a substantially similar method to the synthesis of Duplex 5.

The following Scheme1-12a and 1-12b depict the synthesis of GalXC with blunt end conjugated with mono-lipid at 3′-end or 5′-end using post-synthetic conjugation approach.

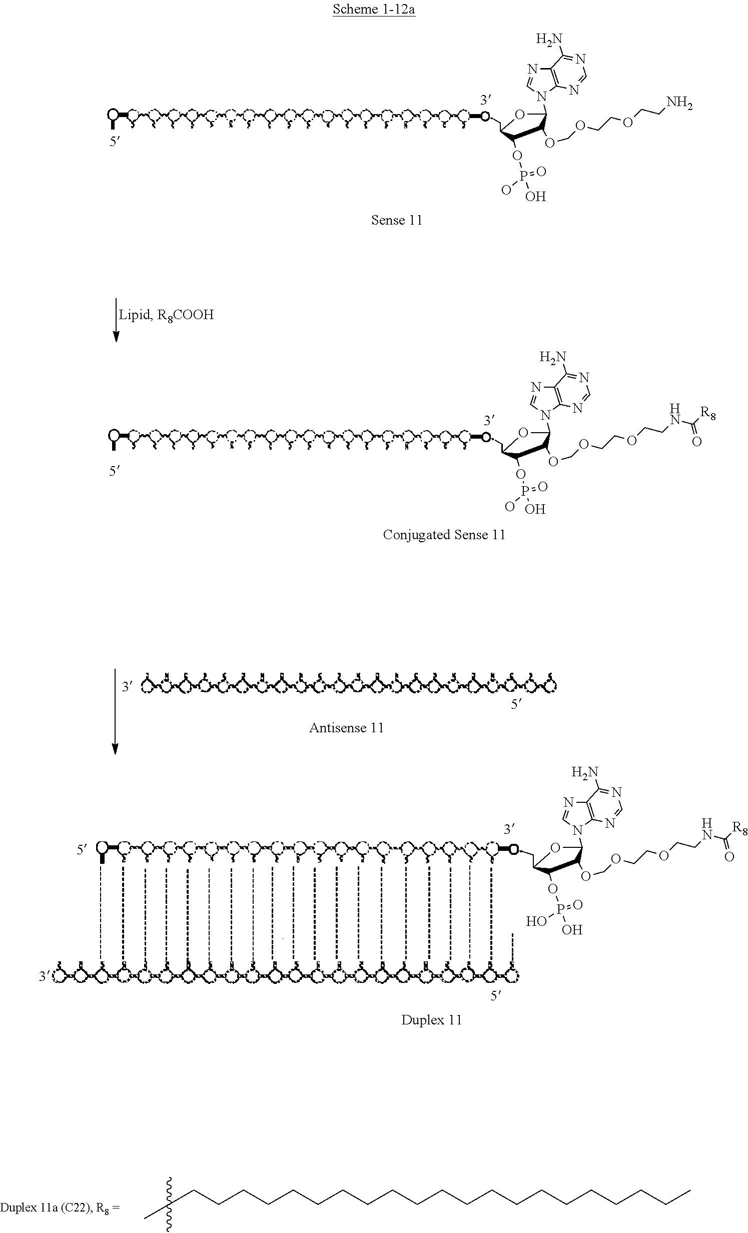

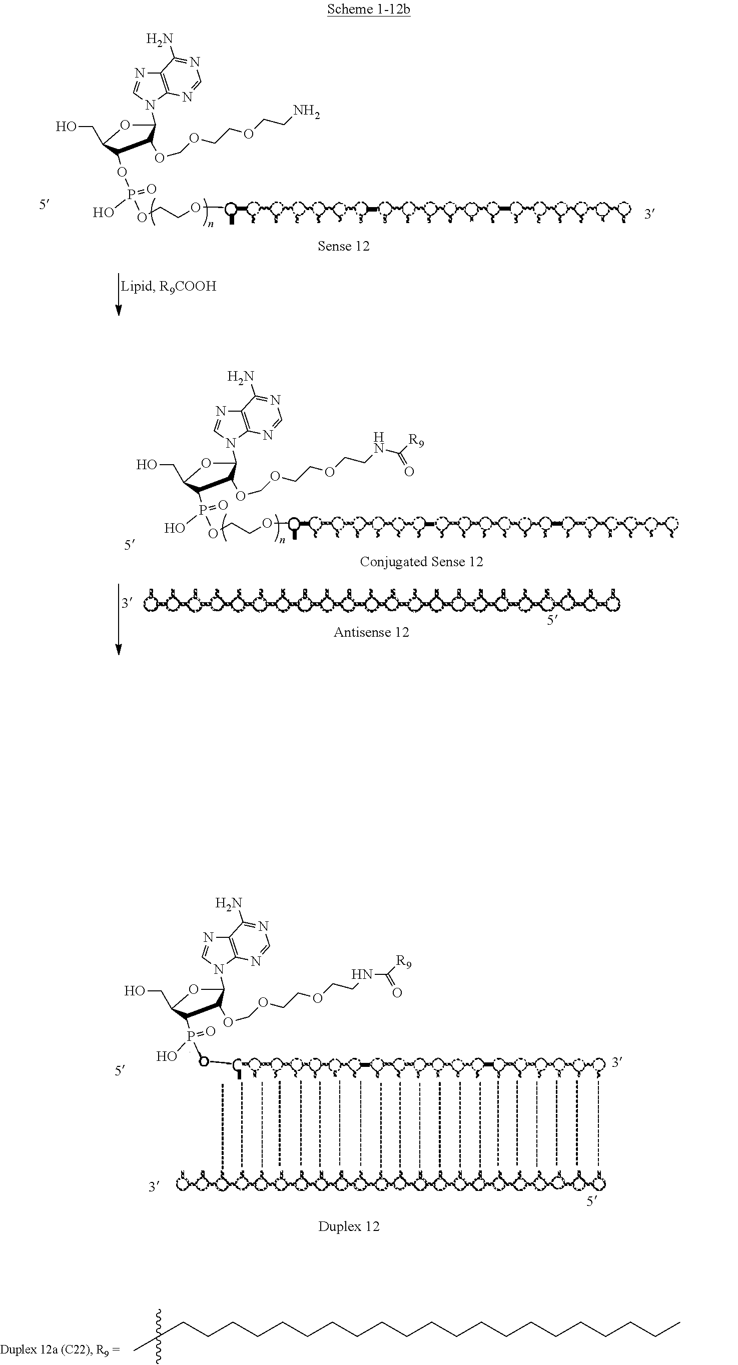

### Synthesis of Conjugated Sense 11a and 12a

Conjugated Sense 11a and 12a were obtained using the same method or a substantially similar method to the synthesis of Conjugated Sense 5.

### Synthesis Example of Duplex 11a and 12a

Duplex 11a and 12a were obtained using the same method or a substantially similar method to the synthesis of Duplex 5.

Conjugates Duplex 8D and Duplex 9D were obtained using the same method or a substantially similar method to the synthesis of Duplex 5.

### Example 4. Biodistribution and Gene Silencing Activity of DRNA GalXC Lipid Conjugates

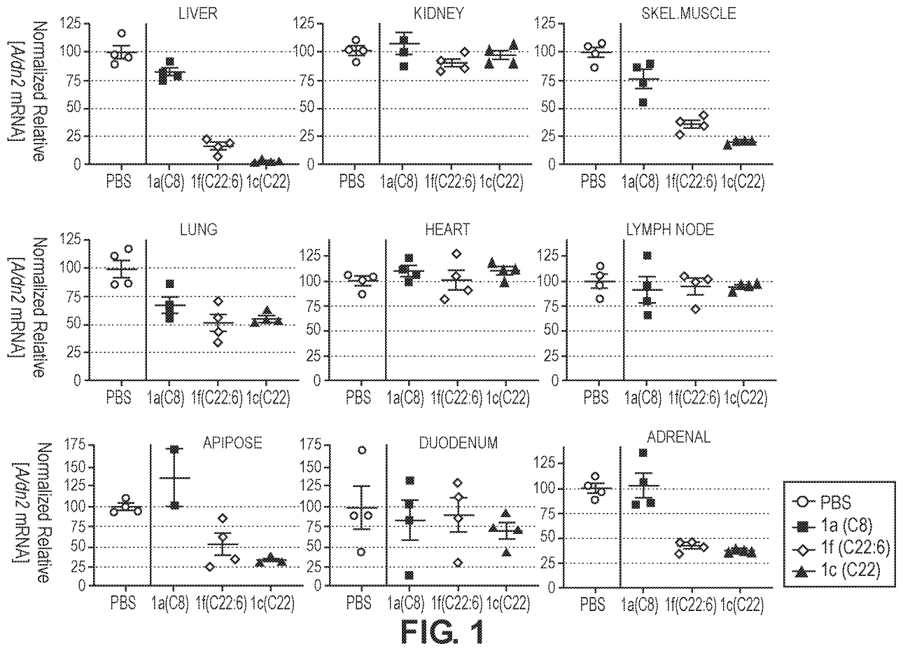

Duplex 1a (C8), 1f (C22:6), and 1c (C22) were prepared as described in Example 3 and tested for biodistribution and gene silencing activity. Duplex 1c (C22) shows broad extrahepatic distribution and robust knockdown activity (50%-75%) in lung, adrenal gland, adipose, and skeletal muscle. Duplex 1f (C22:6) also shows 50%-60% gene silencing activity in these extrahepatic tissues, as shown in FIG. 1.

### Example 5. Dose-Response of GalXC Lipid Conjugate Duplex 1c (C22) in Extrahepatic Tissues

Duplex 1c (C22) was prepared as described in Example 3 and tested for extrahepatic tissue response.

CD-1 female mice were administrated intravenously with 15 mg/kg GalXC lipid conjugates. A control group was dosed with phosphate buffered saline (PBS). Animals were sacrificed 120 hours post-treatment. Liver and extrahepatic tissues including lung, adrenal gland, skeletal muscle, adipose, heart, kidney, duodenum, and lymph node were collected. 1-4 mm punches from each tissue were removed and placed into a 96-well plate on dry ice for mRNA analysis. Reduction of target mRNA was measured by qPCR using CFX384 TOUCH™ Real-Time PCR Detection System (BioRad Laboratories, Inc., Hercules, Calif.). All samples were normalized to the PBS treated control animals and plotted using GraphPad Prism software (GraphPad Software Inc., La Jolla, Calif.).

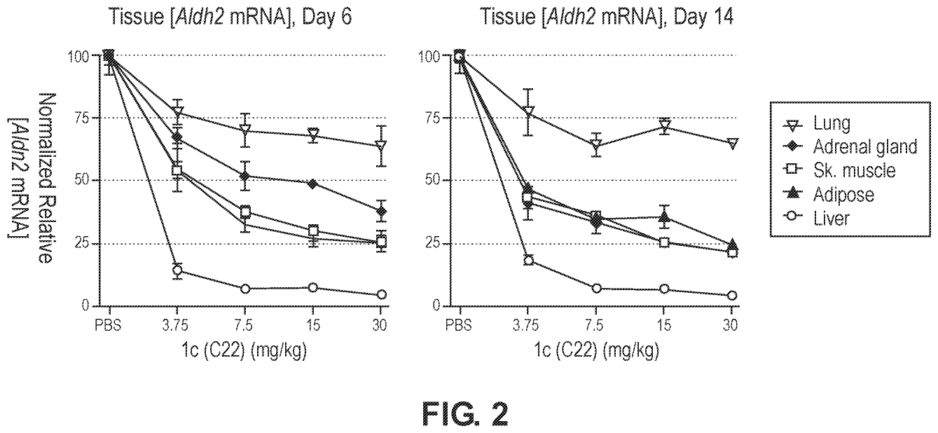

Duplex 1c (C22) demonstrates robust dose-dependent activity of gene silencing of ALDH2 mRNA from 3.75 to 30 mg/kg dosing in lung, adrenal gland, skeletal muscle, and adipose, at both day 6 and day 14 after dosing. ˜75% gene silencing is observed in skeletal muscle and adipose with 15 mg/kg dosing at both time points, as shown in FIG. 2.

### Example 6. Duration of Gene Silencing Activity of GalXC Lipid Conjugate Duplex 1c (C22) in Extrahepatic Tissues

Duplex 1c (C22) was prepared as described in Example 3.

In vivo gene silencing activity of Duplex 1c (C22) was measured using the methods as described in Example 5.

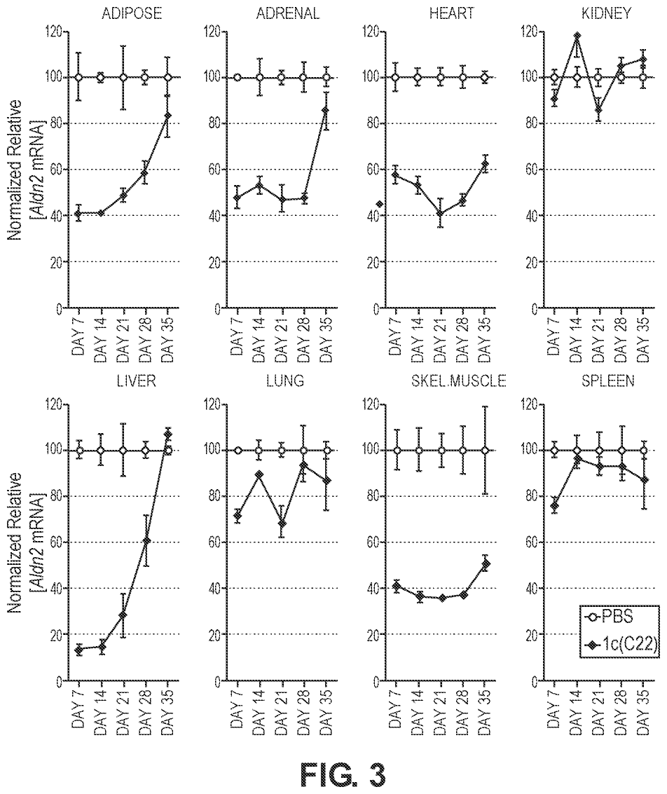

CD-1 female mice were administrated subcutaneously with indicated doses of Duplex 1c (C22). A control group was dosed with phosphate buffered saline (PBS). Animals were sacrificed 6 days or 14 days post-treatment. Liver and extrahepatic tissues including lung, adrenal gland, skeletal muscle, and adipose were collected. Target mRNA in each tissue was measured as described in Example 4. Durable ALDH2 mRNA silencing activity (˜50% knockdown) is observed in skeletal muscle and heart in 5 weeks after one single subcutaneous dosing of 15 mg/kg of Duplex 1c (C22). Significant gene silencing (40-60% knockdown) is also seen in adipose and adrenal gland during 4 weeks after one single administration, as shown in the FIG. 3.

### Example 7. Gene Silencing Activity of GalXC Diacyl Lipid Conjugates and Mono Lipid C18 Conjugate in Extrahepatic Tissues

Duplex 1h (diacyl C16), 1i (diacyl C18:1), 1j (PEG2K-diacyl C18) and 1b (C18) were prepared as described in Example 3.

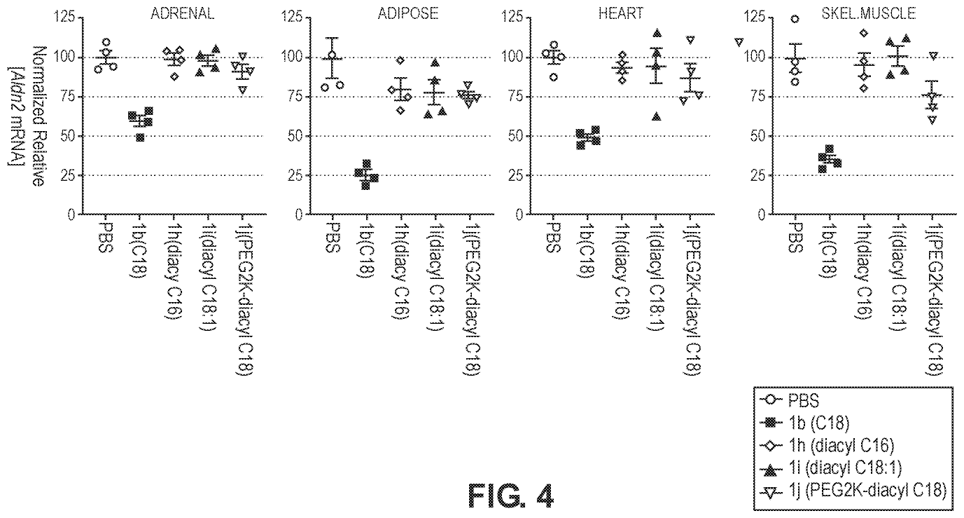

In vivo gene silencing activity of Duplex 1h (diacyl C16), 1i (diacyl C18:1), 1j (PEG2K-diacyl C18) was measured using the methods as described in Example 5. Duplex 1b (C18) shows robust gene silencing activity of ALDH2 mRNA in adrenal gland, adipose, heart, and skeletal muscle at day 7 after a single 15 mg/kg subcutaneous injection. Duplex 1h (diacyl C16), 1i (diacyl C18:1), 1j (PEG2K-diacyl C18) demonstrate less gene silencing activity in these tissues through subcutaneous administration, as shown in FIG. 4.

### Example 8. Gene Silencing Activity of GalXC Long-Lipid Conjugates and Adamantane Conjugates

GalXC long-lipid conjugates Duplex 1d (C24), 1e (C26), 1g (C24:1) and adamantane conjugate Duplex 5b (3Xacetyladamantane) were prepared as described in Example 3.

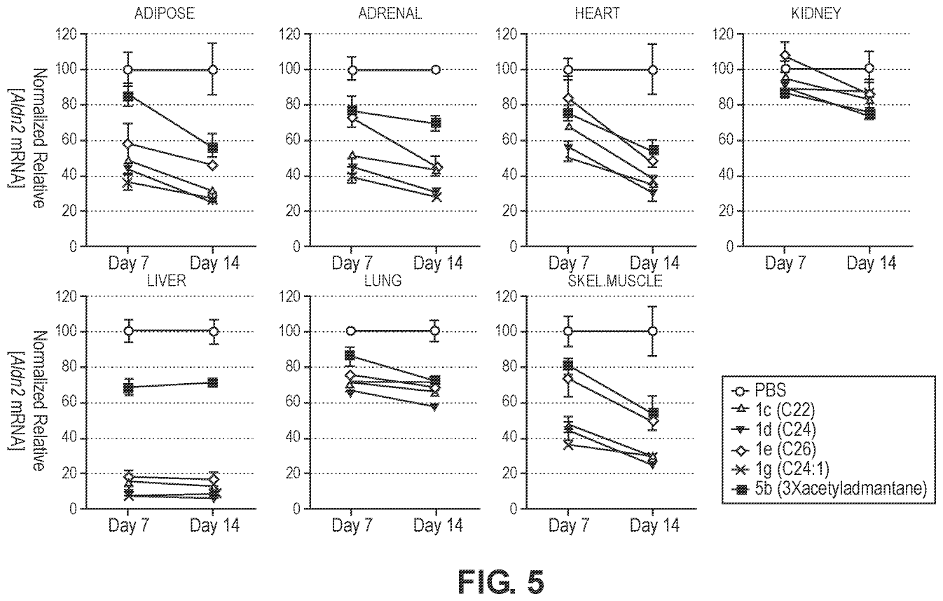

In vivo gene silencing activity of Duplex 1d (C24), 1e (C26), 1g (C24:1) and adamantane conjugate Duplex 5b (3Xacetyladamantane) was measured using the methods as described in Example 5, GalXC lipid conjugates with different lipid length demonstrate different gene silencing activity in various tissues. Duplex 1d (C24) and 1g (C24:1) show slightly improved gene silencing activity compared with Duplex 1c (C22) with 50%-75% knockdown of ALDH2 mRNA in skeletal muscle, adipose, adrenal, and heart. Stronger gene silencing activity in these tissues is observed at day 14, as shown in FIG. 5.

### Example 9. The Impact of RNA Chemical Modifications on the Gene Silencing Activity of GalXC Lipid Conjugates

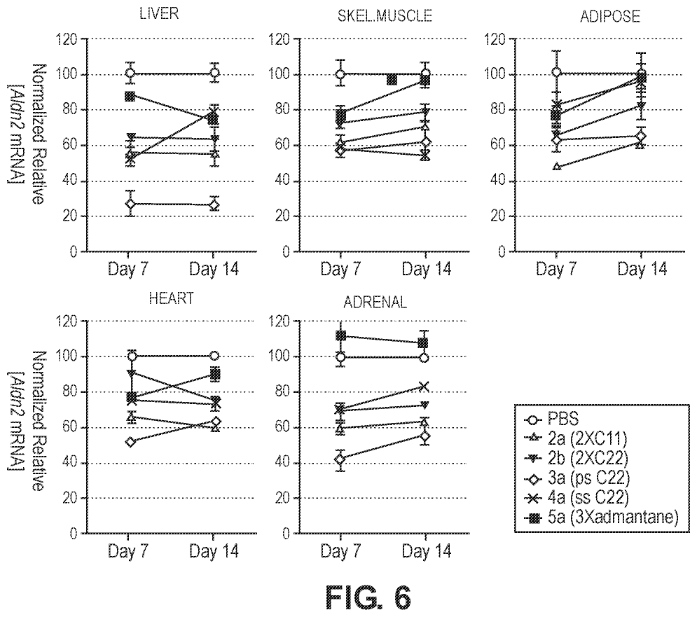

FIG. 6 shows the gene silencing activity of GalXC lipid conjugates with RNA chemical modifications, including Duplex 3a (PS-C22) of full phosphorothioate stemloop and Duplex 4a (SS-C22) of short sense, and GalXC lipid conjugates with di-lipid, including Duplex 2a (2XC11) and Duplex 2b (2XC22), and GalXC tri-adamantane conjugate Duplex 5a (3Xadamantane).

GalXC lipid conjugates Duplex 2a (2XC11), 2b (2XC22), 3a (PS-C22), 4a (SS-C22), and GalXC tri-adamantane conjugate Duplex 5a (3Xadamantane) were prepared as described in Example 3.

In vivo gene silencing activity of Duplex 2a (2XC11), 2b (2XC22), 3a (PS-C22), 4a (SS-C22), and GalXC tri-adamantane conjugate Duplex 5a (3Xadamantane) was measured using the methods as described in Example 5. As shown in the FIG. 6, significant gene silencing with 40%-60% knockdown of ALDH2 mRNA is observed in adrenal gland, adipose, heart, and skeletal muscle at day 7 and day 14 after subcutaneous dosing of Duplex 3a (PS-C22). Duplex 2a (2XC11) also shows comparable gene silencing activity in these extrahepatic tissues. Duplex 4a (SS-C22) demonstrates selectivity of silencing ALDH2 in skeletal muscle (45% knockdown) over that in liver (20% knockdown) at day 14.

While we have described several embodiments of this disclosure, it is apparent that the basic examples provided herein may be altered to provide other embodiments that utilize the nucleic acid or analogues thereof and methods of this disclosure. Therefore, it will be appreciated that the scope of this disclosure is to be defined by the specification and appended claims rather than by the specific embodiments that have been represented by way of example.

We claim:

1. A nucleic acid-ligand conjugate represented by formula I: 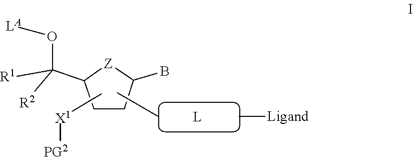
or a pharmaceutically acceptable salt thereof, wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃, or
R¹ and R² on the same carbon are taken together with their intervening atoms to form a 3-membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
L^{A} is independently PG¹, or -L-ligand;
PG¹ is hydrogen or a suitable hydroxyl protecting group;
each ligand is independently -(LC)ₙ, and/or an adamantyl group;
each LC is independently a lipid conjugate moiety comprising a saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, or —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, adamantanenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —N(R)—C(O)—, —S—, —C(O)—, —S(O)—, —S(O)₂—, —P(O)OR—, —P(S)OR—, —V¹CR²W¹— or 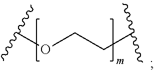
m is 1-50;
X¹, V¹ and W¹ are independently —C(R)₂—, —OR, —O—, —S—, —Se—, or —NR—;
Z is —O—, —S—, —NR—, or —CR₂—; and
PG² is hydrogen, a phosphoramidite analogue, or a suitable protecting group.

2. The nucleic acid-ligand conjugate of claim 1 represented by formula I-a:

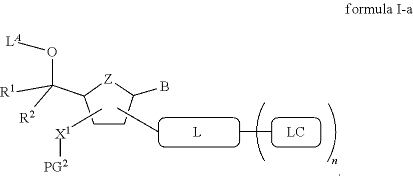

3. The nucleic acid-ligand conjugate of claim 1, wherein the conjugate is represented by formula I-b or I-c: 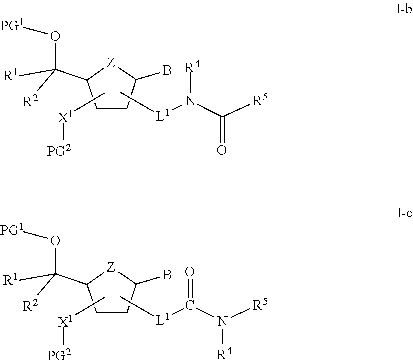
or a pharmaceutically acceptable salt thereof, wherein
L¹ is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or 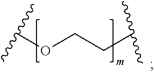
R⁴ is hydrogen, R^{A}, or a suitable amine protection group; and
R⁵ is adamantyl, or a saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, or —P(S)OR—.

4. A nucleic acid-ligand conjugate, wherein the conjugate is represented by formula I-d or I-e: 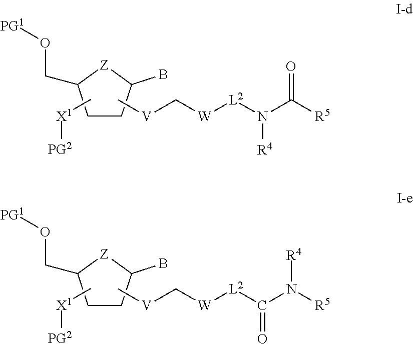
or a pharmaceutically acceptable salt thereof, wherein
B is a nucleobase or hydrogen;
PG¹ and PG² are independently a hydrogen, a phosphoramidite analogue, or a suitable protecting group; and
R⁵ is adamantyl, or a saturated or unsaturated, straight or branched C₁₋₅₀ hydrocarbon chain, wherein 0-10 methylene units of the hydrocarbon chain are independently replaced by —O—, —NR—, —S—, —C(O)—, —C(O)O—, —S(O)—, —S(O)₂—, —P(O)OR—, or —P(S)OR—;
V is a bivalent group selected from —O—, —S—, and —NR—;
W is a bivalent group selected from —O—, —S—, —NR—, —C(O)NR—, —OC(O)NR—, —SC(O)NR—, 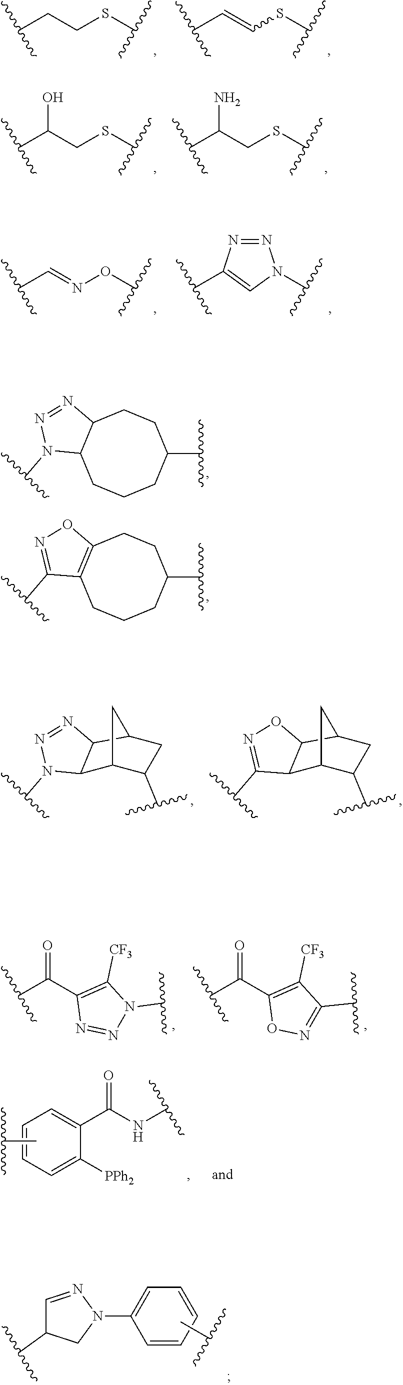
L² is a covalent bond or a bivalent saturated or unsaturated, straight or branched C₁₋₅₀ hydrocarbon chain, wherein 0-10 methylene units of the hydrocarbon chain are independently replaced by —O—, —NR—, —S—, —C(O)—, —S(O)—, —S(O)₂—, —P(O)OR—, —P(S)OR—, or 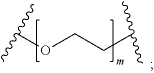
m is 1-50;
X¹ is —C(R)₂—, —OR, —O—, —S—, —Se—, or —NR—;
R⁴ is hydrogen, R^{A}, or a suitable amine protection group; and
R⁵ is adamantyl, or a saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

5. The nucleic acid-ligand conjugate of claim 4, wherein:
V is —O—;
L² is a covalent bond or a bivalent saturated or unsaturated, straight or branched C₁₋₅₀ hydrocarbon chain, wherein 0-10 methylene units of the hydrocarbon chain are independently replaced by —O—, —C(O)—, 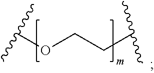
R⁴ is hydrogen;
w is —O—, —NR—, —C(O)NR—, —OC(O)NR 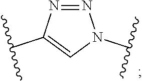 [SMILES: *c1cn(*)nn1]
and
R⁵ is a saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, or —C(O)O—.

6. A nucleic acid-ligand conjugate represented by formula I-Ib or I-Ic: 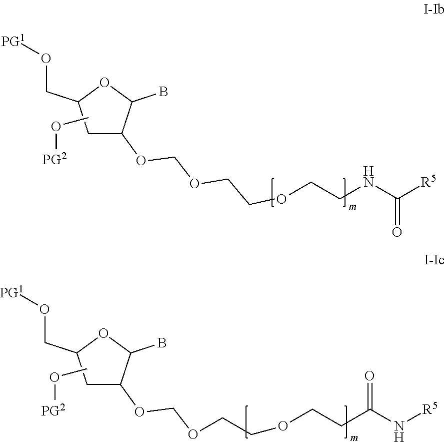
or a pharmaceutically acceptable salt thereof, wherein
B is a nucleobase or hydrogen;
m is 1-50;
PG¹ and PG² are independently a hydrogen, a phosphoramidite analogue, or a suitable protecting group; and
R⁵ is adamantyl, or a saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—.

7. The nucleic acid-ligand conjugate of claim 6, wherein:
R⁵ is selected from

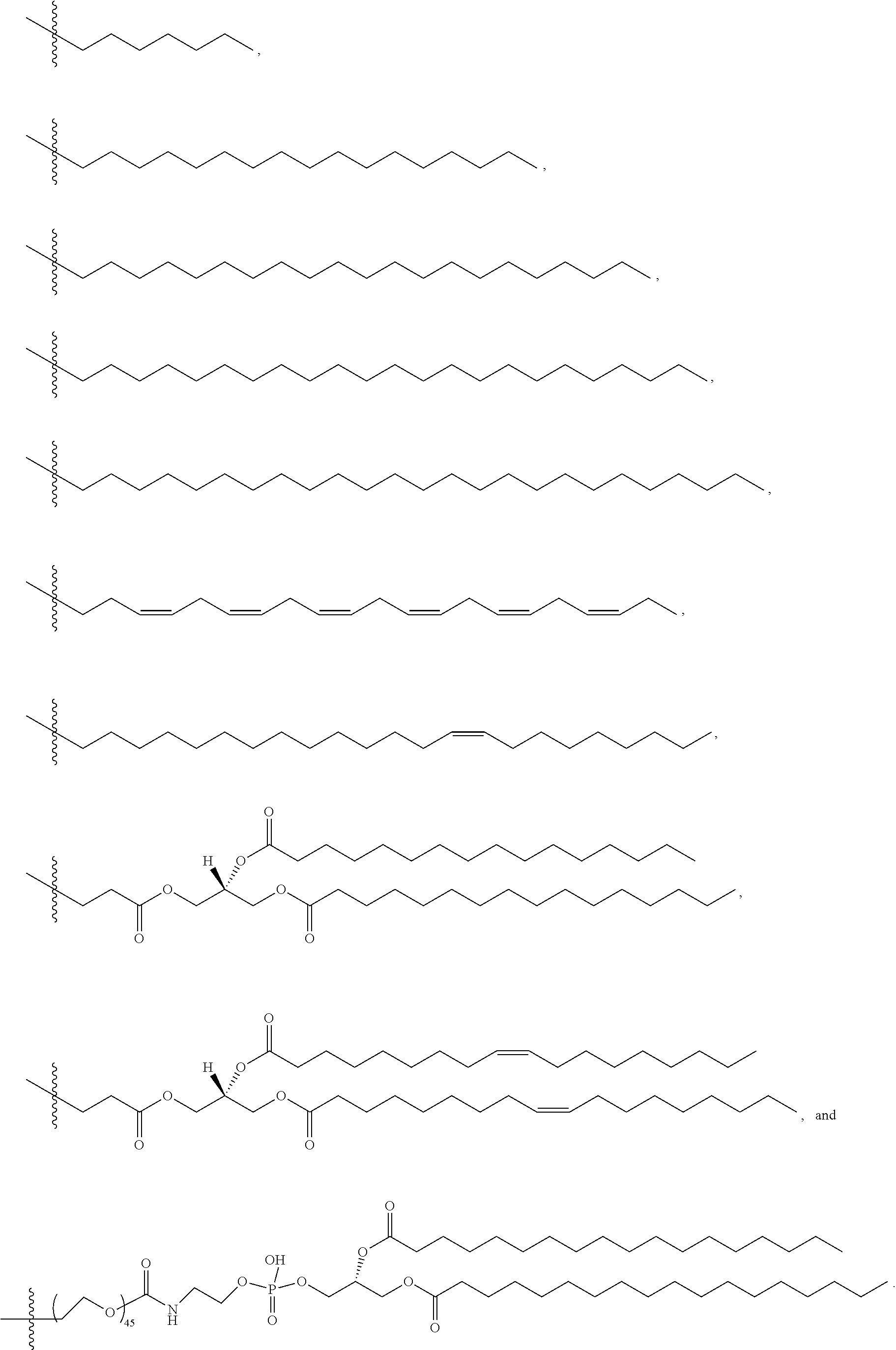

8. An oligonucleotide-ligand conjugate comprising one or more nucleic acid-ligand conjugate units of any one of claims 1 to 8.

9. The oligonucleotide-ligand conjugate of claim 9, wherein the conjugate comprises 1, 2, 3, 4, 5, 6, 7, 8, 9 or 10 nucleic acid-ligand conjugate units.

10. An oligonucleotide-ligand conjugate comprising one or more nucleic acid-ligand conjugates represented by formula II: 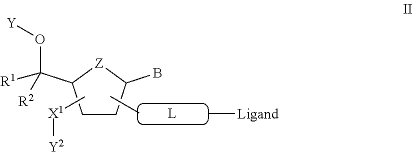
or a pharmaceutically acceptable salt thereof, wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃; or
R¹ and R² on the same carbon are taken together with their intervening atoms to form a 3-7 membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur; or
two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
ligand is independently -(LC)ₙ, or an adamantyl group;
each LC is independently a lipid conjugate moiety comprising a saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, —V¹CR²W¹—, or 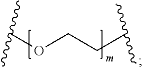
m is 1-50;
X¹, V¹ and W¹ are independently —C(R)₂—, —OR, —O—, —S—, —Se—, or —NR—;
Y is hydrogen, a suitable hydroxyl protecting group, 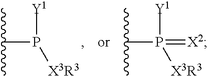
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, S, or NR;
X³ is —O—, —S—, —BH₂—, or a covalent bond;
Y¹ is a linking group attaching to the 2′- or 3′-terminal of a nucleoside, a nucleotide, or an oligonucleotide;
Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support; and
Z is —O—, —S—, —NR—, or —CR₂—.

11. The oligonucleotide-ligand conjugate of claim 10, wherein the conjugate is represented by formula II-a:

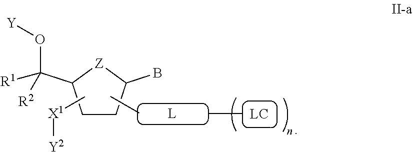

12. The oligonucleotide-ligand conjugate of claim 10, wherein the conjugate is represented by formula II-b or II-c: 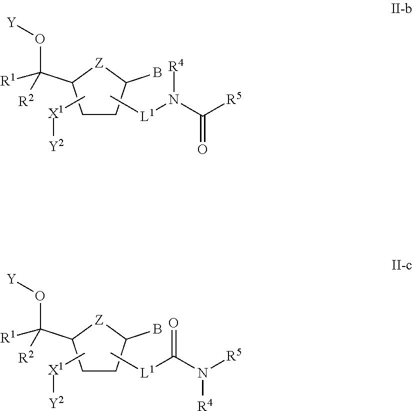
or a pharmaceutically acceptable salt thereof, wherein:
L¹ is a covalent bond, a monovalent or a bivalent saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, —P(S)OR—, or 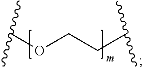
R⁴ is hydrogen, R^{A}, or a suitable amine protection group; and
R⁵ is adamantyl, or a saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR.

13. The oligonucleotide-ligand conjugate of claim 10, wherein the conjugate is represented by formula II-d or II-e: 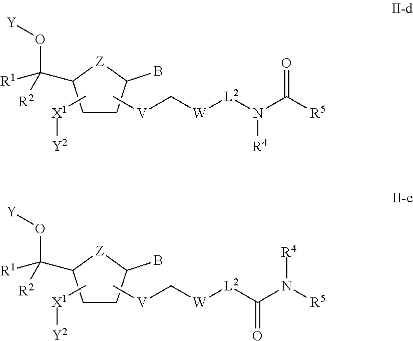
or a pharmaceutically acceptable salt thereof;
V is a bivalent group selected from —O—, —S—, and —NR—;
W is a bivalent group selected from —O—, —S—, —NR—, —C(O)NR—, —OC(O)NR—, —SC(O)NR—, 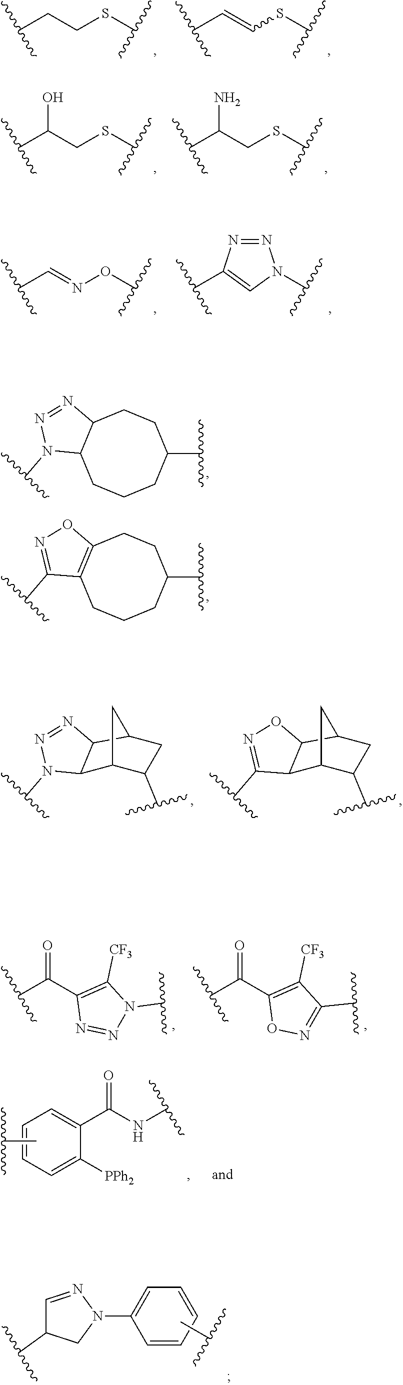
L² is a covalent bond or a bivalent saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, —P(S)OR—, or 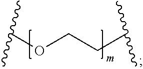
R⁴ is hydrogen, R^{A}, or a suitable amine protection group; and
R⁵ is a saturated or unsaturated, straight or branched C₁₋₅₀ hydrocarbon chain, wherein 0-10 methylene units of the hydrocarbon chain are independently replaced by -Cy-, —O—, —C(O)NR—, —NR—, —S—, —C(O)—, —C(O)O—, —S(O)—, —S(O)₂—, —P(O)OR—, or —P(S)OR—.

14. An oligonucleotide-ligand conjugate represented by formula II-Id or II-Ie: 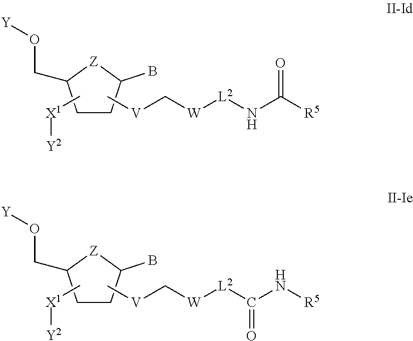
or a pharmaceutically acceptable salt thereof; wherein:
m is 1-50;
B is H, or a nucleobase;
X¹ is —C(R)₂—, —OR, —O—, —S—, or —NR—;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
w is a bivalent group selected from —O—, —S—, —NR—, —C(O)NR—, —OC(O)NR—, 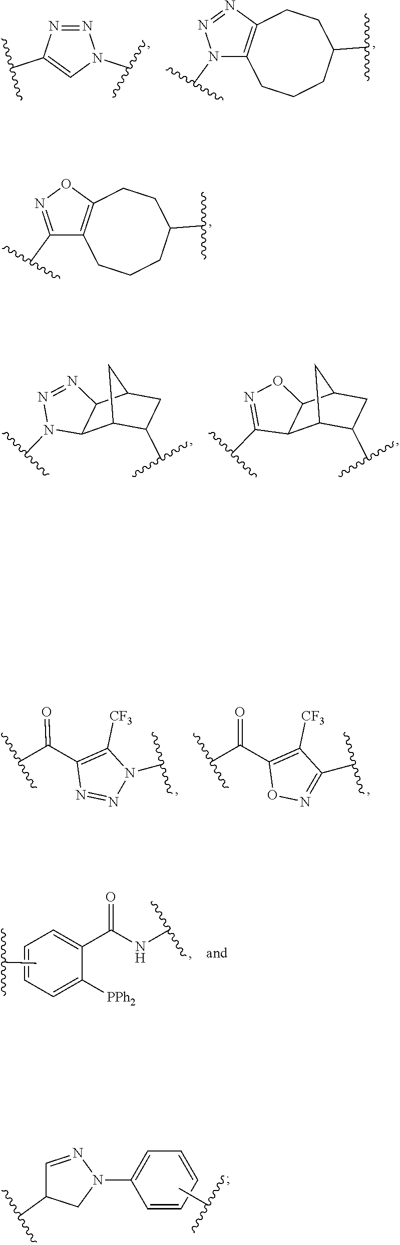
L² is a covalent bond or a bivalent saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, —P(S)OR—, or 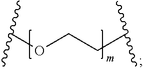
Y is hydrogen, 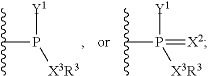
R³ is hydrogen, or a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, or S;
X³ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support; and
R⁵ is adamantyl, or a saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—.

15. The oligonucleotide-ligand conjugate of claim 14, wherein:
R⁵ is selected from

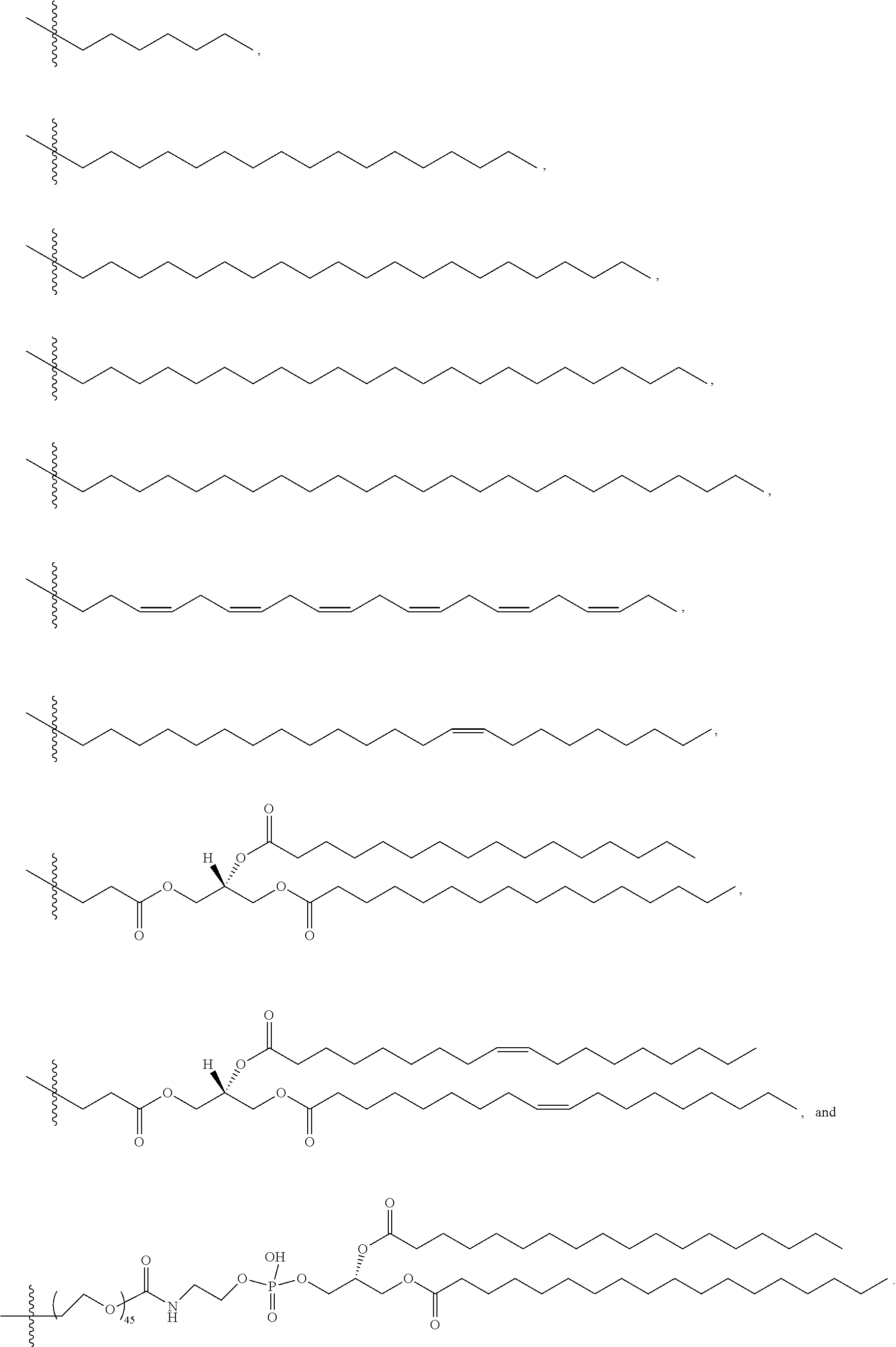

16. An oligonucleotide-ligand conjugate represented by formula II-Ib or II-Ic: 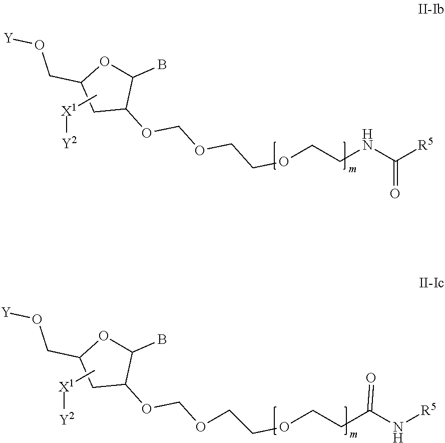
or a pharmaceutically acceptable salt thereof, wherein
B is a nucleobase or hydrogen;
m is 1-50;
X¹ is —O—, or —S—;
Y is hydrogen, 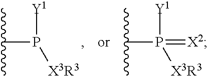
R³ is hydrogen, or a suitable protecting group;
X² is O, or S;
X³ is —O—, —S—, or a covalent bond;
Y¹ is a linking group attaching to the 2′- or 3′-terminal of a nucleoside, a nucleotide, or an oligonucleotide;
Y² is hydrogen, a phosphoramidite analogue, an internucleotide linking group attaching to the 5′-terminal of a nucleoside, a nucleotide, or an oligonucleotide, or a linking group attaching to a solid support;
R⁵ is adamantyl, or a saturated or unsaturated, straight or branched C₁₋₅₀ hydrocarbon chain, wherein 0-10 methylene units of the hydrocarbon chain are independently replaced by —O—, —C(O)NR—, —NR—, —S—, —C(O)—, —C(O)O—, —S(O)—, —S(O)₂—, —P(O)OR—, or —P(S)OR—; and
R is hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur.

17. The oligonucleotide-ligand conjugate of claim 16, wherein:
R⁵ is selected from

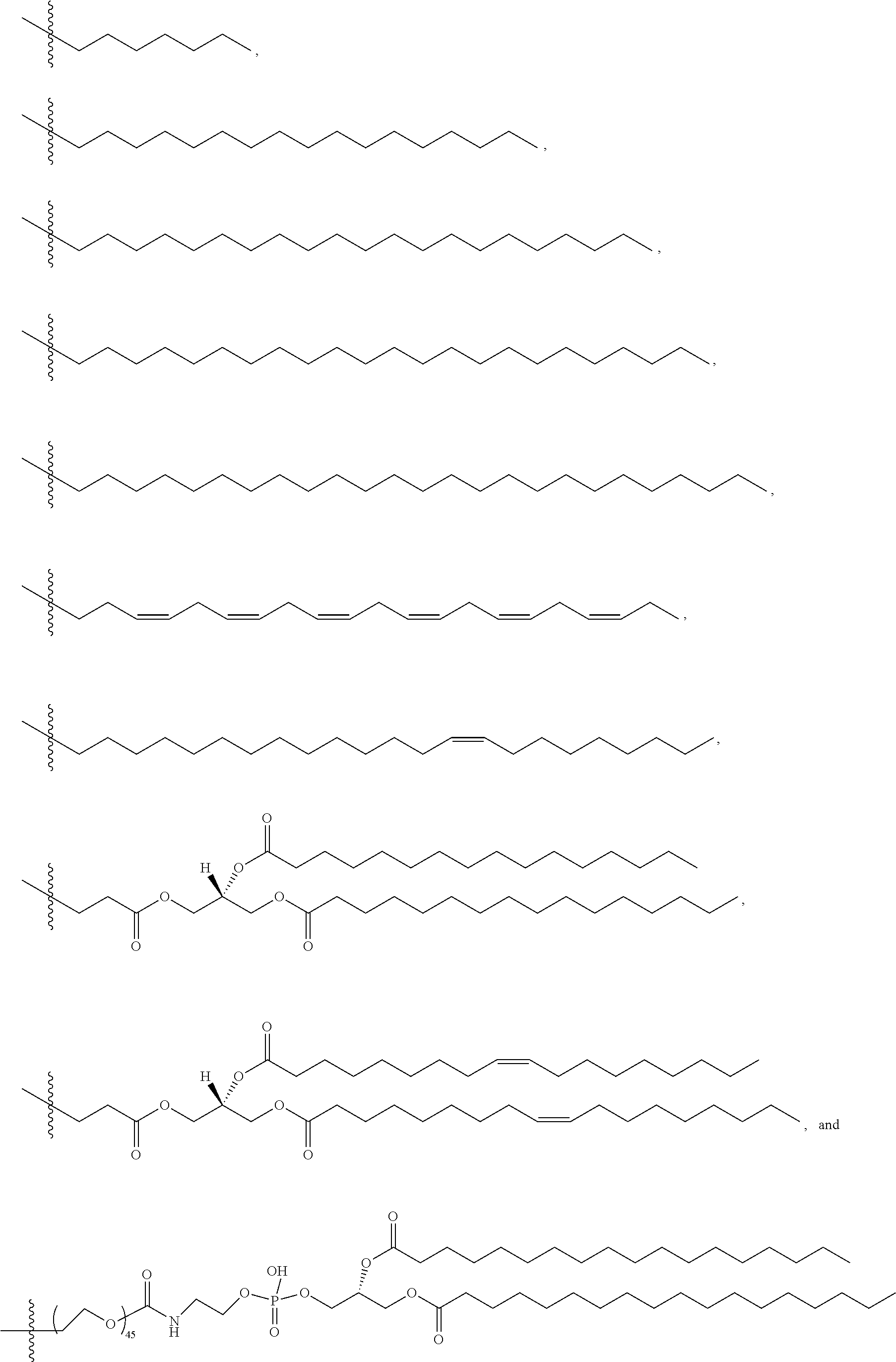

18. The oligonucleotide-ligand conjugate of any one of claims 10-17, wherein the conjugate comprises 1-10 nucleic acid-ligand conjugate units.

19. The oligonucleotide-ligand conjugate of any one of claims 10-17, wherein the conjugate comprises 1, 2, 3, 4, 5, 6, 7, 8 or 9 nucleic acid-ligand conjugate units.

20. The oligonucleotide-ligand conjugate of any one of claims 10-17, wherein the conjugate comprises 1, 2 or 3 nucleic acid-ligand conjugate units.

21. The oligonucleotide-ligand conjugate of any one of claims 8-20, wherein the oligonucleotide comprises a sense strand of 10-53 nucleotides in length and an antisense strand of 15-53 nucleotides in length, wherein the antisense oligonucleotide strand has sequence complementary to at least 15 consecutive nucleotides of a target gene sequence and reduces the gene expression when the oligonucleotide-conjugate is introduced into a mammalian cell.

22. An oligonucleotide-ligand conjugate for reducing expression of a target gene, wherein the nucleic acid-conjugate unit is represented by formula II: 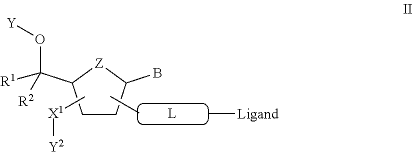
or a pharmaceutically acceptable salt thereof, wherein:
B is a nucleobase or hydrogen;
R¹ and R² are independently hydrogen, halogen, R^{A}, —CN, —S(O)R, —S(O)₂R, —Si(OR)₂R, —Si(OR)R₂, or —SiR₃; or
R¹ and R² on the same carbon are taken together with their intervening atoms to form a 3-7 membered saturated or partially unsaturated ring having 0-3 heteroatoms, independently selected from nitrogen, oxygen, and sulfur;
each R^{A} is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently hydrogen, a suitable protecting group, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur; or
two R groups on the same atom are taken together with their intervening atoms to form a 4-7 membered saturated, partially unsaturated, or heteroaryl ring having 0-3 heteroatoms, independently selected from nitrogen, oxygen, silicon, and sulfur;
ligand is independently -(LC)ₙ, or an adamantyl group;
each LC is independently a lipid conjugate moiety comprising a saturated or unsaturated, straight or branched C₁₋₅₀ hydrocarbon chain, wherein 0-10 methylene units of the hydrocarbon chain are independently replaced by -Cy-, —O—, —NR—, —S—, —C(O)—, —S(O)—, —S(O)₂—, —P(O)OR—, —P(S)OR—;
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-3 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
n is 1-10;
L is a covalent bond or a bivalent saturated or unsaturated, straight or branched C₁₋₅₀ hydrocarbon chain, wherein 0-10 methylene units of the hydrocarbon chain are independently replaced by -Cy-, —O—, —NR—, —N(R)—C(O)—, —S—, —C(O)—, —S(O)—, —S(O)₂—, —P(O)OR—, —P(S)OR—, —V¹CR²W¹—, or 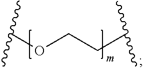
m is 1-50;
X¹, V¹ and W¹ are independently —C(R)₂—, —OR, —O—, —S—, —Se—, or —NR—;
Y is hydrogen, a suitable hydroxyl protecting group, 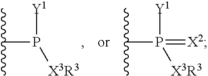
R³ is hydrogen, a suitable protecting group, a suitable prodrug, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur;
X² is O, S, or NR;
X³ is —O—, —S—, —BH₂—, or a covalent bond;
Y¹ is a linking group attaching to the 2′- or 3′-terminal of a nucleoside, a nucleotide, or an oligonucleotide;
Y² is hydrogen, a suitable protecting group, a phosphoramidite analogue, an internucleotide linking group attaching to the 5′-terminal of a nucleoside, a nucleotide, or an oligonucleotide, or a linking group attaching to a solid support;
Z is —O—, —S—, —NR—, or —CR₂—; and
wherein the oligonucleotide comprises a sense strand of 15-53 nucleotides in length and an antisense strand of 19-53 nucleotides in length, wherein the antisense oligonucleotide strand has sequence complementary to at least 15 consecutive nucleotides of a target gene sequence;
and wherein the antisense strand and the sense strand form a duplex structure but are not covalently linked.

23. The oligonucleotide-ligand conjugate of claim 21 or 22, wherein the nucleic acid-ligand conjugate units are present in the sense strand.

24. The oligonucleotide-ligand conjugate of claim 21 or 22, wherein the antisense strand is 19 to 27 nucleotides in length.

25. The oligonucleotide-ligand conjugate of claim 21 or 22, wherein the sense strand is 12 to 40 nucleotides in length.

26. The oligonucleotide-ligand conjugate of any one of claims 21 to 25, wherein the sense strand forms a duplex region with the antisense strand.

27. The oligonucleotide-ligand conjugate of claim 21, wherein the region of complementarity is fully complementary to the target sequence.

28. The oligonucleotide-ligand conjugate of any one of claims 21 to 27, wherein the sense strand comprises at its 3′-end a stem-loop set forth as: S₁-L-S₂, wherein S₁ is complementary to S₂, and wherein L forms a loop between S₁ and S₂ of 3 to 5 nucleotides in length.

29. The oligonucleotide-ligand conjugate of claim 28, wherein L is a tetraloop.

30. The oligonucleotide-ligand conjugate of claim 28, wherein L comprises a sequence set forth as GAAA.

31. The oligonucleotide-ligand conjugate of any one of claims 21 to 30, further comprising a 3′-overhang sequence on the antisense strand of two nucleotides in length.

32. The oligonucleotide-ligand conjugate of any one of claims 21 to 30, wherein the oligonucleotide further comprises a 3′-overhang sequence of one or more nucleotides in length, wherein the 3′-overhang sequence is present on the antisense strand, the sense strand, or the antisense strand and sense strand.

33. The oligonucleotide-ligand conjugate of any one of claims 21 to 32, wherein the oligonucleotide comprises at least one modified nucleotide.

34. The oligonucleotide-ligand conjugate of claim 33, wherein the modified nucleotide comprises a 2′-modification.

35. The oligonucleotide-ligand conjugate of claim 34, wherein the 2′-modification is a modification selected from: 2′-aminoethyl, 2′-fluoro, 2′-O-methyl, 2′-O-methoxyethyl, 2′-deoxy-2′-fluoro, and 2′-deoxy-2′-fluoro-p-d-arabino.

36. The oligonucleotide-ligand conjugate of any one of claims 21 to 32, wherein all the nucleotides of the oligonucleotide are modified.

37. The oligonucleotide-ligand conjugate of any one of claims 21 to 36, wherein the oligonucleotide comprises at least one modified internucleotide linkage.

38. The oligonucleotide-ligand conjugate of claim 37, wherein the at least one modified internucleotide linkage is a phosphorothioate linkage.

39. The oligonucleotide-ligand conjugate of any one of claims 21 to 36, wherein the 4′-carbon of the sugar of the 5′-nucleotide of the antisense strand comprises a phosphate analog.

40. The oligonucleotide-ligand conjugate of claim 39, wherein the phosphate analog is oxymethylphosphonate, vinylphosphonate, or malonylphosphonate.

41. A composition comprising an oligonucleotide-ligand conjugate of any one of claims 21-40 and an excipient.

42. A method of delivering an oligonucleotide-ligand conjugate to a subject, the method comprising administering the composition of claim 41 to the subject.

43. An oligonucleotide-ligand conjugate of any one of claims 21-40 for reducing expression of a target gene.
